(19) United States
(12) Patent Application Publication
(10) Pub. No.: US 2026/0256925 A1
(43) Pub. Date: Sep. 3, 2026

(54) INDAZOLE DERIVATIVES AS ESTROGEN RECEPTOR DEGRADERS

(71) Applicants: REGENTS OF THE UNIVERSITY OF MICHIGAN, Ann Arbor, MI (US); Oncopia Therapeutics, Inc. d/b/a SK Life Science Labs, King Of Prussia, PA (US)

(72) Inventors: Zhixiang Chen, Ann Arbor, MI (US); Rohan Rej, Ann Arbor, MI (US); Guozhang Xu, New York, NY (US); Dimin Wu, Ann Arbor, MI (US); Ranjan Kumar Acharyya, Ann Arbor, MI (US); Mingliang Wang, Ann Arbor, MI (US); Biao Hu, Ann Arbor, MI (US); Jianfeng Lu, Ann Arbor, MI (US); Shaomeng Wang, Superior Township, MI (US)

(21) Appl. No.: 18/992,035

(22) PCT Filed: Jul. 12, 2023
(86) PCT No.: PCT/US2023/027434

§ 371 Date: Jan. 7, 2025

Related U.S. Application Data
(60) Provisional application No. 63/388,299, filed on Jul. 12, 2022.
(60) Provisional application No. 63/408,633, filed on Sep. 21, 2022.

(51) Int. Cl. A61K 47/55; A61K 47/54; A61P 35/00

(52) U.S. Cl. CPC A61K 47/55; A61K 47/545; A61P 35/00

(57) ABSTRACT

Described herein are compounds of Formula I and pharmaceutically acceptable salts, solvates, or stereoisomers thereof, as well as their uses (e.g., as estrogen receptor degraders).

### RELATED APPLICATIONS

This application claims the benefit of and priority to U.S. Provisional Application No. 63/388,299, filed Jul. 12, 2022; and U.S. Provisional Application No. 63/408,633, filed Sep. 21, 2022; the contents of each of which are incorporated herein by reference in their entireties.

### BACKGROUND

Estrogen Receptors (ERs) belong to the steroid/nuclear receptor superfamily involved in the regulation of eukaryotic gene expression, cellular proliferation, and differentiation in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes. ERα is mainly expressed in reproductive tissues such as uterus, ovary, breast, bone, and white adipose tissue. It is well known that deregulation of ER signaling, specifically through ERα, results in uncontrolled cellular proliferation which eventually results into cancer. ER+ breast cancer accounts for approximately 75% of all breast cancers diagnosed, as well as some ovarian and endometrial cancers.

Current therapy for ER+ breast cancer including agents that inhibit the ER activity through direct binding to the ligand binding domain of the receptor (e.g., tamoxifen); blocking the synthesis of estrogen (e.g., aromatase inhibitor such as anastrozole and letrozole); or inducing the degradation of ER. Selective estrogen receptor degraders (SERD) are small molecules that target ERα for proteasome-dependent degradation. Fulvestrant is the only SERD that has been approved for the treatment of postmenopausal women with advanced ER+ breast cancer with standard endocrine therapies. Because it has poor solubility and is not orally bioavailable, fulvestrant is administered clinically by a monthly intramuscular injection. To address the shortcomings of fulvestrant, oral bioavailable SERDs are being developed. However, the SERDs are only able to achieve partial degradation of the ER protein despite they are typically potent and effective in inducing degradation of ER protein in ER+ breast cancer cells.

It is believed that ERα degradation may occur when both ER and a ubiquitin ligase (e.g., cereblon E3 ligase (CRBN)) are bound and brought into close proximity for ubiquitination and subsequent degradation by proteasomes. A new approach would be to utilize the naturally occurring cellular ubiquitin-mediated degradation to develop a completely new class of therapeutics for the treatment of ER+ metastatic breast cancer with nearly complete degradation of ER protein.

### SUMMARY

In certain aspects, the present disclosure provides compounds of Formula I:

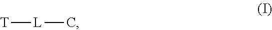

and pharmaceutically acceptable salts, solvates, or stereoisomers thereof,
wherein:

C is of Formula I-1

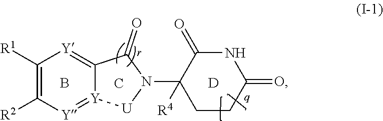

T is of Formula I-2:

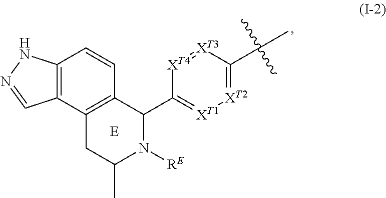

L is of Formula I-3:

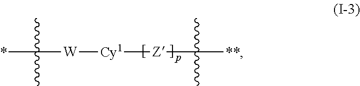

wherein each of the variables in Formulae I, I-1, 1-2, and I-3, is described, embodied, and exemplified herein.

In certain aspects, the present disclosure provides pharmaceutical composition comprising a compound disclosed herein, and a pharmaceutically acceptable excipient.

In certain aspects, the present disclosure provides methods of degrading an estrogen receptor in a subject, comprising administering to the subject a compound disclosed herein.

In certain aspects, the present disclosure provides uses of a compound disclosed herein in the manufacture of a medicament for degrading an estrogen receptor in a subject.

In certain aspects, the present disclosure provides compounds disclosed herein for use in degrading an estrogen receptor in a subject.

In certain aspects, the present disclosure provides methods of treating or preventing a disease or disorder in a subject in need thereof, comprising administering to the subject a compound disclosed herein (e.g., in a therapeutically effective amount).

In certain aspects, the present disclosure provides methods of treating a disease or disorder in a subject in need thereof, comprising administering to the subject a compound disclosed herein (e.g., in a therapeutically effective amount).

In certain aspects, the present disclosure provides uses of a compound disclosed herein in the manufacture of a medicament for treating or preventing a disease or disorder in a subject in need thereof.

In certain aspects, the present disclosure provides uses of a compound disclosed herein in the manufacture of a medicament for treating a disease or disorder in a subject in need thereof.

In certain aspects, the present disclosure provides compounds disclosed herein for use in treating or preventing a disease or disorder in a subject in need thereof.

In certain aspects, the present disclosure provides compounds disclosed herein for use in treating a disease or disorder in a subject in need thereof.

### DETAILED DESCRIPTION

The present disclosure relates to compounds and methods of degrading an estrogen receptor comprising contacting the estrogen receptor with a therapeutically effective amount of an estrogen receptor degrader disclosed herein. The present disclosure also relates to methods of treating an estrogen receptor-mediated disease or condition in a subject in need thereof by administering a therapeutically effective amount of an estrogen receptor degrader disclosed herein. The present disclosure further relates to methods of treating an estrogen receptor-mediated disease or condition in a subject in need thereof, comprising administering a pharmaceutical composition comprising a therapeutically effective amount of an estrogen receptor degrader disclosed herein.

### Compounds of the Application

In certain aspects, the present disclosure provides compounds of Formula I:

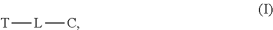

and pharmaceutically acceptable salts, solvates, or stereoisomers thereof,
wherein:

C is of Formula I-1

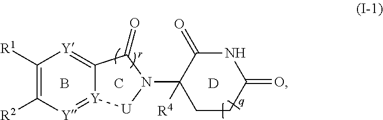

wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R² is *-Cy²-, wherein * denotes attachment to L;
Cy² is 3- to 12-membered heterocyclylene, wherein the heterocyclylene is optionally substituted with one or more R^{u}; or
R¹ and R², together with the intervening carbon atoms, form Ring A attached to L, wherein Ring A is optionally substituted C₃₋₁₂ carbocycle or 5- to 16-membered heterocycle;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form Ring A attached to L, wherein Ring A is optionally substituted 5- to 16-membered heterocycle;
provided that R¹ and R², and R^{z} and R³, do not both form Ring A attached to L;
Y′ is N or CR^{Y}′;
R^{Y}′ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R″;
[symbol] denotes an optional covalent bond between Y and U;
when the bond between Y and U is absent:
r is 0 or 1;
Y is N or CR^{Y};
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
U is hydrogen or C₁₋₆ alkyl optionally substituted with one or more R^{u};
when the bond between Y and U is present:
r is 1;
Y is C;
U is —CH₂—, —C(═O)—, —(C═O)—N(R^{U})—*, or —N═C(R^{U})—*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl; and
q is an integer from 0 to 2,

### T is of Formula I-2:

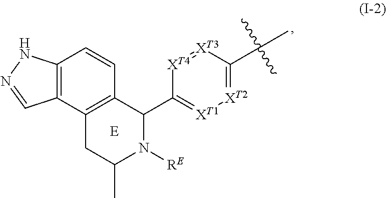

wherein:
each of X^{T1}, X^{T2}, X^{T3}, and X^{T4} is independently N or CR^{T};
each occurrence of R^{T} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
R^{E} is C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene-C₃₋₁₂ carbocyclyl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};

### L is of Formula I-3:

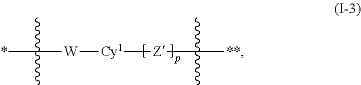

wherein:
* denotes attachment to T and ** denotes attachment to C;
W is absent; or
W is C₁₋₃ alkylene, —O—, —NR^{W}—, or —(C═O)—, wherein the alkylene is optionally substituted by one or more R^{u};
Cy¹ is absent; or
Cy¹ is 6-membered heteroarylene, C₆ arylene, C₃₋₁₂ membered carbocyclylene, or 3- to 12-membered heterocyclylene, wherein the arylene, heteroarylene, carbocyclylene, or heterocyclylene is optionally substituted by one or more R^{u};
Z′ is absent; or
each Z′ is independently C₁₋₃ alkylene, —O—, —NR^{W}—, —(C═O)—, C₃₋₁₂ membered carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted with one or more R^{u}; and
p is an integer selected from 0 to 8,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR°R^{a}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered
wherein each of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain aspects, the present disclosure provides compounds of Formula I:

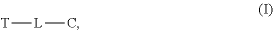

and pharmaceutically acceptable salts, solvates, or stereoisomers thereof,
wherein:

### C is of Formula I-1

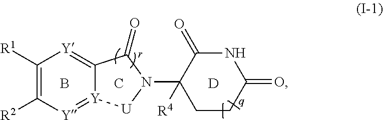

wherein:
[symbol] denotes an optional covalent bond between Y and U;
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{a}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R² is *-Cy²-, wherein * denotes attachment to L;
Cy² is 3- to 12-membered heterocyclylene, wherein the heterocyclylene is optionally substituted with one or more R^{u}; or
R¹ and R², together with the intervening carbon atoms, form Ring A attached to L, wherein Ring A is C₃₋₁₀ carbocycle or 5- to 16-membered heterocycle optionally substituted with one or more Rⁱ;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form Ring A attached to L, wherein Ring A is 5- to 16-membered heterocycle optionally substituted with one or more Rⁱ;
provided that R¹ and R², and R² and R³, do not both form Ring A attached to L; each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
Y′ is N or CR^{Y}′;
R^{Y} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R″;
Y is N or CR^{Y} when the bond between Y and U is absent; or Y is C when the bond between Y and U is present;
R^{Y} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
r is 0 or 1;
U is hydrogen or C₁₋₆ alkyl when the bond between Y and U is absent; or
U is —CH₂—, —C(═O)—, —(C═O)—N(R^{U})—*, or —N═C(R^{U})—* when the bond between Y and U is present;
R^{U} is H or C₁₋₆ alkyl, and * denotes attachment to Ring B;
R⁴ is hydrogen, deuterium, C₁₋₆ haloalkyl, or C₁₋₆ alkyl; and
q is an integer from 0 to 2;

### T is of Formula I-2:

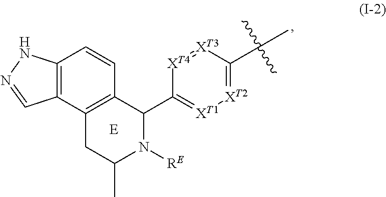

wherein:
each of X^{T1}, X^{T2}, X^{T3}, and X^{T4} is independently N or CRT;
each occurrence of R^{T} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
R^{E} is halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₄ aryl, 5- to 14-membered heteroaryl, C₃₋₁₀ carbocyclyl, 3- to 10-membered heterocyclyl, —(C₁₋₆ alkyl-C₃₋₁₀ carbocyclyl), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)ORD, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};

### L is of Formula I-3:

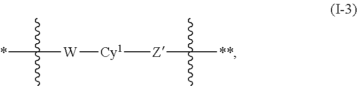

wherein:
W is absent; or
W is —CH₂—, —O—, —NR^{W}—, or —(C═O)—;
R^{W} is hydrogen or C₁₋₆ alkyl;
* denotes attachment to T and ** denotes attachment to C;
Cy¹ is 6-membered heteroarylene, C₆ arylene, C₃₋₁₂ membered carbocyclylene, or 3- to 12-membered heterocyclylene, wherein the arylene, heteroarylene, carbocyclylene, or heterocyclylene is optionally substituted by one or more R^{u};
Z′ is absent; or
Z′ is —(C(═O))ₚ—(O)ₚ′—(C₁₋₆ alkylene)ᵤ-(3- to 6-membered heterocyclylene)ᵥ-(C(═O))ₚ—(C₁₋₆ alkylene)u-(3- to 6-membered heterocyclylene)ᵥ-(C(═O))ₚ, wherein the alkylene or heterocyclylene is optionally substituted by one or more R^{u};
each occurrence of p, p′, and u is independently 0 or 1; and
each v is an integer independently selected from 0 to 3,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₀ carbocyclyl, 3- to 10-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC (—O)NR^{c}R^{d}, —C(═O)R^{a}, —C(═O)ORD, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₀ carbocyclyl or 3- to 10-membered heterocyclyl;
each R^{a} is independently C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₀ carbocyclyl, 3- to 10-membered heterocyclyl, C₆₋₁₀ aryl, or 5- to 10-membered heteroaryl; or
R^{c} and R^{d}, together with the nitrogen atom to which they are attached, form 3- to 10-membered heterocyclyl,
wherein each of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl.

In certain embodiments, when the bond between Y and U is present, U is —CH₂— or —C(═O)—, and r is 1, then either R¹ and R², or R² and R³, together with the intervening carbon atoms, form Ring A attached to L.

In certain embodiments, when the bond between Y and U is present, U is —CH₂— or —C(═O)—, and r is 1, then Ring A is not

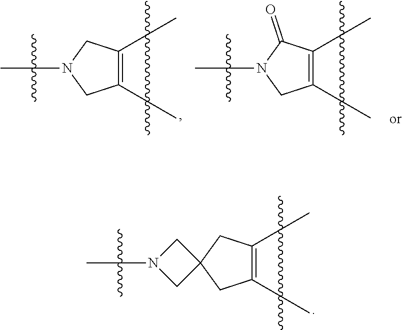

In certain embodiments, when the bond between Y and U is present, U is —CH₂— or —C(═O)—, and r is 1, then Ring A is not

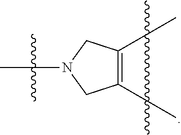

In certain embodiments, the compound is not

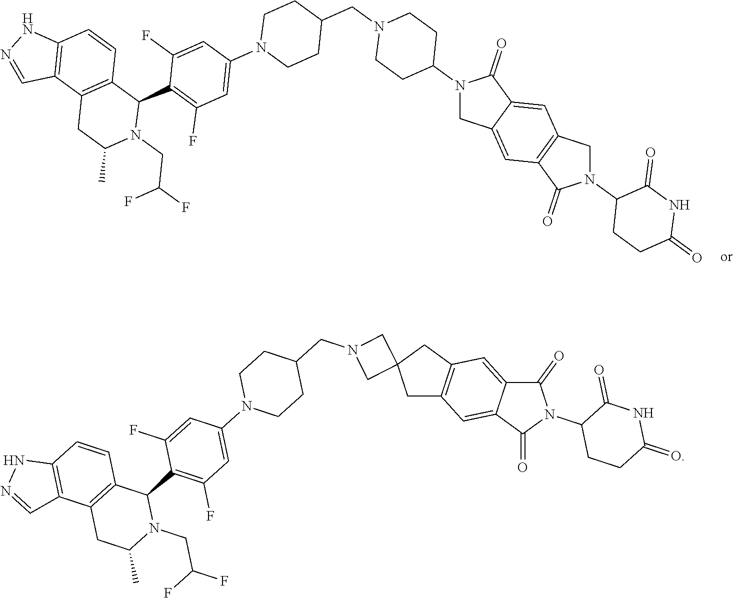 [SMILES: C[C@@H]1Cc2c(ccc3[nH]ncc23)[C@@H](c2c(F)cc(N3CCC(CN4CCC(N5Cc6cc7c(cc6C5=O)CN(C5CCC(=O)NC5=O)C7=O)CC4)CC3)cc2F)N1CC(F)F ; "or" ; C[C@@H]1Cc2c(ccc3[nH]ncc23)[C@@H](c2c(F)cc(N3CCC(CN4CC5(Cc6cc7c(cc6C5)C(=O)N(C5CCC(=O)NC5=O)C7=O)C4)CC3)cc2F)N1CC(F)F]

In certain embodiments, when the bond between Y and U is present, U is —CH₂— or —C(═O)—, and r is 1, then Ring A is not

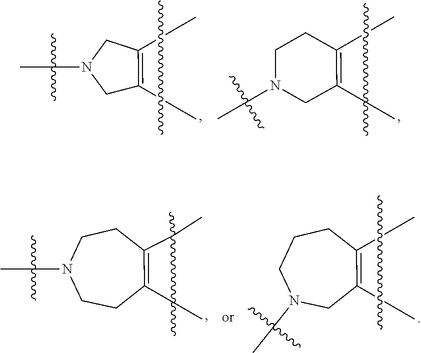

In certain embodiments, when the bond between Y and U is present, U is —CH₂— or —C(═O)—, and r is 1, then cither R¹ and R², or R² and R³, together with the intervening carbon atoms, form Ring A attached to L.

In certain embodiments, C is of Formula I-1-i

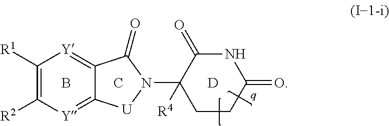

In certain embodiments, U is —CH₂— or —C(═O)—. In certain embodiments, U is —CH₂— or —C(═O)— when the bond between Y and U is present. In certain embodiments, U is —(C═O)—N(R^{U})—* or —N═C(R^{U})—* when the bond between Y and U is present.

In certain embodiments, C is of Formula I-1-ii

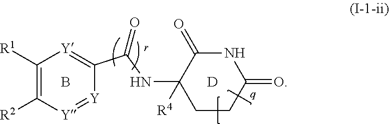

In certain embodiments, R¹ and R², together with the intervening carbon atoms, form Ring A attached to L, wherein the Ring A is optionally substituted 5- to 16-membered hetrocyclic.

In certain embodiments, Ring A is optionally substituted 7- to 16-membered fused heterocycle.

In certain embodiments, Ring A is

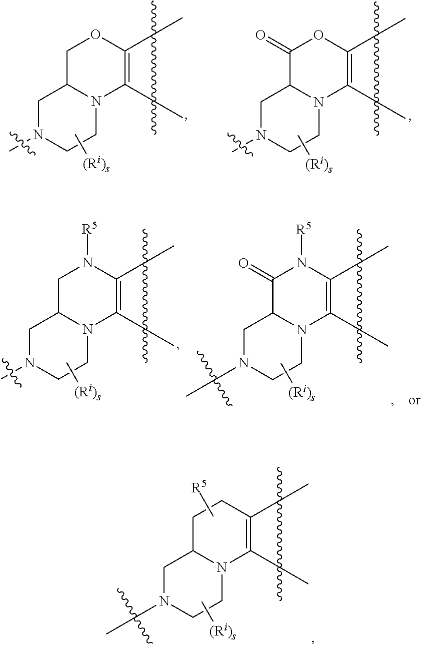

wherein:
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R², —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)ORD, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and s is an integer selected from 0 to 8, as valency permits.

In certain embodiments, R⁵ is hydrogen. In certain embodiments, R⁵ is C₁₋₆ alkyl.

In certain embodiments, Ring A is optionally substituted 7- to 16-membered spiro heterocycle.

In certain embodiments, Ring A is:

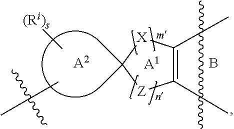

wherein:
Ring A² is C₃₋₈ carbocycle or 3- to 8-membered heterocycle;
each X is independently —C(R^{X1})₂—, —NR^{X2}—, —O—, —S—, —S(═O)—, or —S(═O)₂—;
each Z is independently —C(R^{Z1})₂—, —NR^{Z2}—, —O—, —S—, —S(═O)—, or —S(═O)₂—;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R^{Z2} is independently hydrogen or C₁₋₆ alkyl optionally substituted with one or more R^{u};
m′ and n′ are independently an integer selected from 0-3, wherein m′ and n′ are not both 0;
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkonyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, when none of m′ and n′ is 0, then Ring A¹ is 4- to 9-membered heterocycle.

In certain embodiments, Ring A² is C₃₋₈ carbocycle. In certain embodiments, Ring A² is 3-to 8-membered heterocycle.

In certain embodiments, each X is independently —C(R^{X1})₂—, —NR^{X2}, or —O—.

In certain embodiments, each Z is independently —C(R^{Z1})₂—, —NR^{Z2}—, or —O—.

In certain embodiments, m′ and n′ are independently an integer selected from 0-2, wherein m′ and n′ are not both 0. In certain embodiments, m′ and n′ are independently an integer selected from 0-2, wherein m′ and n′ are not both 0. In certain embodiments, m′ and n′ are independently an integer selected from 0 and 1, wherein m′ and n′ are not both 0. In certain embodiments, m′ and n′ are independently an integer selected from 0 and 1, wherein m′ and n′ are not both 0.

In certain embodiments, each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};

In certain embodiments, two geminal R^{X1} or two geminal R^{Z1} together form oxo.

In certain embodiments, two R^{X1} or two R^{Z1}, together with the intervening carbon atom(s), form C₃₋₁₂ carbocyclyl or 3- to 12-membered heterocyclyl, wherein the carbocyclyl or hetcrocyclyl is optionally substituted with one or more R^{u}.

In certain embodiments, Ring A is:

1)

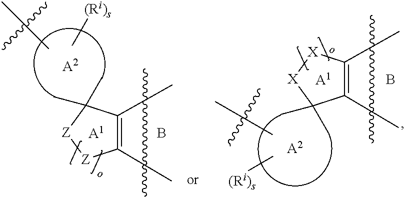

wherein o is 0 or 1; or
   2)

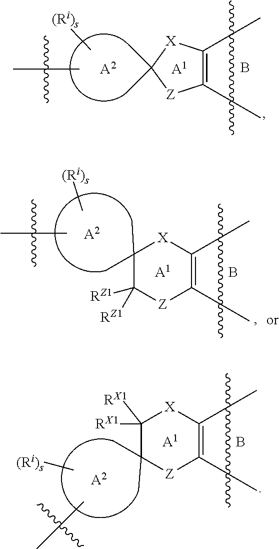

In certain embodiments, o is 0. In certain embodiments, o is 1.

In certain embodiments, Ring A is optionally substituted 5- to 6-membered heterocycle.

In certain embodiments, Ring A is

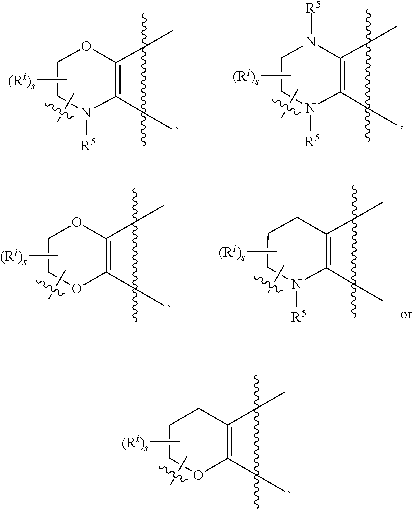

wherein:
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)ORD, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and s is an integer selected from 0 to 8, as valency permits.

In certain embodiments, R⁵ is hydrogen. In certain embodiments, R⁵ is C₁₋₆ alkyl.

In certain embodiments, Y″ is N.

In certain embodiments, Y″ is CR³, and R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R³ is hydrogen, halogen, or C₁₋₆ alkoxy, wherein the alkoxy is optionally substituted with one or more R^{u}.

In certain embodiments, R^{z} and R³, together with the intervening carbon atoms, form Ring A attached to L, wherein the Ring A is optionally substituted 5- to 16-membered heterocycle.

In certain embodiments, Ring A is optionally substituted 7- to 16-membered fused heterocycle.

In certain embodiments, Ring A is

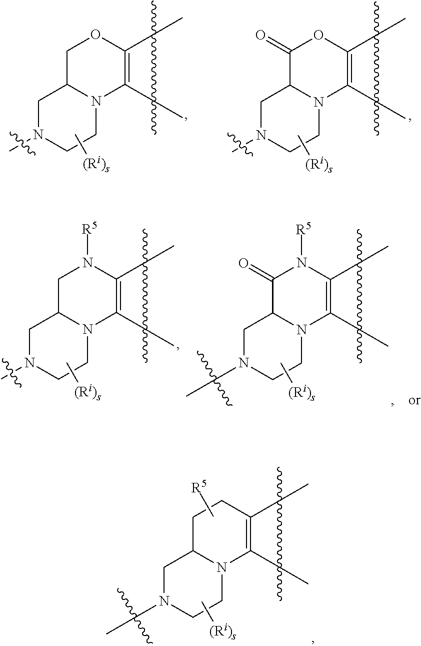

wherein:
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂ORD, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, R⁵ is hydrogen. In certain embodiments, R⁵ is C₁₋₆ alkyl.

In certain embodiments, Ring A is optionally substituted 7- to 16-membered spiro heterocycle.

In certain embodiments, Ring A is:

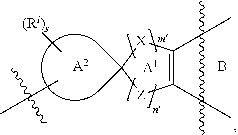

wherein:
Ring A² is C₃₋₈ carbocycle or 3- to 8-membered heterocycle;
each X is independently —C(R^{X1})₂—, —NR^{X2}—, —O—, —S—, —S(═O)—, or —S(═O)₂—;
each Z is independently —C(R^{Z1})₂—, —NR^{Z2}—, —O—, —S—, —S(═O)—, or —S(═O)₂—;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R″;
each occurrence of R^{X2} and R^{Z2} is independently hydrogen or C₁₋₆ alkyl optionally substituted with one or more R^{u};
m′ and n′ are independently an integer selected from 0-3, wherein m′ and n′ are not both 0; and
s is an integer selected from 0 to 8, as valency permits.

In certain embodiments, when none of m′ and n′ is 0, then Ring A¹ is 4- to 9-membered heterocycle.

In certain embodiments, Ring A² is C₃₋₈ carbocycle. In certain embodiments, Ring A² is 3-to 8-membered heterocycle.

In certain embodiments, each X is independently —C(R^{X1})₂—, —NR^{X2}—, or —O—.

In certain embodiments, each Z is independently —C(R^{Z1})₂—, —NR^{Z2}—, or —O—.

In certain embodiments, m′ and n′ are independently an integer selected from 0-2, wherein m′ and n′ are not both 0. In certain embodiments, m′ and n′ are independently an integer selected from 0-2, wherein m′ and n′ are not both 0. In certain embodiments, m′ and n′ are independently an integer selected from 0 and 1, wherein m′ and n′ are not both 0. In certain embodiments, m′ and n′ are independently an integer selected from 0 and 1, wherein m′ and n′ are not both 0.

In certain embodiments, each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(—O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};

In certain embodiments, two geminal R^{X1} or two geminal R^{Z1} together form oxo.

In certain embodiments, two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u}.

In certain embodiments, Ring A is:

1)

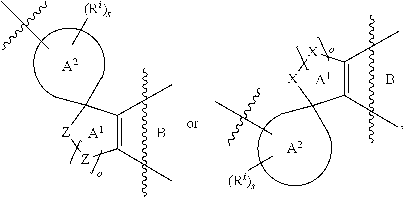

wherein o is 0 or 1; or
   2)

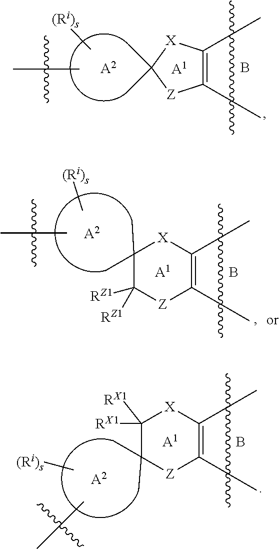

In certain embodiments, o is 0. In certain embodiments, o is 1.

In certain embodiments, Ring A is optionally substituted 5- to 6-membered heterocycle.

In certain embodiments, Ring A is

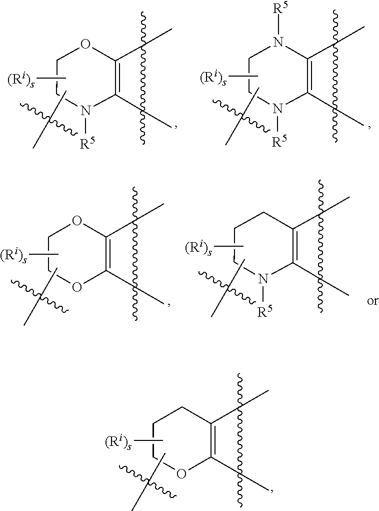

wherein:
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, R⁵ is hydrogen. In certain embodiments, R⁵ is C₁₋₆ alkyl.

In certain embodiments, R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, wherein R¹ is hydrogen, halogen, or C₁₋₆ alkoxy, wherein the alkoxy is optionally substituted with one or more R^{u}.

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, Ring A is optionally substituted with one or more R^{u}.

In certain embodiments, R^{u} is R⁵. In certain embodiments, R^{u} is Rⁱ. In certain embodiments, R^{u} is R^{X1}. In certain embodiments, R^{u} is R^{X2}. In certain embodiments, R^{u} is R^{Z1}. In certain embodiments, R^{u} is R^{Z2}.

In certain embodiments, Ring A is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, C is of Formula I-1-ii

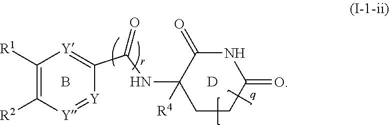

In certain embodiments, R^{z} is *-Cy²-, wherein * denotes attachment to L.

In certain embodiments, *-Cy²- is 3-membered heterocyclylene. In certain embodiments, *-Cy²- is 4-membered heterocyclylene. In certain embodiments, *-Cy²- is 5-membered heterocyclylene. In certain embodiments, *-Cy²- is 6-membered heterocyclylene. In certain embodiments, *-Cy²- is 7-membered heterocyclylene. In certain embodiments, *-Cy²- is 8-membered heterocyclylene. In certain embodiments, *-Cy²- is 9-membered heterocyclylene. In certain embodiments, *-Cy²- is 10-membered heterocyclylene. In certain embodiments, *-Cy²- is 11-membered heterocyclylene. In certain embodiments, *-Cy²- is 12-membered heterocyclylene. In certain embodiments, the above *-Cy²- is optionally substituted with one or more R^{u}.

In certain embodiments, *-Cy²- is 3- to 12-membered heterocyclylene. In certain embodiments, *-Cy²- is 3- to 11-membered heterocyclylene. In certain embodiments, *-Cy²- is 3-to 10-membered heterocyclylene. In certain embodiments, *-Cy²- is 3- to 9-membered heterocyclylene. In certain embodiments, *-Cy²- is 3- to 8-membered heterocyclylene. In certain embodiments, *-Cy²- is 3- to 7-membered heterocyclylene. In certain embodiments, *-Cy²- is 3-to 6-membered heterocyclylene. In certain embodiments, *-Cy²- is 3- to 5-membered heterocyclylene. In certain embodiments, *-Cy²- is 3- to 4-membered heterocyclylene. In certain embodiments, *-Cy²- is 4- to 12-membered heterocyclylene. In certain embodiments, *-Cy²- is 4-to 11-membered heterocyclylene. In certain embodiments, *-Cy²- is 4- to 10-membered heterocyclylene. In certain embodiments, *-Cy²- is 4- to 9-membered heterocyclylene. In certain embodiments, *-Cy²- is 4- to 8-membered heterocyclylene. In certain embodiments, *-Cy²- is 4-to 7-membered heterocyclylene. In certain embodiments, *-Cy²- is 4- to 6-membered heterocyclylene. In certain embodiments, *-Cy²- is 4- to 5-membered heterocyclylene. In certain embodiments, *-Cy²- is 5- to 12-membered heterocyclylene. In certain embodiments, *-Cy²- is 5-to 11-membered heterocyclylene. In certain embodiments, *-Cy²- is 5- to 10-membered heterocyclylene. In certain embodiments, *-Cy²- is 5- to 9-membered heterocyclylene. In certain embodiments, *-Cy²- is 5- to 8-membered heterocyclylene. In certain embodiments, *-Cy²- is 5-to 7-membered heterocyclylene. In certain embodiments, *-Cy²- is 5- to 6-membered heterocyclylene. In certain embodiments, *-Cy²- is 6- to 12-membered heterocyclylene. In certain embodiments, *-Cy²- is 6- to 11-membered heterocyclylene. In certain embodiments, *-Cy²- is 6-to 10-membered heterocyclylene. In certain embodiments, *-Cy²- is 6- to 9-membered heterocyclylene. In certain embodiments, *-Cy²- is 6- to 8-membered heterocyclylene. In certain embodiments, *-Cy²- is 6- to 7-membered heterocyclylene. In certain embodiments, *-Cy²- is 8-to 12-membered heterocyclylene. In certain embodiments, *-Cy²- is 8- to 11-membered heterocyclylene. In certain embodiments, *-Cy²- is 8- to 10-membered heterocyclylene. In certain embodiments, *-Cy²- is 8- to 9-membered heterocyclylene. In certain embodiments, *-Cy²- is 9-to 12-membered heterocyclylene. In certain embodiments, *-Cy²- is 9- to 11-membered heterocyclylene. In certain embodiments, *-Cy²- is 9- to 10-membered heterocyclylene. In certain embodiments, *-Cy²- is 10- to 12-membered heterocyclylene. In certain embodiments, *-Cy²- is 10- to 11-membered heterocyclylene. In certain embodiments, *-Cy²- is 11- to 12-membered heterocyclylene. In certain embodiments, the above *-Cy²- is optionally substituted with one or more R^{u}.

In certain embodiments, *-Cy²- is heterocyclylene comprising 1 heteroatom selected from nitrogen, oxygen, and sulfur. In certain embodiments, *-Cy²- is heterocyclylene comprising 2 heteroatoms selected from nitrogen, oxygen, and sulfur. In certain embodiments, *-Cy²- is heterocyclylene comprising 3 heteroatoms selected from nitrogen, oxygen, and sulfur. In certain embodiments, *-Cy²- is heterocyclylene comprising 4 heteroatoms selected from nitrogen, oxygen, and sulfur. In certain embodiments, the above *-Cy²- is optionally substituted with one or more R^{u}.

In certain embodiments, *-Cy²- is heterocyclylene comprising 1 to 4 heteroatoms selected from nitrogen, oxygen, and sulfur. In certain embodiments, *-Cy²- is heterocyclylene comprising 1 to 3 heteroatoms selected from nitrogen, oxygen, and sulfur. In certain embodiments, *-Cy²- is heterocyclylene comprising 1 to 2 heteroatoms selected from nitrogen, oxygen, and sulfur. In certain embodiments, *-Cy²- is heterocyclylene comprising 2 to 4 heteroatoms selected from nitrogen, oxygen, and sulfur. In certain embodiments, *-Cy²- is heterocyclylene comprising 2 to 3 heteroatoms selected from nitrogen, oxygen, and sulfur. In certain embodiments, *-Cy²- is heterocyclylene comprising 3 to 4 heteroatoms selected from nitrogen, oxygen, and sulfur. In certain embodiments, the above *-Cy²- is optionally substituted with one or more R^{u}.

In certain embodiments, *-Cy²- is C₃ carbocyclylene. In certain embodiments, *-Cy²- is C₄ carbocyclylene. In certain embodiments, *-Cy²- is C₅ carbocyclylene. In certain embodiments, *-Cy²- is C₆ carbocyclylene. In certain embodiments, *-Cy²- is C₇ carbocyclylene. In certain embodiments, *-Cy²- is C₈ carbocyclylene. In certain embodiments, *-Cy²- is C₉ carbocyclylene. In certain embodiments, *-Cy²- is C₁₀ carbocyclylene. In certain embodiments, *-Cy²- is C₁₁ carbocyclylene. In certain embodiments, *-Cy²- is C₁₋₂ carbocyclylene. In certain embodiments, the above *-Cy²- is optionally substituted with one or more R^{u}.

In certain embodiments, *-Cy²- is C₃₋₁₂ carbocyclylene. In certain embodiments, *-Cy²- is C₃₋₁₁ carbocyclylene. In certain embodiments, *-Cy²- is C₃₋₁₀ carbocyclylene. In certain embodiments, *-Cy²- is C₃₉ carbocyclylene. In certain embodiments, *-Cy²- is C₃₋₈ carbocyclylene. In certain embodiments, *-Cy²- is C₃₋₇ carbocyclylene. In certain embodiments, *-Cy²- is C₃₋₆ carbocyclylene. In certain embodiments, *-Cy²- is C₃₋₅ carbocyclylene. In certain embodiments, *-Cy²- is C₃₄ carbocyclylene. In certain embodiments, *-Cy²- is C₄₋₁₂ carbocyclylene. In certain embodiments, *-Cy²- is C₄₋₁₁ carbocyclylene. In certain embodiments, *-Cy²- is C₄₋₁₀ carbocyclylenc. In certain embodiments, *-Cy²- is C₄₉ carbocyclylenc. In certain embodiments, *-Cy²- is C₄₈ carbocyclylene. In certain embodiments, *-Cy²- is C₄₇ carbocyclylene. In certain embodiments, *-Cy²- is C₄₋₆ carbocyclylene. In certain embodiments, *-Cy²- is C₄₋₅ carbocyclylene. In certain embodiments, *-Cy²- is C₅₋₁₂ carbocyclylene. In certain embodiments, *-Cy²- is C₅₋₁₁ carbocyclylene. In certain embodiments, *-Cy²- is C₅₋₁₀ carbocyclylene. In certain embodiments, *-Cy²- is C₅₋₉ carbocyclylene. In certain embodiments, *-Cy²- is C₅₋₈ carbocyclylene. In certain embodiments, *-Cy²- is C₅₋₇ carbocyclylene. In certain embodiments, *-Cy²- is C₅₋₆ carbocyclylene. In certain embodiments, *-Cy²- is C₆₋₁₂ carbocyclylene. In certain embodiments, *-Cy²- is C₆₋₁₁ carbocyclylene. In certain embodiments, *-Cy²- is C₆₋₁₀ carbocyclylene. In certain embodiments, *-Cy²- is C₆₋₉ carbocyclylene. In certain embodiments, *-Cy²- is C₆₋₈ carbocyclylene. In certain embodiments, *-Cy²- is C₆₋₇ carbocyclylene. In certain embodiments, *-Cy²- is C₇₋₁₂ carbocyclylene. In certain embodiments, *-Cy²- is C₇₋₁₁ carbocyclylene. In certain embodiments, *-Cy²- is C₇₋₁₀ carbocyclylene. In certain embodiments, *-Cy²- is C₇₋₉ carbocyclylene. In certain embodiments, *-Cy²- is C₇₋₈ carbocyclylene. In certain embodiments, *-Cy²- is C₈₋₁₂ carbocyclylene. In certain embodiments, *-Cy²- is C₈₋₁₁ carbocyclylene. In certain embodiments, *-Cy²- is C₈₋₁₀ carbocyclylene. In certain embodiments, *-Cy²- is C₈₋₉ carbocyclylene. In certain embodiments, *-Cy²- is C₉₋₁₂ carbocyclylene. In certain embodiments, *-Cy²- is C₉₋₁₁ carbocyclylene. In certain embodiments, *-Cy²- is C₉₋₁₀ carbocyclylene. In certain embodiments, *-Cy²- is C₁₀₋₁₂ carbocyclylene. In certain embodiments, *-Cy²- is C₁₀₋₁₁ carbocyclylene. In certain embodiments, *-Cy²- is C₁₁₋₁₂ carbocyclylene. In certain embodiments, the above *-Cy²- is optionally substituted with one or more R^{u}.

In certain embodiments, *-Cy²- is C₅₋₁₂ fused carbocyclene or 5- to 12-membered fused heterocyclylene, wherein the carbocyclene or heterocyclylene is optionally substituted with one or more R^{u}.

In certain embodiments, *-Cy²- is 5- to 12-membered fused heterocyclylene comprising 1 or 2 nitrogen atoms, wherein the heterocyclene is optionally substituted with one or more R^{u}.

In certain embodiments, *-Cy²- is

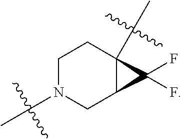 [SMILES: *N1CC[C@]2(*)[C@H](C1)C2(F)F]

In certain embodiments, R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R.

In certain embodiments, R¹ is hydrogen, halogen, or C₁₋₆ alkoxy, wherein the alkoxy is optionally substituted with one or more R^{u}.

In certain embodiments, Y″ is N.

In certain embodiments, Y″ is CR³.

In certain embodiments, R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R³ is hydrogen, halogen, or C₁₋₆ alkoxy, wherein the alkoxy is optionally substituted with one or more R^{u}.

In certain embodiments, Y is N.

In certain embodiments, Y is CR^{Y}.

In certain embodiments, R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{Y} is hydrogen, halogen, or C₁₋₆ alkoxy, wherein the alkoxy is optionally substituted with one or more R^{u}.

In certain embodiments, r is 0. In certain embodiments, r is 1.

In certain embodiments, R⁴ is hydrogen. In certain embodiments, R⁴ is deuterium. In certain embodiments, R⁴ is C₁₋₆ haloalkyl. In certain embodiments, R⁴ is C₁₋₆ alkyl.

In certain embodiments, q is 0. In certain embodiments, q is 1. In certain embodiments, q is 2.

In certain embodiments, each of X^{T1}, X^{T2}, X^{T3}, and X^{T4} is CRT.

In certain embodiments, X^{T1} and X^{T4} are CF, and X^{T2} and X^{T3} are CH. In certain embodiments, one of X^{T1} and X^{T4} is CF or C(OCH₃), the other one of X^{T1} and X^{T4} is CH, and each of X^{T2} and X^{T3} is CH.

In certain embodiments, one of X^{T1}, X12, X′13, and X14 is N.

In certain embodiments, one of X^{T1} and X^{T4} is N, the other one of X^{T1} and X^{T4} is CH, and each of X^{T2} and X^{T3} is CH. In certain embodiments, one of X^{T2} and X^{T3} is N, the other one of X^{T2} and X^{T3} is CH, and each of X^{T1} and X^{T4} is CH.

In certain embodiments, two of X^{T1}, X^{T2}, X^{T3}, and X^{T4} are N.

In certain embodiments, each of X^{T1} and X^{T4} is CH, and each of X^{T2} and X^{T3} is N.

In certain embodiments, each R^{T} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, each R^{T} is independently hydrogen, C₁₋₆ alkoxy, or halogen.

In certain embodiments, R^{E} is C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, or —(C₁₋₆ alkylene-C₃₋₁₂ carbocyclyl), wherein the alkyl, alkenyl, alkynyl, aryl, heteroaryl, carbocyclyl, or heterocyclyl is optionally substituted with one or more R^{u}.

In certain embodiments, R^{E} is C₁₋₆ alkyl or —(C₁₋₆ alkylene-C₃₋₁₂ carbocyclyl), wherein the alkyl or carbocyclyl is optionally substituted with one or more R^{u}.

In certain embodiments, R^{E} is

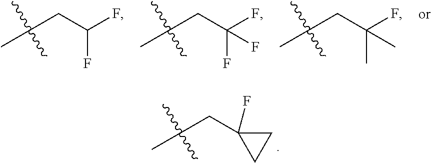

In certain embodiments, Cy¹ is C₃₋₁₂ carbocyclylene or 3- to 12-membered heterocyclylene, wherein the carbocyclylene or heterocyclylene is optionally substituted by one or more R^{u}. In certain embodiments, Cy¹ is 6-membered heteroarylene or C₆ arylene, wherein the heterocyclylene is optionally substituted by one or more R^{u}.

In certain embodiments, Cy¹ is 3- to 12-membered heterocyclylene, wherein the heterocyclylene is optionally substituted by one or more R^{u}.

In certain embodiments, Cy¹ is 3- to 12-membered heterocyclylene is selected from morpholinylene, piperidinylene, piperazinylene, 7-azaspiro[3.5]nonanylene, 2,7-diazaspiro[3.5]nonanylene, 2-azaspiro[3.5]nonanylene, 2,7-diazaspiro[3.5]nonanylene, 1-oxa-8-azaspiro[4.5]decenylene, 2-oxa-8-azaspiro[4.5]decenylene, 5-oxa-2-azaspiro[3.4]octanylene, 6-oxa-2-azaspiro[3.4]octanylene, 3,9-diazaspiro[5.5]undecanylene, 5-oxa-2-azaspiro[3.5]nonanylene, 1-oxa-9-azaspiro[5.5]undecanylene, 1-oxa-4,9-diazaspiro[5.5]undecanylene, 2,6-diazaspiro[3.3]heptanylene, 2-azaspiro[3.3]heptanylene, 1,5-dioxa-9-azaspiro[5.5]undecanylene, 1,4-dioxa-9-azaspiro[5.5]undecanylene, 5,9-dioxa-2-azaspiro[3.5]nonanylene, 5,8-dioxa-2-azaspiro[3.5]nonanylene, 6-oxa-2-azaspiro[3.5]nonanylene, 1-oxa-7-azaspiro[3.5]nonanylene, 5-oxa-2-azaspiro[3.6]decenylene, 5-oxa-2-azaspiro[3.6]decenylene, 5,9-dioxa-2-azaspiro[3.6]decenylene, 5,8-dioxa-2-azaspiro[3.6]decenylene, and 6,9-dioxa-2-azaspiro[3.6]decenylene, wherein the heterocyclylene is optionally substituted by one or more R^{u}.

In certain embodiments, Cy¹ is 3- to 12-membered heterocyclylene is selected from:

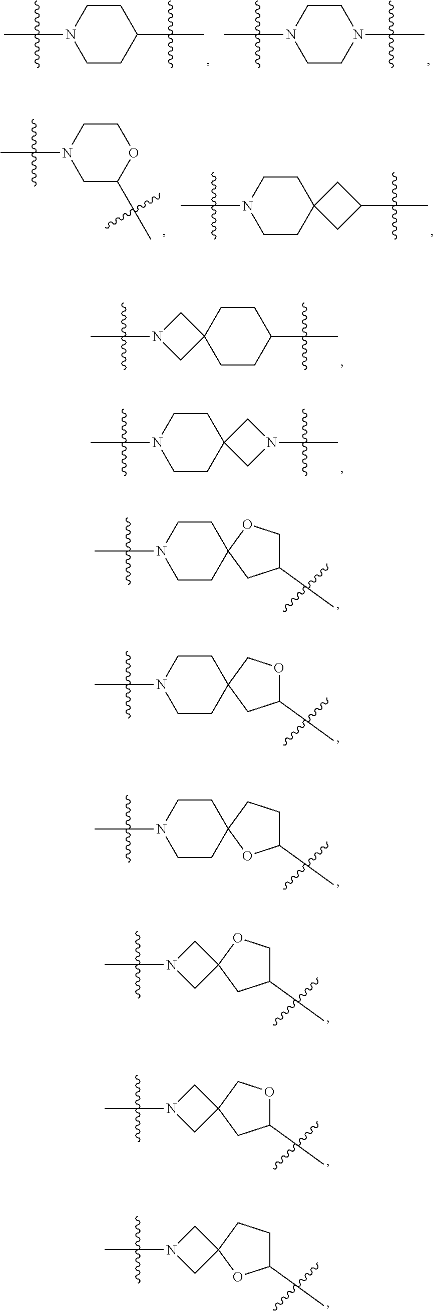

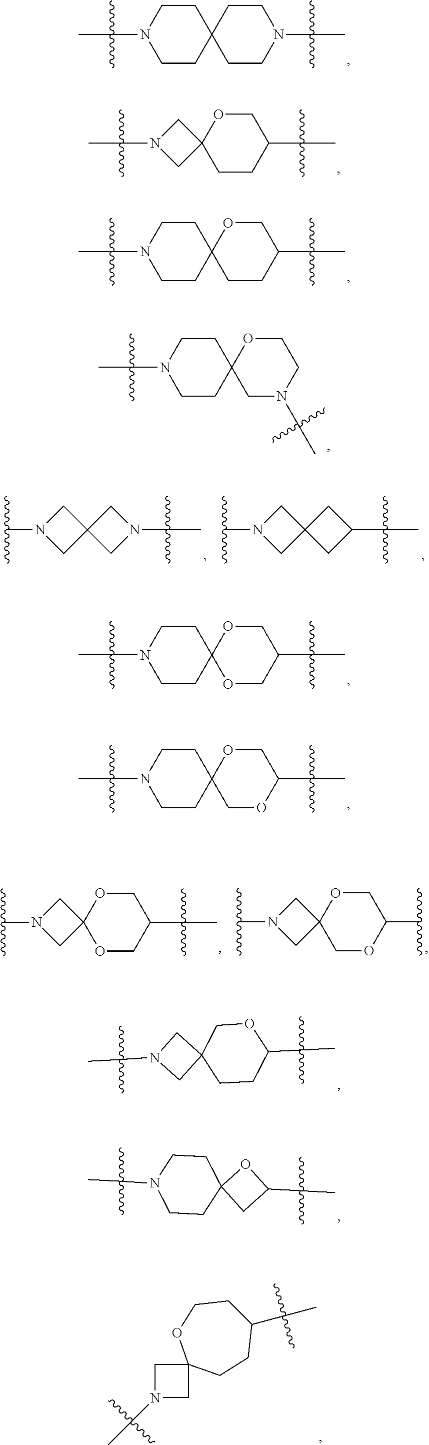

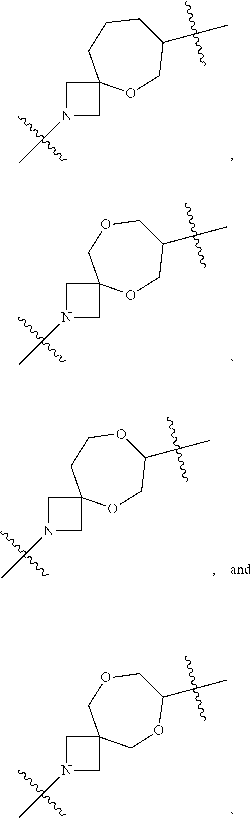 [SMILES: *C1CCCC2(CN(*)C2)OC1 ; *C1COCC2(CN(*)C2)OC1 ; *C1COC2(CCO1)CN(*)C2 ; *C1COCC2(CO1)CN(*)C2]

wherein the heterocyclylene is optionally substituted by one or more R^{u}.

In certain embodiments, W is absent.

In certain embodiments, Z′ is absent.

In certain embodiments, Z′ is —C(═O)—, C₁₋₆ alkylene, *—O—(C₁₋₆ alkylene)-, *—(C₁₋₆ alkylene)-O—, *—C(═O)—(C₁₋₆ alkylene)-, *—(C₁₋₆ alkylene)-C(═O)—, 3- to 12-membered heterocyclylene, *—C(═O)-(3- to 12-membered heterocyclylene)-, *-(3- to 12-membered heterocyclylene)-C(═O)—, *-(3- to 12-membered heterocyclylene)-(C₁₋₆ alkylene)-, *—(C₁₋₆ alkylene)-(3- to 12-membered heterocyclylene)-, *—(C₁₋₆ alkylene)-(3- to 12-membered heterocyclylene)-(C₁₋₆ alkylene)-, *—(C₁₋₆ alkylene)-(3- to 12-membered heterocyclylene)-(C(═O))—, *—(C(═O))-(3- to 12-membered heterocyclylene)-(C₁₋₆ alkylene)-, *-(3- to 12-membered heterocyclylene)-(C₁₋₆ alkylene)-(C(═O))—, *—(C(═O))—(C₁₋₆ alkylene)-(3- to 12-membered heterocyclylene)-, *—(C₁₋₆ alkylene)-(C(═O))-(3- to 12-membered heterocyclylene)-, or *-(3- to 12-membered heterocyclylene)-(C(═O))—(C₁₋₆ alkylene)-, wherein the alkylene or heterocyclylene is optionally substituted by one or more R^{u}, and *denotes attachment to C.

In certain embodiments, Z′ is C₁₋₆ alkylene, *—C(═O)—(C₁₋₆ alkylene)-, *—(C₁₋₆ alkylene)-C(═O)—, 3- to 12-membered heterocyclylene, or *-(3- to 12-membered heterocyclylene)-(C₁₋₆ alkylene)-, wherein the alkylene or heterocyclylene is optionally substituted by one or more R^{u}, and *denotes attachment to C.

In certain embodiments, the compound is selected from the compounds in Tables 1-3, or a pharmaceutically acceptable salt thereof.

In certain embodiments, the compound is selected from the compounds in Tables 1-3.

In certain embodiments, the compound is selected from the compounds in Table 1, or a pharmaceutically acceptable salt thereof.

In certain embodiments, the compound is selected from the compounds in Table 1.

In certain embodiments, the compound is selected from the compounds in Table 2, or a pharmaceutically acceptable salt thereof.

In certain embodiments, the compound is selected from the compounds in Table 2.

In certain embodiments, the compound is selected from the compounds in Table 3, or a pharmaceutically acceptable salt thereof.

In certain embodiments, the compound is selected from the compounds in Table 3.

TABLE 1
Compound A1-A26

| Com- | | |
|---|---|---|
| pound | | |
| No | Chemical Structure | Chemical Name |
| A1 | 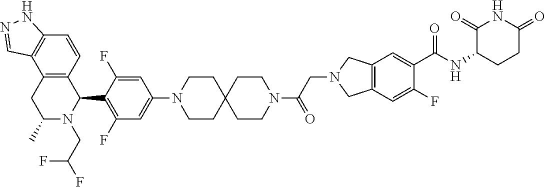 [SMILES: C[C@@H]1Cc2c(ccc3[nH]ncc23)[C@@H](c2c(F)cc(N3CCC4(CCN(C(=O)CN5Cc6cc(F)c(C(=O)N[C@H]7CCC(=O)NC7=O)cc6C5)CC4)CC3)cc2F)N1CC(F)F] | 2-(2-(9-(4-((6S,8R)-7-(2,2-difluoroethyl)-8- methyl-6,7,8,9-tetrahydro-3H-pyrazolo[4,3- f]isoquinolin-6-yl)-3,5-difluorophenyl)-3,9- diazaspiro[5.5]undecan-3-yl)-2-oxoethyl)-N- ((S)-2,6-dioxopiperidin-3-yl)-6- fluoroisoindoline-5-carboxamide |
| A2 | 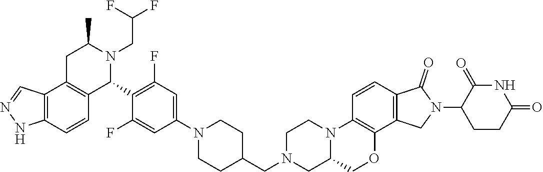 [SMILES: C[C@@H]1Cc2c(ccc3[nH]ncc23)[C@@H](c2c(F)cc(N3CCC(CN4CCN5c6ccc7c(c6OC[C@@H]5C4)CN(C4CCC(=O)NC4=O)C7=O)CC3)cc2F)N1CC(F)F] | 3-((S)-7-((1-(4-((6S,8R)-7-(2,2-difluoroethyl)- 8-methyl-6,7,8,9-tetrahydro-3H-pyrazolo[4,3- f]isoquinolin-6-yl)-3,5- difluorophenyl)piperidin-4-yl)methyl)-1-oxo- 1,3,5,5a,6,7,8,9-octahydro-2H- pyrazino[1′,2′:4,5][1,4]oxazino[2,3-e]isoindol- 2-yl)piperidine-2,6-dione |
| A4 | 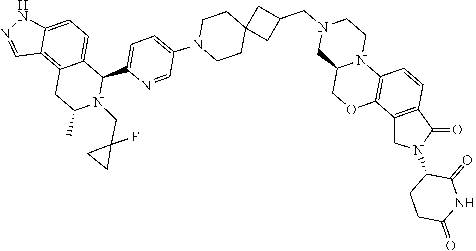 [SMILES: C[C@@H]1Cc2c(ccc3[nH]ncc23)[C@@H](c2ccc(N3CCC4(CC3)CC(CN3CCN5c6ccc7c(c6OC[C@H]5C3)CN([C@H]3CCC(=O)NC3=O)C7=O)C4)cn2)N1CC1(F)CC1] | (S)-3-((R)-7-((7-(6-((6S,8R)-7-((1- fluorocyclopropyl)methyl)-8-methyl-6,7,8,9- tetrahydro-3H-pyrazolo[4,3-f]isoquinolin-6- yl)pyridin-3-yl)-7-azaspiro[3.5]nonan-2- yl)methyl)-1-oxo-1,3,5,5a,6,7,8,9-octahydro- 2H-pyrazino[1′,2′:4,5][1,4]oxazino[2,3- e]isoindol-2-yl)piperidine-2,6-dione |
| A5 | 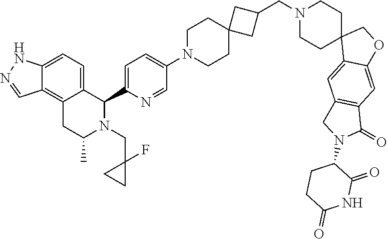 [SMILES: C[C@@H]1Cc2c(ccc3[nH]ncc23)[C@@H](c2ccc(N3CCC4(CC3)CC(CN3CCC5(CC3)COc3cc6c(cc35)CN([C@H]3CCC(=O)NC3=O)C6=O)C4)cn2)N1CC1(F)CC1] | (S)-3-(1′-((7-(6-((6S,8R)-7-((1- fluorocyclopropyl)methyl)-8-methyl-6,7,8,9- tetrahydro-3H-pyrazolo[4,3-f]isoquinolin-6- yl)pyridin-3-yl)-7-azaspiro[3.5]nonan-2- yl)methyl)-7-oxo-5,7-dihydro-2H,6H- spiro[furo[2,3-f]isoindole-3,4′-piperidin]-6- yl)piperidine-2,6-dione |
| A6 | 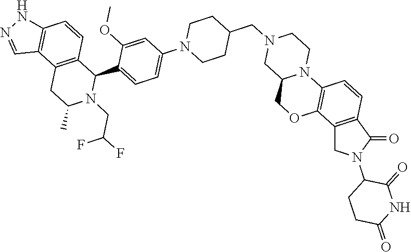 [SMILES: COc1cc(N2CCC(CN3CCN4c5ccc6c(c5OC[C@H]4C3)CN(C3CCC(=O)NC3=O)C6=O)CC2)ccc1[C@@H]1c2ccc3[nH]ncc3c2C[C@@H](C)N1CC(F)F] | 3-((R)-7-((1-(4-((6S,8R)-7-(2,2-difluoroethyl)- 8-methyl-6,7,8,9-tetrahydro-3H-pyrazolo[4,3- f]isoquinolin-6-yl)-3- methoxyphenyl)piperidin-4-yl)methyl)-1-oxo- 1,3,5,5a,6,7,8,9-octahydro-2H- pyrazino[1′,2′:4,5][1,4]oxazino[2,3-e]isoindol- 2-yl)piperidine-2,6-dione |
| A8 | 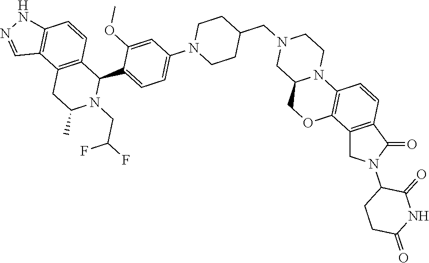 [SMILES: COc1cc(N2CCC(CN3CCN4c5ccc6c(c5OC[C@H]4C3)CN(C3CCC(=O)NC3=O)C6=O)CC2)ccc1[C@@H]1c2ccc3[nH]ncc3c2C[C@@H](C)N1CC(F)F] | (R)-3-((S)-7-((1-(4-((6R,8S)-7-(2,2- difluoroethyl)-8-methyl-6,7,8,9-tetrahydro- 3H-pyrazolo[4,3-f]isoquinolin-6-yl)-3- methoxyphenyl)piperidin-4-yl)methyl)-1-oxo- 1,3,5,5a,6,7,8,9-octahydro-2H- pyrazino[1′,2′:4,5][1,4]oxazino[2,3-e]isoindol- 2-yl)piperidine-2,6-dione |
| A9 | 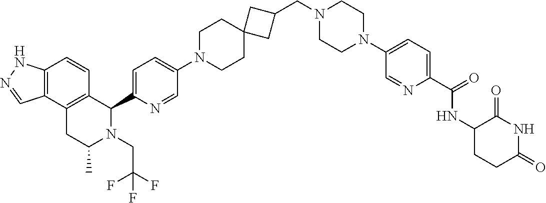 [SMILES: C[C@@H]1Cc2c(ccc3[nH]ncc23)[C@@H](c2ccc(N3CCC4(CC3)CC(CN3CCN(c5ccc(C(=O)NC6CCC(=O)NC6=O)nc5)CC3)C4)cn2)N1CC(F)(F)F] | N-(2,6-dioxopiperidin-3-yl)-5-(4-((7-(6- ((6R,8S)-8-methyl-7-(2,2,2-trifluoroethyl)- 6,7,8,9-tetrahydro-3H-pyrazolo[4,3- f]isoquinolin-6-yl)pyridin-3-yl)-7- azaspiro[3.5]nonan-2-yl)methyl)piperazin-1- yl)picolinamide |
| A10 | 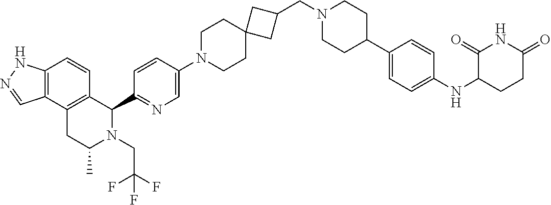 [SMILES: C[C@@H]1Cc2c(ccc3[nH]ncc23)[C@@H](c2ccc(N3CCC4(CC3)CC(CN3CCC(c5ccc(NC6CCC(=O)NC6=O)cc5)CC3)C4)cn2)N1CC(F)(F)F] | 3-((4-(1-((7-(6-((6R,8S)-8-methyl-7-(2,2,2- trifluoroethyl)-6,7,8,9-tetrahydro-3H- pyrazolo[4,3-f]isoquinolin-6-yl)pyridin-3-yl)- 7-azaspiro[3.5]nonan-2-yl)methyl)piperidin-4- yl)phenyl)amino)piperidine-2,6-dione |
| A11 | 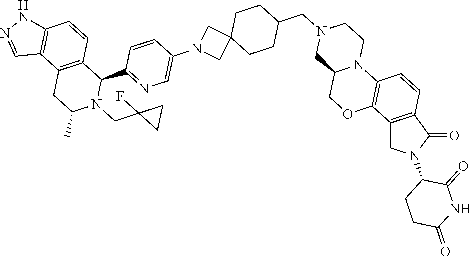 [SMILES: C[C@@H]1Cc2c(ccc3[nH]ncc23)[C@@H](c2ccc(N3CC4(CCC(CN5CCN6c7ccc8c(c7OC[C@H]6C5)CN([C@H]5CCC(=O)NC5=O)C8=O)CC4)C3)cn2)N1CC1(F)CC1] | (R)-3-((S)-7-((2-(6-((6R,8S)-7-((1- fluorocyclopropyl)methyl)-8-methyl-6,7,8,9- tetrahydro-3H-pyrazolo[4,3-f]isoquinolin-6- yl)pyridin-3-yl)-2-azaspiro[3.5]nonan-7- yl)methyl)-1-oxo-1,3,5,5a,6,7,8,9-octahydro- 2H-pyrazino[1′,2′:4,5][1,4]oxazino[2,3- e]isoindol-2-yl)piperidine-2,6-dione |
| A12 | 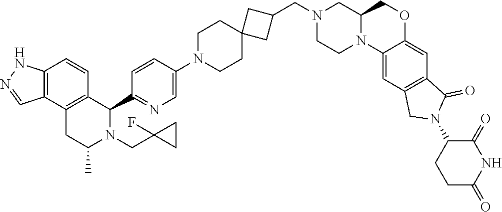 | (S)-3-((S)-3-((7-(6-((6S,8R)-7-((1- fluorocyclopropyl)methyl)-8-methyl-6,7,8,9- tetrahydro-3H-pyrazolo[4,3-f]isoquinolin-6- yl)pyridin-3-yl)-7-azaspiro[3.5]nonan-2- yl)methyl)-8-oxo-1,2,3,4,4a,5,8,10-octahydro- 9H-pyrazino[1′,2′:4,5][1,4]oxazino[2,3- f]isoindol-9-yl)piperidine-2,6-dione |
| A13 | 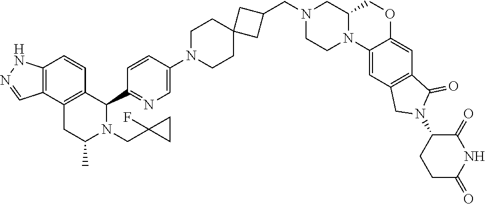 [SMILES: C[C@@H]1Cc2c(ccc3[nH]ncc23)[C@@H](c2ccc(N3CCC4(CC3)CC(CN3CCN5c6cc7c(cc6OC[C@H]5C3)C(=O)N([C@H]3CCC(=O)NC3=O)C7)C4)cn2)N1CC1(F)CC1] | (R)-3-((S)-3-((7-(6-((6R,8S)-7-((1- fluorocyclopropyl)methyl)-8-methyl-6,7,8,9- tetrahydro-3H-pyrazolo[4,3-f]isoquinolin-6- yl)pyridin-3-yl)-7-azaspiro[3.5]nonan-2- yl)methyl)-8-oxo-1,2,3,4,4a,5,8,10-octahydro- 9H-pyrazino[1′,2′:4,5][1,4]oxazino[2,3- f]isoindol-9-yl)piperidine-2,6-dione |
| A14 | 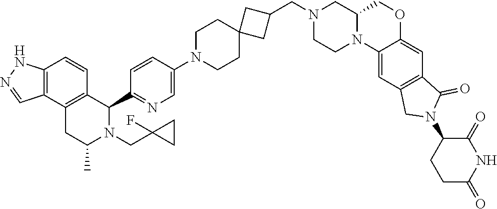 [SMILES: C[C@@H]1Cc2c(ccc3[nH]ncc23)[C@@H](c2ccc(N3CCC4(CC3)CC(CN3CCN5c6cc7c(cc6OC[C@H]5C3)C(=O)N([C@@H]3CCC(=O)NC3=O)C7)C4)cn2)N1CC1(F)CC1] | (R)-3-((R)-3-((7-(6-((6S,8R)-7-((1- fluorocyclopropyl)methyl)-8-methyl-6,7,8,9- tetrahydro-3H-pyrazolo[4,3-f]isoquinolin-6- yl)pyridin-3-yl)-7-azaspiro[3.5]nonan-2- yl)methyl)-8-oxo-1,2,3,4,4a,5,8,10-octahydro- 9H-pyrazino[1′,2′:4,5][1,4]oxazino[2,3- f]isoindol-9-yl)piperidine-2,6-dione |
| A15 | 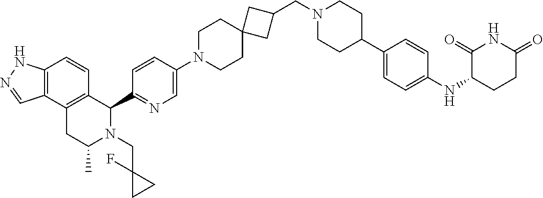 [SMILES: C[C@@H]1Cc2c(ccc3[nH]ncc23)[C@@H](c2ccc(N3CCC4(CC3)CC(CN3CCC(c5ccc(N[C@H]6CCC(=O)NC6=O)cc5)CC3)C4)cn2)N1CC1(F)CC1] | (R)-3-((4-(1-((7-(6-((6R,8S)-7-((1- fluorocyclopropyl)methyl)-8-methyl-6,7,8,9- tetrahydro-3H-pyrazolo[4,3-f]isoquinolin-6- yl)pyridin-3-yl)-7-azaspiro[3.5]nonan-2- yl)methyl)piperidin-4- yl)phenyl)amino)piperidine-2,6-dione |
| A16 | 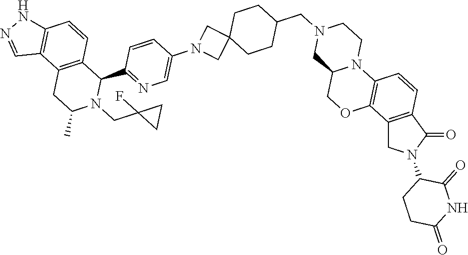 | (S)-3-((R)-7-((2-(6-((6S,8R)-7-((1- fluorocyclopropyl)methyl)-8-methyl-6,7,8,9- tetrahydro-3H-pyrazolo[4,3-f]isoquinolin-6- yl)pyridin-3-yl)-2-azaspiro[3.5]nonan-7- yl)methyl)-1-oxo-1,3,5,5a,6,7,8,9-octahydro- 2H-pyrazino[1′,2′:4,5][1,4]oxazino[2,3- e]isoindol-2-yl)piperidine-2,6-dione |
| A17 | 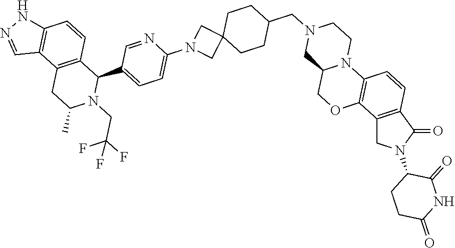 [SMILES: C[C@@H]1Cc2c(ccc3[nH]ncc23)[C@@H](c2ccc(N3CC4(CCC(CN5CCN6c7ccc8c(c7OC[C@H]6C5)CN([C@H]5CCC(=O)NC5=O)C8=O)CC4)C3)nc2)N1CC(F)(F)F] | (R)-3-((S)-7-((2-(5-((6R,8S)-8-methyl-7- (2,2,2-trifluoroethyl)-6,7,8,9-tetrahydro-3H- pyrazolo[4,3-f]isoquinolin-6-yl)pyridin-2-yl)- 2-azaspiro[3.5]nonan-7-yl)methyl)-1-oxo- 1,3,5,5a,6,7,8,9-octahydro-2H- pyrazino[1′,2′:4,5][1,4]oxazino[2,3-e]isoindol- 2-yl)piperidine-2,6-dione |
| A18 | 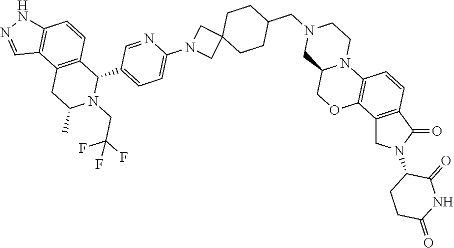 [SMILES: C[C@@H]1Cc2c(ccc3[nH]ncc23)[C@H](c2ccc(N3CC4(CCC(CN5CCN6c7ccc8c(c7OC[C@H]6C5)CN([C@H]5CCC(=O)NC5=O)C8=O)CC4)C3)nc2)N1CC(F)(F)F] | (R)-3-((S)-7-((2-(5-((6S,8S)-8-methyl-7- (2,2,2-trifluoroethyl)-6,7,8,9-tetrahydro-3H- pyrazolo[4,3-f]isoquinolin-6-yl)pyridin-2-yl)- 2-azaspiro[3.5]nonan-7-yl)methyl)-1-oxo- 1,3,5,5a,6,7,8,9-octahydro-2H- pyrazino[1′,2′:4,5][1,4]oxazino[2,3-e]isoindol- 2-yl)piperidine-2,6-dione |
| A19 | 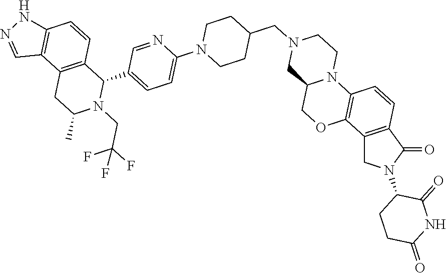 [SMILES: C[C@@H]1Cc2c(ccc3[nH]ncc23)[C@H](c2ccc(N3CCC(CN4CCN5c6ccc7c(c6OC[C@H]5C4)CN([C@H]4CCC(=O)NC4=O)C7=O)CC3)nc2)N1CC(F)(F)F] | (R)-3-((S)-7-((1-(5-((65,8S)-8-methyl-7- (2,2,2-trifluoroethyl)-6,7,8,9-tetrahydro-3H- pyrazolo[4,3-f]isoquinolin-6-yl)pyridin-2- yl)piperidin-4-yl)methyl)-1-oxo- 1,3,5,5a,6,7,8,9-octahydro-2H- pyrazino[1′,2′:4,5][1,4]oxazino[2,3-e]isoindol- 2-yl)piperidine-2,6-dione |
| A20 | 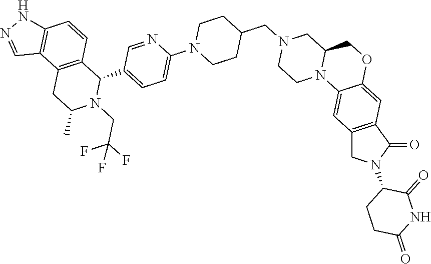 [SMILES: C[C@@H]1Cc2c(ccc3[nH]ncc23)[C@H](c2ccc(N3CCC(CN4CCN5c6cc7c(cc6OC[C@@H]5C4)C(=O)N([C@H]4CCC(=O)NC4=O)C7)CC3)nc2)N1CC(F)(F)F] | (R)-3-((R)-3-((1-(5-((6S,8S)-8-methyl-7- (2,2,2-trifluoroethyl)-6,7,8,9-tetrahydro-3H- pyrazolo[4,3-f]isoquinolin-6-yl)pyridin-2- yl)piperidin-4-yl)methyl)-8-oxo- 1,2,3,4,4a,5,8,10-octahydro-9H- pyrazino[1′,2′:4,5][1,4]oxazino[2,3-f]isoindol- 9-yl)piperidine-2,6-dione |
| A21 | 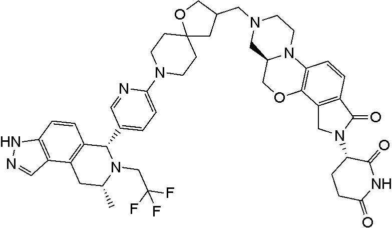 [SMILES: C[C@@H]1Cc2c(ccc3[nH]ncc23)[C@H](c2ccc(N3CCC4(CC3)CC(CN3CCN5c6ccc7c(c6OC[C@H]5C3)CN([C@H]3CCC(=O)NC3=O)C7=O)CO4)nc2)N1CC(F)(F)F] | (3S)-3-((5aR)-7-((8-(5-((6R,8R)-8-methyl-7- (2,2,2-trifluoroethyl)-6,7,8,9-tetrahydro-3H- pyrazolo[4,3-f]isoquinolin-6-yl)pyridin-2-yl)- 1-oxa-8-azaspiro[4.5]decan-3-yl)methyl)-1- oxo-1,3,5,5a,6,7,8,9-octahydro-2H- pyrazino[1′,2′:4,5][1,4]oxazino[2,3-e]isoindol- 2-yl)piperidine-2,6-dione |
| A22 | 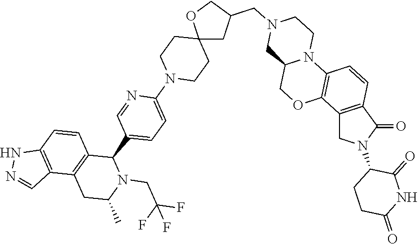 [SMILES: C[C@@H]1Cc2c(ccc3[nH]ncc23)[C@@H](c2ccc(N3CCC4(CC3)CC(CN3CCN5c6ccc7c(c6OC[C@H]5C3)CN([C@H]3CCC(=O)NC3=O)C7=O)CO4)nc2)N1CC(F)(F)F] | (3S)-3-((5aR)-7-((8-(5-((6S,8R)-8-methyl-7- (2,2,2-trifluoroethyl)-6,7,8,9-tetrahydro-3H- pyrazolo[4,3-f]isoquinolin-6-yl)pyridin-2-yl)- 1-oxa-8-azaspiro[4.5]decan-3-yl)methyl)-1- oxo-1,3,5,5a,6,7,8,9-octahydro-2H- pyrazino[1′,2′:4,5][1,4]oxazino[2,3-e]isoindol- 2-yl)piperidine-2,6-dione |
| A23 | 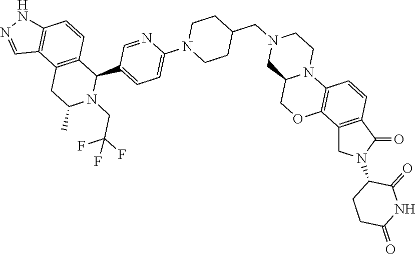 | (S)-3-((R)-7-((1-(5-((6S,8R)-8-methyl-7- (2,2,2-trifluoroethyl)-6,7,8,9-tetrahydro-3H- pyrazolo[4,3-f]isoquinolin-6-yl)pyridin-2- yl)piperidin-4-yl)methyl)-1-oxo- 1,3,5,5a,6,7,8,9-octahydro-2H- pyrazino[1′,2′:4,5][1,4]oxazino[2,3-c]isoindol- 2-yl)piperidine-2,6-dione |
| A24 | 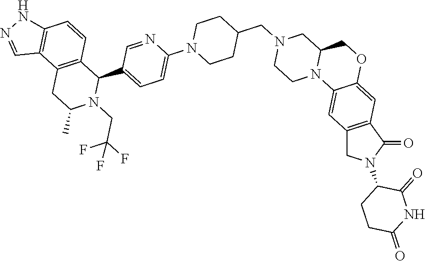 | (R)-3-((R)-3-((1-(5-((6R,8S)-8-methyl-7- (2,2,2-trifluoroethyl)-6,7,8,9-tetrahydro-3H- pyrazolo[4,3-f]isoquinolin-6-yl)pyridin-2- yl)piperidin-4-yl)methyl)-8-oxo- 1,2,3,4,4a,5,8,10-octahydro-9H- pyrazino[1′,2′:4,5][1,4]oxazino[2,3-f]isoindol- 9-yl)piperidine-2,6-dione |
| A25 | 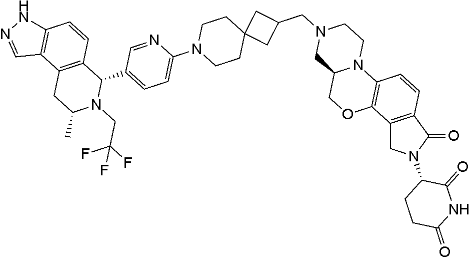 [SMILES: C[C@@H]1Cc2c(ccc3[nH]ncc23)[C@H](c2ccc(N3CCC4(CC3)CC(CN3CCN5c6ccc7c(c6OC[C@H]5C3)CN([C@H]3CCC(=O)NC3=O)C7=O)C4)nc2)N1CC(F)(F)F] | (R)-3-((S)-7-((7-(5-((6S,8S)-8-methyl-7- (2,2,2-trifluoroethyl)-6,7,8,9-tetrahydro-3H- pyrazolo[4,3-f]isoquinolin-6-yl)pyridin-2-yl)- 7-azaspiro[3.5]nonan-2-yl)methyl)-1-oxo- 1,3,5,5a,6,7,8,9-octahydro-2H- pyrazino[1′,2′:4,5][1,4]oxazino[2,3-e]isoindol- 2-yl)piperidine-2,6-dione |
| A26 | 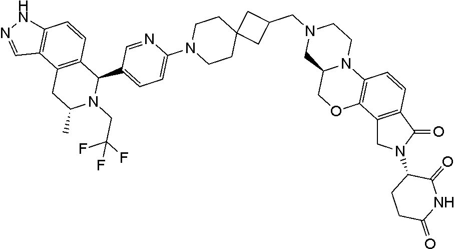 [SMILES: C[C@@H]1Cc2c(ccc3[nH]ncc23)[C@@H](c2ccc(N3CCC4(CC3)CC(CN3CCN5c6ccc7c(c6OC[C@H]5C3)CN([C@H]3CCC(=O)NC3=O)C7=O)C4)nc2)N1CC(F)(F)F] | (R)-3-((S)-7-((7-(5-((6R,8S)-8-methyl-7- (2,2,2-trifluoroethyl)-6,7,8,9-tetrahydro-3H- pyrazolo[4,3-f]isoquinolin-6-yl)pyridin-2-yl)- 7-azaspiro[3.5]nonan-2-yl)methyl)-1-oxo- 1,3,5,5a,6,7,8,9-octahydro-2H- pyrazino[1′,2′:4,5][1,4]oxazino[2,3-e]isoindol- 2-yl)piperidine-2,6-dione |

TABLE 2
Selected Compound of B1-B126

| Compound No | Chemical Structure | Chemical Name |
|---|---|---|
| B16 | 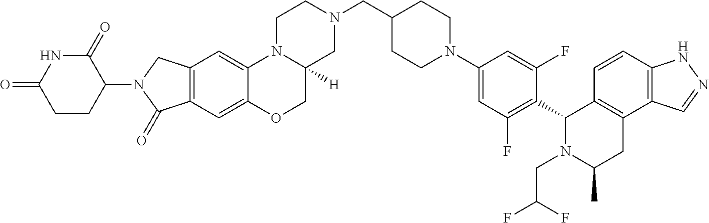 [SMILES: C[C@@H]1Cc2c(ccc3[nH]ncc23)[C@@H](c2c(F)cc(N3CCC(CN4CCN5c6cc7c(cc6OC[C@@H]5C4)C(=O)N(C4CCC(=O)NC4=O)C7)CC3)cc2F)N1CC(F)F] | 3-((S)-3-((1-(4-((6S,8R)-7-(2,2- difluoroethyl)-8-methyl-6,7,8,9- tetrahydro-3H-pyrazolo[4,3- flisoquinolin-6-yl)-3,5- difluorophenyl)piperidin-4- yl)methyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B18 | 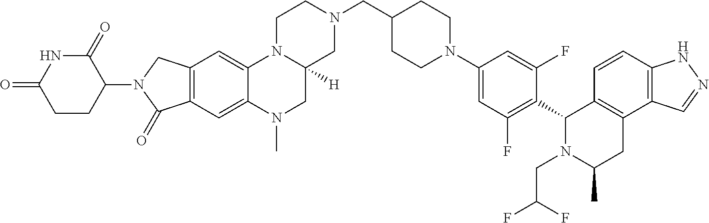 [SMILES: C[C@@H]1Cc2c(ccc3[nH]ncc23)[C@@H](c2c(F)cc(N3CCC(CN4CCN5c6cc7c(cc6N(C)C[C@@H]5C4)C(=O)N(C4CCC(=O)NC4=O)C7)CC3)cc2F)N1CC(F)F] | 3-((S)-3-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-6-methyl-8-oxo- 2,3,4,4a,5,6,8,10- octahydropyrazino[1,2- a]pyrrolo[3,4-g]quinoxalin-9(1H)- yl)piperidine-2,6-dione |
| B20 | 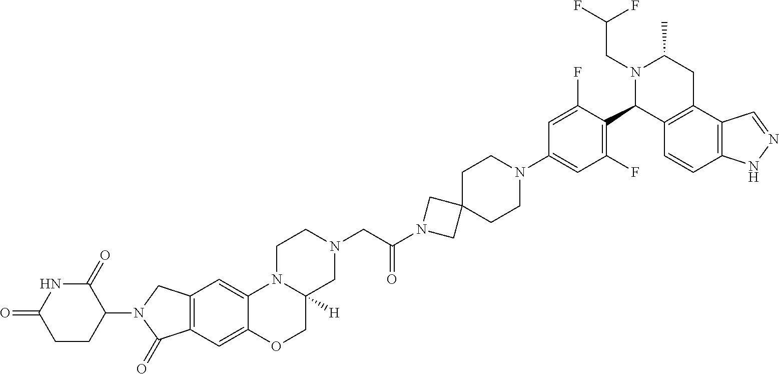 [SMILES: C[C@@H]1Cc2c(ccc3[nH]ncc23)[C@@H](c2c(F)cc(N3CCC4(CC3)CN(C(=O)CN3CCN5c6cc7c(cc6OC[C@@H]5C3)C(=O)N(C3CCC(=O)NC3=O)C7)C4)cc2F)N1CC(F)F] | 3-((S)-3-(2-(7-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,7- diazaspiro[3.5]nonan-2-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B21 | 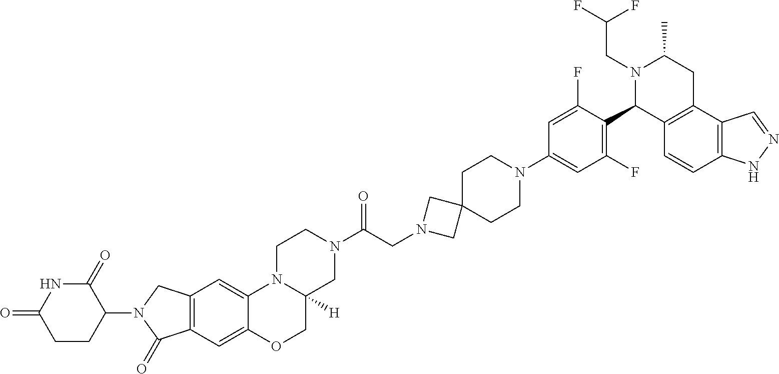 [SMILES: C[C@@H]1Cc2c(ccc3[nH]ncc23)[C@@H](c2c(F)cc(N3CCC4(CC3)CN(CC(=O)N3CCN5c6cc7c(cc6OC[C@@H]5C3)C(=O)N(C3CCC(=O)NC3=O)C7)C4)cc2F)N1CC(F)F] | 3-((S)-3-(2-(7-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,7- diazaspiro[3.5]nonan-2-yl)acetyl)- 8-oxo-1,2,3,4,4a,5,8,10-octahydro- 9H- pyrazino[1′,2′:4,5][1,4]oxazino[2,3- f]isoindol-9-yl)piperidine-2,6- dione |
| B22 | 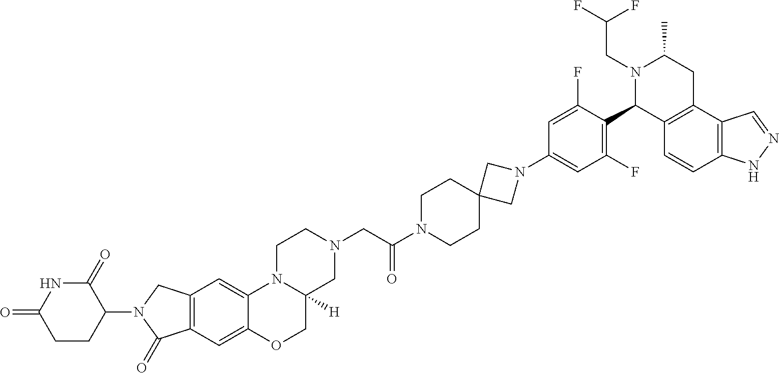 [SMILES: C[C@@H]1Cc2c(ccc3[nH]ncc23)[C@@H](c2c(F)cc(N3CC4(CCN(C(=O)CN5CCN6c7cc8c(cc7OC[C@@H]6C5)C(=O)N(C5CCC(=O)NC5=O)C8)CC4)C3)cc2F)N1CC(F)F] | 3-((S)-3-(2-(2-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,7- diazaspiro[3.5]nonan-7-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B23 | 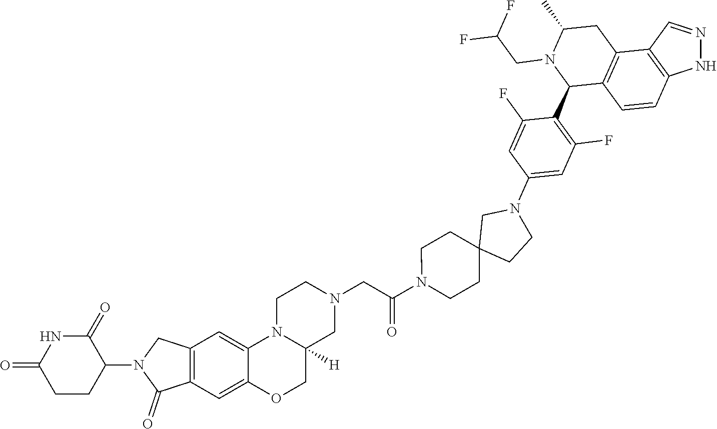 [SMILES: C[C@@H]1Cc2c(ccc3[nH]ncc23)[C@@H](c2c(F)cc(N3CCC4(CCN(C(=O)CN5CCN6c7cc8c(cc7OC[C@@H]6C5)C(=O)N(C5CCC(=O)NC5=O)C8)CC4)C3)cc2F)N1CC(F)F] | 3-((S)-3-(2-(2-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,8- diazaspiro[4.5]decan-8-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B26 | 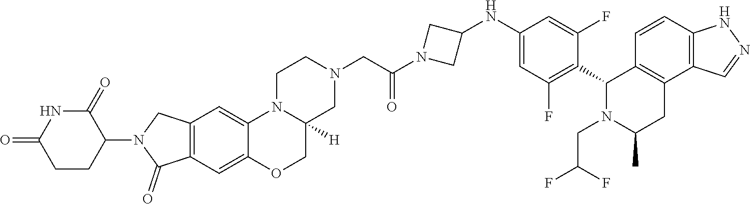 [SMILES: C[C@@H]1Cc2c(ccc3[nH]ncc23)[C@@H](c2c(F)cc(NC3CN(C(=O)CN4CCN5c6cc7c(cc6OC[C@@H]5C4)C(=O)N(C4CCC(=O)NC4=O)C7)C3)cc2F)N1CC(F)F] | 3-((S)-3-(2-(3-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)azetidin-1- yl)-2-oxoethyl)-8-oxo- 1,2,3,4,4a,5,8,10-octahydro-9H- pyrazino[1′,2′:4,5][1,4]oxazino[2,3- f]isoindol-9-yl)piperidine-2,6- dione |
| B27 | 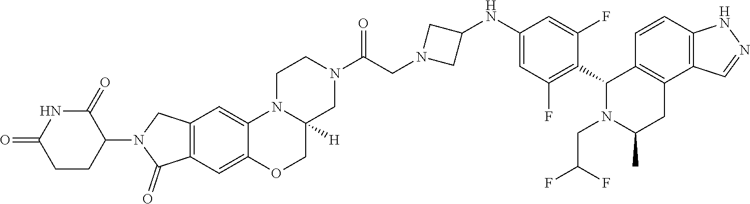 [SMILES: C[C@@H]1Cc2c(ccc3[nH]ncc23)[C@@H](c2c(F)cc(NC3CN(CC(=O)N4CCN5c6cc7c(cc6OC[C@@H]5C4)C(=O)N(C4CCC(=O)NC4=O)C7)C3)cc2F)N1CC(F)F] | 3-((S)-3-(2-(3-((4-((6S,8R)-7-(2,2- difluoroethyl)-8-methyl-6,7,8,9- tetrahydro-3H-pyrazolo[4,3- flisoquinolin-6-yl)-3,5- difluorophenyl)amino)azetidin-1- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B28 | 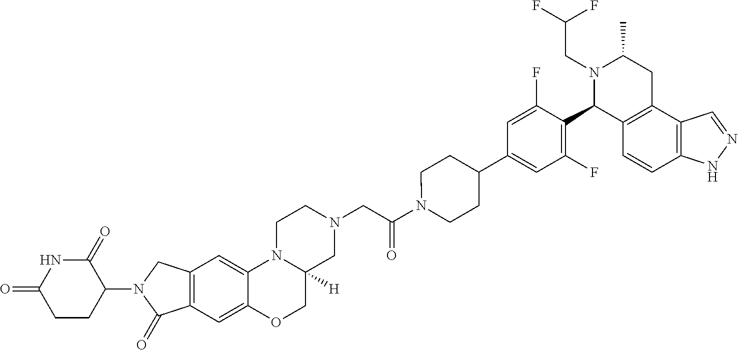 [SMILES: C[C@@H]1Cc2c(ccc3[nH]ncc23)[C@@H](c2c(F)cc(C3CCN(C(=O)CN4CCN5c6cc7c(cc6OC[C@@H]5C4)C(=O)N(C4CCC(=O)NC4=O)C7)CC3)cc2F)N1CC(F)F] | 3-((S)-3-(2-(4-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-1-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B29 | 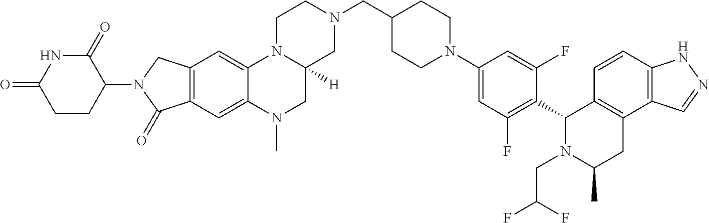 [SMILES: C[C@@H]1Cc2c(ccc3[nH]ncc23)[C@@H](c2c(F)cc(N3CCC(CN4CCN5c6cc7c(cc6N(C)C[C@@H]5C4)C(=O)N(C4CCC(=O)NC4=O)C7)CC3)cc2F)N1CC(F)F] | 3-((S)-3-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-6-methyl-8-oxo- 2,3,4,4a,5,6,8,10- octahydropyrazino[1,2- a]pyrrolo[3,4-g]quinoxalin-9(1H)- yl)piperidine-2,6-dione |
| B30 | 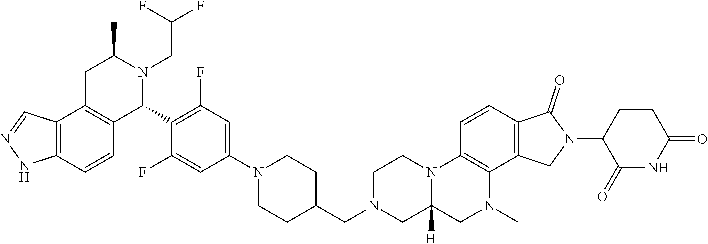 [SMILES: C[C@@H]1Cc2c(ccc3[nH]ncc23)[C@@H](c2c(F)cc(N3CCC(CN4CCN5c6ccc7c(c6N(C)C[C@@H]5C4)CN(C4CCC(=O)NC4=O)C7=O)CC3)cc2F)N1CC(F)F] | 3-((S)-7-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-4-methyl-1-oxo- 3,4,5,5a,6,7,8,9- octahydropyrazino[1,2- a]pyrrolo[3,4-f]quinoxalin-2(1H)- yl)piperidine-2,6-dione |
| B31 | 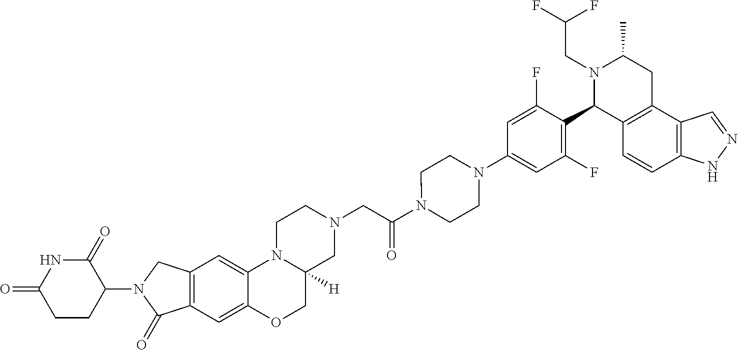 [SMILES: C[C@@H]1Cc2c(ccc3[nH]ncc23)[C@@H](c2c(F)cc(N3CCN(C(=O)CN4CCN5c6cc7c(cc6OC[C@@H]5C4)C(=O)N(C4CCC(=O)NC4=O)C7)CC3)cc2F)N1CC(F)F] | 3-((S)-3-(2-(4-(4-((6S,8R)-7-(2,2- difluoroethyl)-8-methyl-6,7,8,9- tetrahydro-3H-pyrazolo[4,3- flisoquinolin-6-yl)-3,5- difluorophenyl)piperazin-1-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B32 | 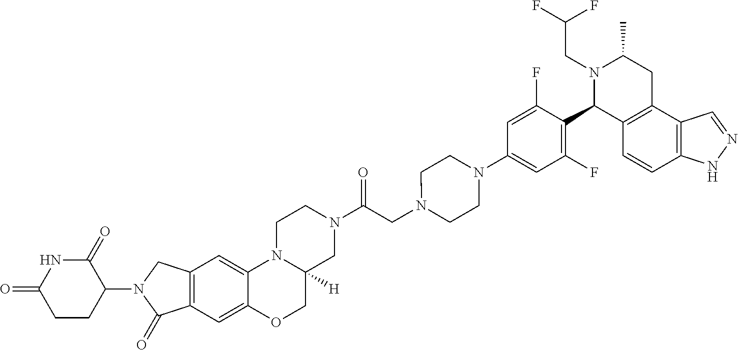 [SMILES: C[C@@H]1Cc2c(ccc3[nH]ncc23)[C@@H](c2c(F)cc(N3CCN(CC(=O)N4CCN5c6cc7c(cc6OC[C@@H]5C4)C(=O)N(C4CCC(=O)NC4=O)C7)CC3)cc2F)N1CC(F)F] | 3-((S)-3-(2-(4-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)pipcrazin-1- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B33 | 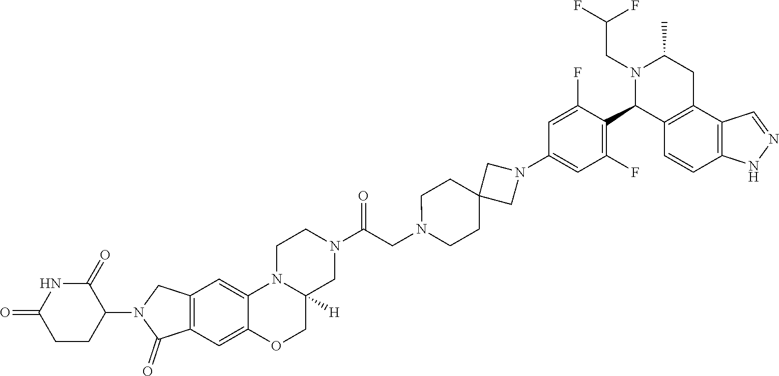 [SMILES: C[C@@H]1Cc2c(ccc3[nH]ncc23)[C@@H](c2c(F)cc(N3CC4(CCN(CC(=O)N5CCN6c7cc8c(cc7OC[C@@H]6C5)C(=O)N(C5CCC(=O)NC5=O)C8)CC4)C3)cc2F)N1CC(F)F] | 3-((S)-3-(2-(2-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,7- diazaspiro[3.5]nonan-7-yl)acetyl)- 8-oxo-1,2,3,4,4a,5,8,10-octahydro- 9H- pyrazino[1′,2′:4,5][1,4]oxazino[2,3- f]isoindol-9-yl)piperidine-2,6- dione |
| B34 | 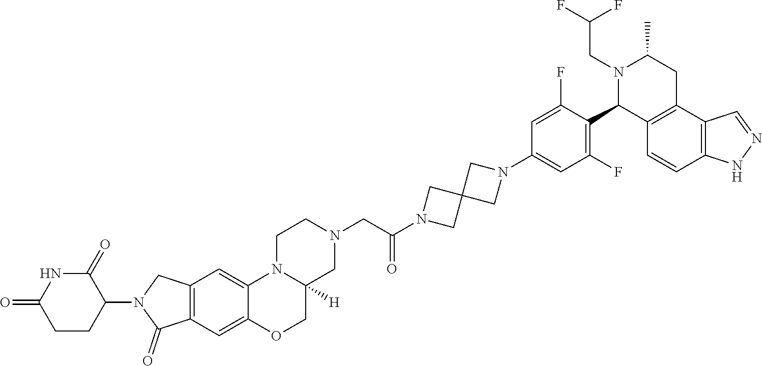 [SMILES: C[C@@H]1Cc2c(ccc3[nH]ncc23)[C@@H](c2c(F)cc(N3CC4(CN(C(=O)CN5CCN6c7cc8c(cc7OC[C@@H]6C5)C(=O)N(C5CCC(=O)NC5=O)C8)C4)C3)cc2F)N1CC(F)F] | 3-((S)-3-(2-(6-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,6- diazaspiro[3.3]heptan-2-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B35 | 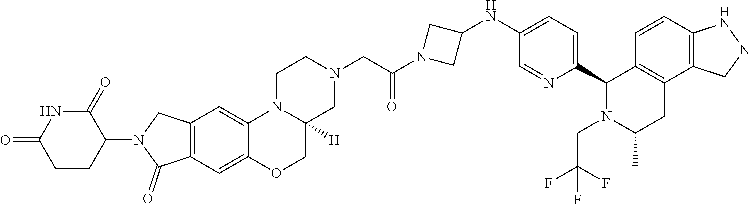 [SMILES: C[C@H]1Cc2c(ccc3c2CNN3)[C@H](c2ccc(NC3CN(C(=O)CN4CCN5c6cc7c(cc6OC[C@@H]5C4)C(=O)N(C4CCC(=O)NC4=O)C7)C3)cn2)N1CC(F)(F)F] | 3-((S)-3-(2-(3-((6-((6R,8S)-8- methyl-7-(2,2,2-trifluoroethyl)- 6,7,8,9-tetrahydro-3H- pyrazolo[4,3-f]isoquinolin-6- yl)pyridin-3-yl)amino)azetidin-1- yl)-2-oxoethyl)-8-oxo- 1,2,3,4,4a,5,8,10-octahydro-9H- pyrazino[1′,2′:4,5][1,4]oxazino[2,3- f]isoindol-9-yl)piperidine-2,6- dione |
| B36 | 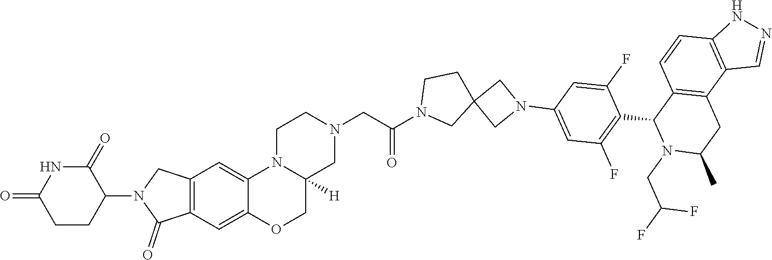 [SMILES: C[C@@H]1Cc2c(ccc3[nH]ncc23)[C@@H](c2c(F)cc(N3CC4(CCN(C(=O)CN5CCN6c7cc8c(cc7OC[C@@H]6C5)C(=O)N(C5CCC(=O)NC5=O)C8)C4)C3)cc2F)N1CC(F)F] | 3-((S)-3-(2-(2-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,6- diazaspiro[3.4]octan-6-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B37 | 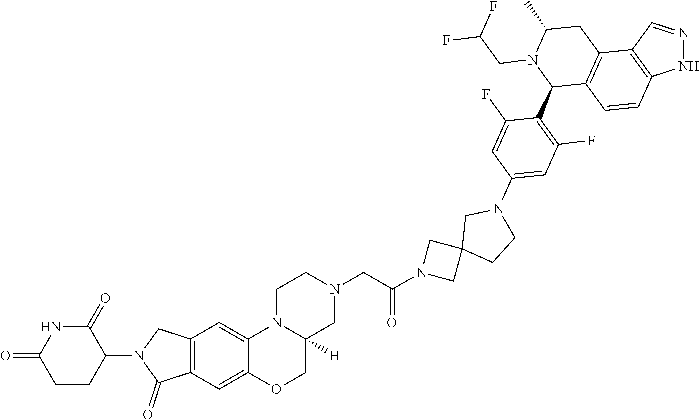 [SMILES: C[C@@H]1Cc2c(ccc3[nH]ncc23)[C@@H](c2c(F)cc(N3CCC4(CN(C(=O)CN5CCN6c7cc8c(cc7OC[C@@H]6C5)C(=O)N(C5CCC(=O)NC5=O)C8)C4)C3)cc2F)N1CC(F)F] | 3-((S)-3-(2-(6-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,6- diazaspiro[3.4]octan-2-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B38 | 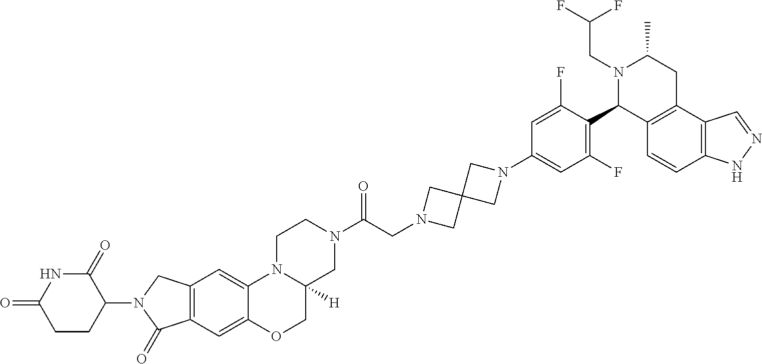 [SMILES: C[C@@H]1Cc2c(ccc3[nH]ncc23)[C@@H](c2c(F)cc(N3CC4(CN(CC(=O)N5CCN6c7cc8c(cc7OC[C@@H]6C5)C(=O)N(C5CCC(=O)NC5=O)C8)C4)C3)cc2F)N1CC(F)F] | 3-((S)-3-(2-(6-(4-((6S,8R)-7-(2,2- difluorocthyl)-8-mcthyl-6,7,8,9- tetrahydro-3H-pyrazolo[4,3- f]isoquinolin-6-yl)-3,5- difluorophenyl)-2,6- diazaspiro[3.3]heptan-2-yl)acetyl)- 8-oxo-1,2,3,4,4a,5,8,10-octahydro- 9H- pyrazino[1′,2′:4,5][1,4]oxazino[2,3- f]isoindol-9-yl)piperidine-2,6- dione |
| B39 | 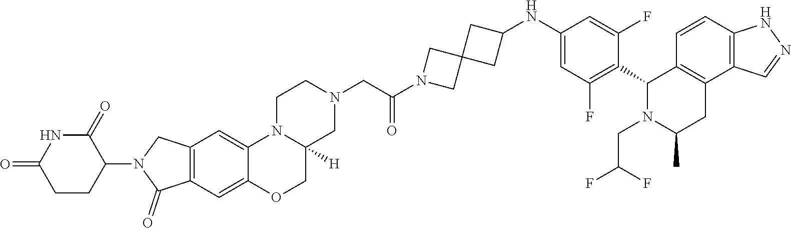 [SMILES: C[C@@H]1Cc2c(ccc3[nH]ncc23)[C@@H](c2c(F)cc(NC3CC4(C3)CN(C(=O)CN3CCN5c6cc7c(cc6OC[C@@H]5C3)C(=O)N(C3CCC(=O)NC3=O)C7)C4)cc2F)N1CC(F)F] | 3-((S)-3-(2-(6-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)-2- azaspiro[3.3]heptan-2-yl)-2- oxoethyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B40 | 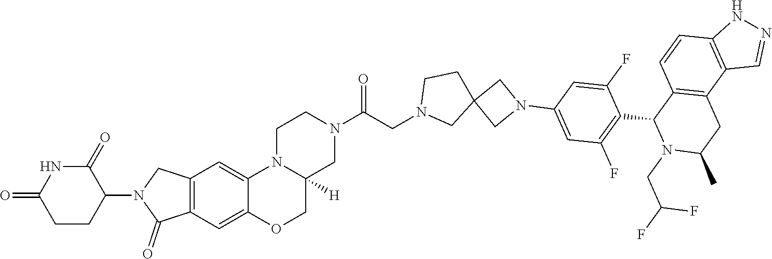 [SMILES: C[C@@H]1Cc2c(ccc3[nH]ncc23)[C@@H](c2c(F)cc(N3CC4(CCN(CC(=O)N5CCN6c7cc8c(cc7OC[C@@H]6C5)C(=O)N(C5CCC(=O)NC5=O)C8)C4)C3)cc2F)N1CC(F)F] | 3-((S)-3-(2-(2-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,6- diazaspiro[3.4]octan-6-yl)acetyl)- 8-oxo-1,2,3,4,4a,5,8,10-octahydro- 9H- pyrazino[1′,2′:4,5][1,4]oxazino[2,3- f]isoindol-9-yl)piperidine-2,6- dione |
| B41 | 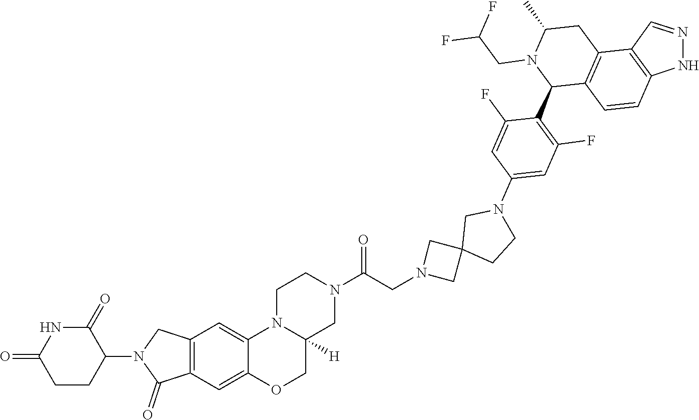 [SMILES: C[C@@H]1Cc2c(ccc3[nH]ncc23)[C@@H](c2c(F)cc(N3CCC4(CN(CC(=O)N5CCN6c7cc8c(cc7OC[C@@H]6C5)C(=O)N(C5CCC(=O)NC5=O)C8)C4)C3)cc2F)N1CC(F)F] | 3-((S)-3-(2-(6-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,6- diazaspiro[3.4]octan-2-yl)acetyl)- 8-oxo-1,2,3,4,4a,5,8,10-octahydro- 9H- pyrazino[1′,2′:4,5][1,4]oxazino[2,3- f]isoindol-9-yl)piperidine-2,6- dione |
| B42 | 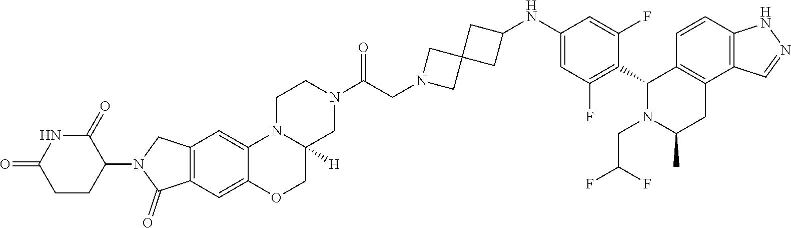 [SMILES: C[C@@H]1Cc2c(ccc3[nH]ncc23)[C@@H](c2c(F)cc(NC3CC4(C3)CN(CC(=O)N3CCN5c6cc7c(cc6OC[C@@H]5C3)C(=O)N(C3CCC(=O)NC3=O)C7)C4)cc2F)N1CC(F)F] | 3-((S)-3-(2-(6-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)-2- azaspiro[3.3]heptan-2-yl)acetyl)-8- oxo-1,2,3,4,4a,5,8,10-octahydro- 9H- pyrazino[1′,2′:4,5][1,4]oxazino[2,3- f]isoindol-9-yl)piperidine-2,6- dione |
| B43 | 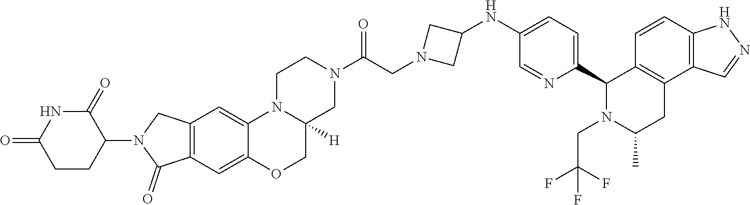 [SMILES: C[C@H]1Cc2c(ccc3[nH]ncc23)[C@H](c2ccc(NC3CN(CC(=O)N4CCN5c6cc7c(cc6OC[C@@H]5C4)C(=O)N(C4CCC(=O)NC4=O)C7)C3)cn2)N1CC(F)(F)F] | 3-((S)-3-(2-(3-((6-((6R,8S)-8- methyl-7-(2,2,2-trifluoroethyl)- 6,7,8,9-tetrahydro-3H- pyrazolo[4,3-f]isoquinolin-6- yl)pyridin-3-yl)amino)azetidin-1- yl)acetyl)-8-oxo-1,2,3,4,4a,5,8,10- octahydro-9H- pyrazino[1′,2′:4,5][1,4]oxazino[2,3- f]isoindol-9-yl)piperidine-2,6- dione |
| B46 | 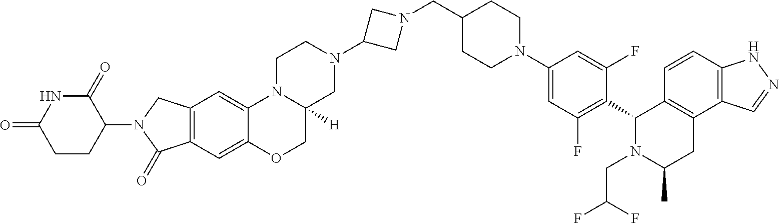 [SMILES: C[C@@H]1Cc2c(ccc3[nH]ncc23)[C@@H](c2c(F)cc(N3CCC(CN4CC(N5CCN6c7cc8c(cc7OC[C@@H]6C5)C(=O)N(C5CCC(=O)NC5=O)C8)C4)CC3)cc2F)N1CC(F)F] | 3-((S)-3-(1-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)azetidin-3-yl)-8-oxo- 1,2,3,4,4a,5,8,10-octahydro-9H- pyrazino[1′,2′:4,5][1,4]oxazino[2,3- f]isoindol-9-yl)piperidine-2,6- dione |
| B47 | 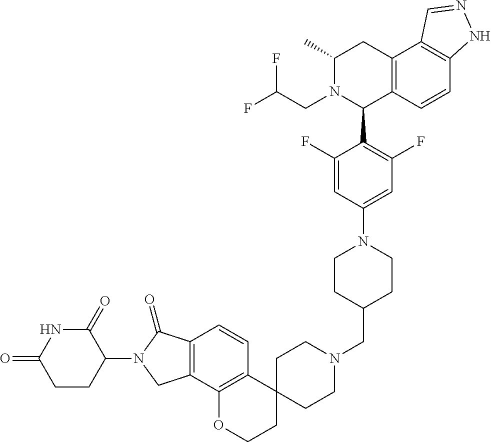 [SMILES: C[C@@H]1Cc2c(ccc3[nH]ncc23)[C@@H](c2c(F)cc(N3CCC(CN4CCC5(CCOc6c5ccc5c6CN(C6CCC(=O)NC6=O)C5=O)CC4)CC3)cc2F)N1CC(F)F] | 3-(1-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-7′-oxo-2′,3′,7′,9′- tetrahydro-8′H-spiro[piperidine- 4,4′-pyrano[2,3-e]isoindol]-8′- yl)piperidine-2,6-dione |
| B52 | 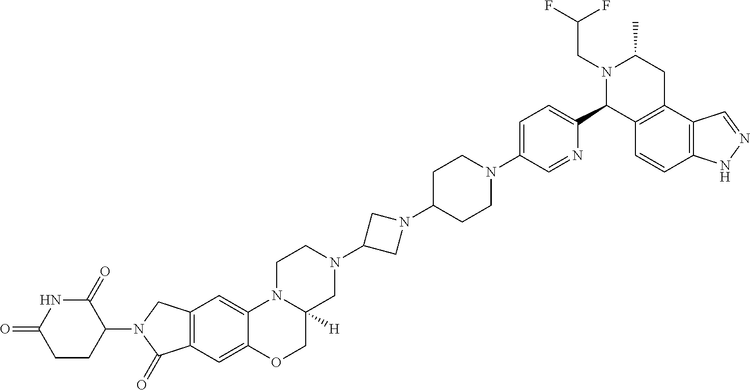 [SMILES: C[C@@H]1Cc2c(ccc3[nH]ncc23)[C@@H](c2ccc(N3CCC(N4CC(N5CCN6c7cc8c(cc7OC[C@@H]6C5)C(=O)N(C5CCC(=O)NC5=O)C8)C4)CC3)cn2)N1CC(F)F] | 3-((S)-3-(1-(1-(6-((6S,8R)-8- methyl-7-(2,2,2-trifluoroethyl)- 6,7,8,9-tetrahydro-3H- pyrazolo[4,3-f]isoquinolin-6- yl)pyridin-3-yl)piperidin-4- yl)azetidin-3-yl)-8-oxo- 1,2,3,4,4a,5,8,10-octahydro-9H- pyrazino[1′,2′:4,5][1,4]oxazino[2,3- f]isoindol-9-yl)piperidine-2,6- dione |
| B53 | 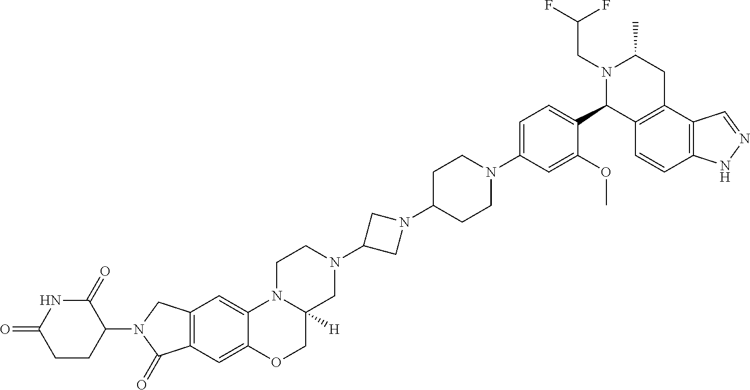 [SMILES: COc1cc(N2CCC(N3CC(N4CCN5c6cc7c(cc6OC[C@@H]5C4)C(=O)N(C4CCC(=O)NC4=O)C7)C3)CC2)ccc1[C@@H]1c2ccc3[nH]ncc3c2C[C@@H](C)N1CC(F)F] | 3-((S)-3-(1-(1-(4-((6S,8R)-7-(2,2- difluoroethyl)-8-methyl-6,7,8,9- tetrahydro-3H-pyrazolo[4,3- f]isoquinolin-6-yl)-3- methoxyphenyl)piperidin-4- yl)azetidin-3-yl)-8-oxo- 1,2,3,4,4a,5,8,10-octahydro-9H- pyrazino[1′,2′:4,5][1,4]oxazino[2,3- f]isoindol-9-yl)piperidine-2,6- dione |
| B54 | 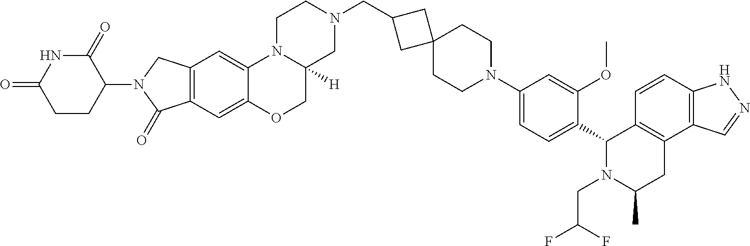 [SMILES: COc1cc(N2CCC3(CC2)CC(CN2CCN4c5cc6c(cc5OC[C@@H]4C2)C(=O)N(C2CCC(=O)NC2=O)C6)C3)ccc1[C@@H]1c2ccc3[nH]ncc3c2C[C@@H](C)N1CC(F)F] | 3-((S)-3-((7-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-7- azaspiro[3.5]nonan-2-yl)methyl)-8- oxo-1,2,3,4,4a,5,8,10-octahydro- 9H- pyrazino[1′,2′:4,5][1,4]oxazino[2,3- f]isoindol-9-yl)piperidine-2,6- dione |
| B55 | 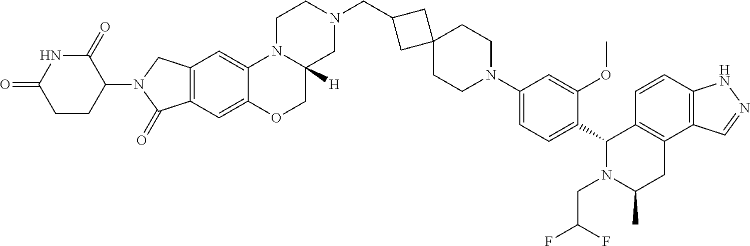 [SMILES: COc1cc(N2CCC3(CC2)CC(CN2CCN4c5cc6c(cc5OC[C@H]4C2)C(=O)N(C2CCC(=O)NC2=O)C6)C3)ccc1[C@@H]1c2ccc3[nH]ncc3c2C[C@@H](C)N1CC(F)F] | 3-((R)-3-((7-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-7- azaspiro[3.5]nonan-2-yl)methyl)-8- oxo-1,2,3,4,4a,5,8,10-octahydro- 9H- pyrazino[1′,2′:4,5][1,4]oxazino[2,3- f]isoindol-9-yl)piperidine-2,6- dione |
| B56 | 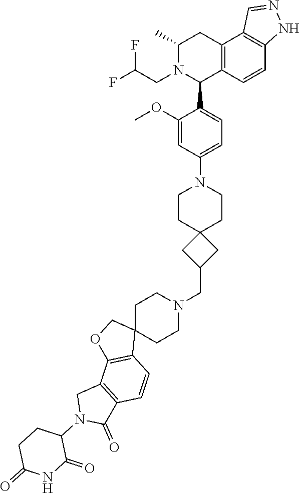 [SMILES: COc1cc(N2CCC3(CC2)CC(CN2CCC4(CC2)COc2c4ccc4c2CN(C2CCC(=O)NC2=O)C4=O)C3)ccc1[C@@H]1c2ccc3[nH]ncc3c2C[C@@H](C)N1CC(F)F] | 3-(1′-((7-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-7- azaspiro[3.5]nonan-2-yl)methyl)-6- oxo-6,8-dihydro-2H,7H- spiro[furo[2,3-e]isoindole-3,4′- piperidin]-7-yl)piperidine-2,6- dione |
| B57 | 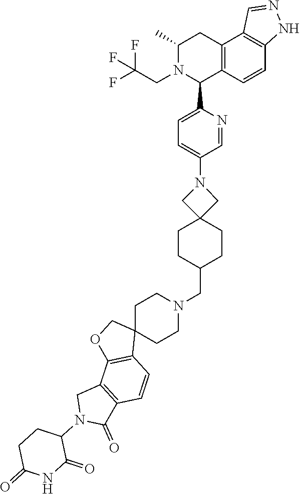 [SMILES: C[C@@H]1Cc2c(ccc3[nH]ncc23)[C@@H](c2ccc(N3CC4(CCC(CN5CCC6(CC5)COc5c6ccc6c5CN(C5CCC(=O)NC5=O)C6=O)CC4)C3)cn2)N1CC(F)(F)F] | 3-(1′-((2-(6-((6S,8R)-8-mcthyl-7- (2,2,2-trifluoroethyl)-6,7,8,9- tetrahydro-3H-pyrazolo[4,3- f]isoquinolin-6-yl)pyridin-3-yl)-2- azaspiro[3.5]nonan-7-yl)methyl)-6- oxo-6,8-dihydro-2H,7H- spiro[furo[2,3-e]isoindole-3,4′- piperidin]-7-yl)piperidine-2,6- dione |
| B58 | 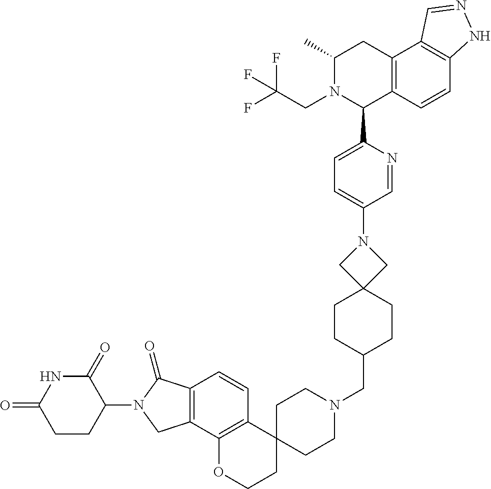 [SMILES: C[C@@H]1Cc2c(ccc3[nH]ncc23)[C@@H](c2ccc(N3CC4(CCC(CN5CCC6(CCOc7c6ccc6c7CN(C7CCC(=O)NC7=O)C6=O)CC5)CC4)C3)cn2)N1CC(F)(F)F] | 3-(1-((2-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)-2- azaspiro[3.5]nonan-7-yl)methyl)- 7′-oxo-2′,3′,7′,9′-tetrahydro-8′H- spiro[piperidine-4,4′-pyrano[2,3- e]isoindol]-8′-yl)piperidine-2,6- dione |
| B59 | 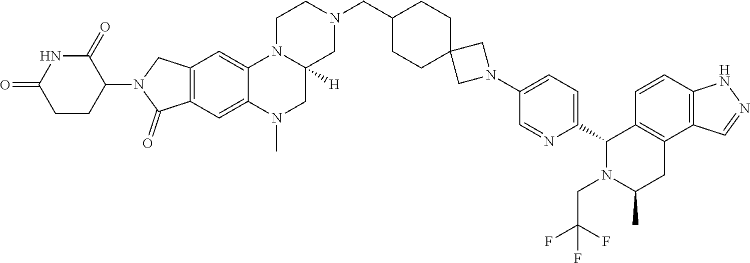 [SMILES: C[C@@H]1Cc2c(ccc3[nH]ncc23)[C@@H](c2ccc(N3CC4(CCC(CN5CCN6c7cc8c(cc7N(C)C[C@@H]6C5)C(=O)N(C5CCC(=O)NC5=O)C8)CC4)C3)cn2)N1CC(F)(F)F] | 3-((S)-6-methyl-3-((2-(6-((6S,8R)- 8-methyl-7-(2,2,2-trifluoroethyl)- 6,7,8,9-tetrahydro-3H- pyrazolo[4,3-f]isoquinolin-6- yl)pyridin-3-yl)-2- azaspiro[3.5]nonan-7-yl)methyl)-8- oxo-2,3,4,4a,5,6,8,10- octahydropyrazino[1,2- a]pyrrolo[3,4-g]quinoxalin-9(1H)- yl)piperidine-2,6-dione |
| B60 | 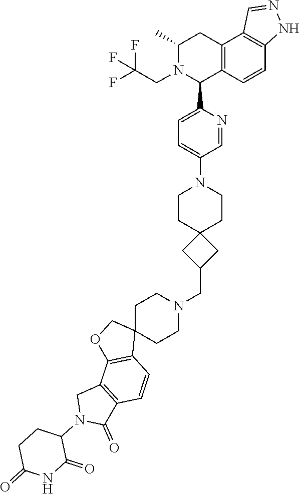 [SMILES: C[C@@H]1Cc2c(ccc3[nH]ncc23)[C@@H](c2ccc(N3CCC4(CC3)CC(CN3CCC5(CC3)COc3c5ccc5c3CN(C3CCC(=O)NC3=O)C5=O)C4)cn2)N1CC(F)(F)F] | 3-(1′-((7-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)-7- azaspiro[3.5]nonan-2-yl)methyl)-6- oxo-6,8-dihydro-2H,7H- spiro[furo[2,3-e]isoindole-3,4′- piperidin]-7-yl)piperidine-2,6- dione |
| B61 | 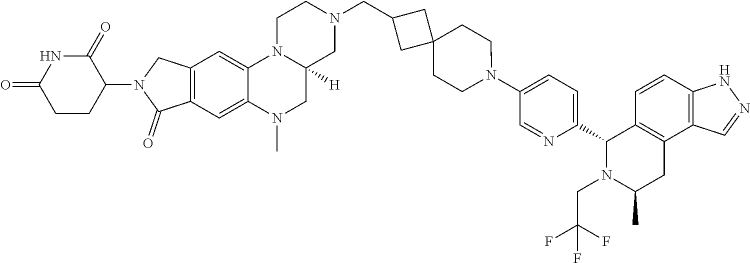 [SMILES: C[C@@H]1Cc2c(ccc3[nH]ncc23)[C@@H](c2ccc(N3CCC4(CC3)CC(CN3CCN5c6cc7c(cc6N(C)C[C@@H]5C3)C(=O)N(C3CCC(=O)NC3=O)C7)C4)cn2)N1CC(F)(F)F] | 3-((S)-6-methyl-3-((7-(6-((6S,8R)- 8-methyl-7-(2,2,2-trifluoroethyl)- 6,7,8,9-tetrahydro-3H- pyrazolo[4,3-f]isoquinolin-6- yl)pyridin-3-yl)-7- azaspiro[3.5]nonan-2-yl)methyl)-8- oxo-2,3,4,4a,5,6,8,10- octahydropyrazino[1,2- a]pyrrolo[3,4-g]quinoxalin-9(1H)- yl)piperidine-2,6-dione |
| B62 | 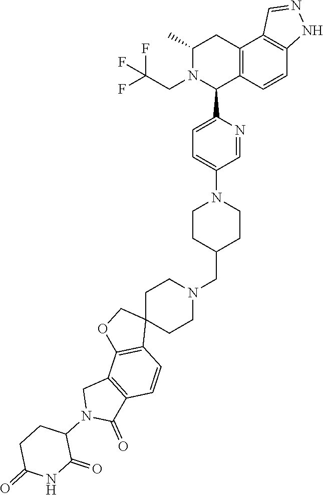 [SMILES: C[C@@H]1Cc2c(ccc3[nH]ncc23)[C@@H](c2ccc(N3CCC(CN4CCC5(CC4)COc4c5ccc5c4CN(C4CCC(=O)NC4=O)C5=O)CC3)cn2)N1CC(F)(F)F] | 3-(1′-((1-(6-((6S,8R)-8-methyl-7- (2,2,2-trifluoroethyl)-6,7,8,9- tetrahydro-3H-pyrazolo[4,3- f]isoquinolin-6-yl)pyridin-3- yl)piperidin-4-yl)methyl)-6-oxo- 6,8-dihydro-2H,7H-spiro[furo[2,3- e]isoindole-3,4′-piperidin]-7- yl)piperidine-2,6-dione |
| B63 | 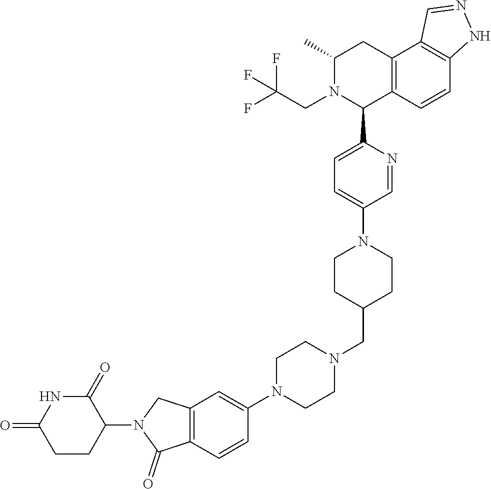 [SMILES: C[C@@H]1Cc2c(ccc3[nH]ncc23)[C@@H](c2ccc(N3CCC(CN4CCN(c5ccc6c(c5)CN(C5CCC(=O)NC5=O)C6=O)CC4)CC3)cn2)N1CC(F)(F)F] | 3-(5-(4-((1-(6-((6S,8R)-8-methyl- 7-(2,2,2-trifluoroethyl)-6,7,8,9- tetrahydro-3H-pyrazolo[4,3- f]isoquinolin-6-yl)pyridin-3- yl)piperidin-4-yl)methyl)piperazin- 1-yl)-1-oxoisoindolin-2- yl)piperidine-2,6-dione |
| B64 | 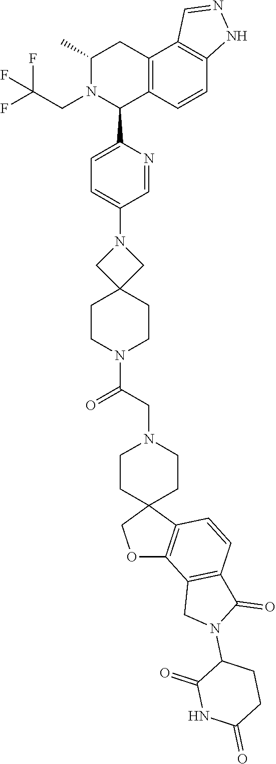 [SMILES: C[C@@H]1Cc2c(ccc3[nH]ncc23)[C@@H](c2ccc(N3CC4(CCN(C(=O)CN5CCC6(CC5)COc5c6ccc6c5CN(C5CCC(=O)NC5=O)C6=O)CC4)C3)cn2)N1CC(F)(F)F] | 3-(1′-(2-(2-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)- 2,7-diazaspiro[3.5]nonan-7-yl)-2- oxoethyl)-6-oxo-6,8-dihydro- 2H,7H-spiro[furo[2,3-e]isoindole- 3,4′-piperidin]-7-yl)piperidine-2,6- dione |
| B65 | 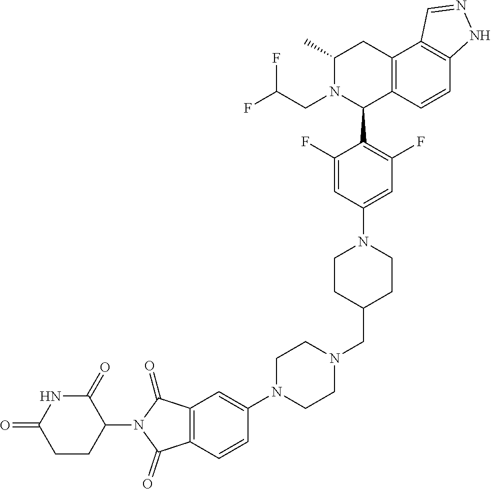 [SMILES: C[C@@H]1Cc2c(ccc3[nH]ncc23)[C@@H](c2c(F)cc(N3CCC(CN4CCN(c5ccc6c(c5)C(=O)N(C5CCC(=O)NC5=O)C6=O)CC4)CC3)cc2F)N1CC(F)F] | 5-(4-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)piperazin-1-yl)-2-(2,6- dioxopiperidin-3-yl)isoindoline- 1,3-dione |
| B66 | 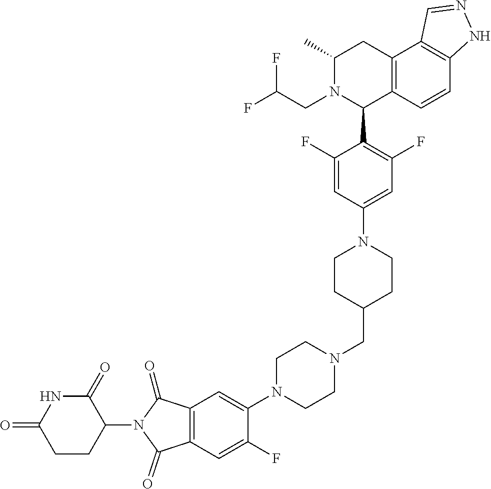 [SMILES: C[C@@H]1Cc2c(ccc3[nH]ncc23)[C@@H](c2c(F)cc(N3CCC(CN4CCN(c5cc6c(cc5F)C(=O)N(C5CCC(=O)NC5=O)C6=O)CC4)CC3)cc2F)N1CC(F)F] | 5-(4-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)mcthyl)pipcrazin-1-yl)-2-(2,6- dioxopiperidin-3-yl)-6- fluoroisoindoline-1,3-dione |
| B67 | 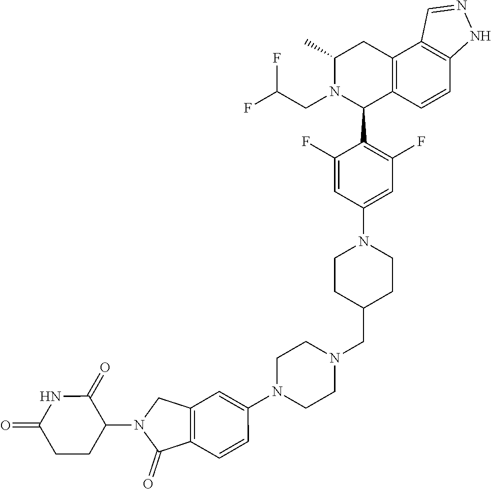 [SMILES: C[C@@H]1Cc2c(ccc3[nH]ncc23)[C@@H](c2c(F)cc(N3CCC(CN4CCN(c5ccc6c(c5)CN(C5CCC(=O)NC5=O)C6=O)CC4)CC3)cc2F)N1CC(F)F] | 3-(5-(4-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)piperazin-1-yl)-1- oxoisoindolin-2-yl)piperidine-2,6- dione |
| B77 | 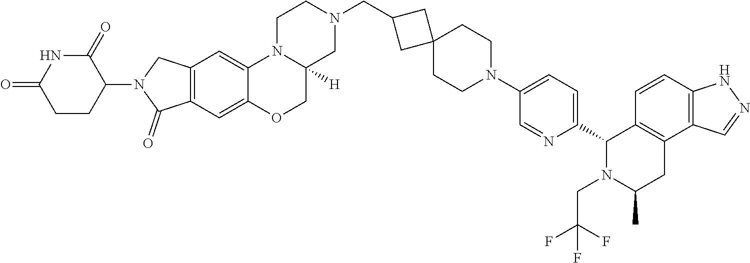 [SMILES: C[C@@H]1Cc2c(ccc3[nH]ncc23)[C@@H](c2ccc(N3CCC4(CC3)CC(CN3CCN5c6cc7c(cc6OC[C@@H]5C3)C(=O)N(C3CCC(=O)NC3=O)C7)C4)cn2)N1CC(F)(F)F] | 3-((S)-3-((7-(6-((6S,8R)-8-methyl- 7-(2,2,2-trifluoroethyl)-6,7,8,9- tetrahydro-3H-pyrazolo[4,3- f]isoquinolin-6-yl)pyridin-3-yl)-7- azaspiro[3.5]nonan-2-yl)methyl)-8- oxo-1,2,3,4,4a,5,8,10-octahydro- 9H- pyrazino[1′,2′:4,5][1,4]oxazino[2,3- f]isoindol-9-yl)piperidine-2,6- dione |
| B78 | 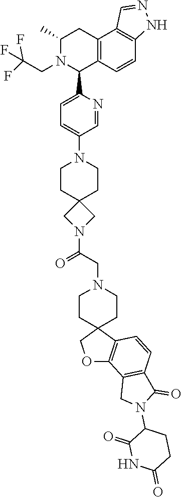 [SMILES: C[C@@H]1Cc2c(ccc3[nH]ncc23)[C@@H](c2ccc(N3CCC4(CC3)CN(C(=O)CN3CCC5(CC3)COc3c5ccc5c3CN(C3CCC(=O)NC3=O)C5=O)C4)cn2)N1CC(F)(F)F] | 3-(1′-(2-(7-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)- 2,7-diazaspiro[3.5]nonan-2-yl)-2- oxoethyl)-6-oxo-6,8-dihydro- 2H,7H-spiro[furo[2,3-e]isoindole- 3,4′-piperidin]-7-yl)piperidine-2,6- dione |
| B79 | 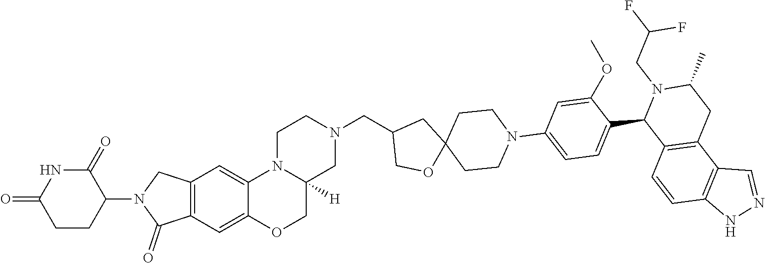 [SMILES: COc1cc(N2CCC3(CC2)CC(CN2CCN4c5cc6c(cc5OC[C@@H]4C2)C(=O)N(C2CCC(=O)NC2=O)C6)CO3)ccc1[C@@H]1c2ccc3[nH]ncc3c2C[C@@H](C)N1CC(F)F] | 3-((4aS)-3-((8-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-1-oxa-8- azaspiro[4.5]decan-3-yl)methyl)-8- oxo-1,2,3,4,4a,5,8,10-octahydro- 9H- pyrazino[1′,2′:4,5][1,4]oxazino[2,3- f]isoindol-9-yl)piperidine-2,6- dione |
| B80 | 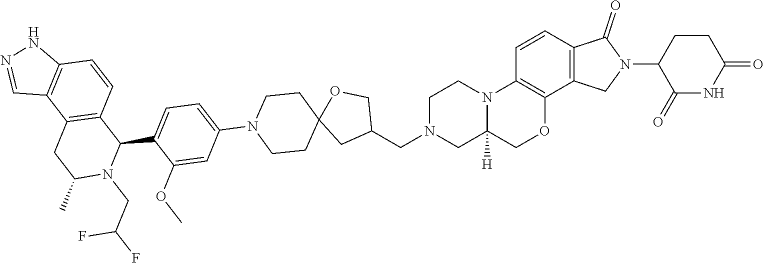 [SMILES: COc1cc(N2CCC3(CC2)CC(CN2CCN4c5ccc6c(c5OC[C@H]4C2)CN(C2CCC(=O)NC2=O)C6=O)CO3)ccc1[C@@H]1c2ccc3[nH]ncc3c2C[C@@H](C)N1CC(F)F] | 3-((5aR)-7-((8-(4-((6S,8R)-7-(2,2- difluoroethyl)-8-methyl-6,7,8,9- tetrahydro-3H-pyrazolo[4,3- f]isoquinolin-6-yl)-3- mcthoxyphenyl)-1-oxa-8- azaspiro[4.5]decan-3-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B81 | 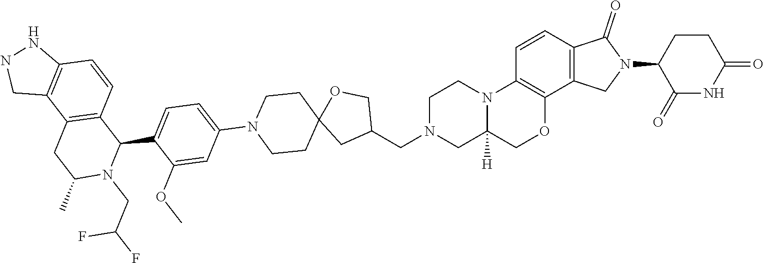 [SMILES: COc1cc(N2CCC3(CC2)CC(CN2CCN4c5ccc6c(c5OC[C@H]4C2)CN([C@H]2CCC(=O)NC2=O)C6=O)CO3)ccc1[C@@H]1c2ccc3c(c2C[C@@H](C)N1CC(F)F)CNN3] | (3S)-3-((5aR)-7-((8-(4-((6S,8R)-7- (2,2-difluoroethyl)-8-methyl- 6,7,8,9-tetrahydro-3H- pyrazolo[4,3-f]isoquinolin-6-yl)-3- methoxyphenyl)-1-oxa-8- azaspiro[4.5]decan-3-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B82 | 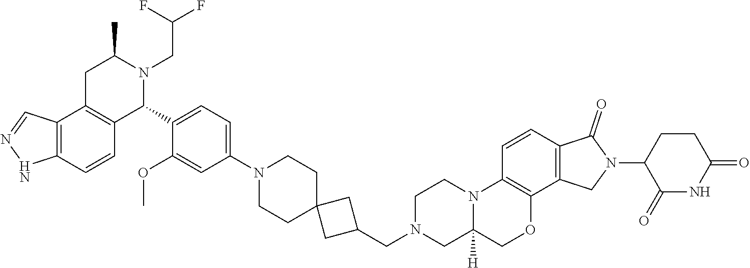 [SMILES: COc1cc(N2CCC3(CC2)CC(CN2CCN4c5ccc6c(c5OC[C@H]4C2)CN(C2CCC(=O)NC2=O)C6=O)C3)ccc1[C@@H]1c2ccc3[nH]ncc3c2C[C@@H](C)N1CC(F)F] | 3-((R)-7-((7-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-7- azaspiro[3.5]nonan-2-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B83 | 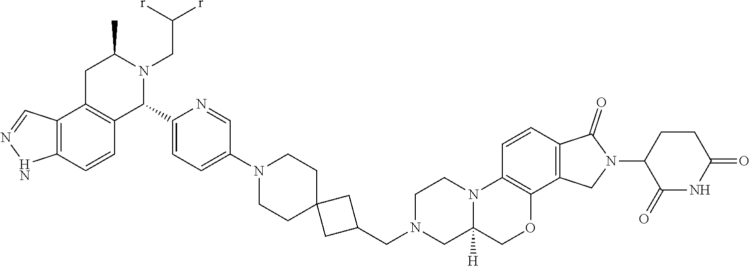 | 3-((R)-7-((7-(6-((6S,8R)-7-(2,2- difluoroethyl)-8-methyl-6,7,8,9- tetrahydro-3H-pyrazolo[4,3- f]isoquinolin-6-yl)pyridin-3-yl)-7- azaspiro[3.5]nonan-2-yl)mcthyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B84 | 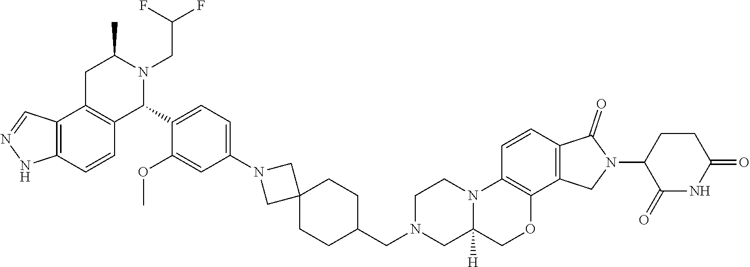 [SMILES: COc1cc(N2CC3(CCC(CN4CCN5c6ccc7c(c6OC[C@H]5C4)CN(C4CCC(=O)NC4=O)C7=O)CC3)C2)ccc1[C@@H]1c2ccc3[nH]ncc3c2C[C@@H](C)N1CC(F)F] | 3-((R)-7-((2-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B85 | 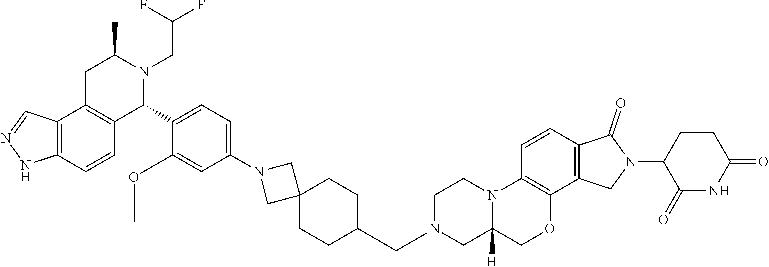 [SMILES: COc1cc(N2CC3(CCC(CN4CCN5c6ccc7c(c6OC[C@@H]5C4)CN(C4CCC(=O)NC4=O)C7=O)CC3)C2)ccc1[C@@H]1c2ccc3[nH]ncc3c2C[C@@H](C)N1CC(F)F] | 3-((S)-7-((2-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B86 | 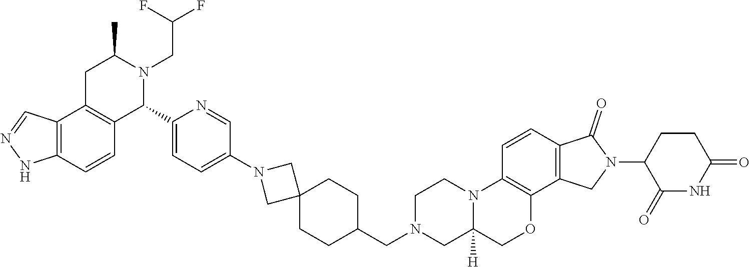 [SMILES: C[C@@H]1Cc2c(ccc3[nH]ncc23)[C@@H](c2ccc(N3CC4(CCC(CN5CCN6c7ccc8c(c7OC[C@H]6C5)CN(C5CCC(=O)NC5=O)C8=O)CC4)C3)cn2)N1CC(F)F] | 3-((R)-7-((2-(6-((6S,8R)-7-(2,2- difluoroethyl)-8-methyl-6,7,8,9- tetrahydro-3H-pyrazolo[4,3- f]isoquinolin-6-yl)pyridin-3-yl)-2- azaspiro[3.5]nonan-7-yl)mcthyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B87 | 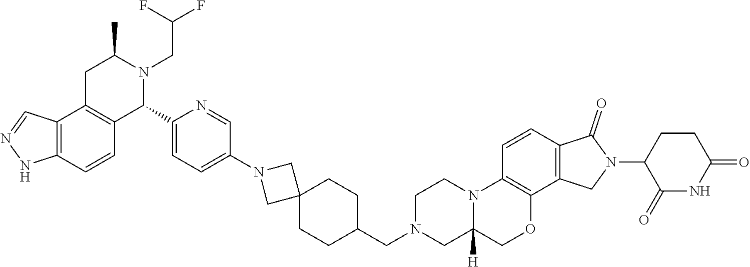 [SMILES: C[C@@H]1Cc2c(ccc3[nH]ncc23)[C@@H](c2ccc(N3CC4(CCC(CN5CCN6c7ccc8c(c7OC[C@@H]6C5)CN(C5CCC(=O)NC5=O)C8=O)CC4)C3)cn2)N1CC(F)F] | 3-((S)-7-((2-(6-((6S,8R)-7-(2,2- difluoroethyl)-8-methyl-6,7,8,9- tetrahydro-3H-pyrazolo[4,3- f]isoquinolin-6-yl)pyridin-3-yl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B88 | 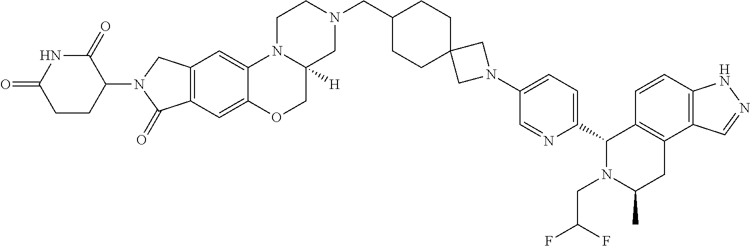 [SMILES: C[C@@H]1Cc2c(ccc3[nH]ncc23)[C@@H](c2ccc(N3CC4(CCC(CN5CCN6c7cc8c(cc7OC[C@@H]6C5)C(=O)N(C5CCC(=O)NC5=O)C8)CC4)C3)cn2)N1CC(F)F] | 3-((S)-3-((2-(6-((6S,8R)-7-(2,2- difluoroethyl)-8-methyl-6,7,8,9- tetrahydro-3H-pyrazolo[4,3- f]isoquinolin-6-yl)pyridin-3-yl)-2- azaspiro[3.5]nonan-7-yl)methyl)-8- oxo-1,2,3,4,4a,5,8,10-octahydro- 9H- pyrazino[1′,2′:4,5][1,4]oxazino[2,3- f]isoindol-9-yl)piperidine-2,6- dione |
| B89 | 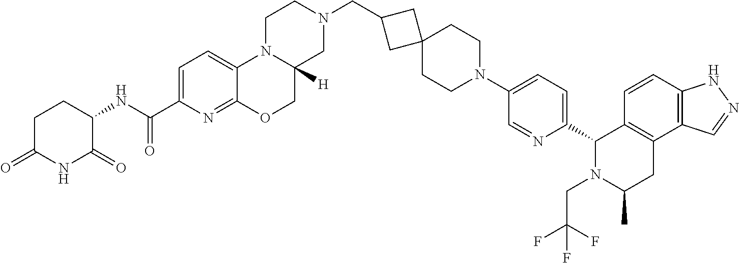 [SMILES: C[C@@H]1Cc2c(ccc3[nH]ncc23)[C@@H](c2ccc(N3CCC4(CC3)CC(CN3CCN5c6ccc(C(=O)N[C@H]7CCC(=O)NC7=O)nc6OC[C@H]5C3)C4)cn2)N1CC(F)(F)F] | (R)-N-((S)-2,6-dioxopiperidin-3- yl)-3-((7-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)-7- azaspiro[3.5]nonan-2-yl)methyl)- 1,2,3,4,4a,5- hexahydropyrazino[1,2- d]pyrido[2,3-b][1,4]oxazine-8- carboxamide |
| B90 | 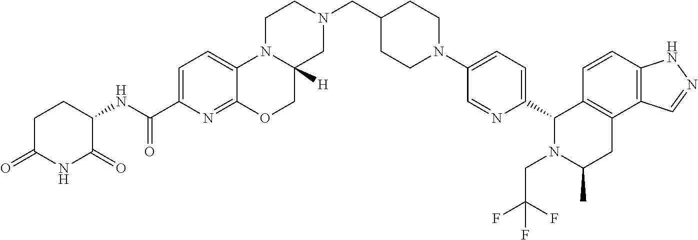 [SMILES: C[C@@H]1Cc2c(ccc3[nH]ncc23)[C@@H](c2ccc(N3CCC(CN4CCN5c6ccc(C(=O)N[C@H]7CCC(=O)NC7=O)nc6OC[C@H]5C4)CC3)cn2)N1CC(F)(F)F] | (R)-N-((S)-2,6-dioxopiperidin-3- yl)-3-((1-(6-((6S,8R)-8-methyl-7- (2,2,2-trifluoroethyl)-6,7,8,9- tetrahydro-3H-pyrazolo[4,3- f]isoquinolin-6-yl)pyridin-3- yl)piperidin-4-yl)methyl)- 1,2,3,4,4a,5- hexahydropyrazino[1,2- d]pyrido[2,3-b][1,4]oxazine-8- carboxamide |
| B91 | 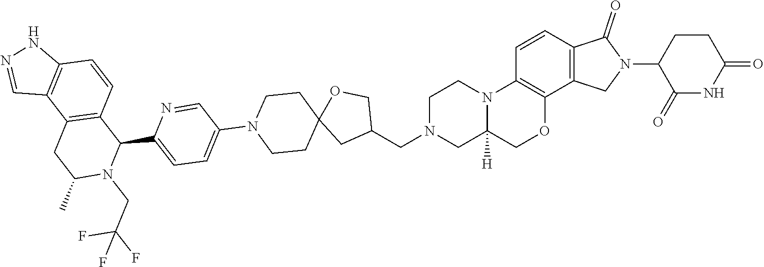 [SMILES: C[C@@H]1Cc2c(ccc3[nH]ncc23)[C@@H](c2ccc(N3CCC4(CC3)CC(CN3CCN5c6ccc7c(c6OC[C@H]5C3)CN(C3CCC(=O)NC3=O)C7=O)CO4)cn2)N1CC(F)(F)F] | 3-((5aR)-7-((8-(6-((6S,8R)-8- methyl-7-(2,2,2-trifluoroethyl)- 6,7,8,9-tetrahydro-3H- pyrazolo[4,3-f]isoquinolin-6- yl)pyridin-3-yl)-1-oxa-8- azaspiro[4.5]decan-3-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B92 | 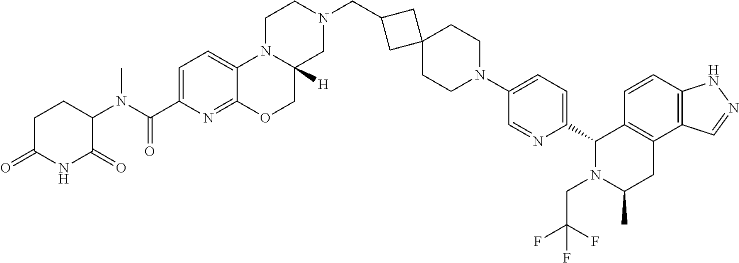 [SMILES: C[C@@H]1Cc2c(ccc3[nH]ncc23)[C@@H](c2ccc(N3CCC4(CC3)CC(CN3CCN5c6ccc(C(=O)N(C)C7CCC(=O)NC7=O)nc6OC[C@H]5C3)C4)cn2)N1CC(F)(F)F] | (4aR)-N-(2,6-dioxopiperidin-3-yl)- N-methyl-3-((7-(6-((6S,8R)-8- methyl-7-(2,2,2-trifluoroethyl)- 6,7,8,9-tetrahydro-3H- pyrazolo[4,3-f]isoquinolin-6- yl)pyridin-3-yl)-7- azaspiro[3.5]nonan-2-yl)methyl)- 1,2,3,4,4a,5- hexahydropyrazino[1,2- d]pyrido[2,3-b][1,4]oxazine-8- carboxamide |
| B93 | 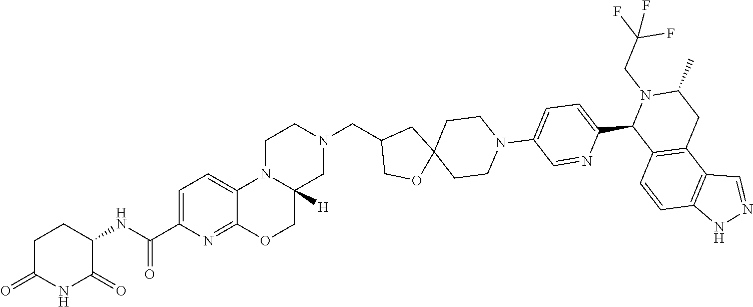 [SMILES: C[C@@H]1Cc2c(ccc3[nH]ncc23)[C@@H](c2ccc(N3CCC4(CC3)CC(CN3CCN5c6ccc(C(=O)N[C@H]7CCC(=O)NC7=O)nc6OC[C@H]5C3)CO4)cn2)N1CC(F)(F)F] | (4aR)-N-((S)-2,6-dioxopiperidin-3- yl)-3-((8-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)-1- oxa-8-azaspiro[4.5]decan-3- yl)methyl)-1,2,3,4,4a,5- hexahydropyrazino[1,2- d]pyrido[2,3-b][1,4]oxazine-8- carboxamide |
| B94 | 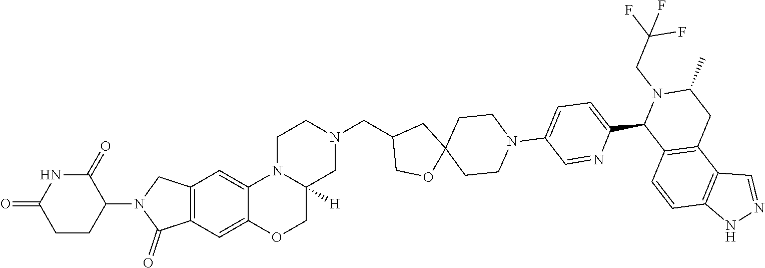 [SMILES: C[C@@H]1Cc2c(ccc3[nH]ncc23)[C@@H](c2ccc(N3CCC4(CC3)CC(CN3CCN5c6cc7c(cc6OC[C@@H]5C3)C(=O)N(C3CCC(=O)NC3=O)C7)CO4)cn2)N1CC(F)(F)F] | 3-((4aS)-3-((8-(6-((6S,8R)-8- methyl-7-(2,2,2-trifluoroethyl)- 6,7,8,9-tetrahydro-3H- pyrazolo[4,3-f]isoquinolin-6- yl)pyridin-3-yl)-1-oxa-8- azaspiro[4.5]decan-3-yl)methyl)-8- oxo-1,2,3,4,4a,5,8,10-octahydro- 9H- pyrazino[1′,2′:4,5][1,4]oxazino[2,3- f]isoindol-9-yl)piperidine-2,6- dione |
| B95 | 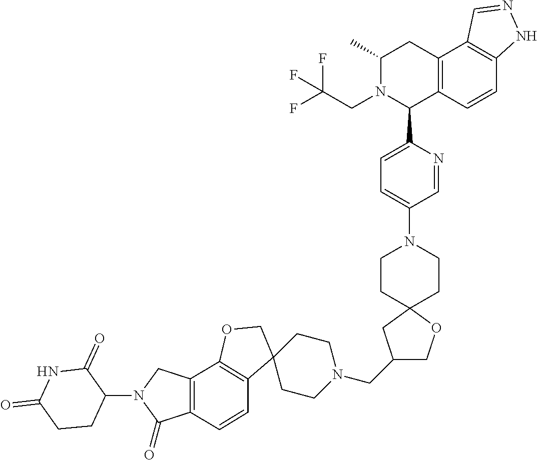 [SMILES: C[C@@H]1Cc2c(ccc3[nH]ncc23)[C@@H](c2ccc(N3CCC4(CC3)CC(CN3CCC5(CC3)COc3c5ccc5c3CN(C3CCC(=O)NC3=O)C5=O)CO4)cn2)N1CC(F)(F)F] | 3-(1′-((8-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)-1- oxa-8-azaspiro[4.5]decan-3- yl)methyl)-6-oxo-6,8-dihydro- 2H,7H-spiro[furo[2,3-e]isoindole- 3,4′-piperidin]-7-yl)piperidine-2,6- dione |
| B96 | 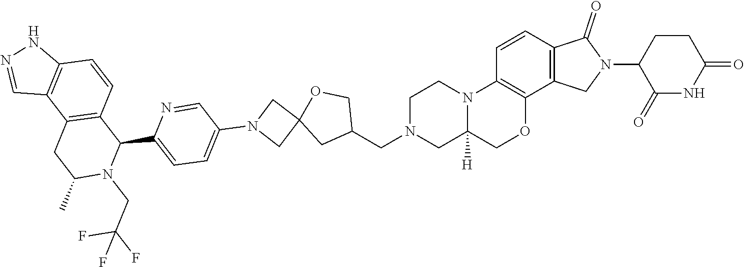 [SMILES: C[C@@H]1Cc2c(ccc3[nH]ncc23)[C@@H](c2ccc(N3CC4(CC(CN5CCN6c7ccc8c(c7OC[C@H]6C5)CN(C5CCC(=O)NC5=O)C8=O)CO4)C3)cn2)N1CC(F)(F)F] | 3-((5aR)-7-((2-(6-((6S,8R)-8- methyl-7-(2,2,2-trifluoroethyl)- 6,7,8,9-tetrahydro-3H- pyrazolo[4,3-f]isoquinolin-6- yl)pyridin-3-yl)-5-oxa-2- azaspiro[3.4]octan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B97 | 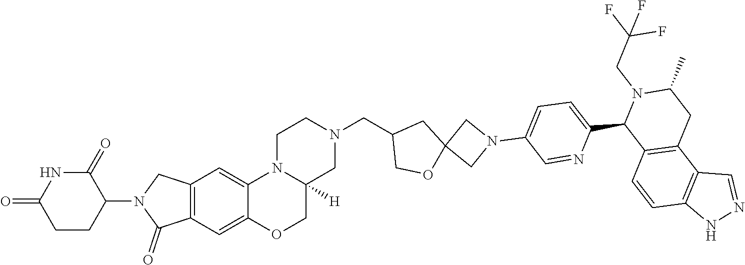 [SMILES: C[C@@H]1Cc2c(ccc3[nH]ncc23)[C@@H](c2ccc(N3CC4(CC(CN5CCN6c7cc8c(cc7OC[C@@H]6C5)C(=O)N(C5CCC(=O)NC5=O)C8)CO4)C3)cn2)N1CC(F)(F)F] | 3-((4aS)-3-((2-(6-((6S,8R)-8- methyl-7-(2,2,2-trifluoroethyl)- 6,7,8,9-tetrahydro-3H- pyrazolo[4,3-f]isoquinolin-6- yl)pyridin-3-yl)-5-oxa-2- azaspiro[3.4]octan-7-yl)methyl)-8- oxo-1,2,3,4,4a,5,8,10-octahydro- 9H- pyrazino[1′,2′:4,5][1,4]oxazino[2,3- f]isoindol-9-yl)piperidine-2,6- dione |
| B98 | 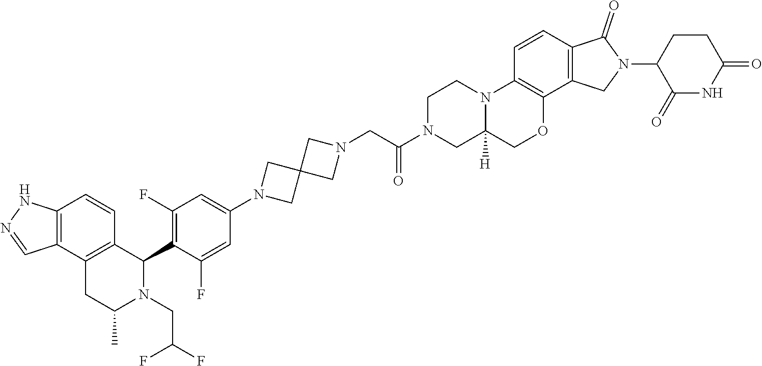 [SMILES: C[C@@H]1Cc2c(ccc3[nH]ncc23)[C@@H](c2c(F)cc(N3CC4(CN(CC(=O)N5CCN6c7ccc8c(c7OC[C@H]6C5)CN(C5CCC(=O)NC5=O)C8=O)C4)C3)cc2F)N1CC(F)F] | 3-((R)-7-(2-(6-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,6- diazaspiro[3.3]heptan-2-yl)acetyl)- 1-oxo-1,3,5,5a,6,7,8,9-octahydro- 2H- pyrazino[1′,2′:4,5][1,4]oxazino[2,3- e]isoindol-2-yl)piperidine-2,6- dione |
| B99 | 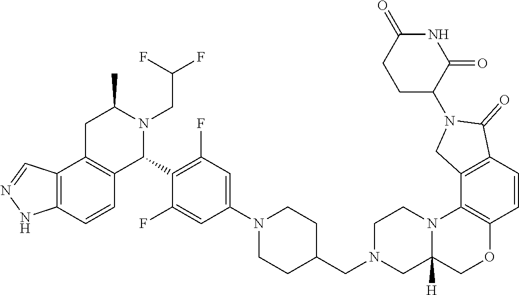 [SMILES: C[C@@H]1Cc2c(ccc3[nH]ncc23)[C@@H](c2c(F)cc(N3CCC(CN4CCN5c6c(ccc7c6CN(C6CCC(=O)NC6=O)C7=O)OC[C@@H]5C4)CC3)cc2F)N1CC(F)F] | 3-((S)-9-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-3-oxo- 1,3,7,7a,8,9,10,11-octahydro-2H- pyrazino[1′,2′:4,5][1,4]oxazino[3,2- e]isoindol-2-yl)piperidine-2,6- dione |
| B100 | 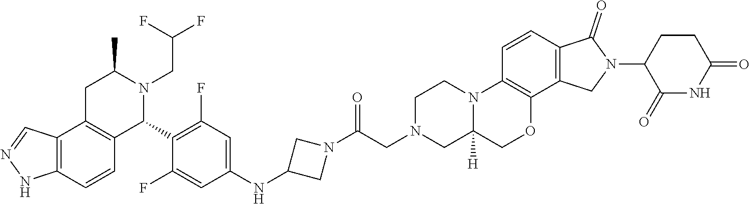 [SMILES: C[C@@H]1Cc2c(ccc3[nH]ncc23)[C@@H](c2c(F)cc(NC3CN(C(=O)CN4CCN5c6ccc7c(c6OC[C@H]5C4)CN(C4CCC(=O)NC4=O)C7=O)C3)cc2F)N1CC(F)F] | 3-((R)-7-(2-(3-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)azetidin-1- yl)-2-oxoethyl)-1-oxo- 1,3,5,5a,6,7,8,9-octahydro-2H- pyrazino[1′,2′:4,5][1,4]oxazino[2,3- e]isoindol-2-yl)piperidine-2,6- dione |
| B101 | 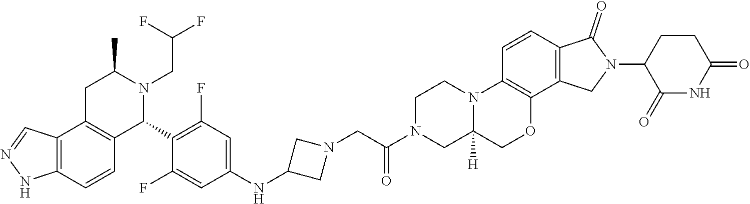 [SMILES: C[C@@H]1Cc2c(ccc3[nH]ncc23)[C@@H](c2c(F)cc(NC3CN(CC(=O)N4CCN5c6ccc7c(c6OC[C@H]5C4)CN(C4CCC(=O)NC4=O)C7=O)C3)cc2F)N1CC(F)F] | 3-((R)-7-(2-(3-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)azctidin-1- yl)acetyl)-1-oxo-1,3,5,5a,6,7,8,9- octahydro-2H- pyrazino[1′,2′:4,5][1,4]oxazino[2,3- e]isoindol-2-yl)piperidine-2,6- dione |
| B104 | 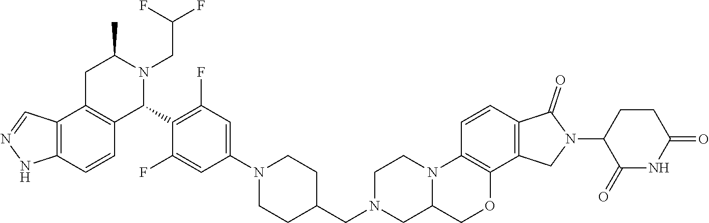 [SMILES: C[C@@H]1Cc2c(ccc3[nH]ncc23)[C@@H](c2c(F)cc(N3CCC(CN4CCN5c6ccc7c(c6OCC5C4)CN(C4CCC(=O)NC4=O)C7=O)CC3)cc2F)N1CC(F)F] | 3-((RS)-7-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-1-oxo-1,3,5,5a,6,7,8,9- octahydro-2H- pyrazino[1′,2′:4,5][1,4]oxazino[2,3- e]isoindol-2-yl)piperidine-2,6- dione |
| B105 | 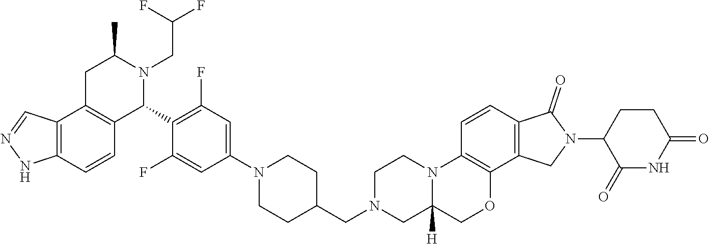 [SMILES: C[C@@H]1Cc2c(ccc3[nH]ncc23)[C@@H](c2c(F)cc(N3CCC(CN4CCN5c6ccc7c(c6OC[C@@H]5C4)CN(C4CCC(=O)NC4=O)C7=O)CC3)cc2F)N1CC(F)F] | 3-((S)-7-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-1-oxo-1,3,5,5a,6,7,8,9- octahydro-2H- pyrazino[1′,2′:4,5][1,4]oxazino[2,3- e]isoindol-2-yl)piperidine-2,6- dione |
| B106 | 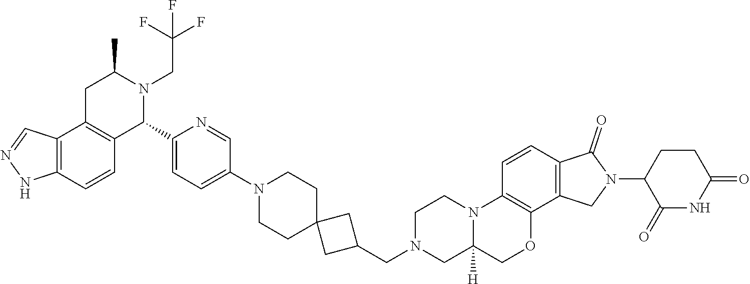 [SMILES: C[C@@H]1Cc2c(ccc3[nH]ncc23)[C@@H](c2ccc(N3CCC4(CC3)CC(CN3CCN5c6ccc7c(c6OC[C@H]5C3)CN(C3CCC(=O)NC3=O)C7=O)C4)cn2)N1CC(F)(F)F] | 3-((R)-7-((7-(6-((6S,8R)-8-methyl- 7-(2,2,2-trifluoroethyl)-6,7,8,9- tetrahydro-3H-pyrazolo[4,3- f]isoquinolin-6-yl)pyridin-3-yl)-7- azaspiro[3.5]nonan-2-yl)mcthyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B107 | 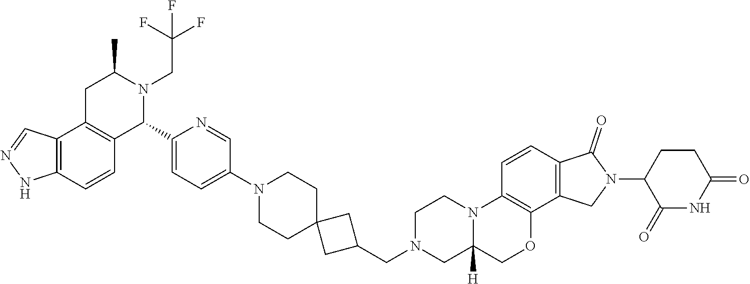 [SMILES: C[C@@H]1Cc2c(ccc3[nH]ncc23)[C@@H](c2ccc(N3CCC4(CC3)CC(CN3CCN5c6ccc7c(c6OC[C@@H]5C3)CN(C3CCC(=O)NC3=O)C7=O)C4)cn2)N1CC(F)(F)F] | 3-((S)-7-((7-(6-((6S,8R)-8-methyl- 7-(2,2,2-trifluoroethyl)-6,7,8,9- tetrahydro-3H-pyrazolo[4,3- f]isoquinolin-6-yl)pyridin-3-yl)-7- azaspiro[3.5]nonan-2-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B108 | 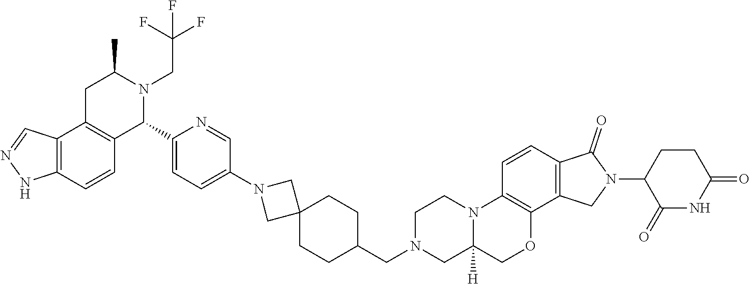 [SMILES: C[C@@H]1Cc2c(ccc3[nH]ncc23)[C@@H](c2ccc(N3CC4(CCC(CN5CCN6c7ccc8c(c7OC[C@H]6C5)CN(C5CCC(=O)NC5=O)C8=O)CC4)C3)cn2)N1CC(F)(F)F] | 3-((R)-7-((2-(6-((6S,8R)-8-methyl- 7-(2,2,2-trifluoroethyl)-6,7,8,9- tetrahydro-3H-pyrazolo[4,3- f]isoquinolin-6-yl)pyridin-3-yl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B109 | 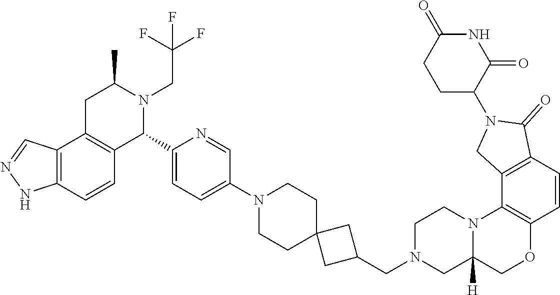 [SMILES: C[C@@H]1Cc2c(ccc3[nH]ncc23)[C@@H](c2ccc(N3CCC4(CC3)CC(CN3CCN5c6c(ccc7c6CN(C6CCC(=O)NC6=O)C7=O)OC[C@@H]5C3)C4)cn2)N1CC(F)(F)F] | 3-((S)-9-((7-(6-((6S,8R)-8-methyl- 7-(2,2,2-trifluoroethyl)-6,7,8,9- tetrahydro-3H-pyrazolo[4,3- f]isoquinolin-6-yl)pyridin-3-yl)-7- azaspiro[3.5]nonan-2-yl)mcthyl)-3- oxo-1,3,7,7a,8,9,10,11-octahydro- 2H- pyrazino[1′,2′:4,5][1,4]oxazino[3,2- e]isoindol-2-yl)piperidine-2,6- dione |
| B110 | 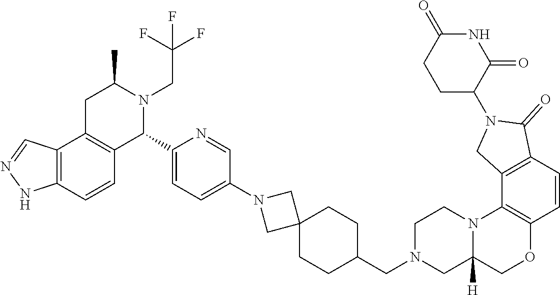 [SMILES: C[C@@H]1Cc2c(ccc3[nH]ncc23)[C@@H](c2ccc(N3CC4(CCC(CN5CCN6c7c(ccc8c7CN(C7CCC(=O)NC7=O)C8=O)OC[C@@H]6C5)CC4)C3)cn2)N1CC(F)(F)F] | 3-((S)-9-((2-(6-((6S,8R)-8-methyl- 7-(2,2,2-trifluoroethyl)-6,7,8,9- tetrahydro-3H-pyrazolo[4,3- f]isoquinolin-6-yl)pyridin-3-yl)-2- azaspiro[3.5]nonan-7-yl)methyl)-3- oxo-1,3,7,7a,8,9,10,11-octahydro- 2H- pyrazino[1′,2′:4,5][1,4]oxazino[3,2- e]isoindol-2-yl)piperidine-2,6- dione |
| B112 | 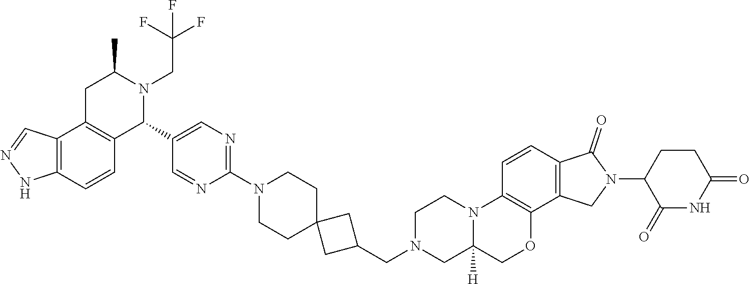 [SMILES: C[C@@H]1Cc2c(ccc3[nH]ncc23)[C@@H](c2cnc(N3CCC4(CC3)CC(CN3CCN5c6ccc7c(c6OC[C@H]5C3)CN(C3CCC(=O)NC3=O)C7=O)C4)nc2)N1CC(F)(F)F] | 3-((R)-7-((7-(5-((6S,8R)-8-methyl- 7-(2,2,2-trifluoroethyl)-6,7,8,9- tetrahydro-3H-pyrazolo[4,3- f]isoquinolin-6-yl)pyrimidin-2-yl)- 7-azaspiro[3.5]nonan-2-yl)methyl)- 1-oxo-1,3,5,5a,6,7,8,9-octahydro- 2H- pyrazino[1′,2′:4,5][1,4]oxazino[2,3- e]isoindol-2-yl)piperidine-2,6- dione |
| B113 | 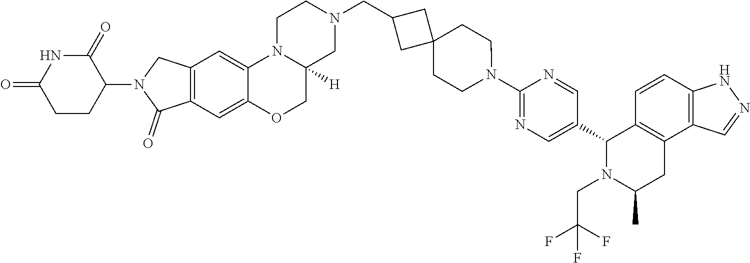 [SMILES: C[C@@H]1Cc2c(ccc3[nH]ncc23)[C@@H](c2cnc(N3CCC4(CC3)CC(CN3CCN5c6cc7c(cc6OC[C@@H]5C3)C(=O)N(C3CCC(=O)NC3=O)C7)C4)nc2)N1CC(F)(F)F] | 3-((S)-3-((7-(5-((6S,8R)-8-methyl- 7-(2,2,2-trifluoroethyl)-6,7,8,9- tetrahydro-3H-pyrazolo[4,3- f]isoquinolin-6-yl)pyrimidin-2-yl)- 7-azaspiro[3.5]nonan-2-yl)mcthyl)- 8-oxo-1,2,3,4,4a,5,8,10-octahydro- 9H- pyrazino[1′,2′:4,5][1,4]oxazino[2,3- f]isoindol-9-yl)piperidine-2,6- dione |
| B114 | 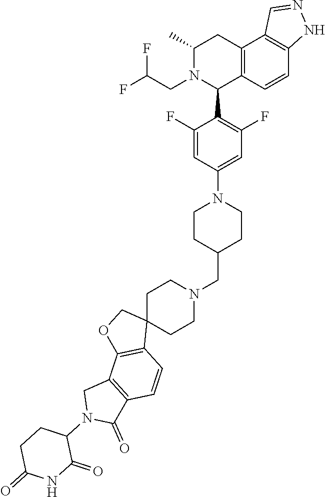 [SMILES: C[C@@H]1Cc2c(ccc3[nH]ncc23)[C@@H](c2c(F)cc(N3CCC(CN4CCC5(CC4)COc4c5ccc5c4CN(C4CCC(=O)NC4=O)C5=O)CC3)cc2F)N1CC(F)F] | 3-(1′-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-6-oxo-6,8-dihydro- 2H,7H-spiro[furo[2,3-e]isoindole- 3,4′-piperidin]-7-yl)piperidine-2,6- dione |
| B115 | 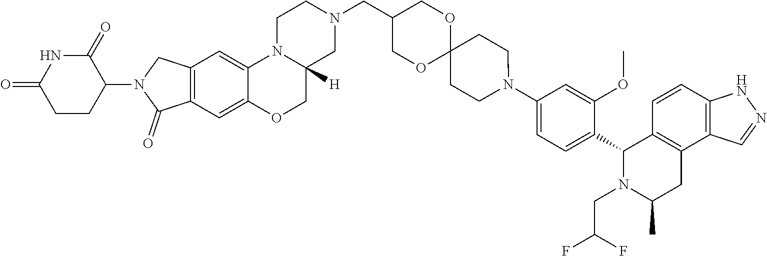 [SMILES: COc1cc(N2CCC3(CC2)OCC(CN2CCN4c5cc6c(cc5OC[C@H]4C2)C(=O)N(C2CCC(=O)NC2=O)C6)CO3)ccc1[C@@H]1c2ccc3[nH]ncc3c2C[C@@H](C)N1CC(F)F] | 3-((R)-3-((9-(4-((6S,8R)-7-(2,2- difluoroethyl)-8-methyl-6,7,8,9- tetrahydro-3H-pyrazolo[4,3- f]isoquinolin-6-yl)-3- mcthoxyphenyl)-1,5-dioxa-9- azaspiro[5.5]undecan-3-yl)methyl)- 8-oxo-1,2,3,4,4a,5,8,10-octahydro- 9H- pyrazino[1′,2′:4,5][1,4]oxazino[2,3- f]isoindol-9-yl)piperidine-2,6- dione |
| B116 | 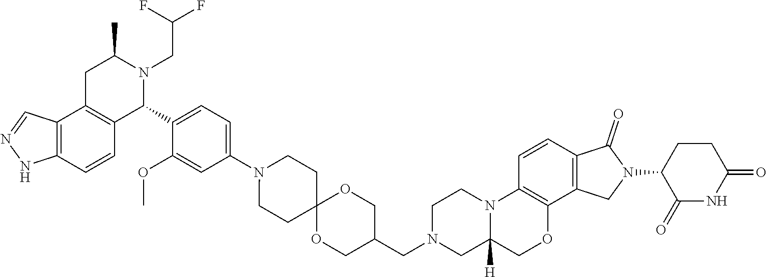 [SMILES: COc1cc(N2CCC3(CC2)OCC(CN2CCN4c5ccc6c(c5OC[C@@H]4C2)CN([C@@H]2CCC(=O)NC2=O)C6=O)CO3)ccc1[C@@H]1c2ccc3[nH]ncc3c2C[C@@H](C)N1CC(F)F] | (R)-3-((S)-7-((9-(4-((6S,8R)-7- (2,2-difluoroethyl)-8-methyl- 6,7,8,9-tetrahydro-3H- pyrazolo[4,3-f]isoquinolin-6-yl)-3- methoxyphenyl)-1,5-dioxa-9- azaspiro[5.5]undecan-3-yl)methyl)- 1-oxo-1,3,5,5a,6,7,8,9-octahydro- 2H- pyrazino[1′,2′:4,5][1,4]oxazino[2,3- e]isoindol-2-yl)piperidine-2,6- dione |
| B117 | 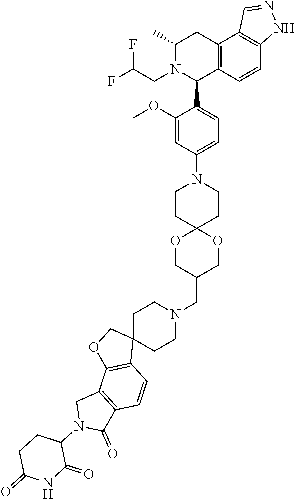 [SMILES: COc1cc(N2CCC3(CC2)OCC(CN2CCC4(CC2)COc2c4ccc4c2CN(C2CCC(=O)NC2=O)C4=O)CO3)ccc1[C@@H]1c2ccc3[nH]ncc3c2C[C@@H](C)N1CC(F)F] | 3-(1′-((9-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-1,5-dioxa-9- azaspiro[5.5]undecan-3-yl)methyl)- 6-oxo-6,8-dihydro-2H,7H- spiro[furo[2,3-e]isoindole-3,4′- piperidin]-7-yl)piperidine-2,6- dione |
| B118 | 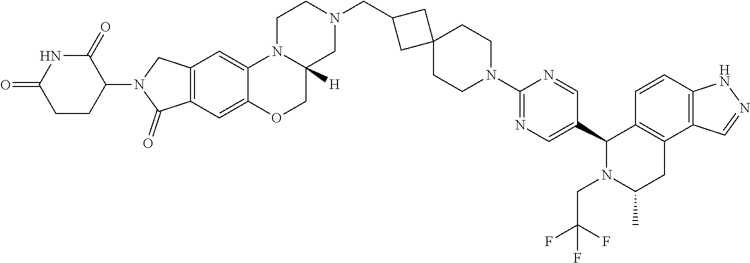 [SMILES: C[C@H]1Cc2c(ccc3[nH]ncc23)[C@H](c2cnc(N3CCC4(CC3)CC(CN3CCN5c6cc7c(cc6OC[C@H]5C3)C(=O)N(C3CCC(=O)NC3=O)C7)C4)nc2)N1CC(F)(F)F] | 3-((R)-3-((7-(5-((6R,8S)-8-methyl- 7-(2,2,2-trifluoroethyl)-6,7,8,9- tetrahydro-3H-pyrazolo[4,3- f]isoquinolin-6-yl)pyrimidin-2-yl)- 7-azaspiro[3.5]nonan-2-yl)methyl)- 8-oxo-1,2,3,4,4a,5,8,10-octahydro- 9H- pyrazino[1′,2′:4,5][1,4]oxazino[2,3- f]isoindol-9-yl)piperidine-2,6- dione |
| B119 | 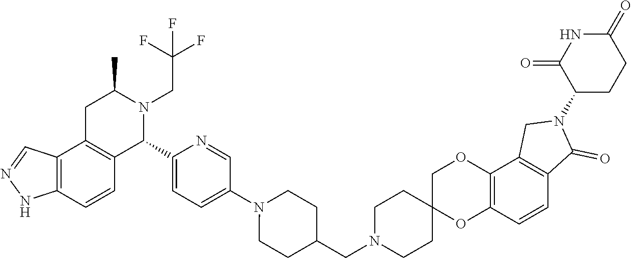 [SMILES: C[C@@H]1Cc2c(ccc3[nH]ncc23)[C@@H](c2ccc(N3CCC(CN4CCC5(CC4)COc4c(ccc6c4CN([C@H]4CCC(=O)NC4=O)C6=O)O5)CC3)cn2)N1CC(F)(F)F] | (S)-3-(1-((1-(6-((6S,8R)-8-methyl- 7-(2,2,2-trifluoroethyl)-6,7,8,9- tetrahydro-3H-pyrazolo[4,3- f]isoquinolin-6-yl)pyridin-3- yl)piperidin-4-yl)mcthyl)-7′-oxo- 7′,9′-dihydro-2′H,8′H- spiro[piperidine-4,3′- [1,4]dioxino[2,3-e]isoindol]-8′- yl)piperidine-2,6-dione |
| B120 | 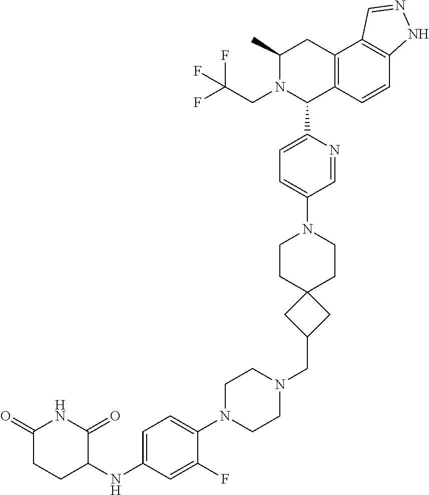 [SMILES: C[C@H]1Cc2c(ccc3[nH]ncc23)[C@H](c2ccc(N3CCC4(CC3)CC(CN3CCN(c5ccc(NC6CCC(=O)NC6=O)cc5F)CC3)C4)cn2)N1CC(F)(F)F] | 3-((3-fluoro-4-(4-((7-(6-((6R,8S)- 8-methyl-7-(2,2,2-trifluoroethyl)- 6,7,8,9-tetrahydro-3H- pyrazolo[4,3-f]isoquinolin-6- yl)pyridin-3-yl)-7- azaspiro[3.5]nonan-2- yl)methyl)piperazin-1- yl)phenyl)amino)piperidine-2,6- dione |
| B121 | 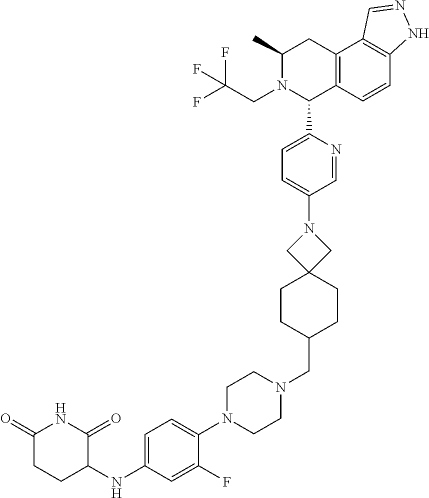 [SMILES: C[C@H]1Cc2c(ccc3[nH]ncc23)[C@H](c2ccc(N3CC4(CCC(CN5CCN(c6ccc(NC7CCC(=O)NC7=O)cc6F)CC5)CC4)C3)cn2)N1CC(F)(F)F] | 3-((3-fluoro-4-(4-((2-(6-((6R,8S)- 8-methyl-7-(2,2,2-trifluoroethyl)- 6,7,8,9-tetrahydro-3H- pyrazolo[4,3-f]isoquinolin-6- yl)pyridin-3-yl)-2- azaspiro[3.5]nonan-7- yl)methyl)piperazin-1- yl)phenyl)amino)piperidine-2,6- dione |
| B122 | 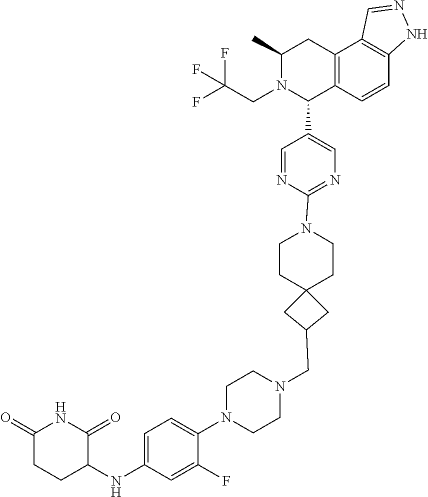 [SMILES: C[C@H]1Cc2c(ccc3[nH]ncc23)[C@H](c2cnc(N3CCC4(CC3)CC(CN3CCN(c5ccc(NC6CCC(=O)NC6=O)cc5F)CC3)C4)nc2)N1CC(F)(F)F] | 3-((3-fluoro-4-(4-((7-(5-((6R,8S)- 8-methyl-7-(2,2,2-trifluoroethyl)- 6,7,8,9-tetrahydro-3H- pyrazolo[4,3-f]isoquinolin-6- yl)pyrimidin-2-yl)-7- azaspiro[3.5]nonan-2- yl)methyl)piperazin-1- yl)phenyl)amino)piperidine-2,6- dione |
| B123 | 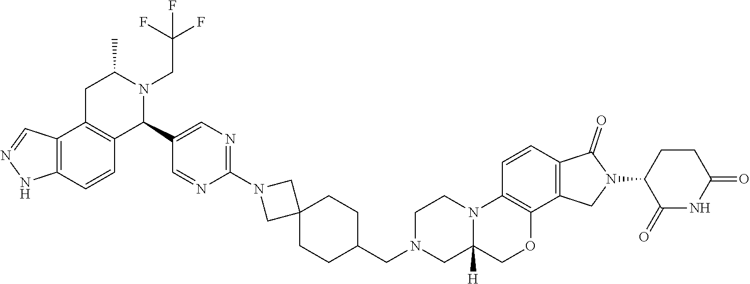 [SMILES: C[C@H]1Cc2c(ccc3[nH]ncc23)[C@H](c2cnc(N3CC4(CCC(CN5CCN6c7ccc8c(c7OC[C@@H]6C5)CN([C@@H]5CCC(=O)NC5=O)C8=O)CC4)C3)nc2)N1CC(F)(F)F] | (R)-3-((S)-7-((2-(5-((6R,8S)-8- methyl-7-(2,2,2-trifluoroethyl)- 6,7,8,9-tetrahydro-3H- pyrazolo[4,3-f]isoquinolin-6- yl)pyrimidin-2-yl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B124 | 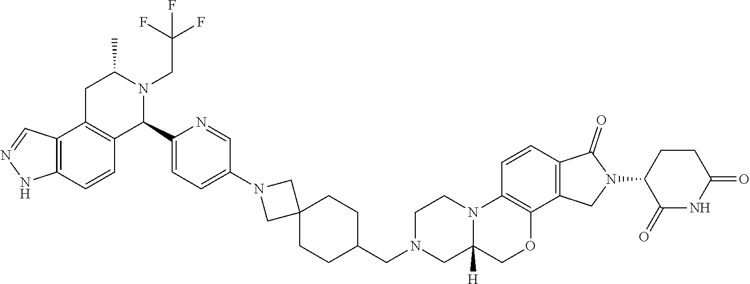 [SMILES: C[C@H]1Cc2c(ccc3[nH]ncc23)[C@H](c2ccc(N3CC4(CCC(CN5CCN6c7ccc8c(c7OC[C@@H]6C5)CN([C@@H]5CCC(=O)NC5=O)C8=O)CC4)C3)cn2)N1CC(F)(F)F] | (R)-3-((S)-7-((2-(6-((6R,8S)-8- methyl-7-(2,2,2-trifluoroethyl)- 6,7,8,9-tetrahydro-3H- pyrazolo[4,3-f]isoquinolin-6- yl)pyridin-3-yl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6- dione |
| B125 | 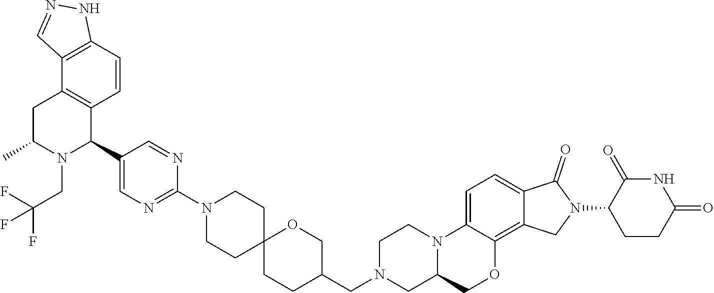 [SMILES: C[C@@H]1Cc2c(ccc3[nH]ncc23)[C@@H](c2cnc(N3CCC4(CCC(CN5CCN6c7ccc8c(c7OC[C@H]6C5)CN([C@H]5CCC(=O)NC5=O)C8=O)CO4)CC3)nc2)N1CC(F)(F)F] | (3S)-3-((5aR)-7-((9-(5-((6S,8R)-8- methyl-7-(2,2,2-trifluoroethyl)- 6,7,8,9-tetrahydro-3H- pyrazolo[4,3-f]isoquinolin-6- yl)pyrimidin-2-yl)-1-oxa-9- azaspiro[5.5]undecan-3-yl)methyl)- 1-oxo-1,3,5,5a,6,7,8,9-octahydro- 2H- pyrazino[1′,2′:4,5][1,4]oxazino[2,3- e]isoindol-2-yl)piperidine-2,6- dione |
| B126 | 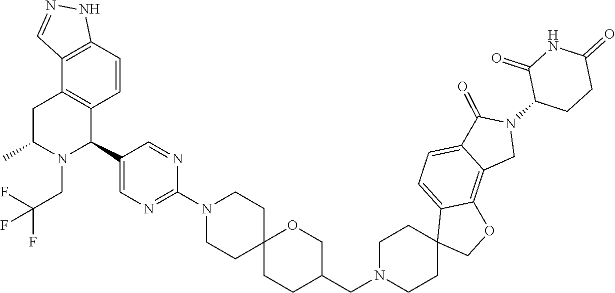 [SMILES: C[C@@H]1Cc2c(ccc3[nH]ncc23)[C@@H](c2cnc(N3CCC4(CCC(CN5CCC6(CC5)COc5c6ccc6c5CN([C@H]5CCC(=O)NC5=O)C6=O)CO4)CC3)nc2)N1CC(F)(F)F] | (3S)-3-(1′-((9-(5-((6S,8R)-8- methyl-7-(2,2,2-trifluoroethyl)- 6,7,8,9-tetrahydro-3H- pyrazolo[4,3-f]isoquinolin-6- yl)pyrimidin-2-yl)-1-oxa-9- azaspiro[5.5]undecan-3-yl)methyl)- 6-oxo-6,8-dihydro-2H,7H- spiro[furo[2,3-e]isoindole-3,4′- piperidin]-7-yl)piperidine-2,6- dione |
| B127 | 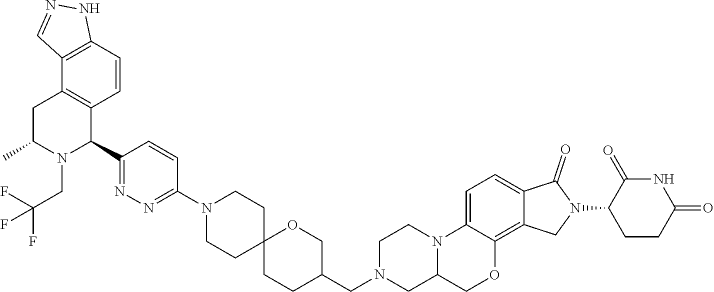 [SMILES: C[C@@H]1Cc2c(ccc3[nH]ncc23)[C@@H](c2ccc(N3CCC4(CCC(CN5CCN6c7ccc8c(c7OCC6C5)CN([C@H]5CCC(=O)NC5=O)C8=O)CO4)CC3)nn2)N1CC(F)(F)F] | (3S)-3-(7-((9-(6-((6S,8R)-8- methyl-7-(2,2,2-trifluoroethyl)- 6,7,8,9-tetrahydro-3H- pyrazolo[4,3-f]isoquinolin-6- yl)pyridazin-3-yl)-1-oxa-9- azaspiro[5.5]undecan-3-yl)methyl)- 1-oxo-1,3,5,5a,6,7,8,9-octahydro- 2H- pyrazino[1′,2′:4,5][1,4]oxazino[2,3- e]isoindol-2-yl)piperidine-2,6- dione |
| B128 | 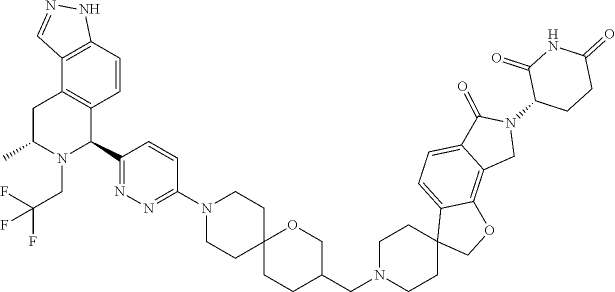 [SMILES: C[C@@H]1Cc2c(ccc3[nH]ncc23)[C@@H](c2ccc(N3CCC4(CCC(CN5CCC6(CC5)COc5c6ccc6c5CN([C@H]5CCC(=O)NC5=O)C6=O)CO4)CC3)nn2)N1CC(F)(F)F] | (3S)-3-(1′-((9-(6-((6S,8R)-8- methyl-7-(2,2,2-trifluoroethyl)- 6,7,8,9-tetrahydro-3H- pyrazolo[4,3-f]isoquinolin-6- yl)pyridazin-3-yl)-1-oxa-9- azaspiro[5.5]undecan-3-yl)methyl)- 6-oxo-6,8-dihydro-2H,7H- spiro[furo[2,3-e]isoindole-3,4′- piperidin]-7-yl)piperidine-2,6- dione |

TABLE 3
Selected Compound of A1-A26 and B1-B126

| Compound | | |
|---|---|---|
| No | Chemical Structure | Chemical Name |
| A3 | 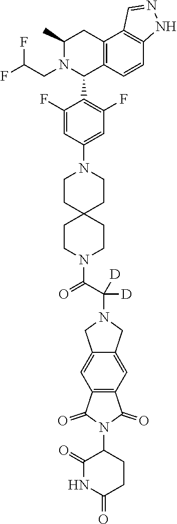 [SMILES: [2H]C([2H])(C(=O)N1CCC2(CC1)CCN(c1cc(F)c([C@H]3c4ccc5[nH]ncc5c4C[C@H](C)N3CC(F)F)c(F)c1)CC2)N1Cc2cc3c(cc2C1)C(=O)N(C1CCC(=O)NC1=O)C3=O] | 6-(2-(9-(4-((6R,8S)-7-(2,2- difluoroethyl)-8-methyl-6,7,8,9- tetrahydro-3H-pyrazolo[4,3- f]isoquinolin-6-yl)-3,5- difluorophenyl)-3,9- diazaspiro[5.5]undecan-3-yl)-2- oxoethyl-1,1-d2)-2-(2,6- dioxopiperidin-3-yl)-6,7- dihydropyrrolo[3,4-f]isoindole- 1,3(2H,5H)-dione |
| B1 | 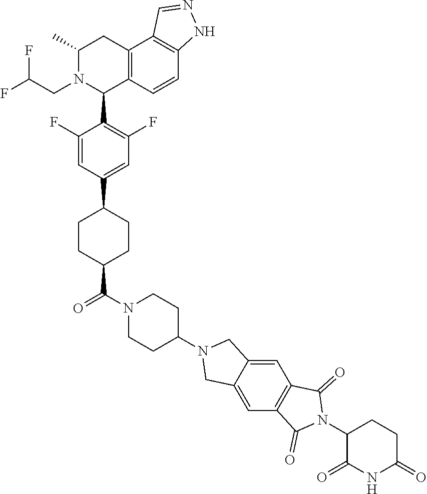 [SMILES: C[C@@H]1Cc2c(ccc3[nH]ncc23)[C@@H](c2c(F)cc([C@H]3CC[C@@H](C(=O)N4CCC(N5Cc6cc7c(cc6C5)C(=O)N(C5CCC(=O)NC5=O)C7=O)CC4)CC3)cc2F)N1CC(F)F] | 6-(1-((1R,4s)-4-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)cyclohexane-1- carbonyl)piperidin-4-yl)-2-(2,6- dioxopiperidin-3-yl)-6,7- dihydropyrrolo[3,4-f]isoindole- 1,3(2H,5H)-dione |
| B2 | 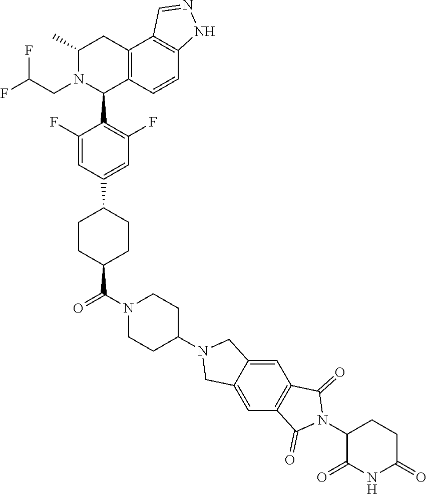 [SMILES: C[C@@H]1Cc2c(ccc3[nH]ncc23)[C@@H](c2c(F)cc([C@H]3CC[C@H](C(=O)N4CCC(N5Cc6cc7c(cc6C5)C(=O)N(C5CCC(=O)NC5=O)C7=O)CC4)CC3)cc2F)N1CC(F)F] | 6-(1-((1S,4r)-4-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)cyclohexane-1- carbonyl)pipcridin-4-yl)-2-(2,6- dioxopiperidin-3-yl)-6,7- dihydropyrrolo[3,4-f]isoindole- 1,3(2H,5H)-dione |
| B3 | 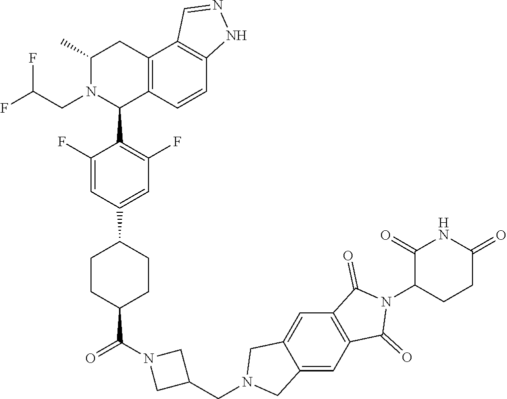 [SMILES: C[C@@H]1Cc2c(ccc3[nH]ncc23)[C@@H](c2c(F)cc([C@H]3CC[C@H](C(=O)N4CC(CN5Cc6cc7c(cc6C5)C(=O)N(C5CCC(=O)NC5=O)C7=O)C4)CC3)cc2F)N1CC(F)F] | 6-((1-((1S,4r)-4-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)cyclohexane-1- carbonyl)azetidin-3-yl)methyl)-2- (2,6-dioxopiperidin-3-yl)-6,7- dihydropyrrolo[3,4-f]isoindole- 1,3(2H,5H)-dione |
| B4 | 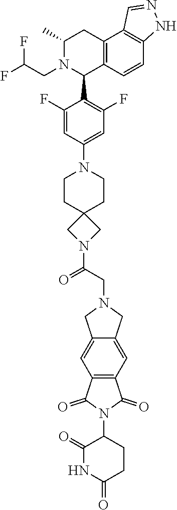 [SMILES: C[C@@H]1Cc2c(ccc3[nH]ncc23)[C@@H](c2c(F)cc(N3CCC4(CC3)CN(C(=O)CN3Cc5cc6c(cc5C3)C(=O)N(C3CCC(=O)NC3=O)C6=O)C4)cc2F)N1CC(F)F] | 6-(2-(7-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,7- diazaspiro[3.5]nonan-2-yl)-2- oxoethyl)-2-(2,6-dioxopiperidin-3- yl)-6,7-dihydropyrrolo[3,4- f]isoindole-1,3(2H,5H)-dione |
| B5 | 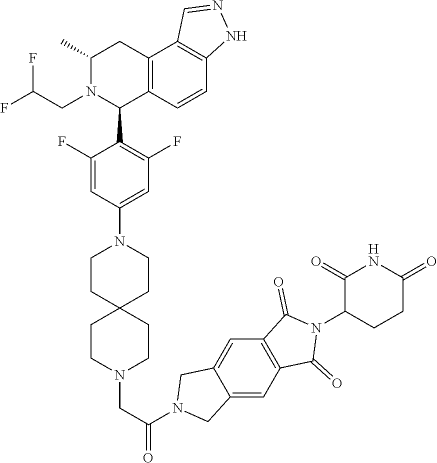 [SMILES: C[C@@H]1Cc2c(ccc3[nH]ncc23)[C@@H](c2c(F)cc(N3CCC4(CCN(CC(=O)N5Cc6cc7c(cc6C5)C(=O)N(C5CCC(=O)NC5=O)C7=O)CC4)CC3)cc2F)N1CC(F)F] | 6-(2-(9-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-3,9- diazaspiro[5.5]undecan-3- yl)acetyl)-2-(2,6-dioxopiperidin-3- yl)-6,7-dihydropyrrolo[3,4- f]isoindole-1,3(2H,5H)-dione |
| B6 | 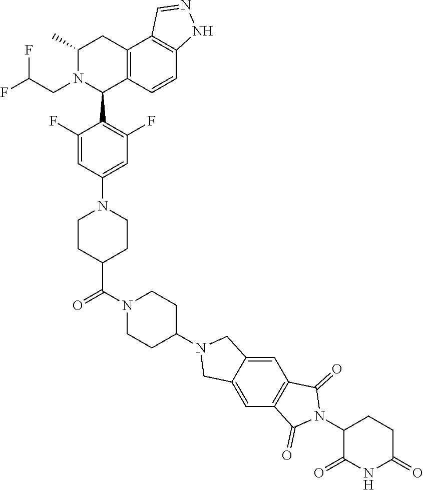 [SMILES: C[C@@H]1Cc2c(ccc3[nH]ncc23)[C@@H](c2c(F)cc(N3CCC(C(=O)N4CCC(N5Cc6cc7c(cc6C5)C(=O)N(C5CCC(=O)NC5=O)C7=O)CC4)CC3)cc2F)N1CC(F)F] | 6-(1-(1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidine-4- carbonyl)piperidin-4-yl)-2-(2,6- dioxopiperidin-3-yl)-6,7- dihydropyrrolo[3,4-f]isoindole- 1,3(2H,5H)-dione |
| B7 | 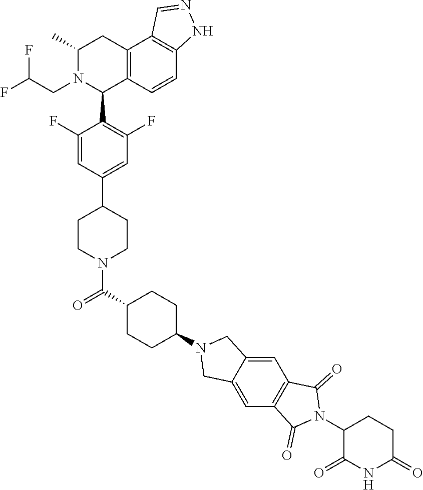 [SMILES: C[C@@H]1Cc2c(ccc3[nH]ncc23)[C@@H](c2c(F)cc(C3CCN(C(=O)[C@H]4CC[C@H](N5Cc6cc7c(cc6C5)C(=O)N(C5CCC(=O)NC5=O)C7=O)CC4)CC3)cc2F)N1CC(F)F] | 6-((1S,4r)-4-(4-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidine-1- carbonyl)cyclohexyl)-2-(2,6- dioxopiperidin-3-yl)-6,7- dihydropyrrolo[3,4-f]isoindole- 1,3(2H,5H)-dione |
| B8 | 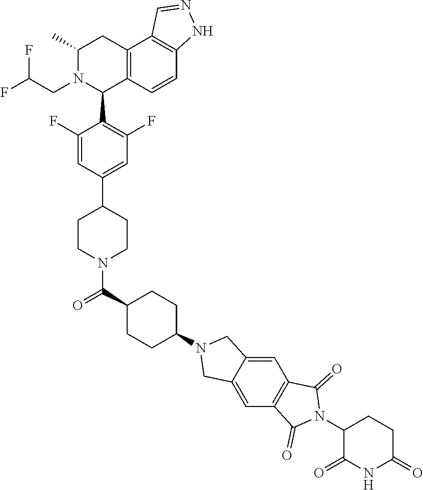 [SMILES: C[C@@H]1Cc2c(ccc3[nH]ncc23)[C@@H](c2c(F)cc(C3CCN(C(=O)[C@H]4CC[C@@H](N5Cc6cc7c(cc6C5)C(=O)N(C5CCC(=O)NC5=O)C7=O)CC4)CC3)cc2F)N1CC(F)F] | 6-((1R,4s)-4-(4-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidine-1- carbonyl)cyclohexyl)-2-(2,6- dioxopiperidin-3-yl)-6,7- dihydropyrrolo[3,4-f]isoindole- 1,3(2H,5H)-dione |
| B9 | 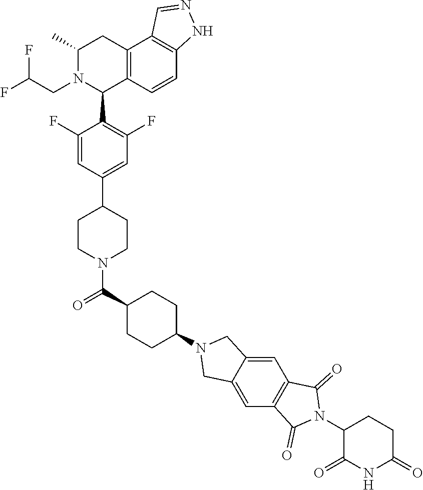 [SMILES: C[C@@H]1Cc2c(ccc3[nH]ncc23)[C@@H](c2c(F)cc(C3CCN(C(=O)[C@H]4CC[C@@H](N5Cc6cc7c(cc6C5)C(=O)N(C5CCC(=O)NC5=O)C7=O)CC4)CC3)cc2F)N1CC(F)F] | 6-((1R,4s)-4-(4-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidine-1- carbonyl)cyclohexyl)-2-(2,6- dioxopiperidin-3-yl)-6,7- dihydropyrrolo[3,4-f]isoindole- 1,3(2H,5H)-dione |
| B10 | 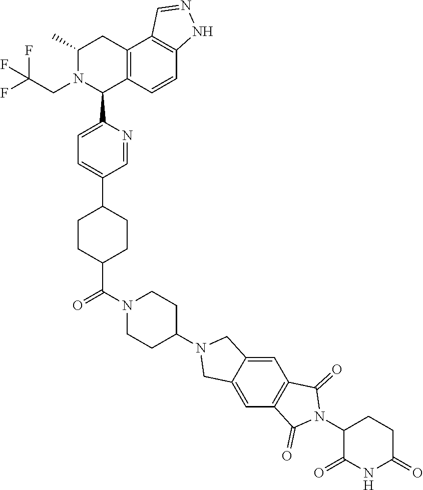 [SMILES: C[C@@H]1Cc2c(ccc3[nH]ncc23)[C@@H](c2ccc(C3CCC(C(=O)N4CCC(N5Cc6cc7c(cc6C5)C(=O)N(C5CCC(=O)NC5=O)C7=O)CC4)CC3)cn2)N1CC(F)(F)F] | 2-(2,6-dioxopiperidin-3-yl)-6-(1-(4- (6-((6S,8R)-8-methyl-7-(2,2,2- trifluoroethyl)-6,7,8,9-tetrahydro- 3H-pyrazolo[4,3-f]isoquinolin-6- yl)pyridin-3-yl)cyclohexane-1- carbonyl)piperidin-4-yl)-6,7- dihydropyrrolo[3,4-f]isoindole- 1,3(2H,5H)-dione |
| B11 | 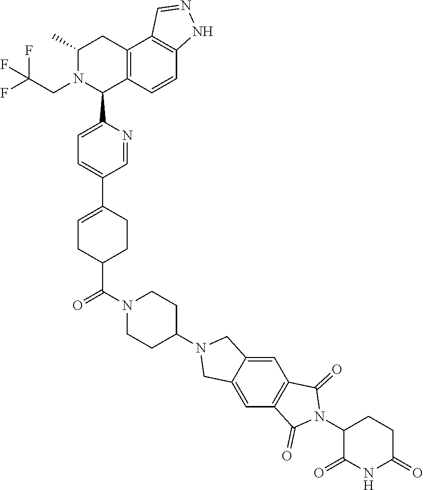 [SMILES: C[C@@H]1Cc2c(ccc3[nH]ncc23)[C@@H](c2ccc(C3=CCC(C(=O)N4CCC(N5Cc6cc7c(cc6C5)C(=O)N(C5CCC(=O)NC5=O)C7=O)CC4)CC3)cn2)N1CC(F)(F)F] | 2-(2,6-dioxopiperidin-3-yl)-6-(1-(4- (6-((6S,8R)-8-methyl-7-(2,2,2- trifluoroethyl)-6,7,8,9-tetrahydro- 3H-pyrazolo[4,3-f]isoquinolin-6- yl)pyridin-3-yl)cyclohex-3-ene-1- carbonyl)piperidin-4-yl)-6,7- dihydropyrrolo[3,4-f]isoindole- 1,3(2H,5H)-dione |
| B12 | 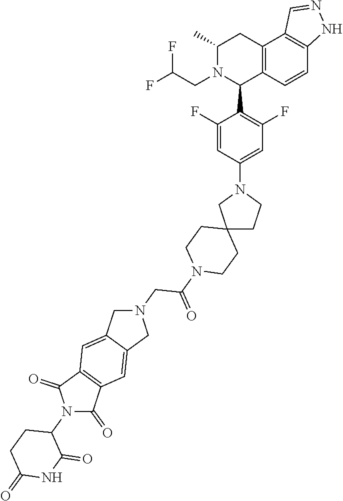 [SMILES: C[C@@H]1Cc2c(ccc3[nH]ncc23)[C@@H](c2c(F)cc(N3CCC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,8- diazaspiro[4.5]decan-8-yl)-2- oxoethyl)-2-(2,6-dioxopiperidin-3- yl)-6,7-dihydropyrrolo[3,4- f]isoindole-1,3(2H,5H)-dione |
| B13 | 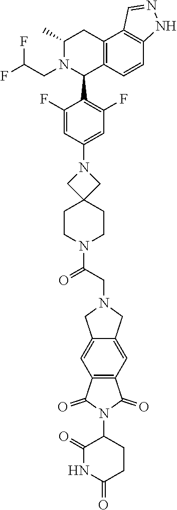 [SMILES: C[C@@H]1Cc2c(ccc3[nH]ncc23)[C@@H](c2c(F)cc(N3CC4(CCN(C(=O)CN5Cc6cc7c(cc6C5)C(=O)N(C5CCC(=O)NC5=O)C7=O)CC4)C3)cc2F)N1CC(F)F] | 6-(2-(2-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,7- diazaspiro[3.5]nonan-7-yl)-2- oxoethyl)-2-(2,6-dioxopiperidin-3- yl)-6,7-dihydropyrrolo[3,4- f]isoindole-1,3(2H,5H)-dione |
| B14 | 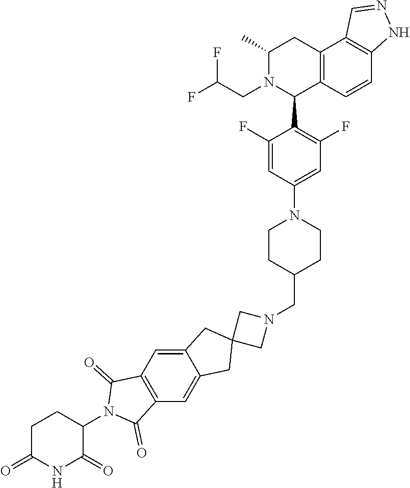 [SMILES: C[C@@H]1Cc2c(ccc3[nH]ncc23)[C@@H](c2c(F)cc(N3CCC(CN4CC5(Cc6cc7c(cc6C5)C(=O)N(C5CCC(=O)NC5=O)C7=O)C4)CC3)cc2F)N1CC(F)F] | 1-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)-2′-(2,6-dioxopiperidin- 3-yl)-5′,7′-dihydro-1′H- spiro[azetidine-3,6′- cyclopenta[f] isoindole]-1′,3′(2′H)- dione |
| B15 | 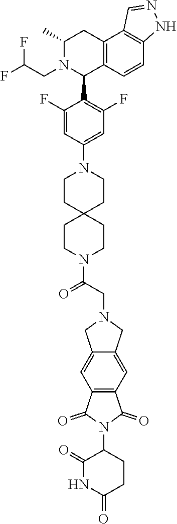 [SMILES: C[C@@H]1Cc2c(ccc3[nH]ncc23)[C@@H](c2c(F)cc(N3CCC4(CCN(C(=O)CN5Cc6cc7c(cc6C5)C(=O)N(C5CCC(=O)NC5=O)C7=O)CC4)CC3)cc2F)N1CC(F)F] | 6-(2-(9-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-3,9- diazaspiro[5.5]undecan-3-yl)-2- oxoethyl)-2-(2,6-dioxopiperidin-3- yl)-6,7-dihydropyrrolo[3,4- f]isoindole-1,3(2H,5H)-dione |
| B17 | 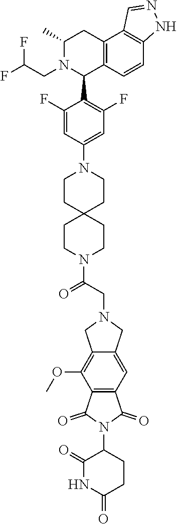 [SMILES: COc1c2c(cc3c1C(=O)N(C1CCC(=O)NC1=O)C3=O)CN(CC(=O)N1CCC3(CC1)CCN(c1cc(F)c([C@@H]4c5ccc6[nH]ncc6c5C[C@@H](C)N4CC(F)F)c(F)c1)CC3)C2] | 6-(2-(9-(4-((6S,8R)-7-(2,2- difluoroethyl)-8-methyl-6,7,8,9- tetrahydro-3H-pyrazolo[4,3- f]isoquinolin-6-yl)-3,5- diazaspiro[5.5]undecan-3-yl)-2- difluorophenyl)-3,9- oxoethyl)-2-(2,6-dioxopiperidin-3- yl)-4-methoxy-6,7- dihydropyrrolo[3,4-f]isoindole- 1,3(2H,5H)-dione |
| B19 | 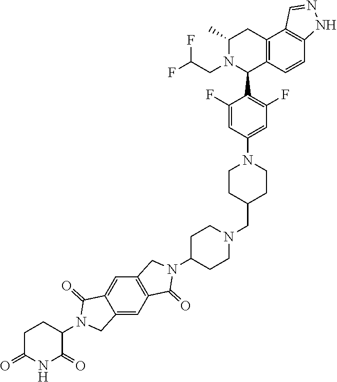 [SMILES: C[C@@H]1Cc2c(ccc3[nH]ncc23)[C@@H](c2c(F)cc(N3CCC(CN4CCC(N5Cc6cc7c(cc6C5=O)CN(C5CCC(=O)NC5=O)C7=O)CC4)CC3)cc2F)N1CC(F)F] | 2-(1-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)piperidin-4-yl)-6-(2,6- dioxopiperidin-3-yl)-2,3,6,7- tetrahydropyrrolo[3,4-f]isoindole- 1,5-dione |
| B24 | 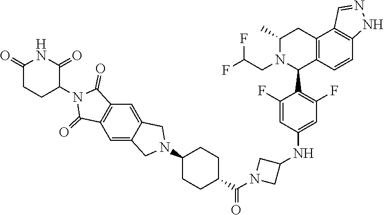 [SMILES: C[C@@H]1Cc2c(ccc3[nH]ncc23)[C@@H](c2c(F)cc(NC3CN(C(=O)[C@H]4CC[C@H](N5Cc6cc7c(cc6C5)C(=O)N(C5CCC(=O)NC5=O)C7=O)CC4)C3)cc2F)N1CC(F)F] | 6-((1S,4r)-4-(3-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)azetidine-1- carbonyl)cyclohexyl)-2-(2,6- dioxopiperidin-3-yl)-6,7- dihydropyrrolo[3,4-f]isoindole- 1,3(2H,5H)-dione |
| B25 | 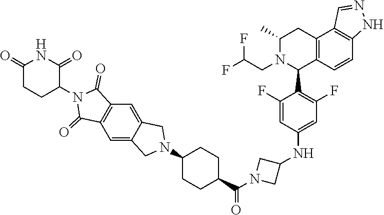 [SMILES: C[C@@H]1Cc2c(ccc3[nH]ncc23)[C@@H](c2c(F)cc(NC3CN(C(=O)[C@H]4CC[C@@H](N5Cc6cc7c(cc6C5)C(=O)N(C5CCC(=O)NC5=O)C7=O)CC4)C3)cc2F)N1CC(F)F] | 6-((1R,4s)-4-(3-((4-((6S,8R)-7- (2,2-difluoroethyl)-8-methyl- 6,7,8,9-tetrahydro-3H- pyrazolo[4,3-f] isoquinolin-6-yl)-3,5- difluorophenyl)amino)azetidine-1- carbonyl)cyclohexyl)-2-(2,6- dioxopiperidin-3-yl)-6,7- dihydropyrrolo[3,4-f]isoindole- 1,3(2H,5H)-dione |
| B44 | 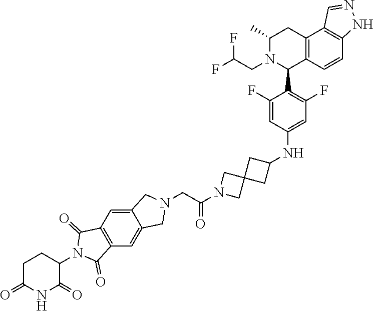 [SMILES: C[C@@H]1Cc2c(ccc3[nH]ncc23)[C@@H](c2c(F)cc(NC3CC4(C3)CN(C(=O)CN3Cc5cc6c(cc5C3)C(=O)N(C3CCC(=O)NC3=O)C6=O)C4)cc2F)N1CC(F)F] | 6-(2-(6-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)-2- azaspiro[3.3]heptan-2-yl)-2- oxoethyl)-2-(2,6-dioxopiperidin-3- yl)-6,7-dihydropyrrolo[3,4- f]isoindole-1,3(2H,5H)-dione |
| B45 | 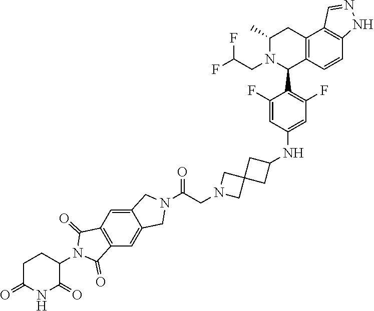 [SMILES: C[C@@H]1Cc2c(ccc3[nH]ncc23)[C@@H](c2c(F)cc(NC3CC4(C3)CN(CC(=O)N3Cc5cc6c(cc5C3)C(=O)N(C3CCC(=O)NC3=O)C6=O)C4)cc2F)N1CC(F)F] | 6-(2-(6-((4-((6S,8R)-7-(2,2- difluoroethyl)-8-methyl-6,7,8,9- tetrahydro-3H-pyrazolo[4,3- f]isoquinolin-6-yl)-3,5- difluorophenyl)amino)-2- azaspiro[3.3] heptan-2-yl)acetyl)-2- (2,6-dioxopiperidin-3-yl)-6,7- dihydropyrrolo[3,4-f]isoindole- 1,3(2H,5H)-dione |
| B48 | 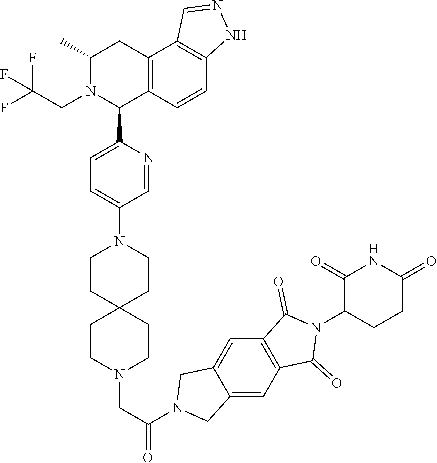 [SMILES: C[C@@H]1Cc2c(ccc3[nH]ncc23)[C@@H](c2ccc(N3CCC4(CCN(CC(=O)N5Cc6cc7c(cc6C5)C(=O)N(C5CCC(=O)NC5=O)C7=O)CC4)CC3)cn2)N1CC(F)(F)F] | 2-(2,6-dioxopiperidin-3-yl)-6-(2-(9- (6-((6S,8R)-8-methyl-7-(2,2,2- trifluoroethyl)-6,7,8,9-tetrahydro- 3H-pyrazolo[4,3-f]isoquinolin-6- yl)pyridin-3-yl)-3,9- diazaspiro[5.5]undecan-3- yl)acetyl)-6,7-dihydropyrrolo[3,4- f]isoindole-1,3(2H,5H)-dione |
| B49 | 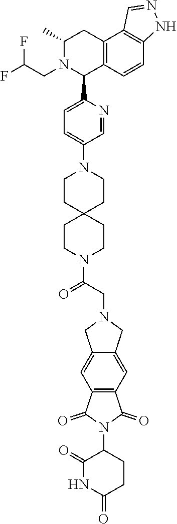 [SMILES: C[C@@H]1Cc2c(ccc3[nH]ncc23)[C@@H](c2ccc(N3CCC4(CCN(C(=O)CN5Cc6cc7c(cc6C5)C(=O)N(C5CCC(=O)NC5=O)C7=O)CC4)CC3)cn2)N1CC(F)F] | 6-(2-(9-(6-((6S,8R)-7-(2,2- difluoroethyl)-8-methyl-6,7,8,9- tetrahydro-3H-pyrazolo[4,3- f]isoquinolin-6-yl)pyridin-3-yl)- 3,9-diazaspiro[5.5]undecan-3-yl)-2- oxoethyl)-2-(2,6-dioxopiperidin-3- yl)-6,7-dihydropyrrolo[3,4- f]isoindole-1,3(2H,5H)-dione |
| B50 | 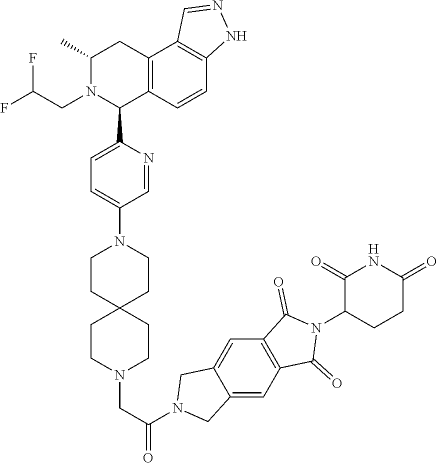 [SMILES: C[C@@H]1Cc2c(ccc3[nH]ncc23)[C@@H](c2ccc(N3CCC4(CCN(CC(=O)N5Cc6cc7c(cc6C5)C(=O)N(C5CCC(=O)NC5=O)C7=O)CC4)CC3)cn2)N1CC(F)F] | 6-(2-(9-(6-((6S,8R)-7-(2,2- difluoroethyl)-8-methyl-6,7,8,9- tetrahydro-3H-pyrazolo[4,3- f]isoquinolin-6-yl)pyridin-3-yl)- 3,9-diazaspiro[5.5]undecan-3- yl)acetyl)-2-(2,6-dioxopiperidin-3- yl)-6,7-dihydropyrrolo[3,4- f]isoindole-1,3(2H,5H)-dione |
| B51 | 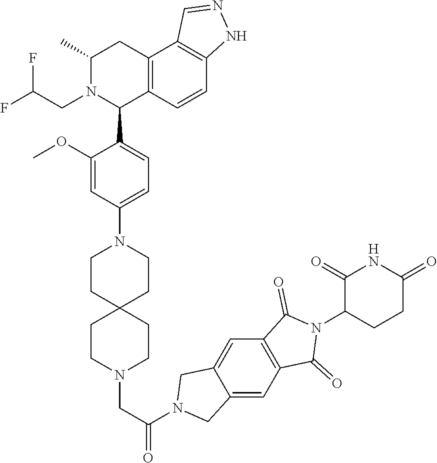 [SMILES: COc1cc(N2CCC3(CCN(CC(=O)N4Cc5cc6c(cc5C4)C(=O)N(C4CCC(=O)NC4=O)C6=O)CC3)CC2)ccc1[C@@H]1c2ccc3[nH]ncc3c2C[C@@H](C)N1CC(F)F] | 6-(2-(9-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-3,9- diazaspiro[5.5]undecan-3- yl)acetyl)-2-(2,6-dioxopiperidin-3- yl)-6,7-dihydropyrrolo[3,4- f]isoindole-1,3(2H,5H)-dione |
| B68 | 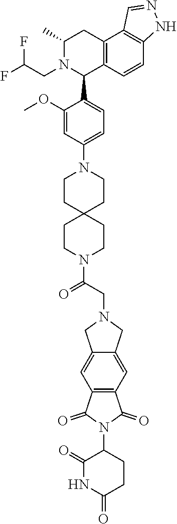 [SMILES: COc1cc(N2CCC3(CCN(C(=O)CN4Cc5cc6c(cc5C4)C(=O)N(C4CCC(=O)NC4=O)C6=O)CC3)CC2)ccc1[C@@H]1c2ccc3[nH]ncc3c2C[C@@H](C)N1CC(F)F] | 6-(2-(9-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-3,9- diazaspiro[5.5]undecan-3-yl)-2- oxoethyl)-2-(2,6-dioxopiperidin-3- yl)-6,7-dihydropyrrolo[3,4- f]isoindole-1,3(2H,5H)-dione |
| B69 | 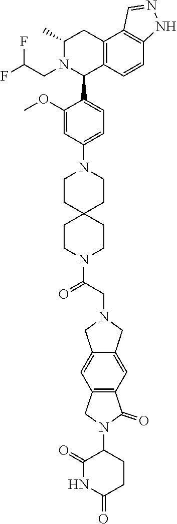 [SMILES: COc1cc(N2CCC3(CCN(C(=O)CN4Cc5cc6c(cc5C4)C(=O)N(C4CCC(=O)NC4=O)C6)CC3)CC2)ccc1[C@@H]1c2ccc3[nH]ncc3c2C[C@@H](C)N1CC(F)F] | 3-(6-(2-(9-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-3,9- diazaspiro[5.5]undecan-3-yl)-2- oxoethyl)-1-oxo-3,5,6,7- tetrahydropyrrolo[3,4-f]isoindol- 2(1H)-yl)piperidine-2,6-dione |
| B70 | 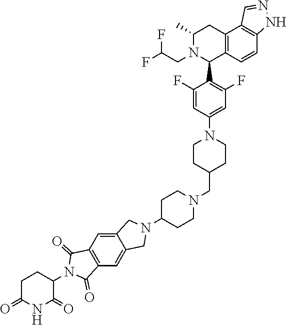 [SMILES: C[C@@H]1Cc2c(ccc3[nH]ncc23)[C@@H](c2c(F)cc(N3CCC(CN4CCC(N5Cc6cc7c(cc6C5)C(=O)N(C5CCC(=O)NC5=O)C7=O)CC4)CC3)cc2F)N1CC(F)F] | 6-(1-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)piperidin-4-yl)-2-(2,6- dioxopiperidin-3-yl)-6,7- dihydropyrrolo[3,4-f]isoindole- 1,3(2H,5H)-dione |
| B71 | 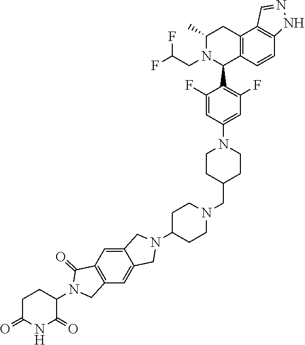 [SMILES: C[C@@H]1Cc2c(ccc3[nH]ncc23)[C@@H](c2c(F)cc(N3CCC(CN4CCC(N5Cc6cc7c(cc6C5)C(=O)N(C5CCC(=O)NC5=O)C7)CC4)CC3)cc2F)N1CC(F)F] | 3-(6-(1-((1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidin-4- yl)methyl)piperidin-4-yl)-1-oxo- 3,5,6,7-tetrahydropyrrolo[3,4- f]isoindol-2(1H)-yl)piperidine-2,6- dione |
| B72 | 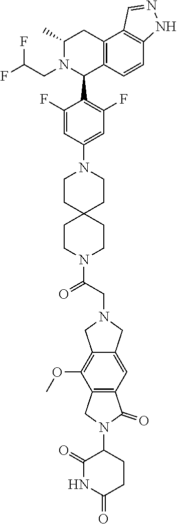 [SMILES: COc1c2c(cc3c1CN(C1CCC(=O)NC1=O)C3=O)CN(CC(=O)N1CCC3(CC1)CCN(c1cc(F)c([C@@H]4c5ccc6[nH]ncc6c5C[C@@H](C)N4CC(F)F)c(F)c1)CC3)C2] | 3-(6-(2-(9-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-3,9- diazaspiro[5.5]undecan-3-yl)-2- oxoethyl)-4-methoxy-1-oxo- 3,5,6,7-tetrahydropyrrolo[3,4- f]isoindol-2(1H)-yl)piperidine-2,6- dione |
| B73 | 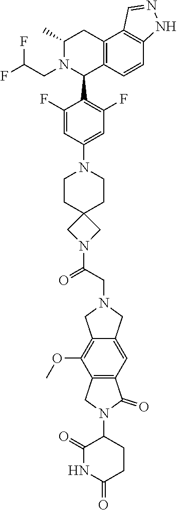 [SMILES: COc1c2c(cc3c1CN(C1CCC(=O)NC1=O)C3=O)CN(CC(=O)N1CC3(CCN(c4cc(F)c([C@@H]5c6ccc7[nH]ncc7c6C[C@@H](C)N5CC(F)F)c(F)c4)CC3)C1)C2] | 3-(6-(2-(7-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-2,7- diazaspiro[3.5]nonan-2-yl)-2- oxoethyl)-4-methoxy-1-oxo- 3,5,6,7-tetrahydropyrrolo[3,4- f]isoindol-2(1H)-yl)piperidine-2,6- dione |
| B74 | 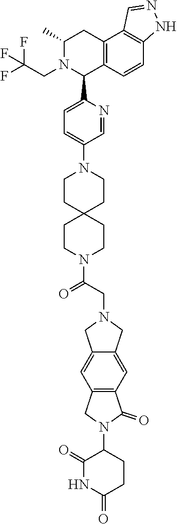 [SMILES: C[C@@H]1Cc2c(ccc3[nH]ncc23)[C@@H](c2ccc(N3CCC4(CCN(C(=O)CN5Cc6cc7c(cc6C5)C(=O)N(C5CCC(=O)NC5=O)C7)CC4)CC3)cn2)N1CC(F)(F)F] | 3-(6-(2-(9-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)- 3,9-diazaspiro[5.5]undecan-3-yl)-2- oxoethyl)-1-oxo-3,5,6,7- tetrahydropyrrolo[3,4-f]isoindol- 2(1H)-yl)piperidine-2,6-dione |
| B75 | 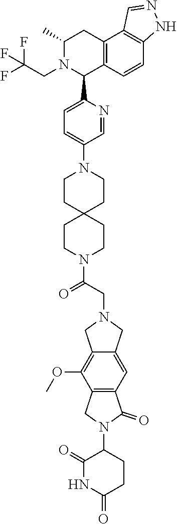 [SMILES: COc1c2c(cc3c1CN(C1CCC(=O)NC1=O)C3=O)CN(CC(=O)N1CCC3(CC1)CCN(c1ccc([C@@H]4c5ccc6[nH]ncc6c5C[C@@H](C)N4CC(F)(F)F)nc1)CC3)C2] | 3-(4-methoxy-6-(2-(9-(6-((6S,8R)- 8-methyl-7-(2,2,2-trifluoroethyl)- 6,7,8,9-tetrahydro-3H- pyrazolo[4,3-f]isoquinolin-6- yl)pyridin-3-yl)-3,9- diazaspiro[5.5]undecan-3-yl)-2- oxoethyl)-1-oxo-3,5,6,7- tetrahydropyrrolo[3,4-f]isoindol- 2(1H)-yl)piperidine-2,6-dione |
| B76 | 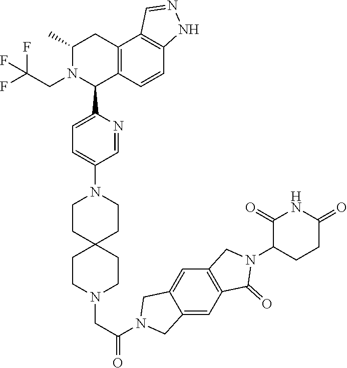 [SMILES: C[C@@H]1Cc2c(ccc3[nH]ncc23)[C@@H](c2ccc(N3CCC4(CCN(CC(=O)N5Cc6cc7c(cc6C5)C(=O)N(C5CCC(=O)NC5=O)C7)CC4)CC3)cn2)N1CC(F)(F)F] | 3-(6-(2-(9-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)- 3,9-diazaspiro[5.5]undecan-3- yl)acetyl)-1-oxo-3,5,6,7- tetrahydropyrrolo[3,4-f]isoindol- 2(1H)-yl)piperidine-2,6-dione |
| B102 | 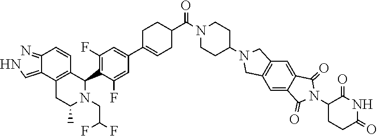 [SMILES: C[C@@H]1Cc2c(ccc3n[nH]cc23)[C@@H](c2c(F)cc(C3=CCC(C(=O)N4CCC(N5Cc6cc7c(cc6C5)C(=O)N(C5CCC(=O)NC5=O)C7=O)CC4)CC3)cc2F)N1CC(F)F] | 6-(1-(4′-((6S,8R)-7-(2,2- difluoroethyl)-8-methyl-6,7,8,9- tetrahydro-3H-pyrazolo[[4,3- f]isoquinolin-6-yl)-3′,5′-difluoro- 2,3,4,5-tetrahydro-[1,1′-biphenyl]- 4-carbonyl)piperidin-4-yl)-2-(2,6- dioxopiperidin-3-yl)-6,7- dihydropyrrolo[3,4-f]isoindole- 1,3(2H,5H)-dione |
| B103 | 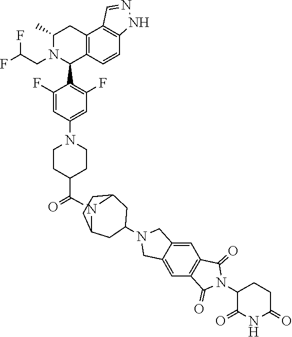 [SMILES: C[C@@H]1Cc2c(ccc3[nH]ncc23)[C@@H](c2c(F)cc(N3CCC(C(=O)N4C5CCC4CC(N4Cc6cc7c(cc6C4)C(=O)N(C4CCC(=O)NC4=O)C7=O)C5)CC3)cc2F)N1CC(F)F] | 6-(8-(1-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)piperidine-4- carbonyl)-8-azabicyclo [3.2.1]octan- 3-yl)-2-(2,6-dioxopiperidin-3-yl)- 6,7-dihydropyrrolo [3,4-f]isoindole- 1,3(2H,5H)-dione |
| B111 | 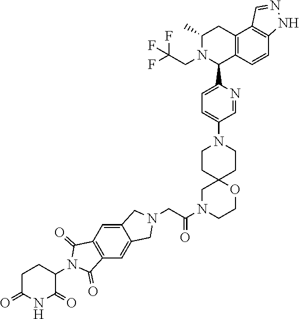 [SMILES: C[C@@H]1Cc2c(ccc3[nH]ncc23)[C@@H](c2ccc(N3CCC4(CC3)CN(C(=O)CN3Cc5cc6c(cc5C3)C(=O)N(C3CCC(=O)NC3=O)C6=O)CCO4)cn2)N1CC(F)(F)F] | (6-((6S,8R)-8-methyl-7-(2,2,2- trifluoroethyl)-6,7,8,9-tetrahydro- 3H-pyrazolo[4,3-f]isoquinolin-6- yl)pyridin-3-yl)-1-oxa-4,9- diazaspiro[5.5]undecan-4-yl)-2- oxoethyl)-6,7-dihydropyrrolo[3,4- f]isoindole-1,3(2H,5H)-dione |

The compounds of the present disclosure may possess advantageous characteristics, as compared to known compounds, such as known estrogen receptor degraders. For example, the compounds of the present disclosure may display more potent estrogen receptor activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In some embodiments, a compound of the present disclosure (e.g., a compound of any of the formulae or any individual compounds disclosed herein) is a solvate. In some embodiments, a compound of the present disclosure (e.g., a compound of any of the formulae or any individual compounds disclosed herein) is a hydrate.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Forms of Compounds Disclosed Herein

### Pharmaceutically Acceptable Salts

In some embodiments, the compounds disclosed herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioatc, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the present disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In some embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers/Stereoisomers

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

In some embodiments, the compounds described herein exist as geometric isomers. In some embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the present disclosure.

In some embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the present disclosure.

In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

In some embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. All tautomeric forms of the compounds disclosed herein are contemplated and are within the scope of the present disclosure. The exact ratio of the tautomers depends on several factors, including temperature, solvent, and pH.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Method

The compounds of the present disclosure can generally be prepared by first preparing pools of intermediates, including a pool of cereblon ligands, a pool of linkers, and a pool of inhibitors, as detailed in the Example section, then followed by subsequent reactions to connect a linker to an inhibitor and a cereblon ligand via metal-catalyzed coupling reactions and reductive amination. Large pool of compounds can be prepared by selecting different combinations of cereblon ligands, linkers, and inhibitors from each pool. General synthetic routes for preparing inhibitor-linker conjugate via metal-catalyzed coupling reactions, which is further coupled to cerebon ligand via reductive amination, are summarize below.

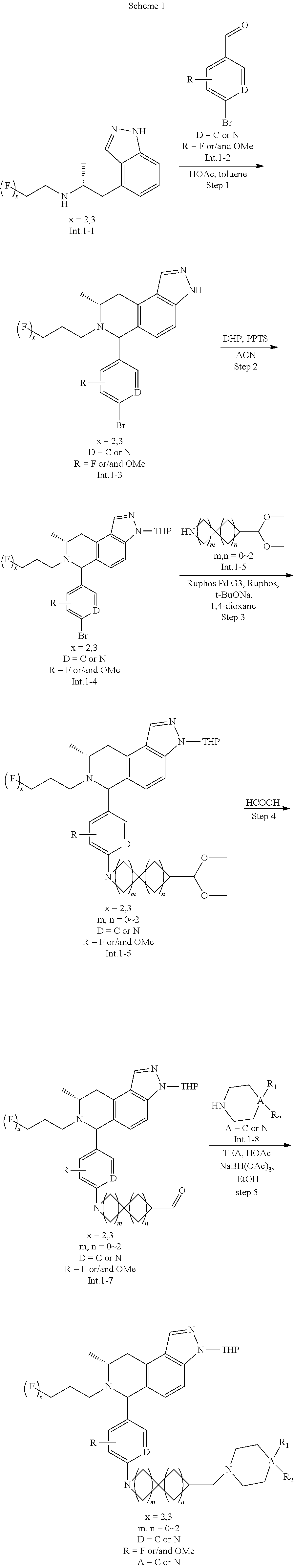

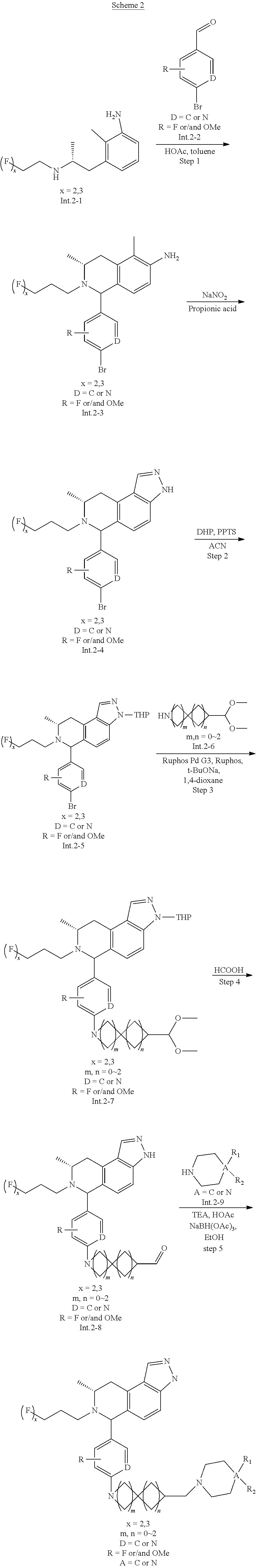

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Elicl, S. H. Wilen, and L. N. Mander (Wilcy-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R.V. “Organic Chemistry, An Intermediate Text” (1996)Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Aequity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Aequity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present application can be assessed with methods and assays known in the art.

The CRBN-DDB1 binding potency of the present disclosure is determined using HTRF assay technology (Perkin Elmer). Compounds are serially diluted and are transferred multi-well plate. The reaction is conducted with addition of His tagged (e.g., CRBN+DDB−DLS7+CXU4) followed by addition of 60 nM fluorescent probe (e.g., Cy5-labeled Thalidomide), and MAb Anti-6HIS Tb cryptate Gold in the assay buffer. After one hour incubation at room temperature, the HTRF signals are read on Envision reader (Perkin Elemer).

ERa degradative activity of compounds can be assessed in MCF-7 and T47D Cells. MCF-7 and T47D cell are seeded and are subsequently treated with the compounds at certain concentrations (e.g., 0.02 to 300 nM). DMSO can be used as vehicle control. Cells are fixed and are blocked with Intercept (PBS) Blocking Buffer (e.g., Li—COR, Odyssey Blocking Buffer), and are stained with ER (e.g., 1:500, Cell signaling) primary antibody for overnight at cold room. Secondary Antibody (e.g., IRDye 800CW Goat anti-Rabbit IgG) and CellTag 700 Stain are added in Intercept (PBS) Blocking Buffer. Finally, cell plate is placed in incubator to dry. Image and signal are captured on Odyssey® DLx Imaging System.

In vitro assay can be accomplished by MCF-7 and T47D Cell Titer Glo (CTG) assay. MCF-7 and T47D cell (From HDB) are cultured in multi-well white plate with phenol red-free RPMI1640+10% CS-FBS+1% P/S medium (e.g., at 1,000cells/well). On day 0: Cells are treated with compound at certain concentrations (e.g., 0.5 to 10000 nM) (DMSO and Staurosporine as control). On day 0 and day 6: add Cell Titer Glo reagent and read on EnVision after 30 min incubation for data generation.

In-cell western blot analysis. Cells are seeded in multi-well plates (e.g., at 40,000 or 10,000 cells/well). Diluted compounds at certain concentration are added (final 0.5% DMSO) and cells are incubated for a certain period of time (e.g., 16 hours). Formaldehyde (e.g., PBS: FA=9:1) is added and followed by washing with PBS. The cells are blocked with Licor blocking buffer (Li-Cor). The relative ER percentage in treated cells are obtained by comparing the values of treated wells to those in untreated and DMSO-treated wells as 100%.

Western Blot Analysis. The cells that are treated with the compounds are lysed in Radioimmunoprecipitation Assay Protein Lysis and Extraction Buffer (e.g., 25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail. Equal amounts of total protein are electrophoresed through 10% SDS-polyacrylamide gels after determination of protein concentration by BCA assay. The separated protein bands are transferred onto PVDF membranes and blotted against different antibodies. The blots are scanned, and the band intensities are quantified (e.g., by using GelQuant.NET software provided by biochemlabsolutions.com). The relative mean intensity of target proteins is expressed after normalization to the intensity of glyceraldehyde-3-phosphate dehydrogenase bands.

Cell Growth Assay. The cells are seeded at certain concentration (e.g., at 1500/well) in multi-well plates overnight. Cells are subsequently treated with the compounds. A certain period of time (e.g., 4 days) after the compound treatment, 10% WST-8 reagent is added to the culture medium and incubate under certain condiction (e.g., in a CO₂ incubator at 37° C. for 2.5 hours). The absorbance is measured on each sample using a microplate reader at certain wavelength (e.g., 450 nm). The relative absorbance is calculated against the vehicle control from three individually repeats.

In vivo pharmacodynamic and efficacy studies. To develop breast cancer cell line xenografts, mice are given 17β-Estradiol in drinking water for a certain period of time. Certain number (e.g., five million) of cells in 50% Matrigel are injected subcutaneously into SCID mice to induce tumor formation. When tumors reach certain size (e.g., 100-400 mm³), mice are treated with vehicle control (e.g., 5% DMSO, 10% solutol, 85% Water) or the compound, and sacrificed at indicated time points. Tumor tissue is harvested for analysis. Tumor sizes and animal weights are measured 2-3 times per week. Tumor volume (mm³)=(length×width2)/2. Tumor growth inhibition is calculated using TGI (%)=(Vc−Vt)/(Vc−Vo)×100, where Vc, Vt are the median of control and treated groups at the end of the study and Vo at the start.

### Methods of Use

In certain aspects, the present disclosure provides methods of degrading an estrogen receptor in a subject, comprising administering to the subject a compound disclosed herein.

In certain aspects, the present disclosure provides uses of a compound disclosed herein in the manufacture of a medicament for degrading an estrogen receptor in a subject.

In certain aspects, the present disclosure provides compounds disclosed herein for use in degrading an estrogen receptor in a subject.

In certain aspects, the present disclosure provides methods of treating or preventing a disease or disorder in a subject in need thereof, comprising administering to the subject a compound disclosed herein (e.g., in a therapeutically effective amount).

In certain aspects, the present disclosure provides methods of treating a disease or disorder in a subject in need thereof, comprising administering to the subject a compound disclosed herein (e.g., in a therapeutically effective amount).

In certain aspects, the present disclosure provides uses of a compound disclosed herein in the manufacture of a medicament for treating or preventing a disease or disorder in a subject in need thereof.

In certain aspects, the present disclosure provides uses of a compound disclosed herein in the manufacture of a medicament for treating a disease or disorder in a subject in need thereof.

In certain aspects, the present disclosure provides compounds disclosed herein for use in treating or preventing a disease or disorder in a subject in need thereof.

In certain aspects, the present disclosure provides compounds disclosed herein for use in treating a disease or disorder in a subject in need thereof.

In certain embodiments, the disease or disorder is an estrogen receptor-mediated disease or disorder.

In certain embodiments, the disease or disorder is cancer.

In certain embodiments, the disease or disorder is breast cancer, lung cancer, ovarian cancer, endometrial cancer, prostate cancer, or esophageal cancer.

In certain embodiments, the cancer includes, but are not limited to, one or more of the cancers of Table A.

TABLE A

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal carcinoma | endocrine gland | endodermal sinus | enteropathy- |
| | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton | mantle cell |
| histiocytoma | nerve sheath tumor | tumor | lymphoma |
| marginal zone B-cell | mast cell leukemia | mediastinal germ cell | medullary carcinoma |
| lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal zone | squamous cell | synovial sarcoma | Sezary's disease |
| lymphoma | carcinoma | | |
| small intestine cancer | squamous carcinoma | stomach cancer | T-cell lymphoma |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In certain embodiments, the cancer is a solid tumor. In certain embodiments, the cancer a hematological cancer. Exemplary hematological cancers include, but are not limited to, the cancers listed in Table B. In certain embodiments, the hematological cancer is acute lymphocytic leukemia, chronic lymphocytic leukemia (including B-cell chronic lymphocytic leukemia), or acute myeloid leukemia.

TABLE B

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In certain embodiments, the subject is a mammal.

In certain embodiments, the subject is a human.

In certain embodiments, the subject is a biological sample (e.g., a cell population).

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jaeques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.F. Eliel, Ed., Univ. of Notre Dame Press, Notre Dame, IN 1972).

The present disclosure additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₄, C₄₋₆, C₄₋₅, and C₅₋₆ alkyl.

The following terms are intended to have the meanings presented therewith below and are useful in understanding the description and intended scope of the present disclosure. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃) 2), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—),butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃) CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadiynyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)=CHCH₂—, —CH═C(CH₃) CH₂—, —CH═CHCH(CH₃)—, —CH═CHC(CH₃)₂—, —CH(CH₃)—CH═CH—, —C(CH₃)₂—CH—CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In certain embodiments, a heteroalkyl group is a saturated group having 1 to 9 carbon atoms and 1, 2, 3, or 4 heteroatoms (“heteroC₁₋₉ alkyl”). In certain embodiments, a heteroalkyl group is a saturated group having 1 to 8 carbon atoms and 1, 2, 3, or 4 heteroatoms (“heteroC₁₋₈ alkyl”). In certain embodiments, a heteroalkyl group is a saturated group having 1 to 7 carbon atoms and 1, 2, 3, or 4 heteroatoms (“heteroC₁₋₇ alkyl”). In certain embodiments, a heteroalkyl group is a group having 1 to 6 carbon atoms and 1, 2, or 3 heteroatoms (“heteroC₁₋₆ alkyl”). In certain embodiments, a heteroalkyl group is a saturated group having 1 to 5 carbon atoms and 1 or 2 heteroatoms (“heteroC₁₋₅ alkyl”). In certain embodiments, a heteroalkyl group is a saturated group having 1 to 4 carbon atoms and/or 2 heteroatoms (“heteroC₁₋₄ alkyl”). In certain embodiments, a heteroalkyl group is a saturated group having 1 to 3 carbon atoms and 1 heteroatom (“heteroC₁₋₃ alkyl”). In certain embodiments, a heteroalkyl group is a saturated group having 1 to 2 carbon atoms and 1 heteroatom (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In certain embodiments, a heteroalkenyl group has 2 to 9 carbon atoms at least one double bond, and 1, 2, 3, or 4 heteroatoms (“heteroC₂₋₉ alkenyl”). In certain embodiments, a heteroalkenyl group has 2 to 8 carbon atoms, at least one double bond, and 1, 2, 3, or 4 heteroatoms (“heteroC₂₋₈ alkenyl”). In certain embodiments, a heteroalkenyl group has 2 to 7 carbon atoms, at least one double bond, and 1, 2, 3, or 4 heteroatoms (“heteroC₂₋₇ alkenyl”). In certain embodiments, a heteroalkenyl group has 2 to 6 carbon atoms, at least one double bond, and 1, 2, or 3 heteroatoms (“heteroC₂₋₆ alkenyl”). In certain embodiments, a heteroalkenyl group has 2 to 5 carbon atoms, at least one double bond, and 1 or 2 heteroatoms (“heteroC₂₋₅ alkenyl”). In certain embodiments, a heteroalkenyl group has 2 to 4 carbon atoms, at least one double bond, and lor 2 heteroatoms (“heteroC₂₋₄ alkenyl”). In certain embodiments, a heteroalkenyl group has 2 to 3 carbon atoms, at least one double bond, and 1 heteroatom (“heteroC₂₋₃ alkenyl”). In certain embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In certain embodiments, a heteroalkynyl group has 2 to 9 carbon atoms, at least one triple bond, and 1, 2, 3, or 4 heteroatoms (“heteroC₂₋₉ alkynyl”). In certain embodiments, a heteroalkynyl group has 2 to 8 carbon atoms, at least one triple bond, and 1, 2, 3, or 4 heteroatoms (“heteroC₂₋₈ alkynyl”). In certain embodiments, a heteroalkynyl group has 2 to 7 carbon atoms, at least one triple bond, and 1, 2, 3, or 4 heteroatoms (“heteroC₂₋₇ alkynyl”). In certain embodiments, a heteroalkynyl group has 2 to 6 carbon atoms, at least one triple bond, and 1, 2, or 3 heteroatoms (“heteroC₂₋₆ alkynyl”). In certain embodiments, a heteroalkynyl group has 2 to 5 carbon atoms, at least one triple bond, and 1 or 2 heteroatoms (“heteroC₂₋₅ alkynyl”). In certain embodiments, a heteroalkynyl group has 2 to 4 carbon atoms, at least one triple bond, and lor 2 heteroatoms (“heteroC₂₋₄ alkynyl”). In certain embodiments, a heteroalkynyl group has 2 to 3 carbon atoms, at least one triple bond, and 1 heteroatom (“heteroC₂₋₃ alkynyl”). In certain embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl, as defined herein, and refers to an optionally substituted alkyl group substituted by an optionally substituted aryl group.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is cither on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl, as defined herein, and refers to an optionally substituted alkyl group substituted by an optionally substituted heteroaryl group.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₅). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share one common bond with, one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or “bridged carbocycle” refers to ring systems wherein the carbocyclyl group, as defined above, form bridged structure with, i.e., share more than one atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the bridged rings. When substitution is indicated, unless otherwise specified, substitution can occur on any of the carbocyclyl rings in which the bridged structure is embedded.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share one common bond with, one or more heterocyclyl or carbocyclyl groups, as defined above, wherein the point of attachment is on any of the fused rings. In such instances, the number of carbons designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than one atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the bridged rings.

“Hetero” when used to describe a compound or a group present on a compound means that one or more carbon atoms in the compound or group have been replaced by a nitrogen, oxygen, sulfur, boron, phosphorus, and silicon heteroatom, as valency permits. Hetero may be applied to any of the hydrocarbyl groups described above having from 1 to 5, and particularly from 1 to 3 heteroatoms.

“Acyl” as used herein, refers to a radical —C(O)R, wherein R is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative acyl groups include, but are not limited to, formyl (—CHO), acetyl (—C(═O)CH₃), cyclohexylcarbonyl, cyclohexylmethylcarbonyl, benzoyl (—C(═O)Ph), and benzylcarbonyl (—C(═O)CH₂Ph).

“Acylamino” as used herein, refers to a radical —NRC(═O)R, wherein each instance of R is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Exemplary “acylamino” groups include, but are not limited to, formylamino, acetylamino, cyclohexylcarbonylamino, cyclohexylmethyl-carbonylamino, benzoylamino and benzylcarbonylamino.

“Acyloxy” as used herein, refers to a radical —OC(═O)R, wherein R is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl and benzylcarbonyl.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Azido” refers to the radical —N₃.

“Amino” refers to the radical —NH₂.

“Hydroxy” refers to the radical —OH.

“Thioketo” refers to the group ═S. “Carboxy” refers to the radical —C(═O)OH.

“Cyano” refers to the radical —CN.

“Halo” or “halogen” refers to fluoro (F), chloro (CI), bromo (Br), and iodo (I). In certain embodiments, the halo group is either fluoro or chloro.

“Nitro” refers to the radical —NO₂.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-f-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic b acid, chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

As used herein, the term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e.g., Berge, et al., J. Pharm. Sci. 66 (1): 1-79 (January 77).

As used herein, “Pharmaceutically acceptable vehicle” refers to a diluent, adjuvant, excipient or carrier with which a compound of the invention is administered.

As used herein, “pharmaceutically acceptable metabolically cleavable group” refers to a group which is cleaved in vivo to yield the parent molecule of the structural formula indicated herein. Examples of metabolically cleavable groups include —COR, —COOR, —CONR₂ and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

As used herein, “solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the present disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

As used herein, a “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, an “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylactically effective amount” refers to the effective amount for prophylactic treatment.

As used herein, “subject in need thereof” refers to a subject having a disease or having an increased risk of developing the disease. A subject in need thereof can be one who has previously been diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, “preventing”, “prevention” or “prophylactic treatment” refers to a reduction in risk of aequiring or developing a disease or disorder (i.e., causing at least one of the clinical symptoms of the disease not to develop in a subject not yet exposed to a disease-causing agent, or in a subject who is predisposed to the disease in advance of disease onset).

As used herein, the term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

As used herein, “treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In some embodiments, “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

It is also to be understood that compounds that have the same molecular formula but differ in the nature or sequence of bonding of their atoms or the arrangement of their atoms in space are termed “isomers.” Isomers that only differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as cither individual enantiomer or as a mixture thereof. A mixture containing equal proportions of the enantiomers is termed a “racemic mixture”.

As used herein “tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight(S) -compound, at least about 99% by weight (R)-compound and at most about 1% by weight(S)-compound, or at least about 99.9% by weight (R)-compound and at most about 0.1% by weight(S) -compound. In certain embodiments, the weights are based upon total weight of compound.

As used herein and unless otherwise indicated, the term “enantiomerically pure(S)-compound” refers to at least about 95% by weight(S)-compound and at most about 5% by weight (R)-compound, at least about 99% by weight(S)-compound and at most about 1% by weight (R)-compound or at least about 99.9% by weight(S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight(S)-compound, by total weight of the compound. For example, a pharmaceutical composition comprising enantiomerically pure(S)-compound can comprise, for example, about 90% excipient and about 10% enantiomerically pure(S)-compound. In certain embodiments, the enantiomerically pure(S)-compound in such compositions can, for example, comprise, at least about 95% by weight(S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

As used herein, the term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range.

As used herein, the term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used herein, the phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in some embodiments, to B only (optionally including elements other than A); in some embodiments, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in some embodiments, to at least one, optionally including more than one, B, with no A present (and optionally including elements other than A); in some embodiments, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

It is understood that the values presented in the examples are approximate values, and they are subject to instrumental and/or experimental variations.

### I. SYNTHESIS AND CHARACTERIZATION OF INTERMEDIATES AND COMPOUNDS A1-A26

The chemical reagents were purchased from commercial sources (such as Alfa, Acros, Sigma Aldrich, TCI and Shanghai Chemical Reagent Company), and used without further purification.

In obtaining the compounds described in the examples below and the corresponding analytical data, the following experimental and analytical protocols were followed unless otherwise indicated.

A summary of LC-MS methods is shown below.

### Method A:

Waters SunFire C18 50*4.6 mm 5 um 2.000 ml/min 2.6 min Column Temperature: 40° C.
Gradient: 5% B hold for 0.2 min, increase to 95% B within 1.40 min, hold at 95% B for 0.9 min, then back to 5% B within 0.01 min
Pump A: 0.1% formic acid (FA) and 10% acetonitrile (ACN) in H₂O
Pump B: 0.1% FA and 10% H₂O in ACN.

### Method B:

Waters SunFire C18 50*4.6 mm 5 um 2.000 ml/min 2.6 min Column Temperature: 40° C.
Gradient: 5% B hold for 0.2 min, increase to 95% B within 1.40 min, hold at 95% B for0.9 min, then back to 5% B within 0.01 min
Pump A: 0.03% trifluoroacetic acid (TFA) in H₂O
Pump B: 0.03% TFA in ACN

### Method C:

Column: Sunfire C18 150*4.6 mm 5 um 1.00 ml/min Column Temperature: 40° C.
Gradient: 10% B hold for 1.8 min, increase to 95% B within 10.2 min, hold at 95% B for 3.0 min, then back to 10% B within 0.01 min
Pump A: 0.03% TFA in H₂O
Pump B: 0.03% TFA in ACN

### Method D:

Column: Luna C18 30*2.0 mm 3 μm 1.200 ml/min 1.5 min Column Temp.: 50° C. 5% B increase to 95% B within 0.7 min, hold at 95% B for 0.4 min, back to 5% B within 0.01 min
Pump A: 0.03% TFA in H₂O
Pump B: 0.03% TFA in ACN

### Method E:

SunFire C18 50*4.6 mm 5 um 2.6 min 2.0 ml/min
Temperature: 40° C.
Gradient: 10% B increase to 30% B for 0.40 min, increase to 95% B within 1.60 min, 95% B hold for 0.90 min, back to 10% B within 0.01 min, A⁷⁰B30

### Method F:

SunFire C18 50*4.6 mm 5 um 2.6 min 2.0 ml/min
Temperature: 40° C.
Gradient: 10% B increase to 30% B for 0.40 min, increase to 95% B within 1.60 min, 95% B hold for 0.90 min, back to 10% B within 0.01 min, A⁵⁰B50.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Compound purification was carried out as needed using a variety of traditional methods including, but not limited to, preparative chromatography under acidic, neutral, or basic conditions using either normal phase or reverse phase HPLC or flash columns or Prep-TLC plates.

Flash chromatography was performed on a Biotage Isolera One via column with silica gel particles of 200-300 mesh. Analytical and preparative thin-layer chromatography was performed using silica gel 60 GF254 plates. Normal-phase silica gel chromatography (FCC) was also performed on silica gel (SiO₂) using prepacked cartridges.

Preparative reverse-phase high performance liquid chromatography (RP HPLC) was performed on either:

### METHOD 1

Prep-HPLC with Waters-Sunfire C18 21.2×250 mm×10 μm, and mobile phase of 10-20% ACN in water (0.1% HCOOH) over 15 min and then hold at 100% ACN for 5 min, at a flow rate of 20 mL/min. or

### METHOD 2

Preparative supercritical fluid high performance liquid chromatography (SFC) was performed either on a Waters 150 Prep-SFC system from Waters. The ABPR was set to 100 bar to keep the CO₂ in SF conditions, and the flow rate may verify according to the compound characteristics, with a flow rate ranging from 70g/min to 140 g/min. The column temperature was ambient temperature

Nuclear magnetic resonance (NMR) spectra were recorded using Brucker AVANCE NEO 400 MHz at around 20-30° C. unless otherwise specified. The following abbreviations are used: s, singlet; d, doublet; t, triplet; q, quartet; m, multiplet; dd, doublet of doublets; ddd, doublet of doublet of doublet; dt, doublet of triplets; bs, broad signal. Chemical shifts were reported in parts per million (ppm, 8) downfield from tetramethylsilane. It will be understood that for compounds comprising an exchangeable proton, said proton may or may not be visible on an NMR spectrum depending on the choice of solvent used for running the NMR spectrum and the concentration of the compound in the solution.

Mass spectra (MS) were obtained on a SHIMADZU LC-MS-2020 MSD using electrospray ionization (ESI) in positive mode unless otherwise indicated. Calculated (calcd.) mass corresponds to the exact mass.

Chemical names were generated using ChemDraw Ultra 12.0, ChemDraw Ultra 14.0 (CambridgeSoft Corp., Cambridge, MA) or ACD/Name Version 10.01 (Advanced Chemistry).

Compounds designated as R* or S* are enantiopure compounds where the absolute configuration was not determined.

### Intermediate 1: (R)-1-(1H-indazol-4-yl)-N-(2,2,2-trifluoroethyl)propan-2-amine

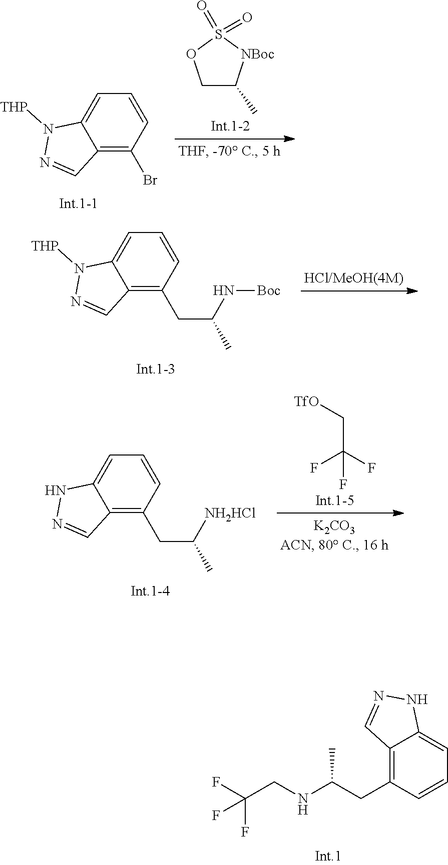

### Step 1-1: tert-butyl ((2R)—1-(1-(tetrahydro-2H-pyran-2-yl)-1H-indazol-4-yl)propan-2-yl)carbamate

To a solution of 4-bromo-1-(tetrahydro-2H-pyran-2-yl)-1H-indazole (2.0 g, 7.14 mmol, 1 eq) in tetrahydrofuran (15 mL) was added slowly butyllithium (4.9 mL, 1.60 molar, 7.86 mmol, 1.1 eq) under Ar atmosphere at −78° C., the solution was stirred at −78° C. for 1 h. Then the solution tetrahydrofuran (9 mL) of tert-butyl (R)-4-methyl-1,2,3-oxathiazolidine-3-carboxylate 2,2-dioxide (1.862 g, 7.86 mmol, 1.leq) was added dropwise into the reaction solution at −78° C. The reaction was stirred at −78° C. for another 5 h. LCMS showed the reaction was completed. The mixture was quenched with saturated NH₄Cl and extracted with EA, combined organic layers, dried over (Na₂SO₄) and filtered. The filtrate was concentrated under reduced pressure. The reside was purified by column (PE:EA=20:1) to afford the product tert-butyl ((2R)-1-(1-(tetrahydro-2H-pyran-2-yl)-1H-indazol-4-yl)propan-2-yl)carbamate (1.64 g. 64.3%) as a white solid. LC purity (L-A⁵⁰B50): 100% (UV at 254 nm), LC-MS: 360.2 [M+H]⁺.

### Step 1-2: (R)-1-(1H-indazol-4-yl)propan-2-amine hydrochloride

To a solution of tert-butyl ((2R)-1-(1-(tetrahydro-2H-pyran-2-yl)-1H-indazol-4-yl)propan-2-yl)carbamate (1.64 g, 4.56 mmol, 1 eq) in MeOH (10 mL), and then HCl/dioxane (4.56 ml, 3M, 13.68 mmol, 3 eq) was added to the mixture under Ar atmosphere, the mixture was stirred at 25° C. for 16 hour. LCMS showed the reaction was completed. The mixture was concentrated under reduced pressure to afford the product (R)-1-(1H-indazol-4-yl)propan-2-amine hydrochloride (790 mg,98%) as white solid.

LC purity (L-A50B50): 100% (UV at 254 nm), LC-MS: 176.1 [M+H]⁺.

### Step 1-3: (R)-1-(1H-indazol-4-yl)-N-(2,2,2-trifluoroethyl)propan-2-amine

To a solution of (R)-1-(1H-indazol-4-yl)propan-2-amine hydrochloride (300 mg, 1.315 mmol, 1 eq) and K₂CO₃ (563.5 mg, 4.077 mmol, 1 eq) in MeCN (5 mL) was added 2,2,2-trifluoroethyl trifluoromethanesulfonate (305.3 mg,1.315 mmol, 1 eq). The mixture heated to 60° C. and stirred for 16 hours under 1 atm. LCMS showed the reaction was completed. The mixture was concentrated under reduced pressure to afford the product (R)-1-(1H-indazol-4-yl)-N-(2,2,2-trifluoroethyl)propan-2-amine (260 mg,83%) as pale yellow solid.

LC-MS purity: 100% (UV at 254 nm), 258.0 [M+H]⁺

### Intermediate 2: (8R)-6-(6-(7-(dimethoxymethyl)-2-azaspiro[3.5]nonan-2-yl)pyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline

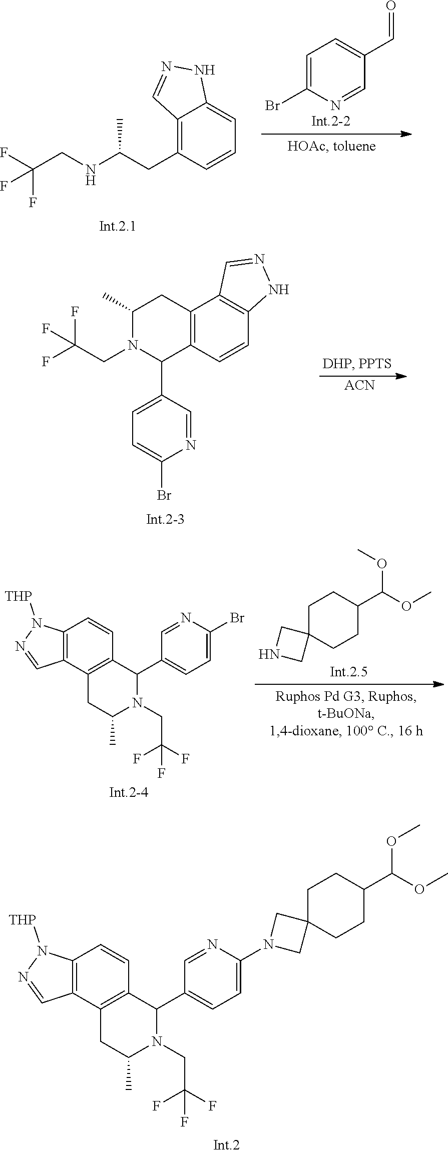

### Step 2-1:(8R)-6-(6-bromopyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline

To a mixture of (R)-1-(1H-indazol-4-yl)-N-(2,2,2-trifluoroethyl)propan-2-amine (800 mg, 2.949 mmol, 1 eq) in HOAC (10 mL) and toluene (10 mL) was added 6-bromonicotinaldehyde (658 mg, 3.539 mmol, 1.2 eq). The mixture was stirred at 110° C. for 16 hours. LCMS showed the reaction was completed. The reaction was concentrated under vacuum to afford (8R)-6-(6-bromopyridin-3-yl)-8-methyl-7-(2,2,2-trifluoroethyl)-6,7,8,9-tetrahydro-3H-pyrazolo[4,3-f]isoquinoline (560 mg, crude) as a white solid

LC-MS purity: 100% (UV at 254 nm), 427.0 [M+H]⁺.

### Step 2-2: (8R)-6-(6-bromopyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline

To a mixture of (8R)-6-(6-bromopyridin-3-yl)-8-methyl-7-(2,2,2-trifluoroethyl)-6,7,8,9-tetrahydro-3H-pyrazolo[4,3-f]isoquinoline (560.0 mg, 1.317 mmol, 1 eq) in DCE (7 mL) was added DHP (553 mg, 6.584 mmol, 5 eq) and stirred at 55° C. for 16 hours. LCMS showed the reaction was completed. The reaction was concentrated under vacuum to afford (8R)-6-(6-bromopyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline (572 mg, crude) as a yellow solid

LC-MS purity: 100% (UV at 254 nm), 509.0 [M+H]⁺.

### Step 2-3: (8R)-6-(6-(7-(dimethoxymethyl)-2-azaspiro[3.5]nonan-2-yl)pyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline

To a mixture of (8R)-6-(6-bromopyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline (300.0 mg, 0.589 mmol, 1 eq), 7-(dimethoxymethyl)-2-azaspiro[3.5]nonane (120.0 mg, 0.589 mmol, 1 eq), t-BuONa (170 mg, 1.767 mmol, 3 eq) and Ruphos (55 mg, 0.118 mmol, 0.2 eq) in 1,4-dixoane (35 mL) was added RuphosPdG3 (99 mg, 0.118 mmol, 0.2 eq) and stirred at 110° C. for 16 hours. LCMS showed the reaction was completed. The reaction was cooled to 20° C. and concentrated under vacuum to afford (8R)-6-(6-(7-(dimethoxymethyl)-2-azaspiro[3.5]nonan-2-yl)pyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline (214 mg, 58%) as a yellow solid.

LC-MS purity: 100% (UV at 254 nm), 628.1 [M+H]⁺.

### Intermediate 3: (8R)-6-(6-(2-(dimethoxymethyl)-7-azaspiro[3.5]nonan-7-yl)pyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline

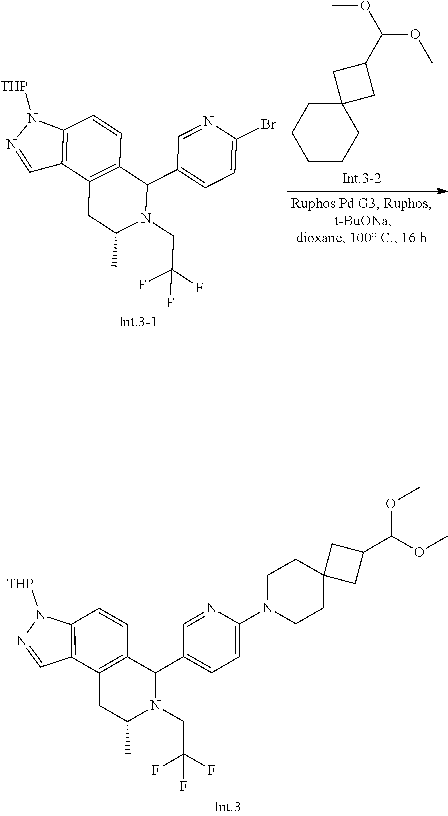

To a solution of (8R)-6-(6-bromopyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline (400 mg, 0.78 mmol, 1 eq), 2-(dimethoxymethyl)-7-azaspiro[3.5]nonane (156.38 mg, 0.78 mmol, 1 eq), t-BuONa (226.56 mg, 2.34 mmol, 3 eq) and Ruphos (73.34 mg, 0.15 mmol, 0.2 eq) in dioxane (15 mL) was added Ruphos G3 (131.45 mg, 0.15 mmol, 0.2 eq). The mixture was stirred at 100° C. for 16 hours under N₂ atmosphere. The solution is yellow and turbid. LCMS showed the starting material was consumed completely and desired compound was detected. The mixture was concentrated to give a residue. The residue was purified by flash to give (8R)-6-(6-(2-(dimethoxymethyl)-7-azaspiro[3.5]nonan-7-yl)pyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline (300 mg, yellow, oil, yield 60.86%).

LC-MS purity: 100% (UV at 254 nm), 628.5 [M+H]⁺.

### Intermediate 4:3-(dimethoxymethyl)-8-(5-((8R)-8-methyl-3-(tetrahydro-2H-pyran-2-yl)-7-(2,2,2-trifluoroethyl)-6,7,8,9-tetrahydro-3H-pyrazolo[4,3-f]isoquinolin-6-yl)pyridin-2-yl)-1-oxa-8-azaspiro[4.5]decane

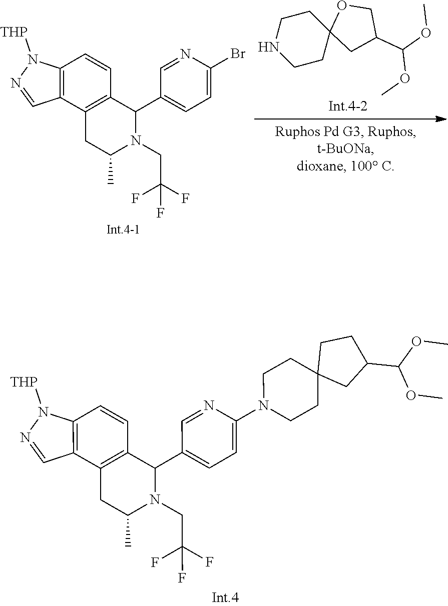

To a mixture of (8R)-6-(6-bromopyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline (122.0 mg, 0.239 mmol, 1 eq), 3-(dimethoxymethyl)-1-oxa-8-azaspiro[4.5]decane (52.0 mg, 0.239 mmol, 1 eq), t-BuONa (69 mg, 0.719 mmol, 3 eq) and Ruphos (23 mg, 0.048 mmol, 0.2 eq) in 1,4-dixoane (35 mL) was added RuphosPdG3 (40 mg, 0.048 mmol, 0.2 eq) and stirred at 110° C. for 16 hours. LCMS showed the reaction was completed. The reaction was cooled to 20° C. and concentrated under vacuum to afford 3-(dimethoxymethyl)-8-(5-((8R)-8-methyl-3-(tetrahydro-2H-pyran-2-yl)-7-(2,2,2-trifluoroethyl)-6,7,8,9-tetrahydro-3H-pyrazolo[4,3-f]isoquinolin-6-yl)pyridin-2-yl)-1-oxa-8-azaspiro[4.5]decane (155 mg, 59%) as a yellow solid.

LC-MS purity: 100% (UV at 254 nm), 644.0 [M+H]⁺.

### Intermediate 5: (8R)-6-(6-(4-(dimethoxymethyl)piperidin-1-yl)pyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline

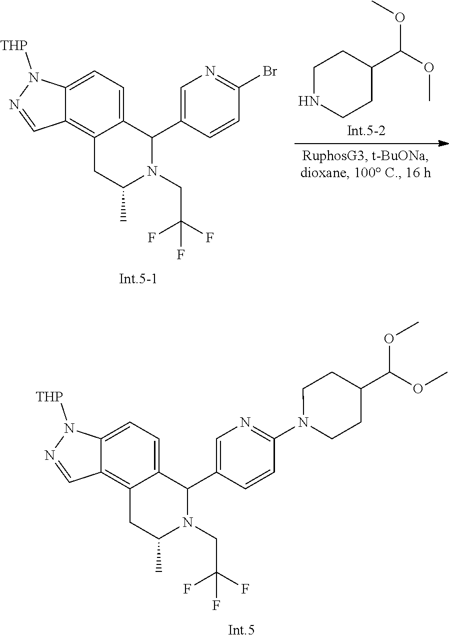

To a solution of (8R)-6-(6-bromopyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline (500 mg, 0.98 mmol, 1 eq), 4-(dimethoxymethyl)piperidine (157 mg, 0.98 mmol, 1 eq), t-BuONa (270 mg, 2.94 mmol, 3 eq) and Ruphos (88.5 mg, 0.19 mmol, 0.2 eq) in dioxane (15 mL) was added Ruphos G3 (164.3 mg, 0.19 mmol, 0.2 eq). The mixture was heated to 100° C. for 16 hours. The solution is yellow and turbid. LCMS showed the starting material was consumed completely and desired compound was detected. The mixture was concentrated to give a residue. The residue was purified by flash to give (8R)-6-(6-(4-(dimethoxymethyl)piperidin-1-yl)pyridin-3-yl)-8-methyl-3-(tetrahydro-2H-pyran-2-yl)-7-(2,2,2-trifluoroethyl)-6,7,8,9-tetrahydro-3H-pyrazolo[4,3-f]isoquinoline (490 mg, yellow, oil, yield 84.94%).

LC-MS purity: 100% (UV at 254 nm), 588.3 [M+H]⁺.

### Intermediate 6:3-((4-(piperidin-4-yl)phenyl)amino) piperidine-2,6-dione

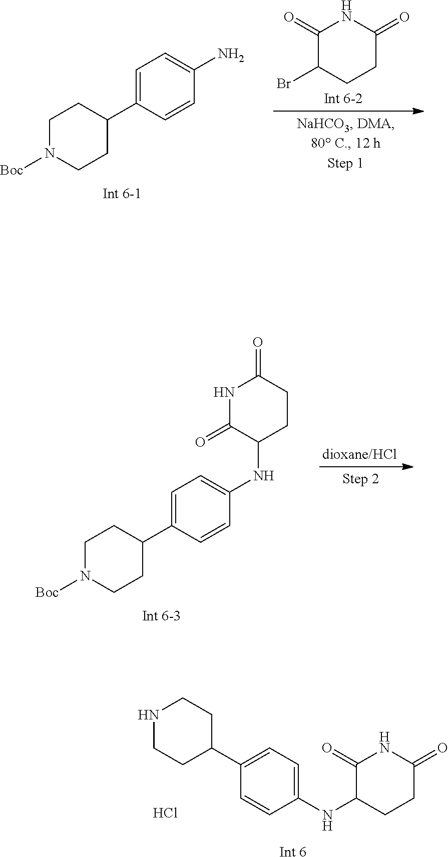

### Step 6-1: tert-butyl 4-(4-((2,6-dioxopiperidin-3-yl)amino)phenyl)piperidine-1-carboxylate

To a mixture of tert-butyl 4-(4-aminophenyl)piperidine-1-carboxylate (1.50 g, 5.428 mmol 1.0 eq) and 3-bromopiperidine-2,6-dione (1.04 g, 5.428 mmol 1.0 eq) in DMA (8 mL) was added NaHCO₃ (456 mg, 5.428 mmol 1.0 eq). The mixture was stirred at 80° C. for 16 hours under N₂. The solvent was evaporated at reduced pressure and the crude product was purified by silica gel column chromatography using 0-100% EtOAc/hexane. The desired tert-butyl 4-(4-((2,6-dioxopiperidin-3-yl)amino)phenyl)piperidine-1-carboxylate was obtained as a light blue solid (1.6 g, 76.0% yield) after lyophilization.

LC-MS purity: 100% (UV at 254 nm), 388.0 [M+H]⁺.

### Step 6-2:3-((4-(piperidin-4-yl)phenyl)amino) piperidine-2,6-dione

To a mixture of tert-butyl 4-(4-((2,6-dioxopiperidin-3-yl)amino)phenyl)piperidine-1-carboxylate (1.6 g, 4.124 mmol, 1.0 eq) in HCl/dioxane (10 mL) was stirred at 20° C. for 2 h. The reaction was direct concentrated to give 3-((4-(piperidin-4-yl)phenyl)amino) piperidine-2,6-dione (1.5 g crude).

LC-MS purity: 100% (UV at 254 nm), 288.0 [M+H]⁺1H NMR (400 MHZ, DMSO) δ 10.83 (s, 1H), 9.11-8.76 (m, 2H), 6.98 (d, J=8.4 Hz, 2H), 6.72 (d, J=8.4 Hz, 2H), 4.33 (dd, J=11.6, 4.8 Hz, 1H), 3.35-3.25 (m, 2H), 3.00-2.84 (m, 2H), 2.79-2.56 (m, 3H), 2.15-2.01 (m, 1H), 1.92-1.73 (m, 5H).

### Intermediate 7: (R/S)-3-((4-(piperidin-4-yl)phenyl)amino) piperidine-2,6-dione hydrochloride salt

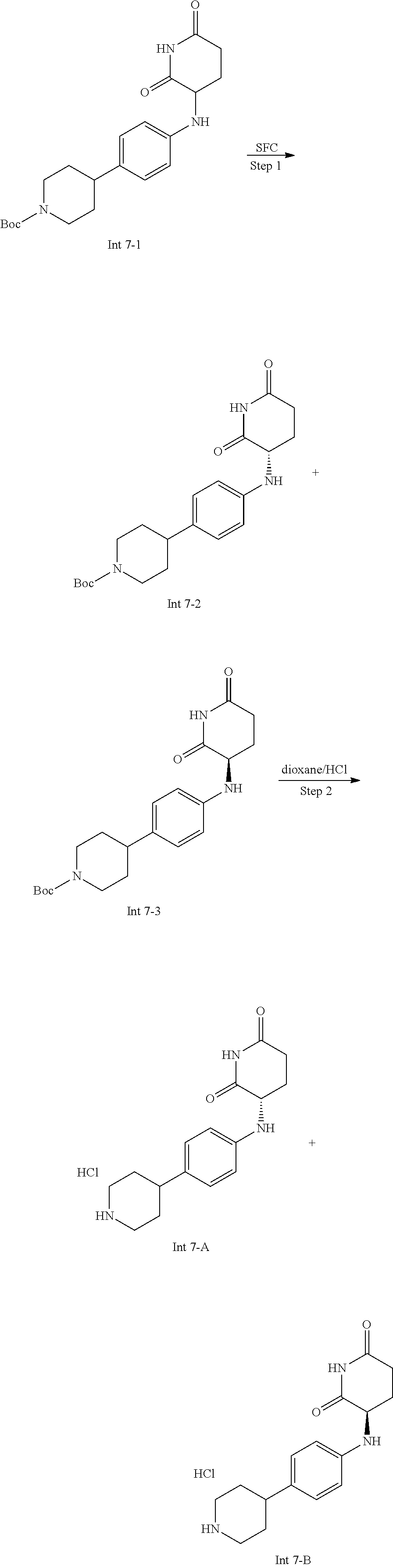

### Step 7-1: (R/S)-tert-butyl 4-(4-((2,6-dioxopiperidin-3-yl)amino)phenyl)piperidine-1-carboxylate

tert-butyl 4-(4-((2,6-dioxopiperidin-3-yl)amino)phenyl)piperidine-1-carboxylate (1.9 g, 5 mmol) was purificd via SFC to afford (R/S)-tert-butyl 4-(4-((2,6-dioxopipcridin-3-yl)amino)phenyl)piperidine-1-carboxylate (P1:450 mg, P2:480 mg).

LC-MS purity: 100% (UV at 254 nm), 388.0 [M+H]⁺.

### Step 7-2: (R/S)-3-((4-(piperidin-4-yl)phenyl)amino) piperidine-2,6-dione hydrochloride salt

A mixture of (R/S)-tert-butyl 4-(4-((2,6-dioxopiperidin-3-yl)amino)phenyl)piperidine-1-carboxylate (100 mg, 0.25 mmol, 1.0 eq) in HCl/dioxane (2 mL) was stirred at 20° C. for 2 h. The reaction was direct concentrated to give (R/S)-3-((4-(piperidin-4-yl)phenyl)amino) piperidine-2,6-dione hydrochloride salt (90 mg, 100% crude yield),. LC-MS purity: 100% (UV at 254 nm), 288.0 [M+H]⁺.

¹H NMR (400 MHZ, DMSO) δ 10.83 (s, 1H), 9.11-8.76 (m, 2H), 6.98 (d, J=8.4 Hz, 2H), 6.72 (d, J=8.4 Hz, 2H), 4.33 (dd, J=11.6, 4.8 Hz, 1H), 3.35-3.25 (m, 2H), 3.00-2.84 (m, 2H), 2.79-2.56 (m, 3H), 2.15-2.01 (m, 1H), 1.92-1.73 (m, 5H).

### Intermediate 8:3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione

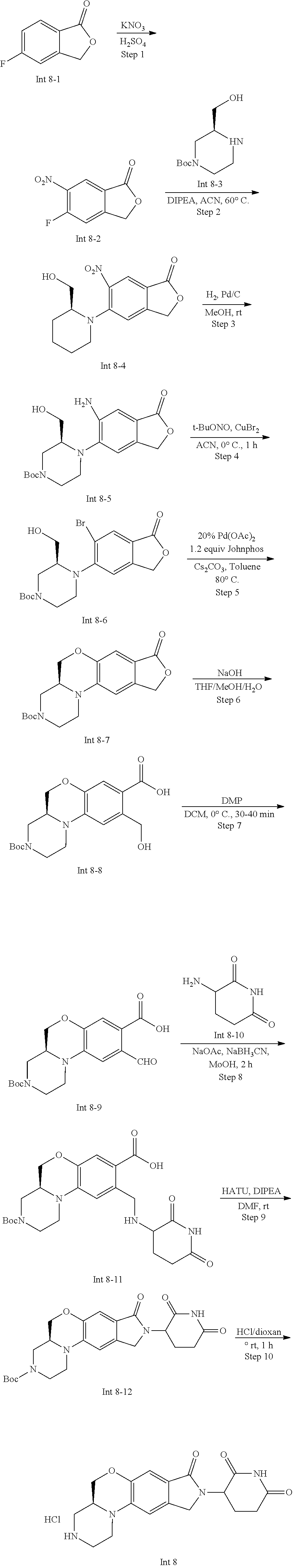

### Step 8-1:5-fluoro-6-nitroisobenzofuran-1 (3H)-one

To a solution of 5-fluoroisobenzofuran-1 (3H)-one (10 g, 1.0 eq) in H₂SO₄ (50 mL) was added KNO₃ (9.97 g, 1.5 eq) in potions. The reaction mixture was stirred at rt for 3 h. TLC (n-hexane: EA=2:1) indicated the starting material was consumed completely and two new spot formed. The reaction mixture was slowly poured into ice water, then extracted with EA, washed with brine, dried (Na₂SO₄) and concentrated under reduced pressure to give a residue which was purified by silica gel flash chromatography (n-Hexane: EA=100:0 to 50:50) to give 5-fluoro-6-nitroisobenzofuran-1 (3H)-one as a white solid (10.4 g, 80%).

Step 8-2: tert-butyl(S)-3-(hydroxymethyl)-4-(6-nitro-1-oxo-1,3-dihydroisobenzofuran-5-yl)piperazine-1-carboxylate

To a solution of 5-fluoro-6-nitroisobenzofuran-1 (3H)-one (1 g, 1.0 eq) and tert-butyl(S)-3-(hydroxymethyl)piperazine-1-carboxylate (1.65 g, 1.5 eq) in acetonitrile (10 mL) was added DIPEA (2.2 mL, 2.5 eq) and stirred at 60° C. for 6 h. TLC (DCM:MeOH=20:1) showed the starting material was completely consumed. Then the reaction mixture was concentrated under reduced pressure and the result residue was purified by silica gel flash chromatography (DCM:MeOH=100:0 to 95:5) to give tert-butyl(S)-3-(hydroxymethyl)-4-(6-nitro-1-oxo-1,3-dihydroisobenzofuran-5-yl)piperazine-1-carboxylate (1.31 g, yield=66%) as a yellow foam.

Step 8-3: tert-butyl(S)-4-(6-amino-1-oxo-1,3-dihydroisobenzofuran-5-yl)-3-(hydroxymethyl)piperazine-1-carboxylate

To a solution of tert-butyl(S)-3-(hydroxymethyl)-4-(6-nitro-1-oxo-1,3-dihydroisobenzofuran-5-yl)piperazine-1-carboxylate (1.0 g, 1.0 eq) in MeOH (15 mL) was added Pd/C (300 mg). The reaction mixture was stirred at rt for 4 h under H₂. UPLC-MS showed the starting material completely conversed to desired product tert-butyl(S)-4-(6-amino-1-oxo-1,3-dihydroisobenzofuran-5-yl)-3-(hydroxymethyl)piperazine-1-carboxylate. Then the reaction mixture was filtered through Celite, and the filtrate was concentrated under reduced pressure to give a light yellow foam (860 mg, 93%), which is pure enough and directly used in the next step. Step 8-4: tert-butyl(S)-4-(6-bromo-1-oxo-1,3-dihydroisobenzofuran-5-yl)-3-(hydroxymethyl)piperazine-1-carboxylate

To a solution of tert-butyl(S)-4-(6-amino-1-oxo-1,3-dihydroisobenzofuran-5-yl)-3-(hydroxymethyl)piperazine-1-carboxylate (468 mg, 1.0 eq) in acetonitrile (25 mL) cooled to 0° C. was added t-BuONO (199 μL, 1.3 eq) and then kept stirring on ice bath for 30 min under N₂. Next, CuBr2 (300 mg, 1.05 eq) was dissolved in 6 mL acetonitrile and added dropwise to the result solution within 30 min. The reaction was kept stirring for 3 h. Then the mixture was diluted with EA (120 mL), washed with NH₄Cl, brine, dried (Na₂SO₄) and concentrated under reduced pressure to give a residue which was purified by silica gel column flash chromatography (DCM:MeOH=100:0 to 95:5) to give tert-butyl(S)-4-(6-bromo-1-oxo-1,3-dihydroisobenzofuran-5-yl)-3-(hydroxymethyl)piperazine-1-carboxylate (415 mg, 75%) as a brown oil.

### Step 8-5: tert-butyl(S)-8-oxo-1,2,4a,5,8,10-hexahydroisobenzofuro[5,6-b]pyrazino[1,2-d][1,4]oxazine-3 (4H)-carboxylate

A mixture of tert-butyl(S)-4-(6-bromo-1-oxo-1,3-dihydroisobenzofuran-5-yl)-3-(hydroxymethyl)piperazine-1-carboxylate (140 mg, 1.0 eq), JohnPhos (118 mg, 1.2 eq) and Cs₂CO₃ (214 mg) in toluene was added Pd (OAc) 2 (36.8 mg, 0.5 eq). The mixture was stirred at 90° C. for 3 h. UPLC-MS indicated the starting material was completely conversion and a new main peak with desired MS was detected. Next, the reaction mixture was cooled to rt, filtered through Celite pad, and the filtrate was concentrated under reduced pressure. The result residue was dissolved in acetonitrile (6 mL) and H₂O (2 mL) (we planned to purify the residue by pre-HPLC, but the desired product is almost insoluble in ACN/H₂O), the insoluble solid was collected after filtered. Product tert-butyl(S)-8-oxo-1,2,4a,5,8,10-hexahydroisobenzofuro[5,6-b]pyrazino[1,2-d][1,4]oxazine-3 (4H)-carboxylate (90 mg, 80%) was obtained as a yellow solid.

### Step 8-6: (S)-3-(tert-butoxycarbonyl)-9-(hydroxymethyl)-1,2,3,4,4a,5-hexahydrobenzo[b]pyrazino[1,2-d][1,4]oxazine-8-carboxylic acid

To a solution of tert-butyl(S)-8-oxo-1,2,4a,5,8,10-hexahydroisobenzofuro[5,6-b]pyrazino[1,2-d][1,4]oxazine-3 (4H)-carboxylate (87 mg, 1.0 eq) in THE/MeOH/H₂O=3/3/1 was added NaOH (60 mg, 6.0 eq), and the mixture was stirred at 40° C. for 6 h. Then the reaction mixture was concentrated under reduced pressure to remove THE/MeOH. The result residue was diluted with water 4 mL and acidified with 2 N HCl to pH 3-4. The mixture was extracted with DCM, and the combined organic layers was washed with brine, dried (Na₂SO₄) and concentrated to give(S)-3-(tert-butoxycarbonyl)-9-(hydroxymethyl)-1,2,3,4,4a,5-hexahydrobenzo[b]pyrazino[1,2-d][1,4]oxazine-8-carboxylic acid (76 mg, 83%) as a white powder.

### Step 8-7: (S)-3-(tert-butoxycarbonyl)-9-formyl-1,2,3,4,4a,5-hexahydrobenzo[b]pyrazino[1,2-d][1,4]oxazine-8-carboxylic acid

To a solution of(S)-3-(tert-butoxycarbonyl)-9-(hydroxymethyl)-1,2,3,4,4a,5-hexahydrobenzo[b]pyrazino[1,2-d][1,4]oxazine-8-carboxylic acid (54 mg, 1.0 eq) in DCM (10 mL) cooled to 0° C. was added DMP (93.7 mg, 1.5 eq). The mixture was stirred at rt for 30-60 minutes until complete conversion. Then the mixture was diluted with DCM, washed with brine, dried (Na₂SO₄) and concentrated under reduced pressure to give(S)-3-(tert-butoxycarbonyl)-9-formyl-1,2,3,4,4a,5-hexahydrobenzo[b]pyrazino[1,2-d][1,4]oxazine-8-carboxylic acid (50 mg, 90%) as a yellow solid, which is directly used in the next step.

### Step 8-8: (4aS)-3-(tert-butoxycarbonyl)-9-(((2,6-dioxopiperidin-3-yl)amino)methyl)-1,2,3,4,4a,5-hexahydrobenzo[b]pyrazino[1,2-d][1,4]oxazine-8-carboxylic acid

A mixture of(S)-3-(tert-butoxycarbonyl)-9-formyl-1,2,3,4,4a,5-hexahydrobenzo[b]pyrazino[1,2-d][1,4]oxazine-8-carboxylic acid (70 mg, 1.0 eq), 3-aminopiperidine-2,6-dione (47.6 mg, 1.5 eq) and NaOAc (23.7 mg, 1.5 eq) was dissolved in MeOH (6 mL) and keep stirred at rt for 20 min. NaBH₃CN (36 mg, 3.0 eq) was added and kept stirring for 1-2 h until the starting material was complete conversion. Then the reaction was quenched with water and the result mixture was concentrated under reduced pressure to give a residue which was purified by pre-HPLC to give (4aS)-3-(tert-butoxycarbonyl)-9-(((2,6-dioxopiperidin-3-yl)amino)methyl)-1,2,3,4,4a,5-hexahydrobenzo[b]pyrazino[1,2-d][1,4]oxazine-8-carboxylic acid (35 mg, 38%) as a white powder.

### Step 8-9: tert-butyl (4aS)-9-(2,6-dioxopiperidin-3-yl)-8-oxo-1,2,4a,5,9,10-hexahydro-8H-pyrazino[1,2′: 4,5][1,4]oxazino[2,3-f]isoindole-3 (4H)-carboxylate

To a solution of (4aS)-3-(tert-butoxycarbonyl)-9-(((2,6-dioxopiperidin-3-yl)amino)methyl)-1,2,3,4,4a,5-hexahydrobenzo[b]pyrazino[1,2-d][1,4]oxazine-8-carboxylic acid (32 mg, 1.0 eq) and DIPEA (35 μL, 3.0 eq) in DMF (2.5 mL) was added HATU (28 mg, 1.1 eq). The reaction mixture was stirred at rt for 10-20 minutes, and then quenched with water (1 mL). The result mixture was directly purified by pre-HPLC to give tert-butyl (4aS)-9-(2,6-dioxopiperidin-3-yl)-8-oxo-1,2,4a,5,9,10-hexahydro-8H-pyrazino[1′,2′: 4,5][1,4]oxazino[2,3-f]isoindole-3 (4H)-carboxylate as a white powder.

### Step 8-10:3-((S)-8-oxo-1,2,3,4,4a,5,8,10-octahydro-9H-pyrazino[1′,2′: 4,5][1,4]oxazino[2,3-f]isoindol-9-yl)piperidine-2,6-dione

A mixture of tert-butyl (4aS)-9-(2,6-dioxopiperidin-3-yl)-8-oxo-1,2,4a,5,9,10-hexahydro-8H-pyrazino[1′,2′: 4,5][1,4]oxazino[2,3-f]isoindole-3 (4H)-carboxylate (30 mg, 1.0 eq) in HCl/dioxane (2 mL) was stirred at 20° C. for 2 h. The after reaction was direct concentration as to give 3-((S)-8-oxo-1,2,3,4,4a,5,8,10-octahydro-9H-pyrazino[11,2′: 4,5][1,4]oxazino[2,3-f]isoindol-9-yl)piperidine-2,6-dione (26 mg, 100%) as white solid. LC-MS: [M+H]⁺=356.90. ¹H NMR (400 MHz, Methanol-d₄) δ 7.15 (s, 1H), 7.11 (d, J=5.7 Hz, 1H), 5.13-5.02 (m, 1H), 4.41-4.27 (m, 3H), 4.20 (d, J=13.6 Hz, 1H), 4.12-4.01 (m, 1H), 3.62-3.43 (m, 3H), 3.30-3.10 (m, 2H), 3.03 -2.94 (m, 1H), 2.94-2.82 (m, 1H), 2.82-2.71 (m, 1H), 2.52-2.38 (m, 1H), 2.20-2.09 (m, 1H). ¹³C NMR (101 MHZ, McOD) δ 174.68, 172.52, 172.49, 171.63, 171.59, 146.42, 146.37, 139.22, 139.20, 138.12, 138.08, 123.85, 123.78, 111.88, 108.70, 108.65, 66.94, 53.72, 53.57, 50.79, 50.75, 48.90, 48.68, 44.36, 44.17, 44.10, 43.63, 43.61, 32.35, 24.08.

### Intermediate 9: N-(2,6-dioxopiperidin-3-yl)-5-(piperazin-1-yl)picolinamide

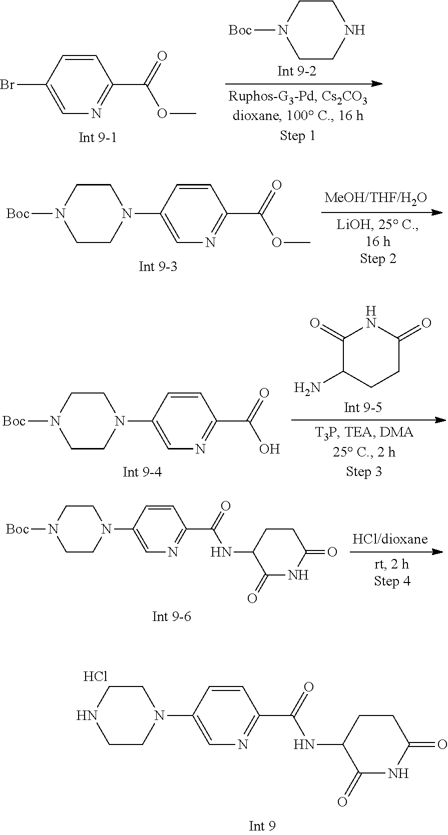

### Step 9-1: tert-butyl 4-(6-(methoxycarbonyl)pyridin-3-yl)piperazine-1-carboxylate

To a mixture of methyl 5-bromopicolinate (15 g, 69.4 mmol 1 eq), tert-butyl piperazine-1-carboxylate (12.9 g, 69.4 mmol, 1 eq.) and Cs₂CO₃ (45 g, 139 mmol, 2 eq.) in dioxane (150 mL) was added Ruphos-G3-Pd (2.2 g, 3.47 mmol 0.05 eq.). The reaction mixture was stirred at 100° C. for 16 h. The mixture was purified via silica gel column to afford tert-butyl 4-(6-(methoxycarbonyl)pyridin-3-yl)piperazine-1-carboxylate (22 g) as a yellow solid.

### Step 9-2:5-(4-(tert-butoxycarbonyl)piperazin-1-yl)picolinic acid

To a mixture of tert-butyl 4-(6-(methoxycarbonyl)pyridin-3-yl)piperazine-1-carboxylate (22 g, 31.5 mmol, 1 eq.) in MeOH (40 ml)/THF (100 ml)/H₂O (40 mL) was added LiOH (6.5 g, 62.2 mmol 2 eq.). The reaction mixture was stirred at 25° C. for 16 h. The mixture was concentrated, and the residue was adjusted to pH=6 with 1N HCl. The precipitate was collected by fitration and dried in vacuo to afford 5-(4-(tert-butoxycarbonyl)piperazin-1-yl)picolinic acid (16.3 g) as a white solid.

### Step 9-3: tert-butyl 4-(6-((2,6-dioxopiperidin-3-yl) carbamoyl)pyridin-3-yl)piperazine-1-carboxylate

To a mixture of 5-(4-(tert-butoxycarbonyl)piperazin-1-yl)picolinic acid (1 g, 3.2 mmol, 1 eq.), 3-aminopiperidine-2,6-dione (537 mg, 3.2 mmol, 0.1 eq.) and TEA (0.8 ml, 6.4 mmol, 2 eq.) in DMA (5 ml) was added T₃P (3 ml, 4.8 mmol, 1.5 eq.). The reaction mixture was stirred at 25° C. for 2 h. The mixture was purified via silica gel column to afford tert-butyl 4-(6-((2,6-dioxopiperidin-3-yl) carbamoyl)pyridin-3-yl)piperazine-1-carboxylate (1.0 g) as a white solid.

### Step 9-4: N-(2,6-dioxopiperidin-3-yl)-5-(piperazin-1-yl)picolinamide

A mixture of tert-butyl 4-(6-((2,6-dioxopiperidin-3-yl) carbamoyl)pyridin-3-yl)piperazine-1-carboxylate (1 g, 1.0 eq) in HCl/dioxane (2 mL) was stirred at 20° C. for 2 h. The reaction was direct concentrated to give N-(2,6-dioxopiperidin-3-yl)-5-(piperazin-1-yl)picolinamide (950 mg, 100%) as white solid.

### Intermediate 10:2-(dimethoxymethyl)-7-azaspiro[3.5]nonane

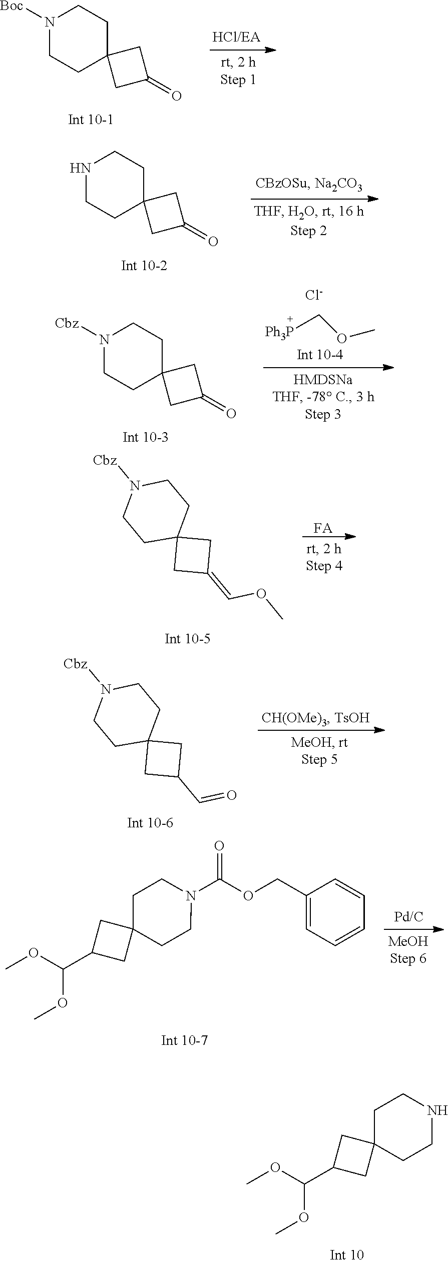

### Step 10-1: benzyl 2-oxo-7-azaspiro[3.5]nonane-7-carboxylate

To a stirred solution of tert-butyl 2-oxo-7-azaspiro[3.5]nonane-7-carboxylate (24 g, 0.1 mol, 1 eq.) in EA (50 mL) at room temperature was added conc. HCl (45 mL, 0.5 mol, 5 eq.) slowly and the reaction mixture was stirred at rt for 1 hour. Once the reaction was completed, the mixture was diluted with EA (150 mL), poured into Na₂CO3 suspension (106 g, 1 mol, 10 eq, in 500 mL of water) and the mixture was stirred for 20 min. To the mixture was added CbzOSu (25 g, 0.1 mmol, 1 eq.) and the mixture was stirred for 1 h. The organic phase was separated, washed with brine, dried, concentrated and the residue was purified by silica column chromatography eluting with 50% EA in PE to give compound benzyl 2-oxo-7-azaspiro[3.5]nonane-7-carboxylate (27 g, 0.1 mol, 100%) as a light yellow oil.

### Step 10-2: benzyl 2-(methoxymethylene)-7-azaspiro[3.5]nonane-7-carboxylate

To a stirred solution of (methoxymethyl)triphenylphosphonium chloride (68 g, 0.2 mol, 2 eq) in dried THF (300 mL) cooled at −70° C. was added NaHMDS (100 mL, 0.2 mol, 2 eq.) dropwise and the mixture was warmed to 0° C. slowly and stirred for 2 h. Then the mixture was cooled at −70° C. and a solution of benzyl 2-oxo-7-azaspiro[3.5]nonane-7-carboxylate (27 g, 0.1 mol, 1 eq.) in THF (50 mL) was added. The mixture was warmed to rt slowly and stirred for 2 h. TLC was done to detect the process of the reaction. Once no starting material was left, the mixture was quenched by NH₄Cl solution (500 mL) and diluted with EA (200 mL). The organic phase was separated, washed with brine, dried, concentrated and the residue was purified by silica column chromatography eluting with 30% EA in PE to give compound benzyl 2-(methoxymethylene)-7-azaspiro[3.5]nonane-7-carboxylate (20 g, 0.067 mol, 67%) as a light yellow oil.

### Step 10-3: benzyl 2-(dimethoxymethyl)-7-azaspiro[3.5]nonane-7-carboxylate

A solution of benzyl 2-(methoxymethylene)-7-azaspiro[3.5]nonane-7-carboxylate (24 g, 0.67 mol, 1 eq.) in FA (50 mL) was stirred at rt for 4 hours. TLC were done to detect the process of the reaction. Once the reaction was completed, the mixture concentrated, and the residue was dissolved in MeOH (120 mL). To the mixture was added CH(OMe) 3 (10.6 g, 0.1 mol, 1.5 eq.) followed by TsOH·H₂O (1.5 g, 0.07 mol, 0.1 eq.) and the mixture was stirred at 70° C. for 12 h. TLC were done to detect the process of the reaction. Once the reaction was completed, the mixture was concentrated and the residue was purified by silica column chromatography eluting with 20% EA in PE to give compound benzyl 2-(dimethoxymethyl)-7-azaspiro[3.5]nonane-7-carboxylate (14.6 g, 0.44 mol, 67%) as light yellow oil.

### Step 10-4:2-(dimethoxymethyl)-7-azaspiro[3.5]nonane

To a solution of compound benzyl 2-(dimethoxymethyl)-7-azaspiro[3.5]nonane-7-carboxylate (14.6 g, 0.44 mol, 1 eq.) in MeOH (100 mL) was added Pd/C (4 g, 10% on carbon, wetted with ca. 55% water) and the mixture was stirred at rt for 12 hours under H₂ (balloon). TLC were done to detect the process of the reaction. Once the reaction was completed, the catalyst was removed by filtration and the filtrate was concentrated to give compound 2-(dimethoxymethyl)-7-azaspiro[3.5]nonane (8.9 g, 0.44 mol, 100%) as a white paste.

LC-MS purity: 100% (UV at 254 nm), 200.0 [M+H]⁺¹H NMR (400 MHZ, DMSO-d₆) δ 4.57 (d, J=6.8 Hz, 1H), 3.20 (m, 6H), 2.61 (s, 2H), 2.47-2.43 (m, 1H), 1.74 (t, 2H), 1.54-1.44 (m, 4H), 1.34 (t, 2H).

### Intermediate 11:4-(dimethoxymethyl)piperidine

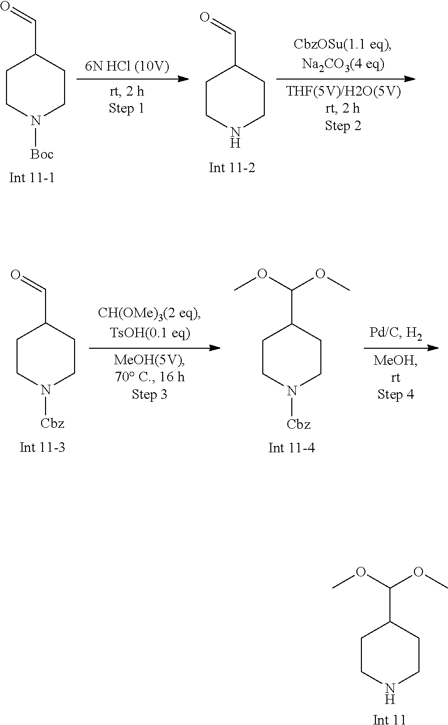

### Step 11-1: benzyl 4-formylpiperidine-1-carboxylate

To a stirred solution of compound tert-butyl 4-formylpiperidine-1-carboxylate (500 g, 2.2 mol, 1 eq.) in EA (500 mL) at room temperature was added conc. HCl (600 mL, 6.6 mol, 3 eq.) slowly and the reaction mixture was stirred at rt for 1 hour. Once the reaction was completed, the mixture was diluted with EA (500 mL), poured into Na₂CO3 suspension (1160 g, 11 mol, 5 eq., in 3000 mL of water) and the mixture was stirred for 20 min. To the mixture was added CbzOSu (550 g, 2.2 mmol, 1 eq.) and the mixture was stirred for 1 h. The organic phase was separated, washed with brine, dried, concentrated and the residue was purified by silica column chromatography eluting with 50% EA in PE to give compound benzyl 4-formylpiperidine-1-carboxylate (550 g, 2.1 mol, 95%) as a light-yellow oil.

### Step 11-2,3: benzyl 4-(dimethoxymethyl)piperidine-1-carboxylate

To a solution of benzyl 4-formylpiperidine-1-carboxylate (150 g, 0.5 mol, 1 eq.) in MeOH (500 mL) was added CH(OMe)₃ (212 g, 1 mol, 2 eq.) followed by TsOH·H₂O (19 g, 0.1 mol, 0.1 eq.) and the mixture was stirred at 70° C. for 12 h. Once the reaction was completed, the mixture was concentrated and the residue was purified by silica column chromatography eluting with 20% EA in PE to give compound benzyl 4-(dimethoxymethyl)piperidine-1-carboxylate (120 g, 0.41 mol, 82%) as light yellow oil.

### Step 11-4:4-(dimethoxymethyl)piperidine

To a solution of compound benzyl 4-(dimethoxymethyl)piperidine-1-carboxylate (120 g, 0.44 mol, 1 eq.) in MeOH (400 mL) was added Pd/C (20 g, 10% on Carbon, wetted with ca. 55% water) and the mixture was stirred at rt for 12 hours under H₂ (balloon). Once the reaction was completed, the catalyst was removed by filtration and the filtrate was concentrated to give compound 4-(dimethoxymethyl)piperidine (65 g, 0.41 mol, 100%) as a white paste.

### Intermediate 12:7-(dimethoxymethyl)-2-azaspiro[3.5]nonane

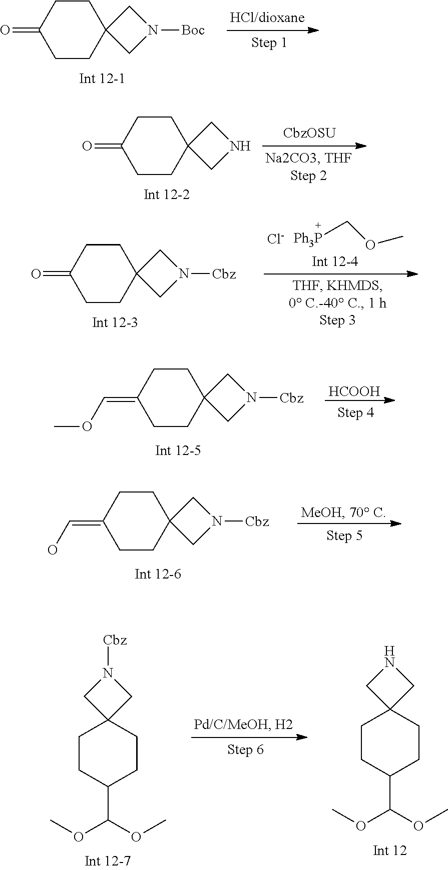

### Step 12-1: benzyl 7-oxo-2-azaspiro[3.5]nonane-2-carboxylate

To a stirred solution of compound tert-butyl 7-oxo-2-azaspiro[3.5]nonane-2-carboxylate (2.4 g, 10 mmol, 1 eq.) in EA (5 mL) at room temperature was added conc. HCl (4.5 mL, 50 mol, 5 eq.) slowly and the reaction mixture was stirred at rt for 1 hour. Once the reaction was completed, the mixture was diluted with EA (15 mL), poured into Na₂CO3 suspension (10.6 g, 0.1 mol, 10 eq, in 50 mL of water) and the mixture was stirred for 20 min. To the mixture was added CbzOSu (2.5 g, 10 mmol, 1 eq.) and the mixture was stirred for 1 h. The organic phase was separated, washed with brine, dried, concentrated and the residue was purified by silica column chromatography eluting with 50% EA in PE to give compound benzyl 7-oxo-2-azaspiro[3.5]nonane-2-carboxylate (2.7 g, 0.1 mol, 100%) as a light yellow oil.

### Step 12-2: benzyl 7-(methoxymethylene)-2-azaspiro[3.5]nonane-2-carboxylate

To a stirred solution of (methoxymethyl)triphenylphosphonium chloride (6.8 g, 20 mol, 2 eq) in dried THF (30 mL) cooled at −70° C. was added NaHMDS (10 mL, 20 mol, 2 eq.) dropwise and the mixture was warmed to 0° C. slowly and stirred for 2 h. Then the mixture was cooled at −70° C. and a solution of benzyl 7-oxo-2-azaspiro[3.5]nonane-2-carboxylate (2.7 g, 0.1 mol, 1 eq.) in THF (5 mL) was added. The mixture was warmed to rt slowly and stirred for 2 h. TLC was done to detect the process of the reaction. Once no starting material was left, the mixture was quenched by NH₄Cl solution (50 mL) and diluted with EA (20 mL). The organic phase was separated, washed with brine, dried, concentrated and the residue was purified by silica column chromatography eluting with 30% EA in PE to give compound benzyl 7-(methoxymethylene)-2-azaspiro[3.5]nonane-2-carboxylate (2.2 g, 7.3 mmol, 73%) as a light yellow oil.

### Step 12-3: benzyl 7-(dimethoxymethyl)-2-azaspiro[3.5]nonane-2-carboxylate

A solution of 7-(methoxymethylene)-2-azaspiro[3.5]nonane-2-carboxylate (2.2 g, 7.3 mol, 1 eq.) in FA (5 mL) was stirred at rt for 4 hours. TLC were done to detect the process of the reaction. Once the reaction was completed, the mixture concentrated, and the residue was dissolved in MeOH (12 mL). To the mixture was added CH(OMe)₃ (1.06 g, 10 mol, 1.5 eq.) followed by TsOH·H₂O (190 mg, 1 mol, 0.1 eq.) and the mixture was stirred at 70° C. for 12 h. TLC were done to detect the process of the reaction. Once the reaction was completed, the mixture was concentrated and the residue was purified by silica column chromatography eluting with 20% EA in PE to give compound benzyl 7-(dimethoxymethyl)-2-azaspiro[3.5]nonane-2-carboxylate (1.5 g, 4.4 mmol, 67%) as light yellow oil.

### Step 12-4:7-(dimethoxymethyl)-2-azaspiro[3.5]nonane

To a solution of compound benzyl 7-(dimethoxymethyl)-2-azaspiro[3.5]nonane-2-carboxylate (1.5 g, 4.4 mol, 1 eq.) in MeOH (10 mL) was added Pd/C (400 mg, 10% on Carbon, wetted with ca. 55% water) and the mixture was stirred at rt for 12 hours under H₂ (balloon). TLC were done to detect the process of the reaction. Once the reaction was completed, the catalyst was removed by filtration and the filtrate was concentrated to give compound 7-(dimethoxymethyl)-2-azaspiro[3.5]nonane (810 mg, 4 mol, 90%) as a white paste.

LC-MS purity: 100% (UV at 254 nm), 200.0 [M+H]⁺.

### Intermediate 13:3-(dimethoxymethyl)-1-oxa-8-azaspiro[4.5]decane

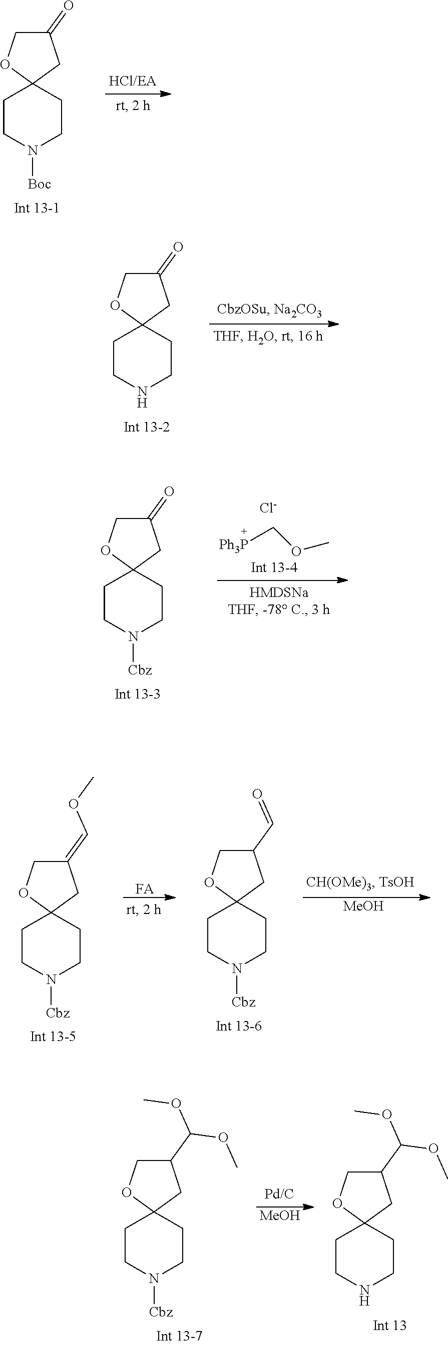

### Step 13-1: benzyl 3-oxo-1-oxa-8-azaspiro[4.5]decane-8-carboxylate

To a stirred solution of tert-butyl 3-oxo-1-oxa-8-azaspiro[4.5]decane-8-carboxylate (11 g, 40 mmol, 1 eq.) in EA (50 mL) at room temperature was added conc. HCl (20 mL, 0.2 mol, 5 eq.) slowly and the reaction mixture was stirred at room temperature for 1 h. Then the mixture was diluted with EA (150 mL), poured into Na₂CO₃ suspension (40 g, 0.4 mol, 10 eq. in 500 mL of water) and the mixture was stirred for 20 min. To the mixture was added CbzOSu (10 g, 40 mmol, 1 eq.) and the mixture was stirred for 1 h. The organic phase was separated, washed with brine, dried, concentrated and the residue was purified by column chromatography on silica gel cluted with 0-40% EtOAc/hexane to afford benzyl 7-oxo-5-oxa-2-azaspiro[3.4]octane-2-carboxylate (11 g, 95% yield) as light yellow oil.

### Step 13-2: benzyl (Z)-3-(methoxymethylene)-1-oxa-8-azaspiro[4.5]decane-8-carboxylate

To a stirred solution of (methoxymethyl)triphenylphosphonium chloride (28.3 g, 80 mmol, 2 eq.) in dried THF (300 mL) cooled at −70° C. was added NaHMDS (40 mL, 160 mmol, 2 eq.) dropwise and the mixture was warmed to 0° C. slowly and stirred for 2 h. Then the mixture was cooled at −70° C. and a solution of benzyl 3-oxo-1-oxa-8-azaspiro[4.5]decane-8-carboxylate (11 g, 40 mmol, 1 eq.) in THF (20 mL) was added. The mixture was warmed to room temperature slowly and stirred for 2 h. The mixture was quenched by NH₄Cl solution (200 mL) and diluted with EA (100 mL). The organic phase was separated, washed with brine, dried, concentrated and the residue was purified by column chromatography on silica gel eluted with 0-40% EtOAc/hexane to afford benzyl (Z)-3-(methoxymethylene)-1-oxa-8-azaspiro[4.5]decane-8-carboxylate (5.4 g, 17 mmol, 44% yield) as light yellow oil.

### Step 13-3: benzyl 3-(dimethoxymethyl)-1-oxa-8-azaspiro[4.5]decane-8-carboxylate

A solution of benzyl (E)-7-(methoxymethylene)-5-oxa-2-azaspiro[3.4]octane-2-carboxylate (5.4 g, 17 mmol, 1 eq.) in FA (20 mL) was stirred at room temperature for 4 h. The mixture was concentrated and the residue was dissolved in MeOH (20 mL). To the mixture was added CH(OMe)₃ (2.5 g, 24 mol, 1.5 eq.) followed by TsOH·H₂O (3.1 g, 1.6 mmol, 0.1 eq.) and the mixture was stirred at 70° C. for 12 h. The mixture was concentrated and the residue was purified by column chromatography on silica gel eluted with 0-40% EtOAc/hexane to afford benzyl 3-(dimethoxymethyl)-1-oxa-8-azaspiro[4.5]decane-8-carboxylate (3.2 g, 50% yield) as light yellow oil.

### Step 13-4:3-(dimethoxymethyl)-1-oxa-8-azaspiro[4.5]decane

To a solution of benzyl 7-(dimethoxymethyl)-5-oxa-2-azaspiro[3.4]octane-2-carboxylate (3.5 g, 10 mmol, 1 eq.) in MeOH (30 mL) was added Pd/C (1 g, 10% on Carbon, wetted with ca. 55% water) and the mixture was stirred at room temperature for 12 h under H₂ (balloon). The catalyst was removed by filtration and the filtrate was concentrated to afford 3-(dimethoxymethyl)-1-oxa-8-azaspiro[4.5]decane (2.1 g, crude) as white paste.

### Intermediate 14: (6S,8R)-7-(2,2-difluoroethyl)-6-(4-(4-(dimethoxymethyl)piperidin-1-yl)-2-methoxyphenyl)-8-methyl-3-(tetrahydro-2H-pyran-2-yl)-6,7,8,9-tetrahydro-3H-pyrazolo[4,3-f]isoquinoline

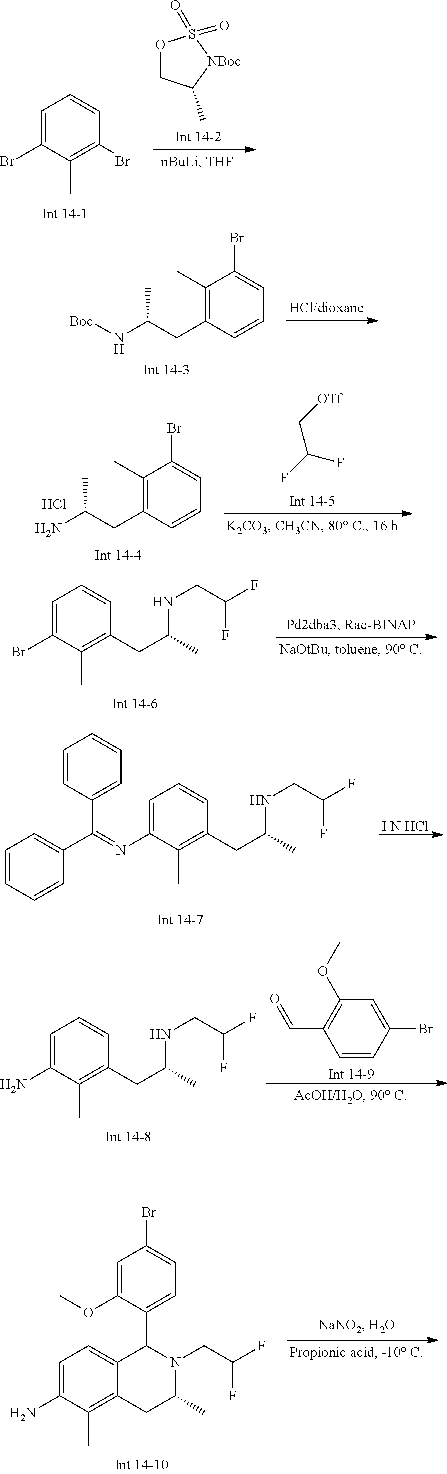

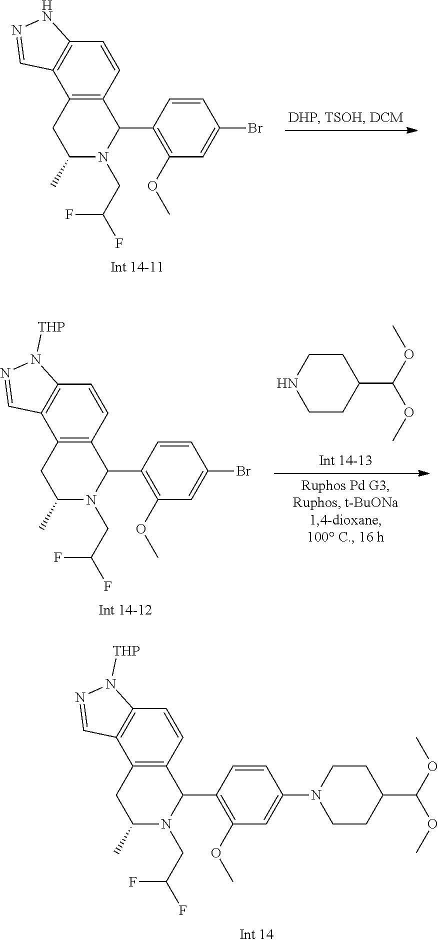

### Step 14-1: tert-butyl (R)-(1-(3-bromo-2-methylphenyl)propan-2-yl)carbamate

To a solution of 1,3-dibromo-2-methylbenzene (3.8 g, 15.17 mmol, 1.2 eq) in tetrahydrofuran (40 mL) cooled to −60° C. was added slowly butyllithium (10 mL, 15.80 mmol, 1.25 eq) and stirred at −60° C. for 1 h. Then the tert-butyl (R)-4-methyl-1,2,3-oxathiazolidine-3-carboxylate 2,2-dioxide (3.0 g, 12.64 mmol, 1.0 eq) in Tetrahydrofuran (20 mL) was added to the reaction and stirred at −60° C. for 3 hours. LCMS showed the reaction was completed. The mixture was quenched with saturated NH₄Cl and extracted with EA to give crude product. The reside was purified by column chromatography on silica gel (PE:EA=10:1) to afford the product tert-butyl (R)-(1-(3-bromo-2-methylphenyl)propan-2-yl)carbamate (3.1 g, 62%) as a white solid. LC-MS purity: 100% (UV at 254 nm),328.1 [M+H]⁺.

### Step 14-2: (R)-1-(3-bromo-2-methylphenyl)propan-2-amine hydrochloride

To a solution of tert-butyl (R)-(1-(3-bromo-2-methylphenyl)propan-2-yl)carbamate (3.1 g, 9.50 mmol, 1 eq,) in HCl/dixoane (50 mL) was stirred at 25° C. for 16 hours. LCMS showed the reaction was completed. The mixture was concentrated under reduced pressure to afford the product (R)-1-(3-bromo-2-methylphenyl)propan-2-amine hydrochloride (2.1 g, 97%) as white solid.

LC-MS purity: 100% (UV at 254 nm), 228.1 [M+H]⁺

### Step 14-3: (R)-1-(3-bromo-2-methylphenyl)-N-(2,2-difluoroethyl)propan-2-amine

To a solution of (R)-1-(3-bromo-2-methylphenyl)propan-2-amine hydrochloride (2.1 g, 8.15 mmol, 1 eq,) and 2,2-difluoroethyl trifluoromethanesulfonate (1.69 g, 7.90 mmol, 0.95 eq) in 1,4-dixoane (40 mL) was added K₂CO₃ (3.68 g, 28.52 mmol, 3 eq) and stirred at 85° C. for 16 hours. LCMS showed the reaction was completed. The mixture was concentrated under reduced pressure to afford the product (R)-1-(3-bromo-2-methylphenyl)-N-(2,2-difluoroethyl)propan-2-amine (1.04 g, 39%) as pale yellow solid.

LC-MS purity: 100% (UV at 254 nm),292.2 [M+H]⁺.

### Step 14-4: (R)—N-(2,2-difluoroethyl)-1-(3-((diphenylmethylene)amino)-2-methylphenyl)propan-2-amine

To a solution of (R)-1-(3-bromo-2-methylphenyl)-N-(2,2-difluoroethyl)propan-2-amine (1.0 g, 3.42 mmol, 1.0 eq), benzophenone imine (683 mg, 3.76 mmol, 1.1 eq), NaOtBu (494 mg, 5.13 mmol, 1.5 eq) and Rac-BINAP (86 mg, 0.14 mmol, 0.4 eq) in toluene (15 mL) was added Pd₂dba₃ (63 mg, 0.07 mmol, 0.2 eq) and stirred at 90° C. for 3 hours. LCMS showed the reaction was completed. The mixture was concentrated under reduced pressure to afford the product (R)—N-(2,2-difluoroethyl)-1-(3-((diphenylmethylene)amino)-2-methylphenyl)propan-2-amine (1.28 g, 95%) as a white solid.

LC-MS purity: 100% (UV at 254 nm),393.3 [M+H]⁺.

### Step 14-5: (R)-3-(2-((2,2-difluoroethyl)amino) propyl)-2-methylaniline

To a solution of (R)—N-(2,2-difluoroethyl)-1-(3-((diphenylmethylene)amino)-2-methylphenyl)propan-2-amine (1.28 g, 3.26 mmol, 1.0 eq) in 1M HCl (15 mL)/DCM (30 mL) was stirred at rt for 1 hour. LCMS showed the reaction was completed. The mixture was concentrated under reduced pressure to afford the product (R)-3-(2-((2,2-difluoroethyl)amino) propyl)-2-methylaniline (700 mg, 94%) as a white solid.

LC-MS purity: 100% (UV at 254 nm),229.3 [M+H]⁺.

### Step 14-6: (3R)-1-(4-bromo-2-methoxyphenyl)-2-(2,2-difluoroethyl)-3,5-dimethyl-1,2,3,4-tetrahydroisoquinolin-6-amine

To a mixture of (R)-3-(2-((2,2-difluoroethyl)amino) propyl)-2-methylaniline (700 mg, 3.07 mmol, 1 eq) and 4-bromo-2-methoxybenzaldehyde (660 mg, 3.07 mmol, 1 eq) in AcOH (2 mL) and toluene (10 ml) and stirred at 90° C. for 3 hours. LCMS showed the reaction was completed.

The reside was purified by column chromatography on silica gel (PE:EA=5:1) to afford the product (3R)-1-(4-bromo-2-methoxyphenyl)-2-(2,2-difluoroethyl)-3,5-dimethyl-1,2,3,4-tetrahydroisoquinolin-6-amine (700 mg, 54%).

LC-MS purity: 100% (UV at 254 nm), 425.4 [M+H]⁺.

### Step 14-7: (6S,8R)-6-(4-bromo-2-methoxyphenyl)-7-(2,2-difluoroethyl)-8-methyl-6,7,8,9-tetrahydro-3H-pyrazolo[4,3-f]isoquinoline

To a mixture of (3R)-1-(4-bromo-2-methoxyphenyl)-2-(2,2-difluoroethyl)-3,5-dimethyl-1,2,3,4-tetrahydroisoquinolin-6-amine (400 mg, 0.94 mmol, 1 eq) in THF (30 mL) and AcOH (35 ml) was added the solution of NaNO₂ (163.7 mg, 1.88 mmol, 2 eq) in H₂O (13 ml) at 0° C. The mixture was stirred at 90° C. for 1 hour. LCMS showed the reaction was completed. The reside was purified by column chromatography on silica gel (PE:EA=5:1) to afford the product (6S,8R)-6-(4-bromo-2-methoxyphenyl)-7-(2,2-difluoroethyl)-8-methyl-6,7,8,9-tetrahydro-3H-pyrazolo[4,3-f]isoquinoline (350 mg, 85%) as a oil liquid.

LC-MS purity: 100% (UV at 254 nm),436.2 [M+H]⁺.

### Step 14-8: (8R)-6-(4-bromo-2-methoxyphenyl)-7-(2,2-difluoroethyl)-8-methyl-3-(tetrahydro-2H-pyran-2-yl)-6,7,8,9-tetrahydro-3H-pyrazolo[4,3-f]isoquinoline

To a mixture of (8R)-6-(4-bromo-2-methoxyphenyl)-7-(2,2-difluoroethyl)-8-methyl-6,7,8,9-tetrahydro-3H-pyrazolo[4,3-f]isoquinoline (350 mg, 0.81 mmol, 1 eq) and DHP (162 mg, 1.21 mmol, 1.5 eq) in DCM (15 mL) was added P-toluensolonic (16 mg, 0.08 mmol, 0.1 eq) and stirred at 40° C. for 16 hours. LCMS showed the reaction was completed. The mixture was concentrated under reduced pressure to afford the product (8R)-6-(4-bromo-2-methoxyphenyl)-7-(2,2-difluoroethyl)-8-methyl-3-(tetrahydro-2H-pyran-2-yl)-6,7,8,9-tetrahydro-3H-pyrazolo[4,3-f]isoquinoline (310 mg, 74%) as a white solid.

LC-MS purity: 100% (UV at 254 nm),520.0 [M+H]⁺.

### Step 14-9: (6S,8R)-7-(2,2-difluoroethyl)-6-(4-(4-(dimethoxymethyl)piperidin-1-yl)-2-methoxyphenyl)-8-methyl-3-(tetrahydro-2H-pyran-2-yl)-6,7,8,9-tetrahydro-3H-pyrazolo[4,3-f]isoquinoline

To a mixture of (8R)-6-(4-bromo-2-methoxyphenyl)-7-(2,2-difluoroethyl)-8-methyl-3-(tetrahydro-2H-pyran-2-yl)-6,7,8,9-tetrahydro-3H-pyrazolo[4,3-f]isoquinoline (310 mg, 0.60 mmol, 1 eq), 4-(dimethoxymethyl)piperidine (238 mg, 1.49 mmol, 2.5 eq), t-BuONa (230 mg, 2.38 mmo, 4 eq) and RuPhos (57 mg, 0.12 mmol, 0.2 eq) in dioxane (25 mL) was added RuPhos Pd G3 (100 mg, 0.12 mmol, 0.2 eq) and stirred at 100° C. for 2 hours under N₂. LCMS showed the reaction was completed. The reside was purified by column chromatography on silica gel (PE:EA=3:1) to afford the product (6S,8R)-7-(2,2-difluoroethyl)-6-(4-(4-(dimethoxymethyl)piperidin-1-yl)-2-methoxyphenyl)-8-methyl-3-(tetrahydro-2H-pyran-2-yl)-6,7,8,9-tetrahydro-3H-pyrazolo[4,3-f]isoquinoline (250 mg, 70%) as a white solid.

LC-MS purity: 100% (UV at 254 nm),599.1 [M+H]⁺.

### Intermediate 15: (8R)-6-(5-(2-(dimethoxymethyl)-7-azaspiro[3.5]nonan-7-yl)pyridin-2-yl)-7-((1-fluorocyclopropyl)methyl)-8-methyl-3-(tetrahydro-2H-pyran-2-yl)-6,7,8,9-tetrahydro-3H-pyrazolo[4,3-f]isoquinoline

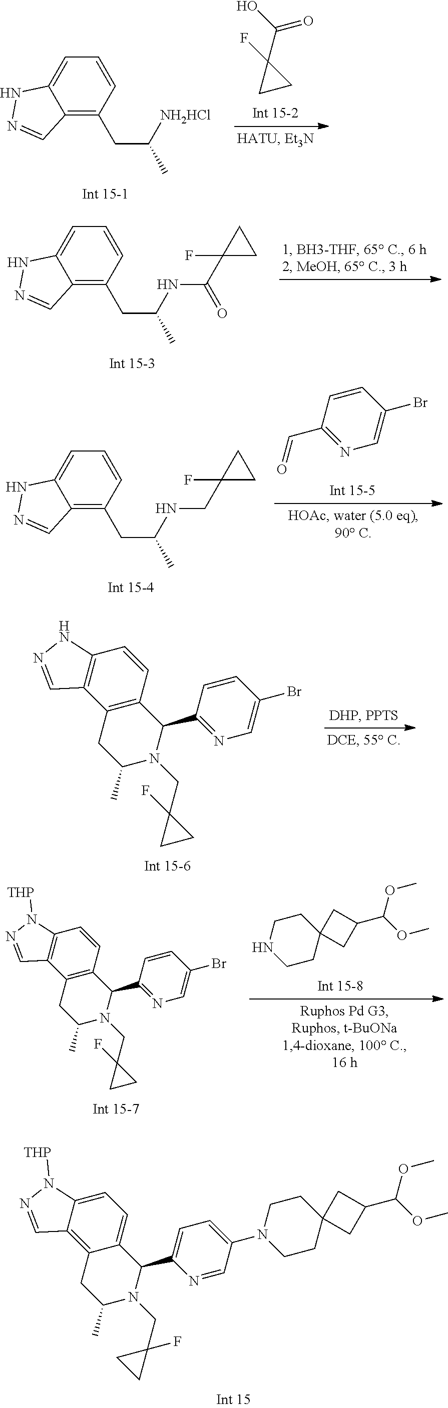

### Step 15-1: (R)—N-(1-(1H-indazol-4-yl)propan-2-yl)-1-fluorocyclopropane-1-carboxamide

To a solution of (R)-1-(1H-indazol-4-yl)propan-2-amine hydrochloride (5.0 g, 23.62 mmol, 1 eq), 1-fluorocyclopropane-1-carboxylic acid (2.5 g, 23.62 mmol, 1 eq) and triethylamine (9.8 mL, 70.86 mmol, 3 eq) in DMF (50 mL) was added slowly HATU (13.5 g, 35.4 mmol, 1.5 eq) and stirred at 25° C. for 16 hours. LCMS showed the reaction was completed. The mixture was quenched with saturated NH₄Cl and extracted with EA to give crude product. The reside was purified by column chromatography on silica gel (PE:EA=10:1) to afford the product (R)—N-(1-(1H-indazol-4-yl)propan-2-yl)-1-fluorocyclopropane-1-carboxamide (4.1 g. 66%) as a oil liquid. LC-MS purity: 100% (UV at 254 nm),262.1 [M+H]⁺.

### Step 15-2: (R)—N-((1-fluorocyclopropyl)methyl)-1-(1H-indazol-4-yl)propan-2-amine

To a solution of (R)—N-(1-(1H-indazol-4-yl)propan-2-yl)-1-fluorocyclopropane-1-carboxamide (4.1 g, 15.69 mmol, 1 eq) in THF (50 mL) cooled to −20° C. was added slowly BH₃·THF (1 M, 47.1 mL, 47.1 mmol, 3 eq) and stirred at 65° C. for 6 hours. LCMS showed the reaction was completed. The mixture was quenched with H₂O (100 mL) and extracted with EA to give crude product. The reside was purified by column chromatography on silica gel (PE:EA=3:1) to afford the product (R)—N-((1-fluorocyclopropyl)methyl)-1-(1H-indazol-4-yl)propan-2-amine (2.1 g. 54%) as an oil liquid.

LC-MS purity: 100% (UV at 254 nm),248.2 [M+H]⁺.

### Step 15-3: (6S,8R)-6-(5-bromopyridin-2-yl)-7-((1-fluorocyclopropyl)methyl)-8-methyl-6,7,8,9-tetrahydro-3H-pyrazolo[4,3-f]isoquinoline

To a mixture of (R)—N-((1-fluorocyclopropyl)methyl)-1-(1H-indazol-4-yl)propan-2-amine (2.1 g, 8.49 mmol, 1 eq) in HOAC (15 mL) and toluene (15 mL) was added 5-bromopicolinaldehyde (1.90 g, 10.19 mmol, 1.2 eq). The mixture was stirred at 90° C. for 16 hours. LCMS showed the reaction was completed. The reaction was concentrated under vacuum to afford (6S,8R)-6-(5-bromopyridin-2-yl)-7-((1-fluorocyclopropyl)methyl)-8-methyl-6,7,8,9-tetrahydro-3H-pyrazolo[4,3-f]isoquinoline (500 mg, 14%) as a oil liquid.

LC-MS purity: 100% (UV at 254 nm),415.3, 417.3 [M+H]⁺.

### Step 15-4: (6S,8R)-6-(5-bromopyridin-2-yl)-7-((1-fluorocyclopropyl)methyl)-8-methyl-3-(tetrahydro-2H-pyran-2-yl)-6,7,8,9-tetrahydro-3H-pyrazolo[4,3-f]isoquinoline

To a mixture of (6S,8R)-6-(5-bromopyridin-2-yl)-7-((1-fluorocyclopropyl)methyl)-8-methyl-6,7,8,9-tetrahydro-3H-pyrazolo[4,3-f]isoquinoline (500.0 mg, 1.20 mmol, 1 eq) and DHP (506.4 mg, 6.02 mmol, 5 eq) in DCE (15 mL) was added PPTS (60.5 mg, 0.24 mmol, 0.2 eq) and stirred at 55° C. for 16 hours. LCMS showed the reaction was completed. The reaction was concentrated under vacuum to afford (6S,8R)-6-(5-bromopyridin-2-yl)-7-((1-fluorocyclopropyl)methyl)-8-methyl-3-(tetrahydro-2H-pyran-2-yl)-6,7,8,9-tetrahydro-3H-pyrazolo[4,3-f]isoquinoline (460 mg, 76%) as a yellow solid

LC-MS purity: 100% (UV at 254 nm),499.1, 501.1 [M+H]⁺.

### Step 15-5: (8R)-6-(5-(2-(dimethoxymethyl)-7-azaspiro[3.5]nonan-7-yl)pyridin-2-yl)-7-((1-fluorocyclopropyl)methyl)-8-methyl-3-(tetrahydro-2H-pyran-2-yl)-6,7,8,9-tetrahydro-3H-pyrazolo[4,3-f]isoquinoline

To a mixture of (6S,8R)-6-(5-bromopyridin-2-yl)-7-((1-fluorocyclopropyl)methyl)-8-methyl-3-(tetrahydro-2H-pyran-2-yl)-6,7,8,9-tetrahydro-3H-pyrazolo[4,3-f]isoquinoline (460.0 mg, 0.92 mmol, 1 eq), 2-(dimethoxymethyl)-7-azaspiro[3.5]nonane (183.6 mg, 0.92 mmol, 1 eq), t-BuONa (265.6 mg, 2.76 mmol, 3 eq) and Ruphos (42.8 mg, 0.09 mmol, 0.1 eq) in 1,4-dixoane (10 mL) was added RuphosPdG3 (76.9 mg, 0.09 mmol, 0.1 eq) and stirred at 100° C. for 16 hours. LCMS showed the reaction was completed. The reaction was cooled to 20° C. and concentrated under vacuum to give crude product. The reside was purified by column chromatography on silica gel (PE:EA=3:1) to afford (8R)-6-(5-(2-(dimethoxymethyl)-7-azaspiro[3.5]nonan-7-yl)pyridin-2-yl)-7-((1-fluorocyclopropyl)methyl)-8-methyl-3-(tetrahydro-2H-pyran-2-yl)-6,7,8,9-tetrahydro-3H-pyrazolo[4,3-f]isoquinoline (281 mg, 49%) as a yellow solid.

LC-MS purity: 100% (UV at 254 nm), 618.4 [M+H]⁺.

### Intermediate 16: (8R)-6-(4-bromo-2,6-difluorophenyl)-7-(2,2-difluoroethyl)-8-methyl-3-(tetrahydro-2H-pyran-2-yl)-6,7,8,9-tetrahydro-3H-pyrazolo[4,3-f]isoquinoline

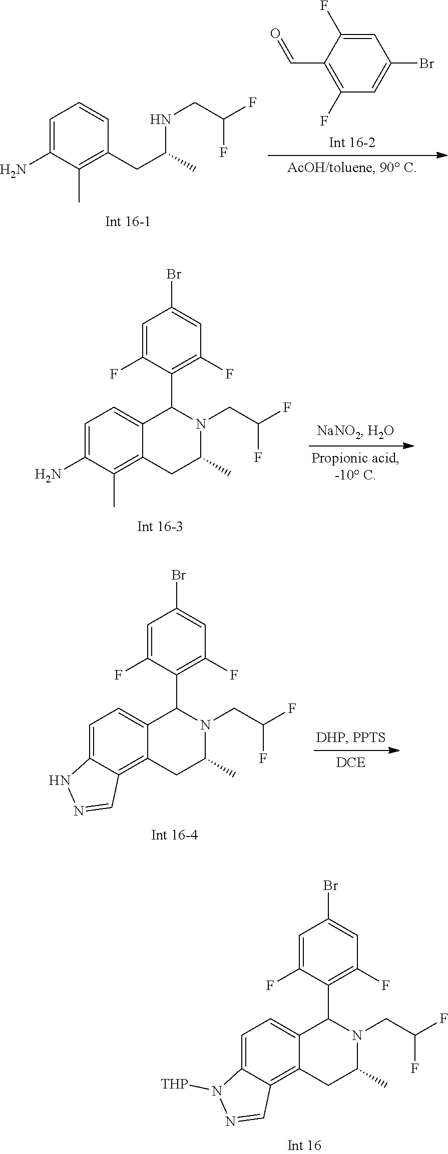

### Step 16-1:(1S,3R)-1-(4-bromo-2,6-difluorophenyl)-2-(2,2-difluoroethyl)-3,5-dimethyl-1,2,3,4-tetrahydroisoquinolin-6-amine

To a mixture of (R)-3-(2-((2,2-difluoroethyl)amino) propyl)-2-methylaniline (2.1 g, 9.21 mmol, 1 eq) in AcOH (20 mL) and toluene (20 mL) was added 4-bromo-2,6-difluorobenzaldehyde (2.4 g, 11.05 mmol, 1.2 eq) and stirred at 90° C. for 16 hours. LCMS showed the reaction was completed. The reaction was concentrated under vacuum to afford (1S,3R)-1-(4-bromo-2,6-difluorophenyl)-2-(2,2-difluoroethyl)-3,5-dimethyl-1,2,3,4-tetrahydroisoquinolin-6-amine (2.9 g, crude) as a white solid

LC-MS purity: 100% (UV at 254 nm),431.1 [M+H]⁺.

### Step 16-2: (8R)-6-(4-bromo-2,6-difluorophenyl)-7-(2,2-difluoroethyl)-8-methyl-6,7,8,9-tetrahydro-3H-pyrazolo[4,3-f]isoquinoline

To a mixture of (1S,3R)-1-(4-bromo-2,6-difluorophenyl)-2-(2,2-difluoroethyl)-3,5-dimethyl-1,2,3,4-tetrahydroisoquinolin-6-amine (500.0 mg, 1.163 mmol, 1 eq) in propionic acid (7 mL) and THF (7 mL) was added the solution of NaNO₂ (240 mg, 3.489 mmol, 3 eq) in H₂O (1 ml) at 0° C. and stirred at −10° C. for 2 hours. LCMS showed the reaction was completed. The reaction was concentrated under vacuum to afford (8R)-6-(4-bromo-2,6-difluorophenyl)-7-(2,2-difluoroethyl)-8-methyl-6,7,8,9-tetrahydro-3H-pyrazolo[4,3-f]isoquinoline (180 mg, 35%) as a yellow solid.

LC-MS purity: 100% (UV at 254 nm),442.3 [M+H]⁺.

### Step 16-3: (8R)-6-(4-bromo-2,6-difluorophenyl)-7-(2,2-difluoroethyl)-8-methyl-3-(tetrahydro-2H-pyran-2-yl)-6,7,8,9-tetrahydro-3H-pyrazolo[4,3-f]isoquinoline

To a mixture of (8R)-6-(4-bromo-2,6-difluorophenyl)-7-(2,2-difluoroethyl)-8-methyl-6,7,8,9-tetrahydro-3H-pyrazolo[4,3-f]isoquinoline (180.0 mg, 0.409 mmol, 1 eq) in DCE (7 mL) was added DHP (172 mg, 2.045 mmol, 5 eq) and stirred at 55° C. for 16 hours. LCMS showed the reaction was completed. The reaction was cooled to 20° C. and concentrated under vacuum to afford (8R)-6-(4-bromo-2,6-difluorophenyl)-7-(2,2-difluoroethyl)-8-methyl-3-(tetrahydro-2H-pyran-2-yl)-6,7,8,9-tetrahydro-3H-pyrazolo[4,3-f]isoquinoline (120 mg, 58%) as a yellow solid.

LC-MS purity: 100% (UV at 254 nm), 526.1 [M+H]⁺

### Intermediate 17: (S)—N-(2,6-dioxopiperidin-3-yl)-6-fluoroisoindoline-5-carboxamide

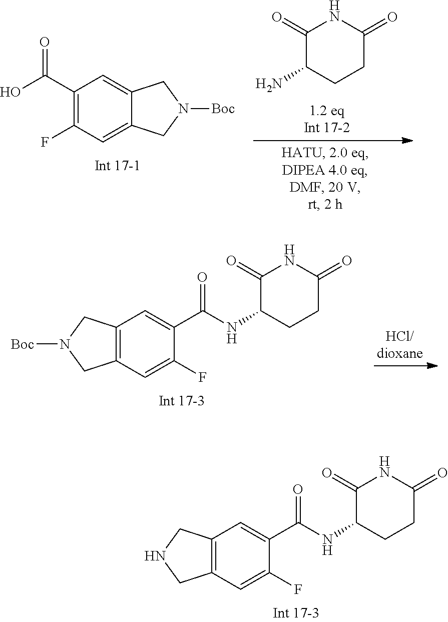 [SMILES: CC(C)(C)OC(=O)N1Cc2cc(F)c(C(=O)O)cc2C1 ; N[C@H]1CCC(=O)NC1=O ; CC(C)(C)OC(=O)N1Cc2cc(F)c(C(=O)N[C@H]3CCC(=O)NC3=O)cc2C1 ; O=C1CC[C@H](NC(=O)c2cc3c(cc2F)CNC3)C(=O)N1]

### Step 1: tert-butyl(S)-5-((2,6-dioxopiperidin-3-yl) carbamoyl)-6-fluoroisoindoline-2-carboxylate

To a mixture of 2-(tert-butoxycarbonyl)-6-fluoroisoindoline-5-carboxylic acid (400 mg, 1.423 mmol, 1 eq.) and(S)-3-aminopiperidine-2,6-dione (281 mg, 1.708 mmol, 1.2 eq) in DMA (5 mL) was added N,N,N′,N′-tetramethylchloroformamidinium hexafluorophosphate (2.0 g, 7.117 mmol, 5 eq.) and 1-methylimidazole (1.2 g, 14.23 mmol, 10 eq.) and the mixture was stirred at room temperature for 4 h. The mixture was purified directly by reverse phase column chromatography (0-90% Acetonitrile/0.05% formic acid)) to afford tert-butyl(S)-5-((2,6-dioxopiperidin-3-yl) carbamoyl)-6-fluoroisoindoline-2-carboxylate (300 mg, 53.1% yield) as white solid.

LC-MS purity: 99.7% (UV at 254 nm), 392.2 [M+H]⁺.

### Step 2: (S)—N-(2,6-dioxopiperidin-3-yl)-6-fluoroisoindoline-5-carboxamide

A mixture of tert-butyl(S)-5-((2,6-dioxopiperidin-3-yl) carbamoyl)-6-fluoroisoindoline-2-carboxylate (300 mg, 0.767 mmol, 1.0 eq.) in HCl/dioxane (10 mL) was stirred at room temperature for 2 h. The reaction mixture was concentrated to afford(S)—N-(2,6-dioxopiperidin-3-yl)-6-fluoroisoindoline-5-carboxamidc (230 mg (crude), 100% yield) as white solid. LC-MS purity: 100% (UV at 254 nm), 291.9 [M+H]⁺.

### Intermediate 19. 3-(7-oxo-5,7-dihydro-2H,6H-spiro[furo [2,3-f]isoindole-3,4′-piperidin]-6-yl)piperidine-2,6-dione hydrochloride

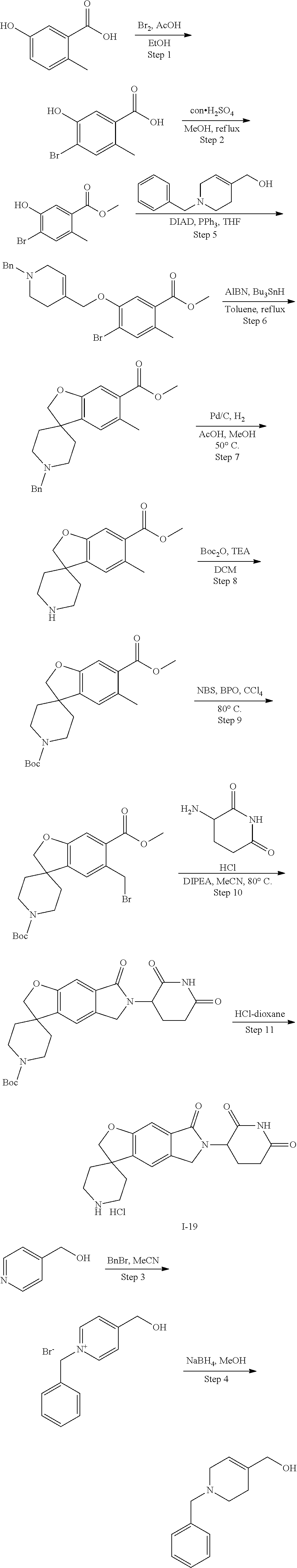

### Step 1:4-bromo-5-hydroxy-2-methylbenzoic acid

To a solution of 5-hydroxy-2-methylbenzoic acid (5.0 g, 32.9 mmol, 1.0 eq) in a mixture of ethanol (20 mL) and acetic acid (10 mL) was added dropwise bromine (3.4 mL, 65.7 mmol, 2.0 eq.). The reaction mixture was stirred for 10 h at room temperature, quenched with aqueous sodium thiosulfate solution (50 mL), and concentrated. The aqueous layer was extracted with ethyl acetate (50 mL×3). The organic layer was dried over magnesium sulfate, filtered, and concentrated under reduced pressure to get crude 4-bromo-5-hydroxy-2-methylbenzoic acid (7.6 g, yield 100%) as a white solid. The crude product was directly used in next step without further purification. LC-MS (ESI): mass calcd, for C₈H₇BrO₃, 229.96; m/z found, 231.2 [M+H]⁺.

### Step 2: methyl 4-bromo-5-hydroxy-2-methylbenzoate

Con. H₂SO₄ (12 mL) was added to a suspension of 4-bromo-5-hydroxy-2-methylbenzoic acid (15 g, 65.72 mmol) in methanol (100 mL). The mixture was refluxed for 16 h. After evaporation, the residue was diluted with water (100 mL) and extracted with EA (100 mL×3). The organic layer was washed with H₂O (100 mL×2), saturated aqueous NaHCO₃ solution (100 mL×2) and brine (100 mL). The organic layer was dried over anhydrous Na₂SO₄, filtered and concentrated under reduced pressure. The residue purified by flash column chromatography on silica gel (PE/EA=4/1) to afford methyl 4-bromo-5-hydroxy-2-methylbenzoate (7.5 g, yield 47%) as a colorless solid. LC-MS (ESI): mass calcd, for C₉H₉BrO₃, 243.97; m/z found, 245.2 [M+H]⁺.

¹HNMR (400 MHZ, CDCl₃) δ 7.56 (s, 1H), 7.36 (s, 1H), 5.52 (s, 1H), 3.88 (s, 3H), 2.50 (s, 3H).

### Step 3:1-benzyl-4-(hydroxymethyl)pyridin-1-ium bromide

To a solution of (pyridin-4-yl)methanol (8.9 g, 81.6 mmol, 1.0 eq) in CH₃CN (80 mL) was added a solution of (bromomethyl)benzene (11.705 mL, 97.9 mmol, 1.2 eq) in CH₃CN (40 mL). The reaction mixture was refluxed stirred at 90° C. for 3 h. After evaporation, the residue was washed with methyl tert-butyl ether, filtered, and dried to afford 1-benzyl-4-(hydroxymethyl)pyridin-1-ium bromide (16.33 g, yield 100%) as a yellow solid. LC-MS (ESI): mass calcd, for C₁₃H₁₄NO, 200.11; m/z found, 200.3 [M]⁺.

### Step 4: (1-benzyl-1,2,3,6-tetrahydropyridin-4-yl)methanol

To a solution of 1-benzyl-4-(hydroxymethyl)pyridin-1-ium bromide (16.3 g, 81.4 mmol, 1.0 eq) in CH₃OH (150 mL) was added NaBH₄ (9.3 g, 244.2 mmol, 3.0 eq) in portions at −20° C. The mixture was stirred at −20° C. for 1 h. The reaction was quenched with brine (100 mL) and extracted with EtOAc (200 mL×3). The organic layer was washed with brine (100 mL×3), dried over anhydrous Na₂SO₄, filtered and concentrated under reduced pressure. The residue was purified by flash column chromatography on silica gel (CH₃OH in DCM, from 0% to 10%) to afford (1-benzyl-1,2,3,6-tetrahydropyridin-4-yl)methanol (15 g, yield 91%) as a red oil. LC-MS (ESI): mass calcd, for C₁₃H₁₇NO, 203.13; m/z found, 204.4 [M+H]⁺.

¹H NMR (400 MHZ, DMSO-d₆) δ 7.24-7.18 (m, 4H), 7.16-7.12 (m, 1H), 5.43 (s, 1H), 4.61 (s, 1H), 3.71 (s, 2H), 3.42 (s, 2H), 2.76 (s, 2H), 2.39 (t, J=5.6 Hz, 2H), 1.91 (s, 2H).

### Step 5: methyl 5-[(1-benzyl-1,2,3,6-tetrahydropyridin-4-yl)methoxy]-4-bromo-2-methylbenzoate

To a solution of methyl 4-bromo-5-hydroxy-2-methylbenzoate (200 mg, 0.82 mmol, 1.0 eq), (1-benzyl-1,2,3,6-tetrahydropyridin-4-yl)methanol (166 mg, 0.82 mmol, 1.0 eq), and PPh₃ (321 mg, 1.22 mmol, 1.5 eq) in dry THF (10 mL) was added dropwise DIAD (0.25 mL, 1.22 mmol. 1.5 eq) at 0° C. under the N2 atmosphere. The solution was stirred for 2 h. After evaporation, the residue was purified by flash column chromatography on silica gel (PE/EA=2/1 to 1/1) to afford methyl 5-[(1-benzyl-1,2,3,6-tetrahydropyridin-4-yl)methoxy]-4-bromo-2-methylbenzoate (300 mg, yield 85%) as a white solid. LC-MS (ESI): mass calcd, for C₂₂H₂₄BrNO₃, 429.09; m/z found, 431.30 [M+H]⁺.

### Step 6: methyl l′-(cyclohexylmethyl)-5-methyl-2H-spiro[1-benzofuran-3,4′-piperidine]-6-carboxylate

Tributyl tin hydride (0.5 mL, 1.84 mmol, 4.0 equiv) was added to a solution of methyl 5-[(1-benzyl-1,2,3,6-tetrahydropyridin-4-yl)methoxy]-4-bromo-2-methylbenzoate (200 mg, 0.46 mmol, 1.0 eq) and AIBN (15 mg, 0.09 mmol, 0.2 eq) in toluene (10 mL). The solution was refluxed in a sealed tube for 6 h. After cooled down to room temperature, The solution was quenched with saturated potassium fluoride solution (40 mL) and stirred at room temperature for 0.5 h. The mixture was extracted with EA (40 mL×3). The organic layer was washed brine (40 mL), dried over anhydrous Na₂SO₄, filtered and concentrated under reduced pressure. The residue was purified by prep-TLC (EA/PE=1/1) to afford methyl 1′-(cyclohexylmethyl)-5-methyl-2H-spiro[1-benzofuran-3,4′-piperidine]-6-carboxylate (20 mg, yield 43%) as a yellow solid. LC-MS (ESI): mass calcd, for C₂₂H₂₅NO₃, 351.18; m/z found, 352.30 [M+H]⁺.

¹H NMR (400 MHZ, CDCl₃) δ 7.37-7.27 (m, 6H), 6.99 (s, 1H), 4.37 (s, 2H), 3.85 (s, 3H), 3.54 (s, 2H), 2.89 (d, J=10.2 Hz, 2H), 2.52 (s, 3H), 2.10-1.95 (m, 4H), 1.70 (d, J=11.4 Hz, 2H).

### Step 7: methyl 5-methyl-2H-spiro[benzofuran-3,4′-piperidine]-6-carboxylate

A mixture of methyl 1′-benzyl-5-methyl-2H-spiro[1-benzofuran-3,4′-piperidine]-6-carboxylate (1.0 g, 2.845 mmol, 1.0 eq), acetic acid (1 mL, 5.7 mmol, 6.1 eq), and 10% Pd/C (200 mg) in MeOH (20 mL) was stirred at 50° C. under H₂ (1 atm) for 3 h. After filtration, the filtrate was concentrated to get methyl 5-methyl-2H-spiro[benzofuran-3,4′-piperidine]-6-carboxylate (970 mg, yield 100%) as a colorless oil, which was directly used in the next step without further purification. LC-MS (ESI): mass calcd, for C₁₋₅H₁₉NO₃, 261.14; m/z found, 262.40 (M+H)⁺.

### Step 8: 1′-(tert-butyl) 6-methyl 5-methyl-2H-spiro[benzofuran-3,4′-piperidine]-1′,6-dicarboxylate

To a stirred solution of methyl 5-methyl-2H-spiro[1-benzofuran-3,4′-piperidine]-6-carboxylate (970 mg, 3.7 mmol, 1.0 eq) and TEA (1 mL, 7.4 mmol, 2.0 eq) in DCM (10 mL) was added dropwise Boc₂O (0.8 mL, 3.7 mmol, 2.0 eq) at 0° C. The mixture was stirred at room temperature for 2 h. The reaction mixture was poured into water (10 mL) and extracted with DCM (30 mL×2). The organic phase was dried over anhydrous Na₂SO₄ and concentrated under reduced pressure to afford 1′-(tert-butyl) 6-methyl 5-methyl-2H-spiro[benzofuran-3,4′-piperidine]-1′,6-dicarboxylate (1.28 g, yield 100%) as a white solid. LC-MS (ESI): mass calcd, for C₂₀H₂₇NO₅, 361.19; m/z found, 306.4 [M+H−56]⁺.

### Step 9: 1′-(tert-butyl) 6-methyl 5-(bromomethyl)-2H-spiro[benzofuran-3,4′-piperidine]-1′,6-dicarboxylate

A mixture of methyl 1′-benzyl-5-methyl-2H-spiro[1-benzofuran-3,4′-piperidine]-6-carboxylate (220 mg, 0.609 mmol, 1 eq), NBS (130 mg, 0.73 mmol, 1.2 eq), and BPO (60 mg, 0.243 mmol, 0.4 eq) in CC14 (10 mL) was refluxed for 4 h. After cooled to room temperature, the mixture was filtered, then the filtration was concentrated and to give l′-tert-butyl 6-methyl 5-(bromomethyl)-2H-spiro[1-benzofuran-3,4′-piperidine]-1′,6-dicarboxylate (100 mg, yield 37%) as a light-yellow solid. LC-MS (ESI): mass calcd, for C₂₀H₂₆BrNO₅, 439.10; m/z found, 462.20, [M+Na]⁺.

Step 10: tert-butyl 6-(2,6-dioxopiperidin-3-yl)-7-oxo-6,7-dihydro-2H,5H-spiro[furo[2,3-f]isoindole-3,4′-piperidine]-1′-carboxylate

DIPEA (0.12 mL, 0.681 mmol, 3.0 eq) was added to l′-tert-butyl 6-methyl 5-(bromomethyl)-2H-spiro[1-benzofuran-3,4′-piperidine]-1′,6-dicarboxylate (100 mg, 0.227 mmol, 1.0 eq) and 3-aminopiperidine-2,6-dione hydrochloride (56 mg, 0.341 mmol, 1.5 eq) in MeCN (5 mL) under nitrogen. The resulting suspension was stirred at 80° C. for 24 h. The reaction mixture was cooled to room temperature and filtered. The solid was washed with MeCN and purified by prep-TLC (100% EtOAc) to afford tert-butyl 6-(2,6-dioxopiperidin-3-yl)-7-oxo-6,7-dihydro-2H,5H-spiro[furo [2,3-f]isoindole-3,4′-piperidine]-1′-carboxylate (50 mg, yield 48%) as a white solid. LC-MS (ESI): mass calcd, for C₂₄H₂₉N₃O₆, 455.51; m/z found, 456.50, (M+H)⁺.

### Step 11:3-(7-oxo-5,7-dihydro-2H,6H-spiro[furo [2,3-f]isoindole-3,4′-piperidin]-6-yl)piperidine-2,6-dione

To a solution of tert-butyl 6-(2,6-dioxopiperidin-3-yl)-7-oxo-2,5,6,7-tetrahydrospiro[furo [2,3-f]isoindole-3,4′-piperidine]-1′-carboxylate (50 mg, 0.11 mmol, 1.0 eq) in DCM (1 mL) was added HCl-dioxane solution (4 M, 1 mL, 4 mmol, 36 eq) and the mixture was stirred for 30 min. After evaporation, the residue was purified by prep-HPLC with YMC-TA C18 (5 μm, 20×250 mm), and mobile phase of 5-95% ACN in water over 10 min, and then hold at 100% ACN for 2 min, at a flow rate of 25 mL/min to get 3-{7-oxo-2,5,6,7-tetrahydrospiro[furo [2,3-f]isoindole-3,4′-piperidine]-6-yl}piperidine-2,6-dione hydrochloride (30 mg, yield 70%) as a white solid. LC-MS (ESI): mass calcd, for C₁₉H₂₁N₃O₄, 355.19; m/z found, 356.20 [M+H]⁺.

¹H NMR (400 MHZ, DMSO-d₆) δ 10.98 (s, 1H), 8.78 (s, 2H), 7.36 (s, 1H), 7.06 (s, 1H), 5.11-5.06 (m, 1H), 4.58 (s, 2H), 4.38 (d, J=17.0 Hz, 1H), 4.25 (d, J=17.0 Hz, 1H), 3.30-3.27 (m, 2H), 3.04-2.92 (m, 2H), 2.93-2.84 (m, 1H), 2.62-2.56 (m, 1H), 2.44-2.29 (m, 1H), 2.09-1.97 (m, 3H), 1.90-1.79 (m, 2H).

### Intermediate 20: (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione

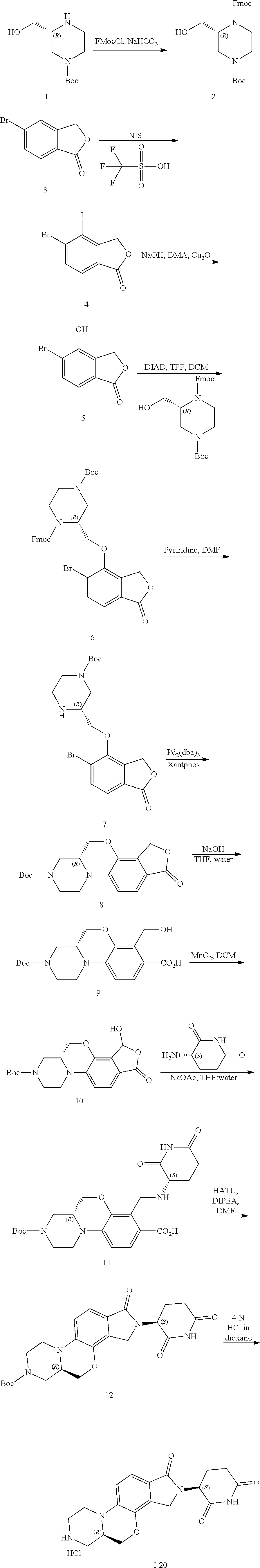

### Step 1:1-((9H-fluoren-9-yl)methyl) 4-(tert-butyl) (R)-2-(hydroxymethyl)piperazine-1,4-dicarboxylate

(R)-1-Boc-3-(Hydroxymethyl)piperazine (1, 10 g, 46.2 mmol) was dissolved in a mixture of DCM (180 mL) and sat. NaHCO₃ (180 mL). FMocCl (46.2 mmol) was dissolved in DCM (15 mL) and added dropwise with vigorous stirring. The mixture was stirred for 1 hour. The layers were separated and the aqueous phase was extracted with DCM. The combined organic phases were washed with brine, dried over Na₂SO₄ and the solvent was removed in vacuo to give an oil. The crude product was purified by silica gel column chromatography using 0-80% EtOAc/hexane (80% yield).

### Step 2:5-bromo-4-iodoisobenzofuran-1 (3H)-one

To a solution of 5-Bromo-3H-isobenzofuran-1-one (3, 5 g, 23.4 mmol, 1 eq.) in trifluoromethanesulfonic acid (68 g, 40 mL, 19.30 eq) was added NIS (5.5 g, 24.6 mmol, 1.05 eq.) at 0° C. in portions. The mixture was allowed to warm to room temperature and stirred overnight. TLC (hexane:ethyl acetate=5:1) showed no starting material remained and two new spots (Rf=0.4, 0.5) formed. The reaction mixture was poured into ice-water (100 mL) and yellow solid precipitated. The mixture was filtered and the filter cake was washed with ice cold water. The filter cake was dissolved in DCM (500 mL) and washed with 1 (M)Na₂S203 followed by dried over sodium sulfate. The mixture was filtered and the filtrate was concentrated to afford a yellow solid. The crude product was purified on a 120 g silica column running a 0-10% EtOAc/hexane gradient over 70 min. ¹H NMR (400 MHZ, CDCl₃): δ 7.83 (d, J=8.0 Hz, 1H), 7.77 (d, J=8.0 Hz, 1H), 5.10 (s, 2H).

### Step 3:5-bromo-4-hydroxyisobenzofuran-1 (3H)-one

To a mixture of 5-Bromo-4-iodo-3H-isobenzofuran-1-one (4, 4 g, 1 eq), sodium hydroxide (2.3 g, 5 eq) in water (40 mL, 1.5 M) and N,N-dimethylacetamide (20 ml) was added cuprous oxide (0.338 g, 0.2 eq). The reaction mixture was heated to 80° C. and held for 12 h. TLC (Hexane: ethyl acetate=1:1, Rf=0.3) showed the reaction was completed. The reaction mixture neutralized using 1 (N) hydrochloride solution and extracted with ethyl acetate (40 mL×2), washed with brine (150 mL), and then dried over sodium sulfate. The crude product was purified by silica gel column chromatography using 0-100% EtOAc/hexane. 5-Bromo-4-hydroxy-3H-isobenzofuran-1-one (5, 50% yield) was obtained as a white solid. 1H NMR (400 MHZ, DMSO) δ 10.90 (s, 1H), 7.72 (d, J=8.0 Hz, 1H), 7.23 (d, J=8.0 Hz, 1H), 5.35 (s, 2H).

Step 4:1-((9H-fluoren-9-yl)methyl) 4-(tert-butyl) (R)-2-(((5-bromo-1-oxo-1,3 dihydroisobenzofuran-4-yl)oxy)methyl)piperazine-1,4-dicarboxylate

To a solution of 5-Bromo-4-hydroxyisobenzofuran-1 (3H)-one (5, 700 mg, 3 mmol, 1 eq.) in 12 mL of THF/DCM, 1-((9H-fluoren-9-yl)methyl) 4-(tert-butyl) (R)-2-(hydroxymethyl)piperazine-1,4-dicarboxylate (2 μm, 4.5 mmol, 1.5 eq.) and PPh₃ (1.17 μm, 4.5 mmol, 1.5 eq.) was added. The reaction mixture was cooled to 0° C. and DIAD (0.9 mL, 4.5 mmol, 1.5 eq.) was added dropwisc. The resultant mixture was then stirred overnight at room temperature. The solvent was evaporated at reduced pressure; the crude product was purified by silica gel column chromatography using 0-100% EtOAc/hexane. LC/MS (ESI) m/z: 649.15 Step 5: tert-butyl (R)-3-(((5-bromo-1-oxo-1,3-dihydroisobenzofuran-4-yl)oxy)methyl)piperazine-I-carboxylate

To a solution of 1-((9H-fluoren-9-yl)methyl) 4-(tert-butyl) (R)-2-(((5-Bromo-1-oxo-1,3-dihydroisobenzofuran-4-yl)oxy)methyl)piperazine-1,4-dicarboxylate (6, 1 μm) was added 20% (v/v) piperidine in DMF (5 mL/gm of SM). The resulting mixture was stirred at room temperature overnight. The mixture was diluted with ethyl acetate and washed with water. The combined organic phases were washed with brine, dried over Na₂SO₄ and the solvent was removed in vacuo to give an oil. The crude product was purified by silica gel column chromatography using 0-5% DCM in methanol. Yield 70%. LC/MS (ESI) m/z: 426.08 [M+1]+.

### Step 6: tert-butyl (R)-1-oxo-1,3,5a,6,8,9-hexahydroisobenzofuro[4,5-b]pyrazino[1,2-d][1,4]oxazine-7 (5H)-carboxylate

A vial was charged with tert-butyl (R)-3-(((5-bromo-1-oxo-1,3-dihydroisobenzofuran-4-yl)oxy)methyl)piperazine-1-carboxylate (7, 170 mg, 0.38 mmol, 1 eq.), Pd₂ (dba) 3 (0.1 eq.), XantPhos (0.2 eq.), Cs₂CO₃ (3 eq.) and dioxane (5 mL). The mixture was purged with nitrogen and heated to 100° C. for 6 h. TLC (ethyl acetate:petroleum ether=1:2) showed reaction was complete. The mixture was diluted with ethyl acetate and washed with water. The organic layer was washed with brine and dried over sodium sulfate. The crude product was purified by silica gel column chromatography using 0-50% EtOAc/hexane. LC/MS (ESI) m/z: 347.15 [M+1]+. Yield 60%

### Step 7: (R)-3-(tert-butoxycarbonyl)-7-(hydroxymethyl)-1,2,3,4,4a,5-hexahydrobenzo[b]pyrazino[1,2-d][1,4]oxazine-8-carboxylic acid

To a solution of tert-butyl (R)-1-oxo-1,3,5a,6,8,9-hexahydroisobenzofuro[4,5-b]pyrazino[1,2-d][1,4]oxazine-7 (5H)-carboxylate (8, 346 mg, 1 mmol, 1 eq) in tetrahydrofuran (4 mL) and water (4 mL) was added sodium hydroxide (200 mg, 5 eq). The mixture was stirred at 20° C. for 16 h. TLC (ethyl acetate:hexane=1:1) showed reaction was complete. The mixture was adjusted to pH=5 with aq. hydrochloric acid (1 M) and extracted with ethyl acetate (10 mL×3). The organic layer was washed with brine (10×2 mL) and dried over sodium sulfate. The crude material was not further purified and used as crude for the next steps. LC/MS (ESI) m/z: 365.16 Step 8: tert-butyl (5aR)-3-hydroxy-1-oxo-1,3,5a,6,8,9-hexahydroisobenzofuro[4,5-b]pyrazino[1,2-d][1,4]oxazine-7(5H)-carboxylate

To a solution of (R)-3-(tert-butoxycarbonyl)-7-(hydroxymethyl)-1,2,3,4,4a,5-hexahydrobenzo[b]pyrazino[1,2-d][1,4]oxazine-8-carboxylic acid (9, 1 eq.) in dichloromethane (10 mL) was added manganese dioxide (15 eq.). The mixture was stirred at 20° C. for overnight. TLC (ethyl acetate:hexane=1:1) showed reaction was complete. The mixture was diluted with dichloromethane (10 mL) and filtered through a pad of Celite. The filtrate was concentrated in vacuum. The crude product was purified by silica gel column chromatography. LC/MS (ESI) m/z: 363.16. ¹H NMR (400 MHZ, CD₃OD) δ 7.32 (d, J=8.3 Hz, 1H), 7.14 (d, J=8.4 Hz, 1H), 6.64-6.40 (m, 1H), 4.42 (dd, J=11.0, 3.0 Hz, 1H), 4.23-4.01 (m, 3H), 3.95 (d, J=12.4 Hz, 1H), 3.34 -3.23 (m, 1H), 3.08 (brs, 1H), 2.87 (td, J=12.2, 3.5 Hz, 1H), 2.74 (s, 1H) 1.50 (S, 9H).

### Step 9: (R)-3-(tert-butoxycarbonyl)-7-((((S)-2,6-dioxopiperidin-3-yl)amino)methyl)-1,2,3,4,4a,5-hexahydrobenzo[b]pyrazino[1,2-d][1,4]oxazine-8-carboxylic acid

To a mixture of(S) 3-aminopiperidine-2,6-dione (10, 1.5 eq., HCl salt) in methanol (2 ml) and dichloromethane (4 ml) was added sodium acetate (4 eq.). The mixture was stirred at 20° C. for 15 min, then tert-butyl (5aR)-3-hydroxy-1-oxo-1,3,5a,6,8,9- hexahydroisobenzofuro[4,5-b]pyrazino[1,2-d][1,4]oxazine-7 (5H)-carboxylate (1 eq.) was added and the mixture was stirred for 30 mins. Sodium cyanoborohydride (2 eq.) was added and the mixture was further stirred for 1 hour. LCMS showed the reaction was complete. The mixture was adjusted to pH=4-5 with an aqueous hydrochloric acid solution (1 M) and extracted with ethyl acetate (10 mL×3). The crude material was not further purified and used as crude for the next steps. LC/MS (ESI) m/z: 475.21

Step 10: tert-butyl (R)-2-((S)-2,6-dioxopiperidin-3-yl)-1-oxo-2,3,5a,6,8,9-hexahydro-1H-pyrazino[1,2′: 4,5][1,4]oxazino[2,3-e]isoindole-7 (5H)-carboxylate

To solution of (R)-3-(tert-butoxycarbonyl)-7-((((S)-2,6-dioxopiperidin-3-yl)amino)methyl)-1,2,3,4,4a,5-hexahydrobenzo[b]pyrazino[1,2-d][1,4]oxazine-8-carboxylic acid (11, 90 mg, 0.18 mmol, 1 eq.) in dimethylformamide (5 mL) was added HATU (72 mg, 1.0 eq.) followed by addition of DIPEA (3 eq.). The solution was stirred for 15 mins, at 0° C. The residue was purified by reverse phase HPLC to get the desired compound 12. LC/MS (ESI) m/z: 457.20¹H NMR (400 MHZ, Methanol-d4) δ 7.32 (d, J=8.3 Hz, 1H), 7.08 (d, J=8.4 Hz, 1H), 5.10 (dd, J=13.3, 5.2 Hz, 1H), 4.46-4.30 (m, 3H), 4.23-3.98 (m, 3H), 3.93 (d, J=12.4 Hz, 1H), 3.22 (ddd, J=11.2, 8.2, 3.0 Hz, 1H), 3.07 (s, 1H), 2.99-2.61 (m, 4H), 2.59-2.42 (m, 1H), 2.21-2.07 (m, 1H), 1.51 (s, 9H).

### Step 11: (S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione hydrochloride

A mixture of tert-butyl (R)-2-((S)-2,6-dioxopiperidin-3-yl)-1-oxo-2,3,5a,6,8,9-hexahydro-1H-pyrazino[1′,2′: 4,5][1,4]oxazino[2,3-e]isoindole-7 (5H)-carboxylate (456 mg, 1.0 mmol, 1 eq.) in HCl/dioxane (10 mL) was stirred at room temperature for 2 h. The reaction mixture was concentrated to afford(S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione hydrochloride (1-20, 400 mg, crude) as white solid. 1H NMR (400 MHZ, DMSO-d₆) δ 10.98 (s, 1H), 8.78 (s, 2H), 7.36 (s, 1H), 7.06 (s, 1H), 5.11-5.06 (m, 1H), 4.58 (s, 2H), 4.38 (d, J=17.0 Hz, 1H), 4.25 (d, J=17.0 Hz, 1H), 3.30-3.27 (m, 2H), 3.04-2.92 (m, 2H), 2.93-2.84 (m, 1H), 2.62-2.56 (m, 1H), 2.44-2.29 (m, 1H), 2.09-1.97 (m, 3H), 1.90-1.79 (m, 2H).

### Intermediate 21:3-(6-oxo-6,8-dihydro-2H,7H-spiro[furo [2,3-e]isoindole-3,4′-piperidin]-7-yl)piperidine-2,6-dione trifluoroacetate salt

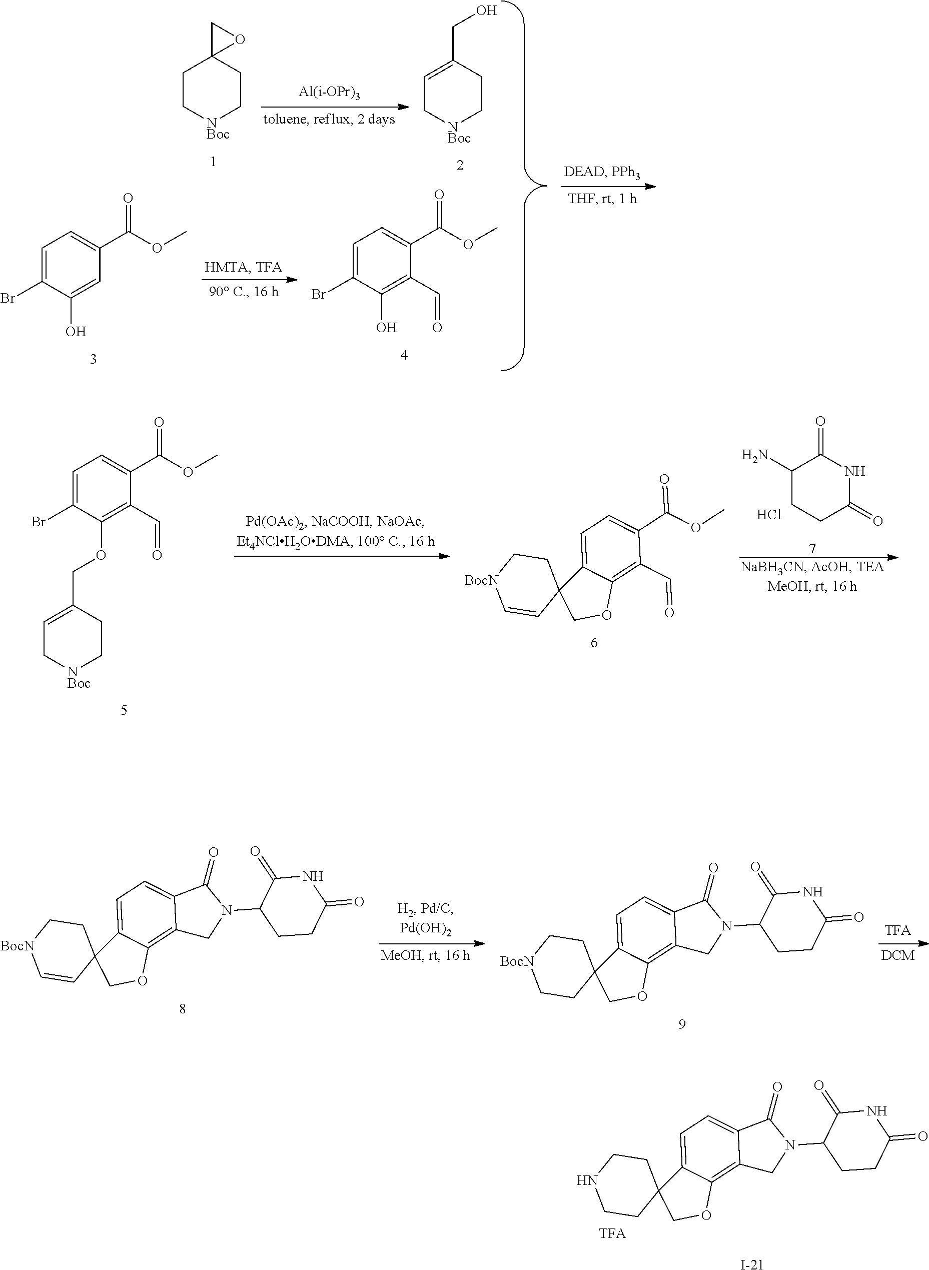

### Step 1: tert-butyl 4-(hydroxymethyl)-3,6-dihydropyridine-1(2H)-carboxylate

A mixture of the tert-butyl 1-oxa-6-azaspiro[2.5]octane-6-carboxylate 1 (25 g, 117.4 mmol) and aluminium isopropoxide (35.9 g, 176 mmol) in anhydrous toluene (300 mL) was heated under reflux for 36 h. The reaction was allowed to cool and then poured into aqueous hydrogen chloride (1 M). The aqueous phase was extracted into EA and the organic extracts were dried (Na₂SO₄) then concentrated under reduced pressure. Chromatography of the residue gave the title compound 2 as a colorless oil (12 g, 48%)

### Step 2: methyl 4-bromo-2-formyl-3-hydroxybenzoate

To a solution of methyl 4-bromo-3-hydroxybenzoate 3 (18 g, 77.9 mmol) in TFA (150 mL) was added HMTA (41.5 g, 296 mmol). The solution was stirred at 90° C. overnight. 2N HCl was added, and a yellow solid formed. The mixture was stirred for 10 min and then additional 1 L water was added and stirred for 1 h. The mixture was filtered. The filter cake was dissolved in DCM and filtered on celite, dried, and then remove most of solvent in vacuo. The result mixture was triturated with MeOH and filtered to afford methyl 4-bromo-2-formyl-3-hydroxybenzoate 4 as a yellow solid (12 g, 59%).

### Step 3: tert-butyl 4-((6-bromo-2-formyl-3-(methoxycarbonyl) phenoxy)methyl)-3,6-dihydropyridine-1 (2H)-carboxylate

To a solution of compound 2 (6 g, 23.2 mmol, 1.0 eq.) in dry THF (50 ml), compound 4 (5.9 g, 27.8 mmol, 1.2 eq.) and PPh₃ (7.9 g, 30.1 mmol, 1.3 eq.) was added. The reaction mixture was cooled to 0° C. and DIAD (6.6 g, 32.4 mmol, 1.4 eq.) was added dropwise. The resultant mixture was then stirred 1 h at room temperature. The solvent was evaporated at reduced pressure and the crude product was purified by silica gel column chromatography using 0-20% EtOAc/hexane. The desired product 5 was obtained as a yellow oil (4 g, 38%).

### Step 4: ‘-(tert-butyl) 6-methyl 7-formyl-2’,3′-dihydro-1′H,2H-spiro[benzofuran-3,4′-pyridine]-1′,6-dicarboxylate

To a solution of compound 5 (4 g, 8.8 mmol, 1.0 eq.) in DMA (30 mL) was added NaCOOH (0.72 g, 10.6 mmol, 1.2 eq.), Et4NCI.H₂O (1.95 g, 10.6 mmol, 1.2 eq), Pd (OAc) 2 (0.2 g, 0.88 mmol, 0.1 eq) and NaOAc (1.44 g, 17.6 mmol, 2 eq.). The mixture was purged with nitrogen and heated to 100° C. overnight. The mixture was diluted with ethyl acetate and washed with water. The organic layer was washed with brine and dried over sodium sulfate. The crude product was purified by silica gel column chromatography using 0-30% EtOAc/hexane to give compound 6 as a yellow oil (720 mg, yield 24%).

### Step 5: tert-butyl 7-(2,6-dioxopiperidin-3-yl)-6-oxo-2′,3′,7,8-tetrahydro-1′H,2H,6H-spiro[furo [2,3-e]isoindole-3,4′-pyridine]-1′-carboxylate

To a solution of compound 6 (780 mg, 2.09 mmol, 1 eq.) and compound 6 (344 mg, 2.09 mmol, 1 eq.) in MeOH (10 mL) was added TEA (211 mg, 2.09 mmol, 1 eq.) and AcOH (627 mg, 10.5 mmol, 5 eq.) followed by NaBH₃CN (395 mg, 6.27 mmol, 3 eq.). The mixture was stirred at room temperature for 16 h, diluted with EA, and washed with brine, then dried over sodium sulfate. The solvent was removed under reduced pressure. The crude product was purified by silica gel column chromatography using 0-100% EtOAc/hexane to give compound 8 as a white solid (400 mg, 42%).

### Step 6: tert-butyl 7-(2,6-dioxopiperidin-3-yl)-6-oxo-7,8-dihydro-2H,6H-spiro[furo [2,3-e]isoindole-3,4′-piperidine]-1′-carboxylate

To a solution of compound 8 (400 mg, 0.88 mmol, 1 eq.) in MeOH was added Pd/C (200 mg, 10% on Carbon, wetted with c.a.55% water) and Pd (OH) 2 (200 mg). The mixture was purged with H₂ and stirred at rt overnight under H₂. The mixture was filtered through Celite and the filtrate was concentrated. The crude product was purified by silica gel chromatography. The desired compound tert-butyl 7-(2,6-dioxopiperidin-3-yl)-6-oxo-7,8-dihydro-2H,6H-spiro[furo [2,3-e]isoindole-3,4′-piperidine]-1′-carboxylate was obtained as white solid (220 mg, 55%). LC/MS (ESI) m/z: 356.2 [M+H]⁺. ¹H NMR (400 MHz, DMSO) δ 10.97 (s, 1H), 7.42 (d, J=7.2 Hz, 1H), 7.26 (d, J=7.6 Hz, 1H), 5.11-5.06 (m, 1H), 4.62-4.57 (m, 2H), 4.38 (d, J=17.2 Hz, 1H), 4.21 (d, J=17.2 Hz, 1H), 3.95-3.92 (m, 2H), 2.95-2.83 (m, 3H), 2.61-2.56 (m, 1H), 2.47-2.39 (m, 1H), 1.98-1.96 (m, 1H), 1.83-1.77 (m, 2H), 1.71-1.65 (m, 2H), 1.42 (s, 9H).

### Step 7:3-(6-oxo-6,8-dihydro-2H,7H-spiro[furo [2,3-e]isoindole-3,4′-piperidin]-7-yl)piperidine-2,6-dione trifluoroacetate salt

Compound 9 was treated with TFA in DCM at room temperature to de-protect the N-Boc group to provide intermediate I-21. LC/MS (ESI) m/z: 356.15. ¹H NMR (400 MHZ, CDCl₃) δ 8.00 (s, 1H), 7.50 (d, J=7.7 Hz, 1H), 7.28 (s, 1H), 5.23 (dd, J=13.3, 5.1 Hz, 1H), 4.55 (d, J=1.4 Hz, 2H), 4.46 (d, J=16.0 Hz, 1H), 4.32 (d, J=16.0 Hz, 1H), 4.15 (s, 2H), 3.01-2.77 (m, 4H), 2.38 (dd, J=13.1, 5.0 Hz, 1H), 2.29-2.17 (m, 1H), 1.92 (t, J=12.5 Hz, 2H), 1.83-1.72 (m, 2H).

### Intermediate 22:3-((R)-8-oxo-1,2,3,4,4a,5,8,10-octahydro-9H-pyrazino[1′,2′: 4,5][1,4]oxazino[2,3-f]isoindol-9-yl)piperidine-2,6-dione trifluoroacetat salt

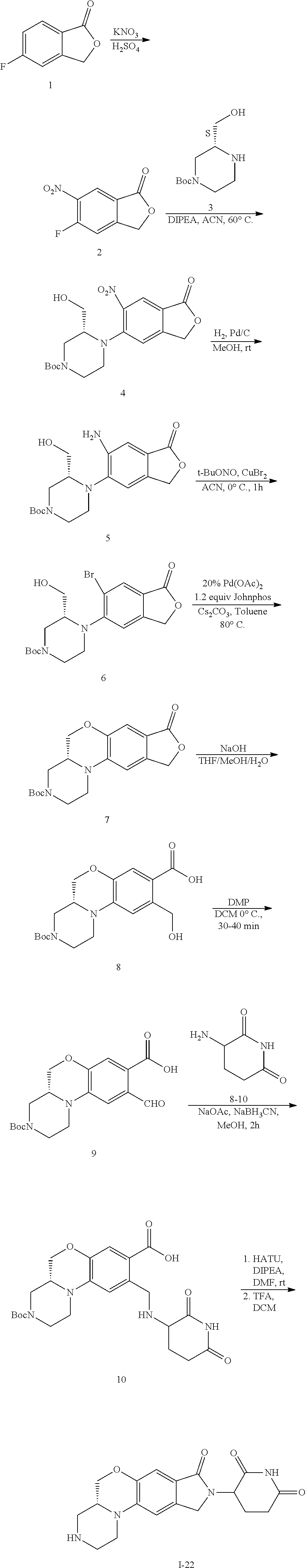

Intermediate I-22 was made using the similar procedure for making intermediate I-8. LC-MS: [M+H]⁺=356.91. ¹H NMR (400 MHZ, Methanol-d₄) δ 7.17 (s, 1H), 7.13 (d, J=1.7 Hz, 1H), 5.14-5.03 (m, 1H), 4.44-4.28 (m, 3H), 4.27-4.17 (m, 1H), 4.12-4.03 (m, 1H), 3.61-3.42 (m, 3H), 3.30-3.21 (m, 1H), 3.21-3.11 (m, 1H), 3.04-2.95 (m, 1H), 2.95-2.83 (m, 1H), 2.81 -2.72 (m, 1H), 2.52-2.38 (m, 1H), 2.19-2.10 (m, 1H). ¹³C NMR (101 MHZ, MeOD) δ 174.66, 172.46, 172.44, 171.64, 171.61, 146.51, 146.47, 139.24, 139.20, 138.14, 123.98, 123.91, 111.93, 108.73, 108.70, 66.97, 53.71, 53.60, 50.88, 50.86, 44.38, 44.24, 44.20, 43.62, 32.38, 24.13.

### Intermediate 23:3-((S)-6-methyl-8-oxo-2,3,4,4a,5,6,8,10-octahydropyrazino[1,2-a]pyrrolo[3,4-g]quinoxalin-9 (1H)-yl)piperidine-2,6-dione trifluoroacetat salt

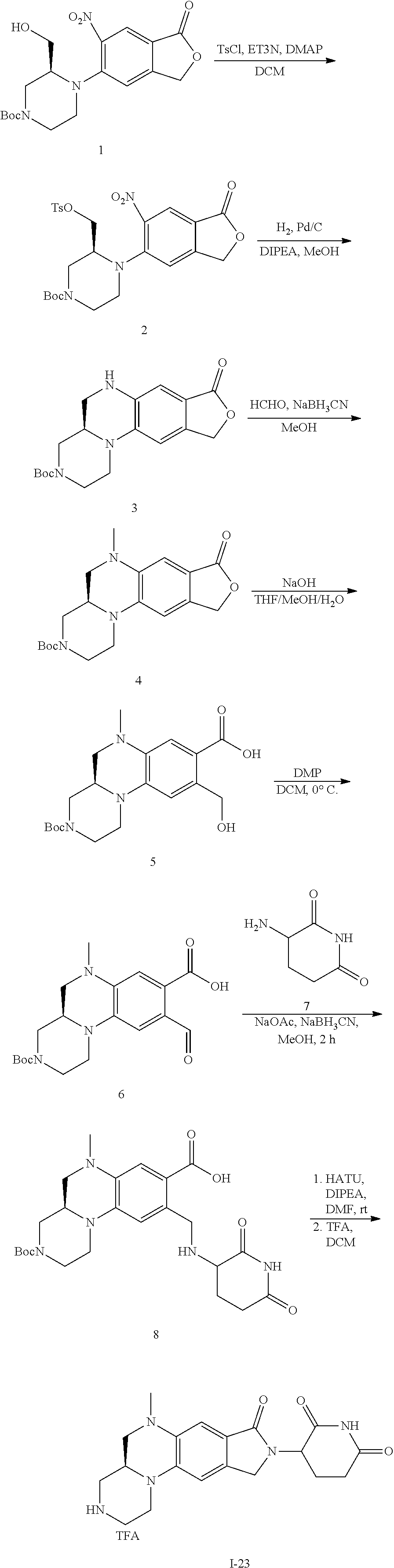

### Step 1: tert-butyl(S)-4-(6-nitro-1-oxo-1,3-dihydroisobenzofuran-5-yl)-3-((tosyloxy)methyl)piperazine-1-carboxylate

To a solution of 1 (1 equiv, 1.49 g) in DCM (30 mL) was TsCI (2.0 equiv, 1.44 g), Et3N (4.0 equiv, 2.11 mL) and DMAP (0.2 equiv, 92 mg), and the mixture was stirred at rt overnight. TLC (n-Hexane: EA=1:1) indicated the starting material 1 was completely conversion and an new spot detected. Then the reaction mixture was diluted with DCM, washed with brine, dried over Na₂SO₄, and concentrated under reduced pressure to give a residue which was purified by silica gel flash chromatography (n-Hexane: EA=100:0 to 60:40). The desired product 2 was obtained as a yellow foam (1.67 g, yield=81%).

### Step 2: tert-butyl (R)-8-oxo-1,2,4,4a,5,6,8,10-octahydro-3H-furo[3,4-g]pyrazino[1,2-a]quinoxaline-3-carboxylate

To a solution of 2 (1.0 equiv, 1.67 g) in MeOH (20 mL) was added DIPEA (2.0 equiv, 1.06 mL), followed by Pd/C (0.5 equiv, 835 mg). The reaction mixture was degassed and purged with H₂ three times and keep stirred at rt overnight. UPLC-MS showed the starting material completely conversed to desired product 3. Then the reaction mixture was filtered through Celite, and the filtrate was concentrated under reduced pressure to give a residue which was purified by silica gel flash chromatography (DCM:MeOH=100:0 to 95:5). The desired product 3 was obtained as a yellow solid (957 mg, yield=91%).

### Step 3: tert-butyl (R)-6-methyl-8-oxo-1,2,4,4a,5,6,8,10-octahydro-3H-furo[3,4-g]pyrazino[1,2-a]quinoxaline-3-carboxylate

To a solution of 3 (1.0 equiv, 410 mg) in MeOH/AcOH/DCM (10 mL/1 mL/3 mL) was added HCHO (5.0 equiv, 470 mg), and the mixture was kept stirring for 2 h. Then NaBH₃CN (5.0 equiv, 361 mg) was added. 15 min Later, UPLC-MS showed the starting material 3 all converted to desired product 4. The reaction mixture was concentrated under reduced pressure, diluted with DCM, washed with brine, dried over Na₂SO₄ and concentrated to give a yellow powder which is directly used in the next step.

### Step 4: (R)-3-(tert-butoxycarbonyl)-9-(hydroxymethyl)-6-methyl-2,3,4,4a,5,6-hexahydro-1H-pyrazino[1,2-a]quinoxaline-8-carboxylic acid

4 (1.0 equiv, 427 mg) was dissolved in THE/MeOH/H₂O (3 mL/3 mL/1 mL), and NaOH (5.0 equiv, 238 mg) was added. The reaction was kept stirring at 40° C. overnight. Then the reaction mixture was concentrated under reduced pressure to remove the solvent. The residue was diluted with 3-4 mL H₂O, followed by acidified with 2 N aq. HCl to PH 3-4. White solid was precipitated, which was collected and dried to give desired product 5 as a white powder 358 mg (yield=80% in two steps).

### Step 5: (R)-3-(tert-butoxycarbonyl)-9-formyl-6-methyl-2,3,4,4a,5,6-hexahydro-1H-pyrazino[1,2-a]quinoxaline-8-carboxylic acid

To a solution of 5 (1.0 equiv, 305 mg) in DCM (20 mL) was added DMP (1.65 equiv, 565 mg) into 3 potions at 0° C. 30 min Later, UPLC-MS indicated that 5 was completely conversion and a new main peak with desired MS formed, then the reaction was immediately diluted with DCM, washed with brine, dried over and concentrated under reduced pressure to give a crude product 6 which is directly used in the next step.

### Step 6: (4aR)-3-(tert-butoxycarbonyl)-9-(((2,6-dioxopiperidin-3-yl)amino)methyl)-6-methyl-2,3,4,4a,5,6-hexahydro-1H-pyrazino[1,2-a]quinoxaline-8-carboxylic acid

A mixture of 6 (1.0 equiv, 303 mg), 7 (1.5 equiv, 199.5 mg) and NaOAc (1.5 equiv, 99.4 mg) was dissolved in MeOH (20 mL), and kept stirring at rt for 20 min. Then NaBH₃CN (3.0 equiv, 151 mg) was added in 3 potions. 2 h Later, UPLC-MS showed the starting material 6 was completely conversion and a new main peak with desired MS formed. Next, the reaction mixture was quenched with 4 mL water and concentrated under reduced pressure to give a residue which was purified by pre-HPLC (20% to 100% acetonitrile (0.1% HCOOH, not TFA) in 80 min, 60 mL/min, 27% acetonitrile come out). The desired product 8 was obtained as a dark solid 138 mg (yield=35% in two steps) after lyophilization.

### Step 7:3-((S)-6-methyl-8-oxo-2,3,4,4a,5,6,8,10-octahydropyrazino[1,2-a]pyrrolo[3,4-g]quinoxalin-9 (1H)-yl)piperidine-2,6-dione trifluoroacetate salt

To a solution of 8 (1.0 equiv, 138 mg) in DMF (5 mL) was added HATU (1.1 equiv, 118 mg) and DIPEA (3.0 equiv, 148 μL), and the reaction was stirred at rt for 20-30 min. UPLC-MS indicated a new main peak with desired MS formed, then quenched with 3 mL water and purified by HPLC-MS (acetonitrile 35% to 100% in 65 min, 60 mL/min, 44% acetonitrile come out). Collected the solution and concentrated to give a solid which was dissolved into TFA/DCM to deprotect the Boc group. The title compound I-23 was obtained as a light purple solid 40 mg (yield is much higher than here because much product was lost when purified) after removed the solvent and lyophilized. LC-MS: [M+H]⁺=370.02. ¹H NMR (400 MHZ, Methanol-d₄) δ 6.99-6.93 (m, 2H), 5.12-5.04 (m, 1H), 4.34-4.29 (m, 1H), 4.27-4.18 (m, 1H), 3.69-3.60 (m, 1H), 3.51-3.40 (m, 2H), 3.39-3.33 (m, 1H), 3.27-3.12 (m, 4H), 3.07-3.00 (m, 1H), 2.92 (s, 3H), 2.88-2.85 (m, 1H), 2.80-2.73 (m, 1H), 2.51-2.38 (m, 1H), 2.18-2.10 (m, 1H).

### Intermediate 24: (3S)-3-(4-methyl-1-oxo-3,4,5,5a,6,7,8,9-octahydropyrazino[1,2-a]pyrrolo[3,4-f]quinoxalin-2 (1H)-yl)piperidine-2,6-dione trifluoroacetate salt

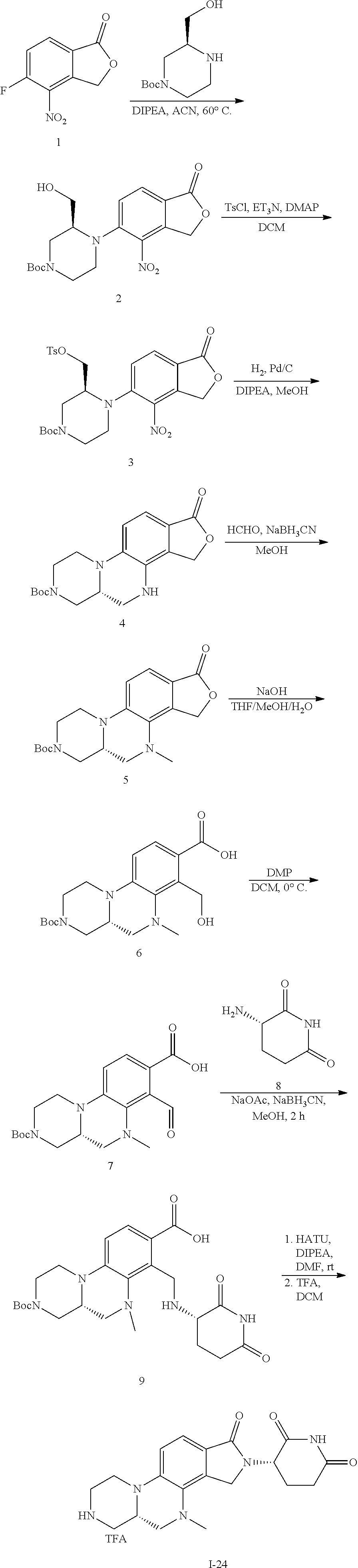

Intermediate I-24 was made using the similar procedure for making intermediate I-23. LC-MS: [M+H]⁺=370.28. ¹H NMR (400 MHZ, Methanol-d₄) δ 7.32 (dd, J=8.4, 2.3 Hz, 1H), 7.09 (dd, J=8.6, 2.5 Hz, 1H), 5.14-5.04 (m, 1H), 4.63-4.44 (m, 2H), 4.30-4.16 (m, 1H), 3.60-3.51 (m, 1H), 3.49-3.39 (m, 2H), 3.29-3.21 (m, 2H), 3.16-3.02 (m, 2H), 2.98-2.83 (m, 5H), 2.82-2.72 (m, 1H), 2.58-2.45 (m, 1H), 2.21-2.10 (m, 1H). ¹³C NMR (101 MHZ, MeOD) δ 174.70, 172.53, 172.50, 171.69, 171.63, 141.77, 141.73, 133.94, 133.82, 133.63, 124.97, 118.36, 118.32, 115.17, 53.85, 53.82, 53.67, 53.63, 47.98, 47.86, 46.28, 44.83, 44.39, 43.81, 43.75, 32.37, 24.03, 23.99.

### Intermediate 25:3-(7′-oxo-2′,3′,7′,9′-tetrahydro-8′H-spiro[piperidine-4,4′-pyrano[2,3-e]isoindol]-8′-yl)piperidine-2,6-dione trifuluoroacetate salt

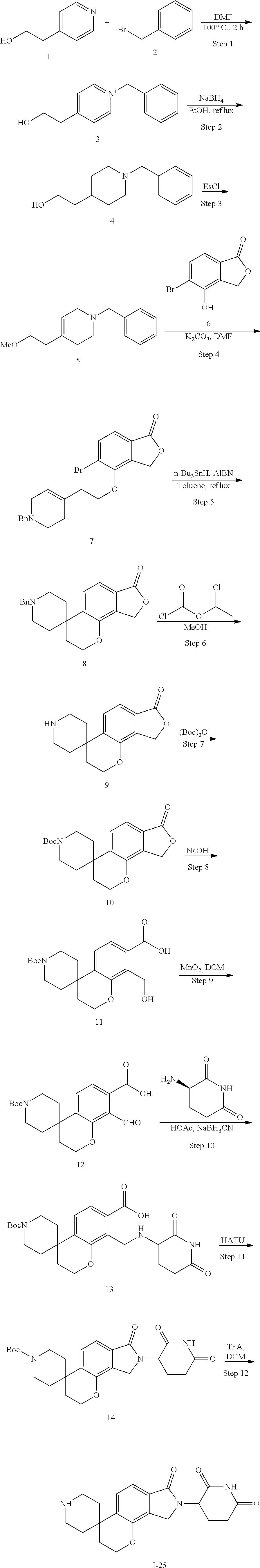

### Step 1-2

To a solution of 2-(pyridin-4-yl) ethan-1-ol (1, 10 g, 91.6 mmol, 1.0 eq.) in DMF (40 mL) was added BnBr (15.3 g, 108 mmol, 1.1 eq.). The mixture was allowed to heat to 100° C. and stirred 3 h. TLC showed no starting material remained and a new spot formed. The residue was dissolved in EtOH (150 mL), then 4.0 g of sodium borohydride (119.1 mmol, 1.3 eq.) was added portionwise at 0° C. The mixture was continued to stir at 0° C. for 1 h and then at reflux for 2 h. The solvent was evaporated under reduced pressure, then water was added, and the mixture was extracted with EA. The combined organic phases were dried over Na₂SO₄ and evaporated. The residue was purified by flash chromatograph (DCM:MeOH=100:0-30:1) to afford 10 g of product 4 (Viscous oil, 2 steps, yield 56%). LC-MS: 218 [M+H]⁺.

### Step 3

To a solution of compound 4 (10 g, 1 eq.) in DCM (200.0 mL) was added DMAP (0.1 eq.) and TEA (2 eq.) at 0° C. Then EsCl (1.5 eq.) was slowly added into and the mixture was stirred at R.T. for 1 h. The reaction was partitioned between EtOAc and water. The combined organic layers were dried over anhydrous Na₂SO₄, filtered, and concentrated in vacuo. The crude residue was purified by flash chromatograph to give compound 5 as a yellow solid (10 g, yield 70%). LC-MS: 310 [M+H]⁺.

### Step 4

To a solution of compound 6 (10 g, 1.0 eq.) in 100 mL of DMF, compound 5 (16.2 g, 1.2 eq.) and K₂CO₃ (1.6 eq.) was added. The reaction mixture was heated to 70° C. and stirred overnight. The reaction mixture was poured into ice-water and extracted with ethyl acetate, washed with brine, and then dried over sodium sulfate. The solvent was evaporated at reduced pressure and the crude product was purified by silica gel column chromatography using 0-100% EtOAc/hexane. The desired product 7 was obtained as a yellow foam (11 g, yield 60%). 428/430 [M+H]⁺.

### Step 5

To a solution of 7 (5 g, 1.0 eq.) in toluene (50 mL) was added n-Bu3SnH (13.6 g, 4.0 eq.) and AIBN (0.4 g, 0.1 eq.). The mixture was heated to reflux and stirred overnight. TLC (PE: EA=1:1) showed no starting material remained and new spots formed. The reaction mixture was poured into saturated aq. KF solution (100 mL) and stirred overnight. Then, the reaction mixture was extracted with ethyl acetate, washed with brine, and then dried over sodium sulfate. The crude product was purified by silica gel column chromatography (DCM:MeOH=50:1) to give compound 8 was obtained as a white solid (2 g, 50% yield). LC-MS: 350 [M+H]⁺.

### Step 6-7

To a solution of 8 (3.0 g, 1.0 eq.) in DCE (100 mL) was added α-chloroethyl chloroformate (ACE-Cl, 1.2 eq.) at 0° C. and then refluxing the mixture for 15 h. The intermediate ACE-piperidine formed and is usually deACEylated directly to 9 by evaporating the reaction mixture in vacuo and then heating the residue in MeOH. The residue was dissolved in THF (100 mL), then trimethylamine (3.0 eq.) and Boc₂O (1.3 eq.) was added. The mixture was continued to stir for 3 h at room temperature. The solvent was evaporated under reduced pressure, then water was added, and the mixture was extracted with EA. The combined organic phases were dried over Na₂SO₄ and evaporated. The residue was purified by flash chromatograph to afford 10 (1.5 g, 2 steps, yield 50%). LC-MS: 360 [M+H]⁺. ¹H NMR (600 MHZ, Chloroform-d) 8 7.47 (d, J=7.6 Hz, 1H), 7.08 (d, J=7.6 Hz, 1H), 5.24 (s, 2H), 4.16 (t, J=6.7 Hz, 2H), 3.88 (m, 2H), 3.51 (m, 2H), 2.52 (t, J=6.8 Hz, 2H), 2.13 (m, 2H), 1.61 (m, 2H), 1.46 (s, 9H).

### Step 8

To a solution of compound 10 (2 g, 1 eq.) in tetrahydrofuran (10 mL) and water (10 mL) was added sodium hydroxide (1.2 g, 5 eq.). The mixture was stirred at 20° C. for 16 h. TLC (ethyl acetate:hexane=1:1) showed reaction was complete. The mixture was adjusted to pH=5-6 with aq. hydrochloric acid (1 M) and extracted with ethyl acetate. The organic layer was washed with brine and dried over sodium sulfate. The crude product 11 was not further purified and used as crude for the next step.

### Step 9

To a solution of compound 11 (2 g, crude, 1 eq.) in dichloromethane (30 mL) was added manganese dioxide (20 eq.). The mixture was stirred at 20° C. for about 1 h. TLC showed reaction was complete. The mixture was diluted with dichloromethane and filtered through a pad of Celite. The filtrate was concentrated in vacuum. The crude product was purified by silica gel column chromatography (DCM:MeOH=10:1). The desired compound 12 was obtained as yellow solid. (1.2 g, 2 steps, 60%). LC-MS: 376 [M+H]⁺.

### Step 10

To a mixture of compound 12 (532 mg, 1.0 eq.) in methanol (5 mL) and dichloromethane (5 mL) was added 3-aminopiperidine-2,6-dione (698 mg, 3 eq., HCl salt), AcONa (698 mg, 6.0 eq.) and AcOH (0.85 mL, 10.0 eq.). The mixture was stirred at 25° C. for 1 h, then sodium cyanoborohydride (268 mg, 3.0 eq.) was added and the mixture was further stirred for 30 min. LCMS showed the reaction was complete. Next, the reaction mixture was quenched with water and concentrated under reduced pressure to give a residue which was purified by pre-HPLC (20%~ 50% ACN, neutral). The desired product 13 as a solid (415 mg, yield=60%) after lyophilization. LC-MS: 488 [M+H]⁺

### Step 11

To a solution of compound 13 (300 mg 1.0 equiv) in DMF (5 mL) was added HATU (300 mg, 1.3 equiv) and DIPEA (0.35 mL, 3.0 equiv), and the reaction was stirred at rt for 30 min. UPLC-MS indicated a new main peak with desired MS formed, then quenched with water and the mixture was extracted with ethyl acetate, washed with brine, and then dried over sodium sulfate. Compound 14 was obtained as a brown solid (230 mg, 75% yield). LC-MS: 470 [M+H]⁺.

### Step 12

Compound 14 was treated with TFA in DCM at room temperature to de-protect the N-Boc group to provide the cereblon ligand I-25. LC/MS (ESI) m/z: 370.17. ¹H NMR (400 MHZ, CDCl₃) δ 8.03 (s, 1H), 7.45 (d, J=1.2 Hz, 2H), 5.22 (dd, J=13.2, 5.1 Hz, 1H), 4.40 (d, J=16.4 Hz, 1H), 4.31-4.20 (m, 3H), 4.11 (brs, 1H), 3.06-2.78 (m, 4H), 2.37 (qd, J=13.1, 5.0 Hz, 1H), 2.22 (dtd, J=13.1, 5.3, 2.7 Hz, 1H), 2.17-2.04 (m, 3H), 1.60-1.50 (s, 4H).

### Intermediate 28: (S)-3-(7′-oxo-7′,9′-dihydro-2′H,8′H-spiro[piperidine-4,3′-[1,4]dioxino[2,3-e]isoindol]-8′-yl)piperidine-2,6-dione

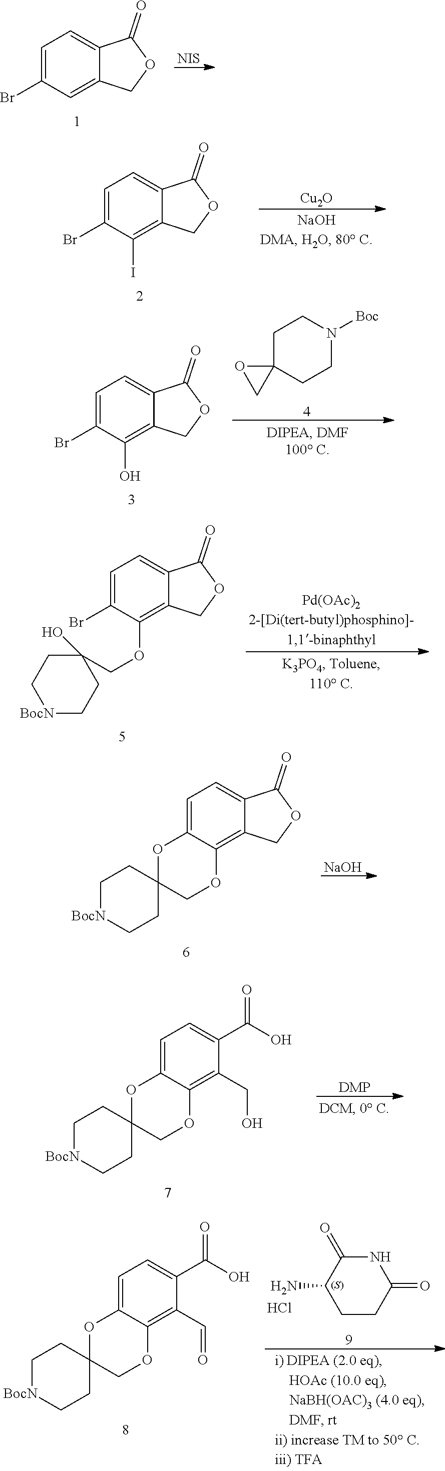

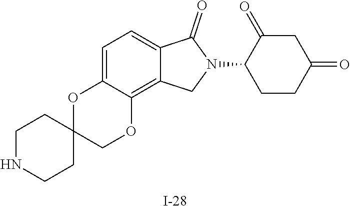

### Step1:5-bromo-4-iodoisobenzofuran-1 (3H)-one

To a solution of 1 (10 g, 1.0 equiv) in CF₃SO₃H (50 mL) was added NIS (1.5 equiv) potionwise at 0° C. The reaction was stirred at rt overnight. Then the reaction mixture was poured into ice-water, and gray solid was precipitated, which is collected by filtration and washed with water. The filter cake was dissolved in DCM, washed with aquous Na₂S203, brine, dried over Na₂SO₄ and concentrated to afford a crude product. Further purification by silica gel column chromatography to give the desired product as a white solid 6.55 g.

### Step 2:5-bromo-4-hydroxyisobenzofuran-1 (3H)-one

A mixture of 2 (6.55 g, 1.0 equiv), Cu₂O (553 mg, 0.2 equiv) and NaOH (3.86 g, 5.0 equiv) in DMA/H₂O (40 mL/20 mL) was degassed with N₂ and stirred at 80° C. under N₂ atmosphere overnight. Then the reaction mixture was cooled to rt, neutralized with 2N aq. HCl, extracted with EA, washed with brine, dried over Na₂SO₄, and concentrated to give the crude product, which is purified by silica gel column chromatography to provide compound 3 as a yellow solid 3.67 g (yield=83%). ¹H NMR (400 MHZ, DMSO-d₆) δ 10.89 (s, 1H), 7.72 (d, J=8.0 Hz, 1H), 7.23 (d, J=8.0 Hz, 1H), 5.34 (s, 2H).

### Step3: tert-butyl 4-(((5-bromo-1-oxo-1,3-dihydroisobenzofuran-4-yl)oxy)methyl)-4-hydroxypiperidine-1-carboxylate

To a solution of 3 (500 mg, 1.0 equiv) and 4 (931 mg, 2.0 equiv) in DMF (15 mL) was added DIPEA (3.8 mL, 10.0 equiv), which was stirred at 100° C. for 2 days. Then the reaction mixture was cooled to rt, diluted with EA, washed with brine, dried over Na₂SO₄ and concenterated. The crude product was purified by silica gel column chromatography to give compound 5 as a brown oil 1.03 g, yicld >95%. LC-MS: 344.01 [M+H]⁺.

### Step 4: tert-butyl 7′-oxo-7′,9′-dihydro-2′H-spiro[piperidine-4,3′-[1,4]dioxino[2,3-e]isobenzofuran]-1-carboxylate

A mixture of 5 (870 mg, 1.0 equiv), Pd (OAc) 2 (51 mg, 0.2 equiv), 2-[Di (tert-butyl)phosphino]-1,l′-binaphthyl (135 mg, 0.3 equiv) and K₃PO₄ (719 mg, 3.0 equiv) in Toluene (12 mL) was degassed with N₂ and then was stirred at 110° C. under N₂ atmosphere overnight. The reaction mixture was filtered through celite, and the filtration was concentrated under reduced pressure. The result mixture was purified by silica gel column chromatography to give compound 6 as a white solid 540 mg, yield=73%. LC-MS: 362.21 [M+H]⁺.

### Step 5: l′-(tert-butoxycarbonyl)-5-(hydroxymethyl)-3H-spiro[benzo[b][1,4]dioxine-2,4′-piperidine]-6-carboxylic acid

To a solution of 6 (298 mg, 1.0 equiv) in THF/MeOH/H₂O (5 mL/5 mL/3 mL) was added NaOH (330 mg, 10 equiv). The reaction was stirred at rt for 8 h, then concentrated to remove most of the THE/MeOH. The residue was diluted with 4 mL water, followed by neutralization with 2 N aq HCl to PH 4-6, then extracted with DCM. Then combined organic layer was washed with brine, dried with Na₂SO₄, and concentrated to give the desired product 7 as a white solid 284 mg, which was directly used in the next step.

### Step 6: 1′-(tert-butoxycarbonyl)-5-formyl-3H-spiro[benzo[b][1,4]dioxine-2,4′-piperidine]-6-carboxylic acid

To a solution of 7 (284 mg, 1.0 equiv) in DCM (15 mL) was added DMP (475 mg, 1.5 equiv) potionwise at 0° C. 5 h Later, the reaction mixture was washed with brine, dried over Na₂SO₄, and concentrated to give the crude product 8, which was directly used in the next step.

### Step 7: (S)-3-(7′-oxo-7′,9′-dihydro-2′H,8′H-spiro[piperidine-4,3′-[1,4]dioxino[2,3-e]isoindol]-8′-yl)piperidine-2,6-dione (I-28)

To a suspension of 9 (46 mg, 2.0 equiv) in DMF (4 mL) was added DIPEA (49 μL, 2.0 equiv), which was stirred at rt for 10 min, followed by addition of AcOH (423 μL, 10.0 equiv). 10 min Later, crude compound 8 (53 mg, 1.0 equiv) was added, and the resulted mixture was stirred at rt for 15 min. Subsequently, NaBH (OAc) 3 (119 mg, 4.0 equiv) was added, and the reaction mixture was stirred overnight. Then the reaction mixture was heated to 50° C. and kept stirring for 12 h. Next, the reaction mixture was concentrated to remove AcOH, and purified by pre-HPLC to give a light purple solid 33 mg. LC-MS: 472.17 [M+H]⁺. Finally, intermediate I-28 was obtained after treatment with TFA. LC-MS: 372.17 [M+H]⁺. ¹H NMR (400 MHZ, Mcthanol-d₄) δ 7.37 (d, J=8.2 Hz, 1H), 7.13 (d, J=8.2 Hz, 1H), 5.11 (dd, J=13.3, 5.1 Hz, 1H), 4.51-4.35 (m, 2H), 4.17 (d, J=2.0 Hz, 2H), 3.42-3.35 (m, 4H), 2.96-2.84 (m, 1H), 2.82-2.72 (m, 1H), 2.56-2.42 (m, 1H), 2.21-2.06 (m, 3H), 2.01-1.88 (m, 2H). ¹³C NMR (101 MHZ, MeOD) δ 174.64, 172.26, 171.11, 145.75, 138.95, 131.85, 126.85, 119.67, 118.32, 71.56, 53.74, 46.27, 40.52, 40.49, 32.37, 28.91, 28.85, 24.05.

### Intermediate 29: (S)-3-((S)-1-oxo-1,3,5,5a,6,7,8,9-octahydro-2H-pyrazino[1′,2′: 4,5][1,4]oxazino[2,3-e]isoindol-2-yl)piperidine-2,6-dione

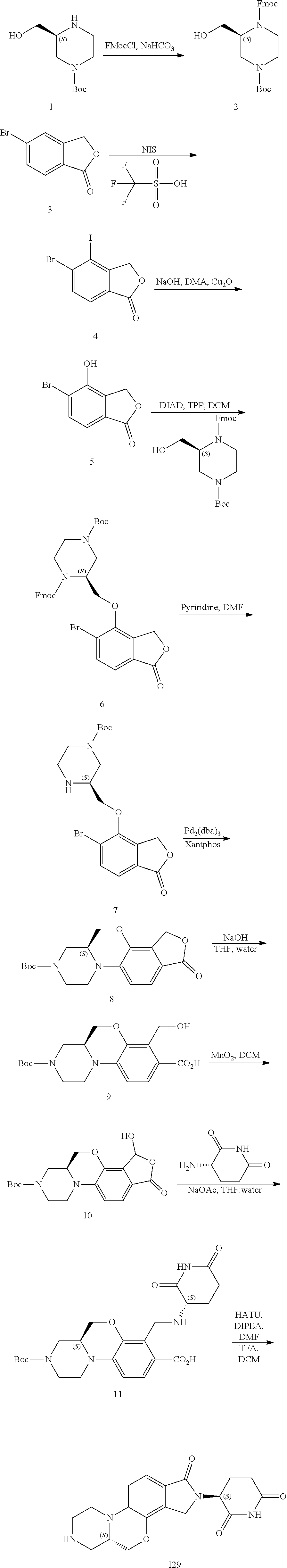

Intermediate I29 was made using the similar procedure for making intermediate I20.

¹H NMR of compound 129 (400 MHZ, Methanol-d₄) δ 7.37 (d, J=8.3 Hz, 1H), 7.14 (d, J=8.5 Hz, 1H), 5.11 (ddd, J=13.3, 5.2, 2.2 Hz, 1H), 4.50-4.34 (m, 3H), 4.34-4.10 (m, 3H), 3.67-3.42 (m, 4H), 3.30-3.22 (m, 1H), 3.22-3.09 (m, 1H), 3.02 (td, J=12.2, 5.7 Hz, 1H), 2.91 (ddd, J=18.5, 13.4, 5.4 Hz, 1H), 2.79 (ddd, J=17.6, 4.7, 2.4 Hz, 1H), 2.57-2.41 (m, 1H), 2.16 (dtd, J=12.9, 5.3, 2.5 Hz, 1H).

### Intermediate 30: (S)-3-((2S,3aS)-2-amino-7-oxo-2,3,3a,4,7,9-hexahydro-1H,8H-pyrrolo[1′,2′: 4,5][1,4]oxazino[2,3-f]isoindol-8-yl)piperidine-2,6-dione

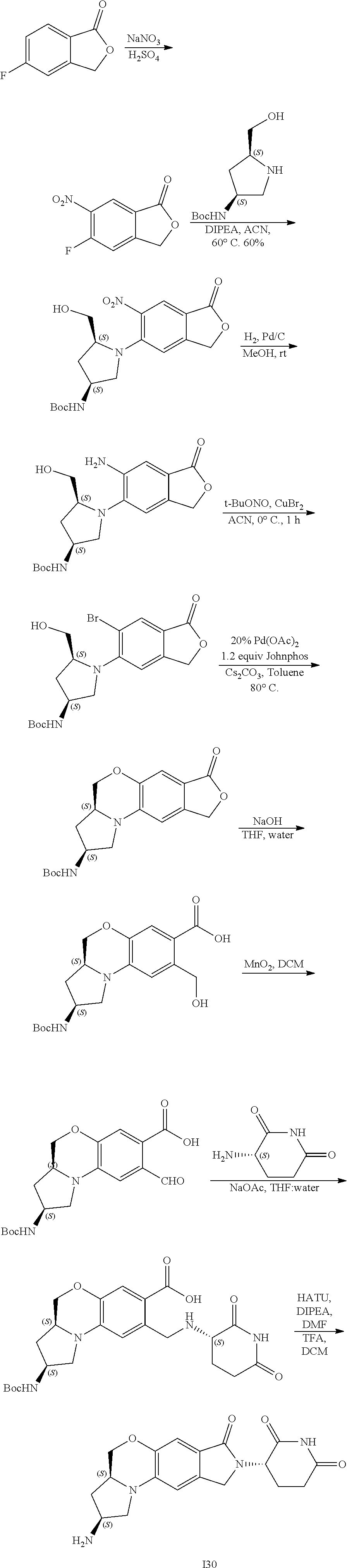

Intermediate 130 was made using the similar procedure for making intermediate 18.

¹H NMR of compound 130 (400 MHZ, Methanol-d₄) d 7.16 (s, 1H), 6.72 (s, 1H), 5.09 (dt, J=13.3, 5.1 Hz, 1H), 4.58 (d, J=7.1 Hz, 2H), 4.36 (d, J=6.7 Hz, 2H), 4.15 (d, J=3.6 Hz, 1H), 3.79 (dd, J=10.4, 7.9 Hz, 1H), 3.72-3.62 (m, 2H), 3.53-3.40 (m, 1H), 2.96-2.84 (m, 1H), 2.78 (ddd, J=17.4, 4.8, 2.5 Hz, 1H), 2.61 (ddd, J=12.5, 8.6, 4.1 Hz, 1H), 2.55-2.37 (m, 1H), 2.16 (ddq, J=10.4, 5.3, 2.7 Hz, 1H), 1.79-1.59 (m, 1H).

### Intermediate 31: (R)-3-((S)-3-oxo-1,3,7,7a,8,9,10,11-octahydro-2H-pyrazino[1′,2′: 4,5][1,4]oxazino[3,2-e]isoindol-2-yl)piperidine-2,6-dione

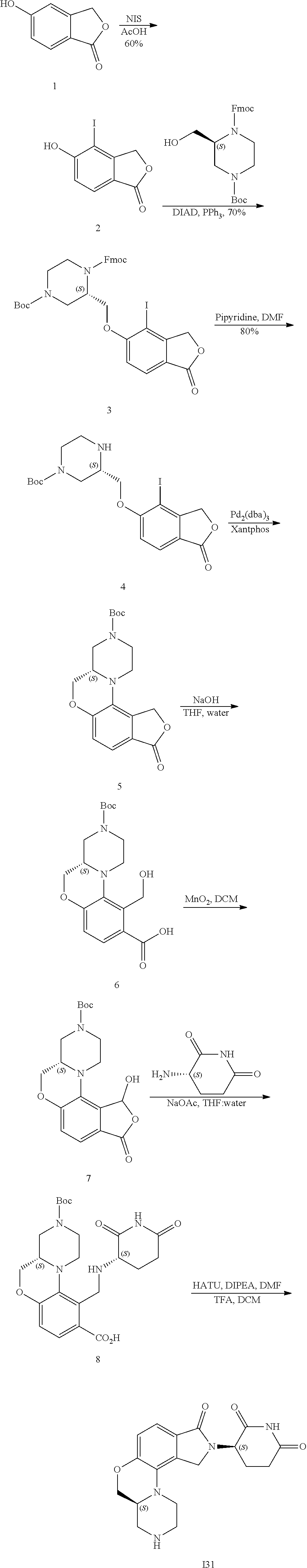

Intermediate I31 was made using the similar procedure for making intermediate 120.

¹H NMR of compound 131 (400 MHZ, Mcthanol-d₄) 8 7.32 (d, J=8.1 Hz, 1H), 7.02 (dd, J=8.1, 0.9 Hz, 1H), 5.15 (dd, J=13.4, 5.2 Hz, 1H), 4.70-4.47 (m, 2H), 4.34 (ddd, J=11.3, 4.2, 2.8 Hz, 1H), 4.14 (ddd, J=11.3, 9.8, 7.2 Hz, 1H), 4.04-3.91 (m, 1H), 3.65 (ddq, J=10.4, 7.1, 3.4, 2.8 Hz, 1H), 3.54-3.39 (m, 2H), 3.30-3.22 (m, 2H), 3.14 (dt, J=12.8, 10.6 Hz, 1H), 2.94 (ddd, J=17.6, 13.5, 5.4 Hz, 1H), 2.80 (ddd, J=17.6, 4.7, 2.4 Hz, 1H), 2.60-2.42 (m, 1H), 2.19 (ddq, J=10.5, 5.4, 2.8 Hz, 1H).

### Compound A3. 6-(2-(9-(4-((6R,8S)-7-(2,2-difluoroethyl)-8-methyl-6,7,8,9-tetrahydro-3H-pyrazolo[4,3-f]isoquinolin-6-yl)-3,5-difluorophenyl)-3,9-diazaspiro[5.5]undecan-3-yl)-2-oxoethyl-1,1-d₂)-2-(2,6-dioxopiperidin-3-yl)-6,7-dihydropyrrolo[3,4-f]isoindole-1,3 (2H,5H)-dione

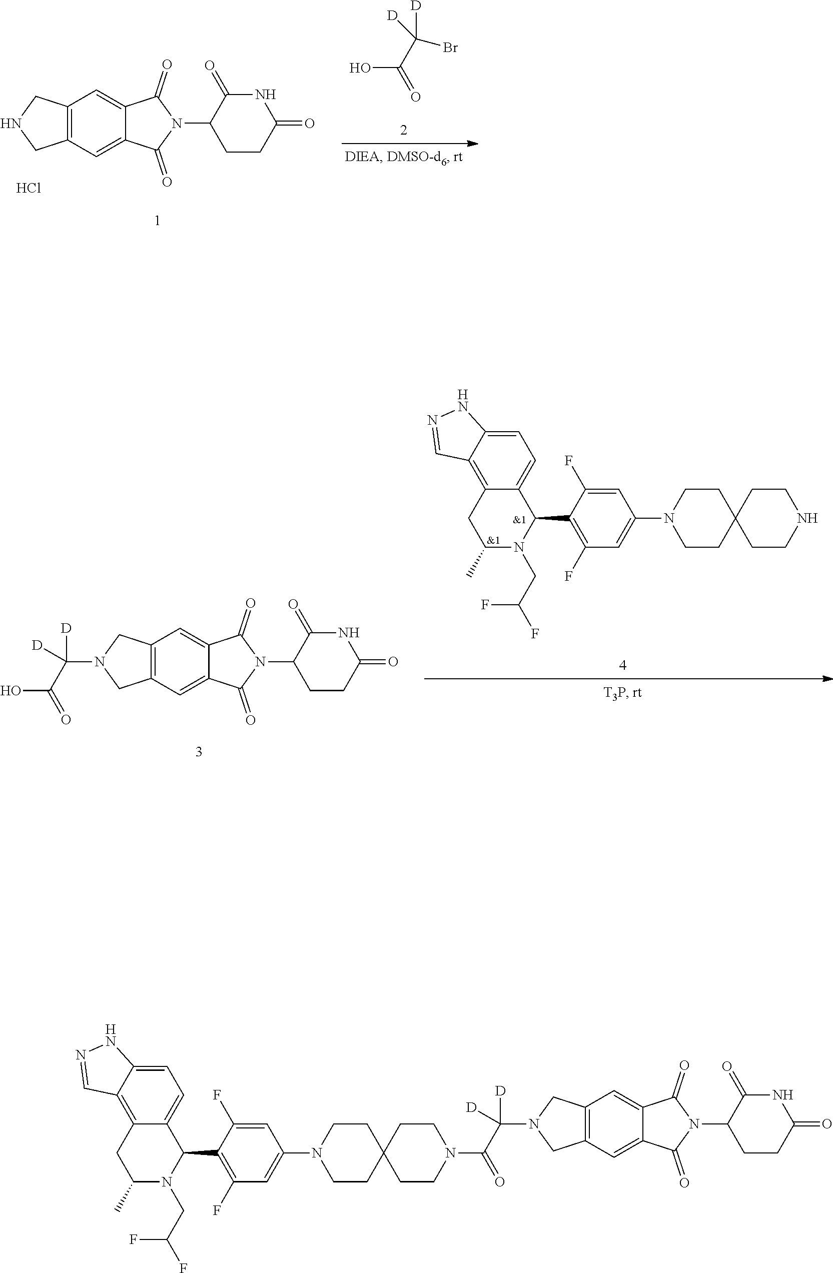 [SMILES: O=C1CCC(N2C(=O)c3cc4c(cc3C2=O)CNC4)C(=O)N1 ; [2H]C([2H])(Br)C(=O)O ; Cl ; [2H]C([2H])(C(=O)O)N1Cc2cc3c(cc2C1)C(=O)N(C1CCC(=O)NC1=O)C3=O ; C[C@@H]1Cc2c(ccc3[nH]ncc23)[C@@H](c2c(F)cc(N3CCC4(CCNCC4)CC3)cc2F)N1CC(F)F ; [2H]C([2H])(C(=O)N1CCC2(CC1)CCN(c1cc(F)c([C@@H]3c4ccc5[nH]ncc5c4C[C@@H](C)N3CC(F)F)c(F)c1)CC2)N1Cc2cc3c(cc2C1)C(=O)N(C1CCC(=O)NC1=O)C3=O]

### Step 1

To a solution of 2-(2,6-dioxopiperidin-3-yl)-6,7-dihydropyrrolo[3,4-f]isoindole-1,3 (2H,5H)-dione hydrochloride (300.0 mg, 0.89 mmol, 1 eq) and DIEA (0.47 mL, 2.68 mmol, 3 eq) in DMSO-d₆ (5 mL) was added 2-bromoacetic-2,2-d₂ acid (125.8 mg, 0.89 mmol, 1 eq) and stirred at room temperature for 2 hours. Then rac-(6R,8S)-6-(2,6-difluoro-4-(3,9-diazaspiro[5.5]undecan-3-yl)phenyl)-7-(2,2-difluoroethyl)-8-methyl-6,7,8,9-tetrahydro-3H-pyrazolo[4,3-f]isoquinoline (460.5 mg, 0.893 mmol, 1 eq.) and T3P (566.1 mg, 1.78 mmol, 2 eq.) was added and stirred at 25° C. for 16 hours. The reaction was quenched with D20 and concentrated under vacuum to give crude product. The residue was purificd by Prop-HPLC (ACN/0.05% FA) to afford 6-(2-(9-(4-((6R,8S)-7-(2,2-difluoroethyl)-8-methyl-6,7,8,9-tetrahydro-3H-pyrazolo[4,3-f]isoquinolin-6-yl)-3,5-difluorophenyl)-3,9-diazaspiro[5.5]undecan-3-yl)-2-oxoethyl-1,1-d₂)-2-(2,6-dioxopiperidin-3-yl)-6,7-dihydropyrrolo[3,4-f]isoindole-1,3 (2H,5H)-dione (1.76 mg, 0.23%) as a white solid.

LC-MS purity: 100% (UV at 254 nm), 857.3 [M+H]⁺.

¹H NMR (400 MHZ, DMSO-d₆) δ 12.98 (s, 1H), 11.14 (s, 1H), 8.06 (s, 1H), 7.99 (s, 2H), 7.24-7.21 (m, 2H), 7.11 (s, 1H), 6.98 (s, 1H), 6.71 (d, J=8.4 Hz, 1H), 6.55 (s, 1H), 6.52 (s, 1H), 5.95-5.68 (m, 1H), 5.16 (t, J=8.8 Hz, 2H), 3.22-3.18 (m, 4H), 3.06-3.00 (m, 1H), 2.92-2.87 (m, 2H), 2.67-2.59 (m, 3H) . . . 2.13-2.05 (m, 2H), 1.57-1.46 (m, 10H), 1.25-1.15 (m, 4H), 1.04 (d, J=6.8 Hz, 3H).

### Compound A11 (S)-3-((R)-7-((2-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

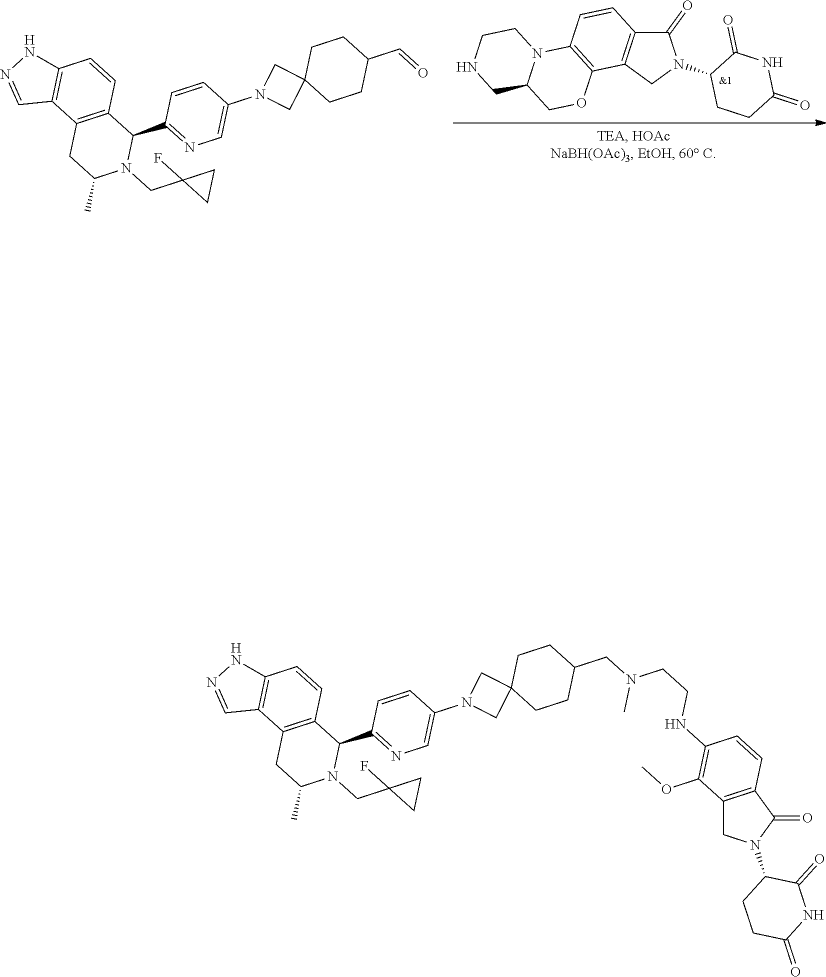 [SMILES: C[C@@H]1Cc2c(ccc3[nH]ncc23)[C@@H](c2ccc(N3CC4(CCC(C=O)CC4)C3)cn2)N1CC1(F)CC1 ; O=C1CC[C@H](N2Cc3c(ccc4c3OC[C@H]3CNCCN43)C2=O)C(=O)N1 ; COc1c(NCCN(C)CC2CCC3(CC2)CN(c2ccc([C@@H]4c5ccc6[nH]ncc6c5C[C@@H](C)N4CC4(F)CC4)nc2)C3)ccc2c1CN([C@H]1CCC(=O)NC1=O)C2=O]

To a mixture of 2-(6-((8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonane-7-carbaldehyde (35 mg, 0.07 mmol, 1.0 eq) and(S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione (28 mg, 0.07 mmol, 1.0 eq) in EtOH (5 mL) was added TEA (0.1 mL) and AcOH (0.2 mL), the mixture was stirred at 45° C. for 0.5 hour. The mixture was added NaBH (OAc) 3 (61 mg, 0.29 mmol, 4.0 eq) and stirred at 45° C. for 3 hours. LCMS showed the reaction was completed. The reaction was cooled to 20° C. and concentrated under vacuum. The residue was purified by Prep-HPLC (ACN/0.05% FA) to afford(S)-3-((R)-7-((2-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (14.46 mg) as a yellow solid.

LC-MS purity: 100% (UV at 254 nm), 828.3 [M+H]⁺.

¹H NMR (400 MHZ, DMSO) δ 12.94 (s, 1H), 10.93 (s, 1H), 8.21 (s, 1H), 8.04 (s, 1H), 7.66 (d, J=2.6 Hz, 1H), 7.17 (d, J=8.4 Hz, 2H), 7.02 (dd, J=8.3, 6.1 Hz, 2H), 6.77 (d, J=8.7 Hz, 1H), 6.72 (dd, J=8.5, 2.6 Hz, 1H), 5.03 (dd, J=13.2, 5.0 Hz, 1H), 4.84 (s, 1H), 4.36 (d, J=8.3 Hz, 1H), 4.26 (d, J=16.8 Hz, 1H), 4.10 (d, J=16.8 Hz, 1H), 4.01-3.91 (m, 1H), 3.82 (d, J=11.6 Hz, 1H), 3.69 (dd, J=11.6, 5.4 Hz, 1H), 3.53 (d, J=19.8 Hz, 4H), 3.21 (dd, J=16.4, 4.6 Hz, 2H), 2.94 (ddd, J=31.0, 19.3, 11.9 Hz, 5H), 2.79-2.69 (m, 1H), 2.68-2.53 (m, 2H), 2.39 (ddd, J=26.5, 13.4, 4.6 Hz, 1H), 2.20-2.03 (m, 3H), 1.93 (dd, J=23.8, 8.7 Hz, 3H), 1.71 (d, J=10.5 Hz, 3H), 1.47 (t, J=11.6 Hz, 2H), 1.01 (d, J=6.5 Hz, 3H), 0.97-0.81 (m, 3H), 0.73-0.63 (m, 1H), 0.52-0.42 (m, 1H).

### Compound A12. (R)-3-((S)-3-((7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

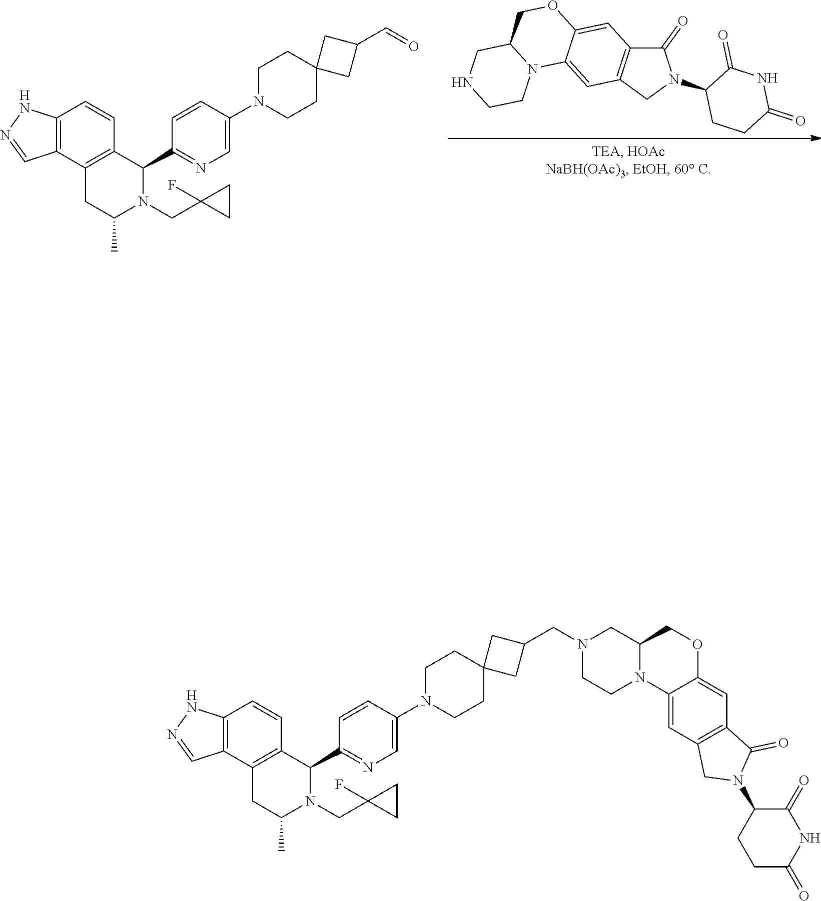 [SMILES: C[C@@H]1Cc2c(ccc3[nH]ncc23)[C@@H](c2ccc(N3CCC4(CC3)CC(C=O)C4)cn2)N1CC1(F)CC1 ; O=C1CC[C@@H](N2Cc3cc4c(cc3C2=O)OC[C@@H]2CNCCN42)C(=O)N1 ; C[C@@H]1Cc2c(ccc3[nH]ncc23)[C@@H](c2ccc(N3CCC4(CC3)CC(CN3CCN5c6cc7c(cc6OC[C@@H]5C3)C(=O)N([C@@H]3CCC(=O)NC3=O)C7)C4)cn2)N1CC1(F)CC1]

To a mixture of 7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonane-2-carbaldehyde (30 mg, 0.06 mmol, 1.0 eq) and (R)-3-((S)-8-oxo-1,2,3,4,4a,5,8,10-octahydro-9H-pyrazino[11,2′: 4,5][1,4]oxazino[2,3-f]isoindol-9-yl)piperidine-2,6-dione (24 mg, 0.06 mmol, 1.0 eq) in EtOH (5 mL) was added TEA (0.1 mL) and HOAc (0.2 mL), the mixture was stirred at 50° C. for 1 hour under N₂. The mixture was added NaBH (OAc) 3 (52 mg, 0.25 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford (R)-3-((S)-3-((7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonan-2-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione (5.70 mg) as a yellow solid.

LC-MS purity: 99.7% (UV at 254 nm), 828.3 [M+H]⁺.

¹H NMR (400 MHZ, DMSO) δ 13.17 (s, 1H), 10.95 (s, 1H), 10.12 (s, 1H), 8.27 (s, 1H), 8.16 (s, 1H), 7.38 (s, 2H), 7.19 (s, 2H), 7.04 (s, 1H), 6.91 (d, J=7.7 Hz, 1H), 5.04 (dd, J=13.3, 4.9 Hz, 1H), 4.41-4.15 (m, 5H), 4.10-3.93 (m, 2H), 3.57 (s, 4H), 3.14 (s, 7H), 2.99-2.79 (m, 3H), 2.71 (dd, J=18.1, 6.2 Hz, 1H), 2.59 (d, J=18.0 Hz, 1H), 2.38 (dd, J=17.9, 9.3 Hz, 1H), 2.10-1.95 (m, 3H), 1.75-1.50 (m, 7H), 1.27 (d, J=19.3 Hz, 3H), 1.14 (dd, J=21.0, 8.6 Hz, 2H), 0.83 (dd, J=32.7, 25.5 Hz, 2H).

### Compound A15. 3-((4-(1-((7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonan-2-yl)methyl)piperidin-4-yl)phenyl)amino) piperidin-2-one

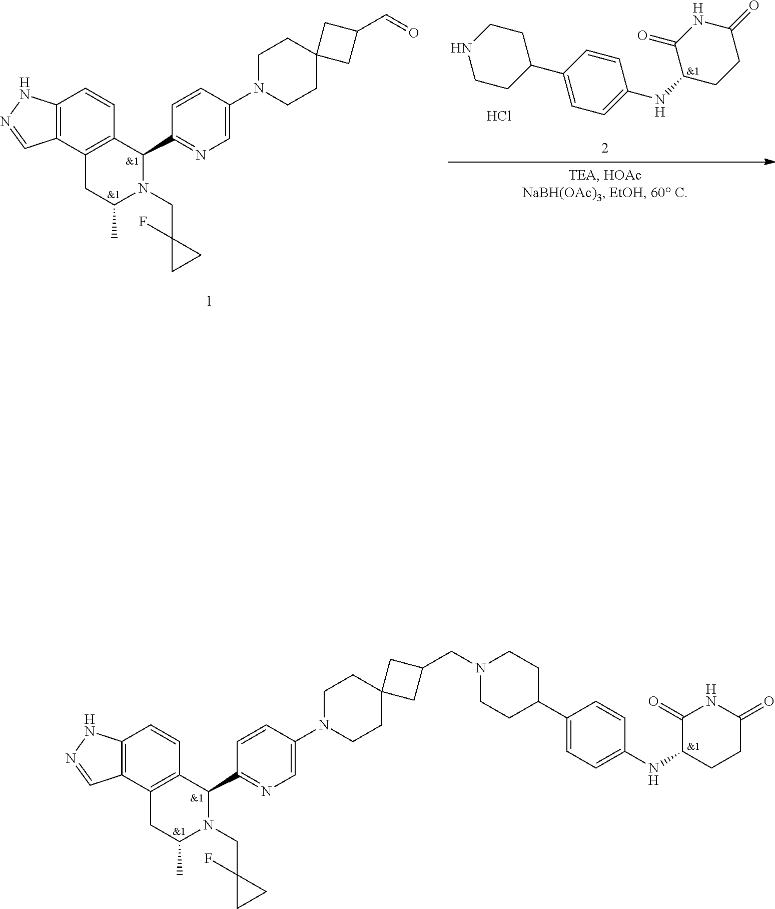 [SMILES: C[C@@H]1Cc2c(ccc3[nH]ncc23)[C@@H](c2ccc(N3CCC4(CC3)CC(C=O)C4)cn2)N1CC1(F)CC1 ; O=C1CC[C@H](Nc2ccc(C3CCNCC3)cc2)C(=O)N1 ; Cl ; C[C@@H]1Cc2c(ccc3[nH]ncc23)[C@@H](c2ccc(N3CCC4(CC3)CC(CN3CCC(c5ccc(N[C@H]6CCC(=O)NC6=O)cc5)CC3)C4)cn2)N1CC1(F)CC1]

To a mixture of 7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonane-2-carbaldehyde (20 mg, 0.04 mmol, 1.0 eq) and 3-((4-(piperidin-4-yl)phenyl)amino) piperidine-2,6-dione (12 mg, 0.04 mmol, 1.0 eq) in THF (5 mL) was added Ti (OiPr) 4 (47 mg, 0.16 mmol, 4 eq), the mixture was stirred at 45° C. for 1 hour under N₂. The mixture was added NaBH (OAc) 3 (35 mg, 0.16 mmol, 4.0 eq) and stirred at 45° C. for 2 hours under N₂. LCMS showed the reaction was completed. The reaction was cooled to 20° C. and concentrated under vacuum. The residue was purified by Prep-HPLC (ACN/0.05% FA) to afford 3-((4-(1-((7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonan-2-yl)methyl)piperidin-4-yl)phenyl)amino) piperidin-2-one (3.46 mg) as a yellow solid.

LC-MS purity: 97.99% (UV at 254 nm), 759.3 [M+H]⁺.

¹H NMR (400 MHZ, MeOD) δ 8.03 (d, J=2.8 Hz, 1H), 8.00 (s, 1H), 7.25 (d, J=6.2 Hz, 1H), 7.14 (t, J=8.4 Hz, 2H), 6.95 (d, J=8.0 Hz, 2H), 6.73 (d, J=9.0 Hz, 1H), 6.62 (d, J=8.5 Hz, 2H), 5.04 (s, 1H), 4.17 (dd, J=11.8, 4.7 Hz, 1H), 3.87 (t, J=10.2 Hz, 1H), 3.43 (dd, J=29.3, 15.0 Hz, 3H), 3.14 (s, 4H), 3.05 (s, 2H), 2.96 (d, J=16.3 Hz, 2H), 2.73-2.60 (m, 5H), 2.22 (d, J=9.7 Hz, 1H), 2.09 (t, J=7.6 Hz, 2H), 1.96 (d, J=14.8 Hz, 2H), 1.83 (d, J=13.0 Hz, 2H), 1.74 (d, J=5.2 Hz, 2H), 1.61 (d, J=8.9 Hz, 3H), 1.19 (s, 2H), 1.07 (d, J=6.5 Hz, 3H), 0.92-0.81 (m, 2H), 0.43 (dt, J=17.1, 10.8 Hz, 2H).

### Compound A16. (S)-3-((R)-7-((2-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

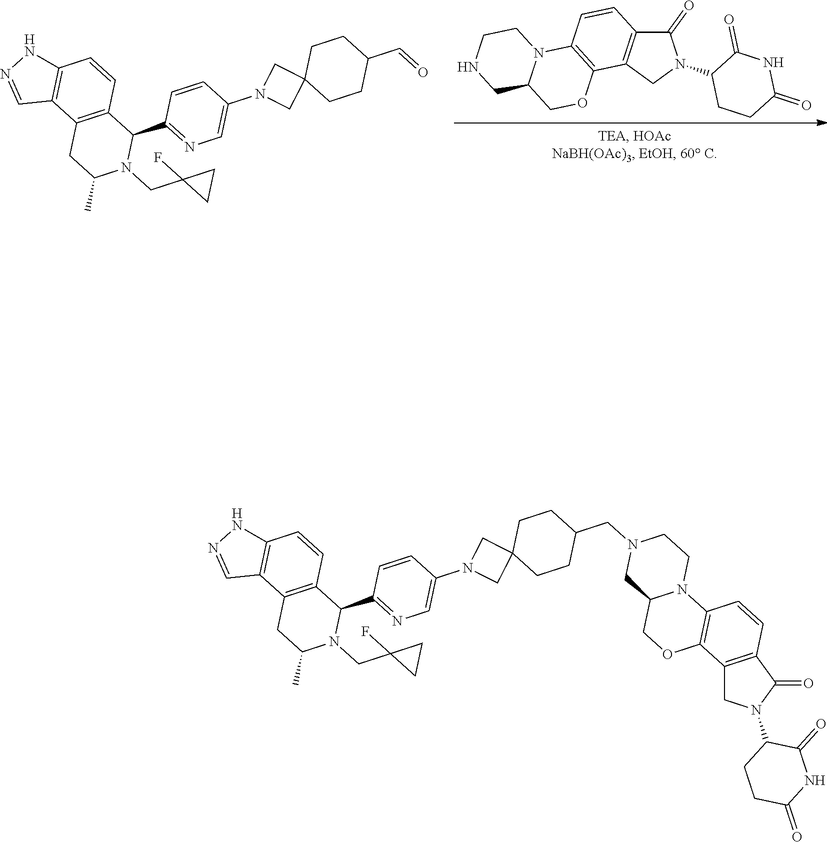

To a mixture of 2-(6-((8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonane-7-carbaldehyde (30 mg, 0.06 mmol, 1.0 eq) and(S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione (24 mg, 0.06 mmol, 1.0 eq) in EtOH (5 mL) was added TEA (0.1 mL) and HOAc (0.2 mL), the mixture was stirred at 45° C. for 0.5 hour under N₂. The mixture was added NaBH (OAc) 3 (52 mg, 0.25 mmol, 4.0 eq) and stirred at 45° C. for 3 hours under N₂. LCMS showed the reaction was completed. The reaction was cooled to 20° C. and concentrated under vacuum. The residue was purified by Prep-HPLC (ACN/0.05% FA) to afford (R)-3-((R)-7-((2-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione (14.87 mg) as a yellow solid.

LC-MS purity: 100% (UV at 254 nm), 828.3 [M+H]⁺.

¹H NMR (400 MHZ, DMSO) δ 12.94 (s, 1H), 10.93 (s, 1H), 8.30 (s, 1H), 8.04 (s, 1H), 7.66 (d, J=2.7 Hz, 1H), 7.17 (d, J=8.4 Hz, 2H), 7.02 (dd, J=8.4, 4.9 Hz, 2H), 6.77 (d, J=8.7 Hz, 1H), 6.72 (dd, J=8.6, 2.7 Hz, 1H), 5.03 (dd, J=13.2, 5.0 Hz, 1H), 4.84 (s, 1H), 4.36 (d, J=8.4 Hz, 1H), 4.26 (d, J=16.8 Hz, 1H), 4.10 (d, J=16.9 Hz, 1H), 4.03-3.94 (m, 1H), 3.83 (d, J=11.7 Hz, 1H), 3.70 (d, J=5.3 Hz, 1H), 3.55 (s, 2H), 3.50 (s, 2H), 3.25-3.14 (m, 3H), 3.05-2.96 (m, 1H), 2.92 (d, J=13.6 Hz, 3H), 2.78-2.58 (m, 3H), 2.38 (dd, J=20.6, 11.9 Hz, 1H), 2.11 (dd, J=26.9, 8.3 Hz, 3H), 1.92 (dd, J=18.9, 9.1 Hz, 3H), 1.71 (t, J=10.4 Hz, 3H), 1.47 (t, J=11.6 Hz, 3H), 1.01 (d, J=6.5 Hz, 3H), 0.97-0.80 (m, 4H), 0.72-0.63 (m, 1H), 0.46 (dt, J=11.3, 8.0 Hz, 1H).

### Compound A17. (S)-3-((R)-7-((2-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-2-azaspiro[3.5]nonan-7-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

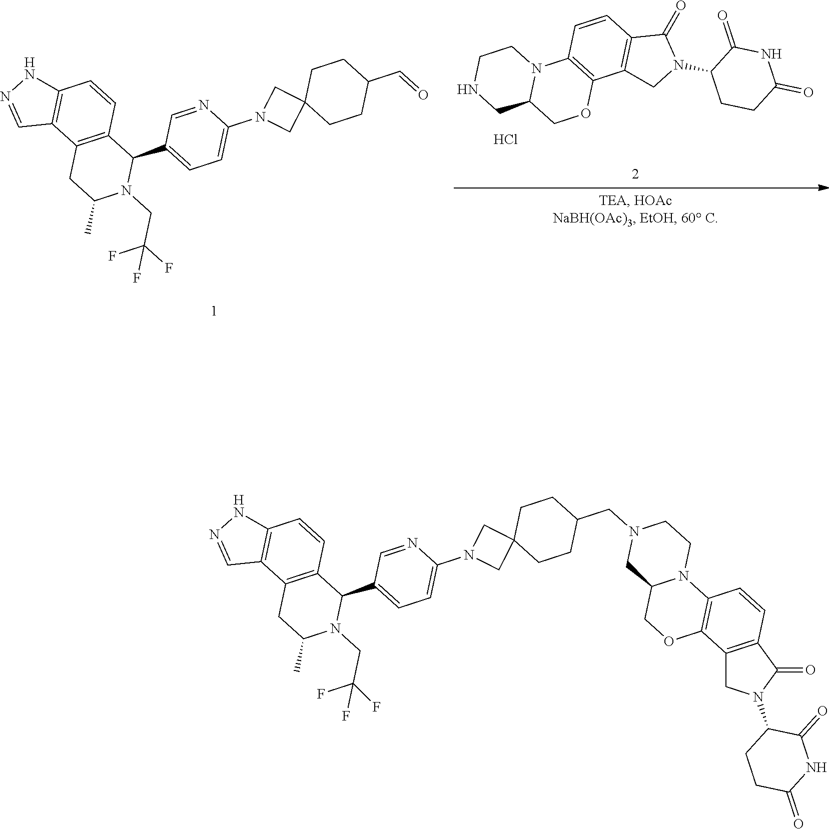 [SMILES: C[C@@H]1Cc2c(ccc3[nH]ncc23)[C@@H](c2ccc(N3CC4(CCC(C=O)CC4)C3)nc2)N1CC(F)(F)F ; O=C1CC[C@H](N2Cc3c(ccc4c3OC[C@H]3CNCCN43)C2=O)C(=O)N1 ; Cl ; C[C@@H]1Cc2c(ccc3[nH]ncc23)[C@@H](c2ccc(N3CC4(CCC(CN5CCN6c7ccc8c(c7OC[C@H]6C5)CN([C@H]5CCC(=O)NC5=O)C8=O)CC4)C3)nc2)N1CC(F)(F)F]

To a mixture of 2-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-2-azaspiro[3.5]nonane-7-carbaldehyde (190.0 mg, 0.38 mmol, 1.0 eq) and(S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione hydrochloride (150.1 mg, 0.38 mmol, 1.0 eq) in EtOH (15 mL) was added triethylamine (0.21 mL, 1.53 mmol, 4.0 eq), followed by the addition of AcOH (0.87 mL, 15.27 mmol, 40 eq) and stirred at 25° C. for 0.5 hour. The mixture was added NaBH (OAc) 3 (323.7 mg, 1.53 mmol, 4.0 eq) and stirred at 60° C. for 2 hours. LCMS showed the reaction was completed. The reaction was cooled to 20° C. and concentrated under vacuum. The residue was purified by Prep-HPLC (ACN/0.05% FA) to afford(S) -3-((R)-7-((2-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-2-azaspiro[3.5]nonan-7-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (188.63 mg, 58.9%) as a white solid.

LCMS purity: 100% (UV at 254 nm), 838.2 [M+1]⁺.

¹H NMR (400 MHZ, DMSO-d₆) δ 13.04 (s, 1H), 10.93 (s, 1H), 8.22 (dt, J=12.1, 10.0 Hz, 1H), 8.08 (s, 1H), 7.73 (d, J=2.0 Hz, 1H), 7.29 (d, J=8.5 Hz, 1H), 7.17 (d, J=8.4 Hz, 1H), 7.01 (d, J=8.5 Hz, 1H), 6.86 (d, J=8.5 Hz, 1H), 6.27 (d, J=8.7 Hz, 1H), 5.32 (t, J=4.9 Hz, 1H), 5.06 -4.93 (m, 2H), 4.38-4.33 (m, 1H), 4.28-4.23 (m, 1H), 4.10 (d, J=17.1 Hz, 1H), 3.99-3.93 (m, 1H), 3.82 (dd, J=11.0, 2.3 Hz, 1H), 3.54 (s, 3H), 3.01-2.84 (m, 7H), 2.67 (s, 1H), 2.33 (s, 1H), 2.13 (dd, J=9.7, 6.4 Hz, 2H), 1.99-1.95 (m, 2H), 1.86 (dd, J=10.6, 3.5 Hz, 2H), 1.70 (t, J=8.2 Hz, 2H), 1.45 (dd, J=18.7, 7.7 Hz, 3H), 1.24 (s, 4H), 1.07 (d, J=6.6 Hz, 3H), 0.96-0.92 (m, 1H), 0.85 (t, J=5.1 Hz, 1H).

### Compound A19. (S)-3-((R)-7-((1-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)piperidin-4-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

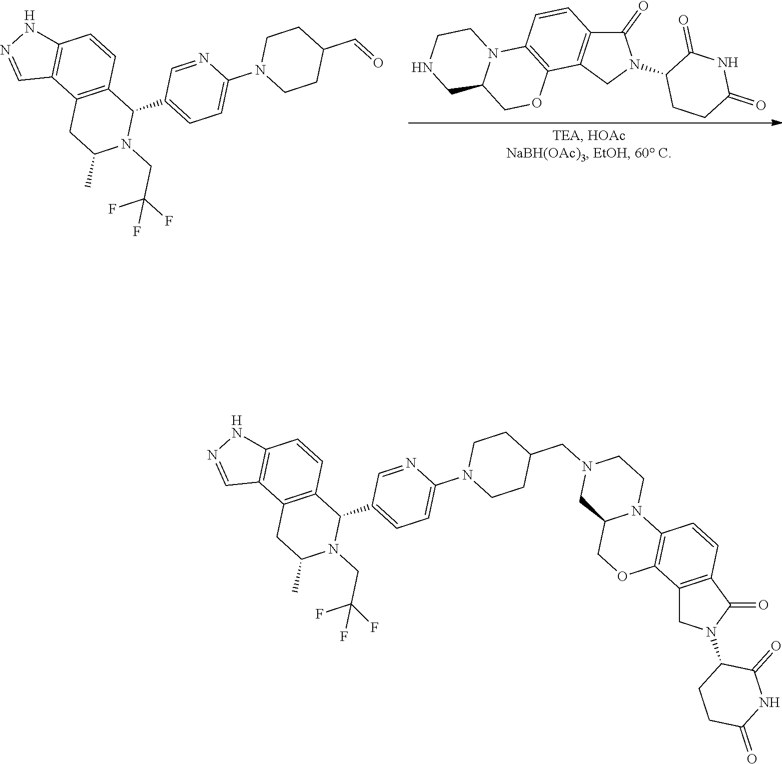 [SMILES: C[C@@H]1Cc2c(ccc3[nH]ncc23)[C@H](c2ccc(N3CCC(C=O)CC3)nc2)N1CC(F)(F)F ; O=C1CC[C@H](N2Cc3c(ccc4c3OC[C@H]3CNCCN43)C2=O)C(=O)N1 ; C[C@@H]1Cc2c(ccc3[nH]ncc23)[C@H](c2ccc(N3CCC(CN4CCN5c6ccc7c(c6OC[C@H]5C4)CN([C@H]4CCC(=O)NC4=O)C7=O)CC3)nc2)N1CC(F)(F)F]

To a mixture of 1-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)piperidine-4-carbaldehyde (30 mg, 0.07 mmol, 1.0 eq) and(S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione (26 mg, 0.07 mmol, 1.0 eq) in EtOH (5 mL) was added TEA (0.1 mL) and HOAc (0.2 mL), the mixture was stirred at 50° C. for 1 hour under N₂. The mixture was added NaBH (OAc) 3 (56 mg, 0.26 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford(S)-3-((R)-7-((1-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)piperidin-4-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (3.85 mg) as a white solid.

LC-MS purity: 100% (UV at 254 nm), 798.1 [M+H]⁺

¹H NMR (400 MHZ, MeOD) δ 8.39 (s, 1H), 7.99 (s, 1H), 7.68 (s, 1H), 7.31 (d, J=7.3 Hz, 1H), 7.24 (d, J=8.3 Hz, 1H), 7.19 (d, J=8.4 Hz, 1H), 6.89 (dd, J=17.7, 8.5 Hz, 2H), 6.68 (d, J=8.8 Hz, 1H), 4.98 (dd, J=13.5, 5.3 Hz, 1H), 4.91 (s, 1H), 4.24 (dd, J=17.3, 9.3 Hz, 3H), 4.12 (d, J=12.3 Hz, 2H), 3.93 (t, J=9.4 Hz, 1H), 3.73 (d, J=11.3 Hz, 1H), 3.33 (dd, J=19.5, 12.4 Hz, 2H), 2.98 (d, J=17.5 Hz, 2H), 2.87 (d, J=9.1 Hz, 2H), 2.83-2.73 (m, 5H), 2.67 (d, J=15.2 Hz, 1H), 2.37 (dd, J=23.7, 15.1 Hz, 1H), 2.19 (d, J=6.0 Hz, 2H), 2.15-2.02 (m, 2H), 1.83-1.73 (m, 4H), 1.17 (d, J=19.3 Hz, 3H), 1.06 (d, J=6.7 Hz, 3H).

### Compound A21. (3S)-3-((5aR)-7-((8-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-1-oxa-8-azaspiro[4.5]decan-3-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

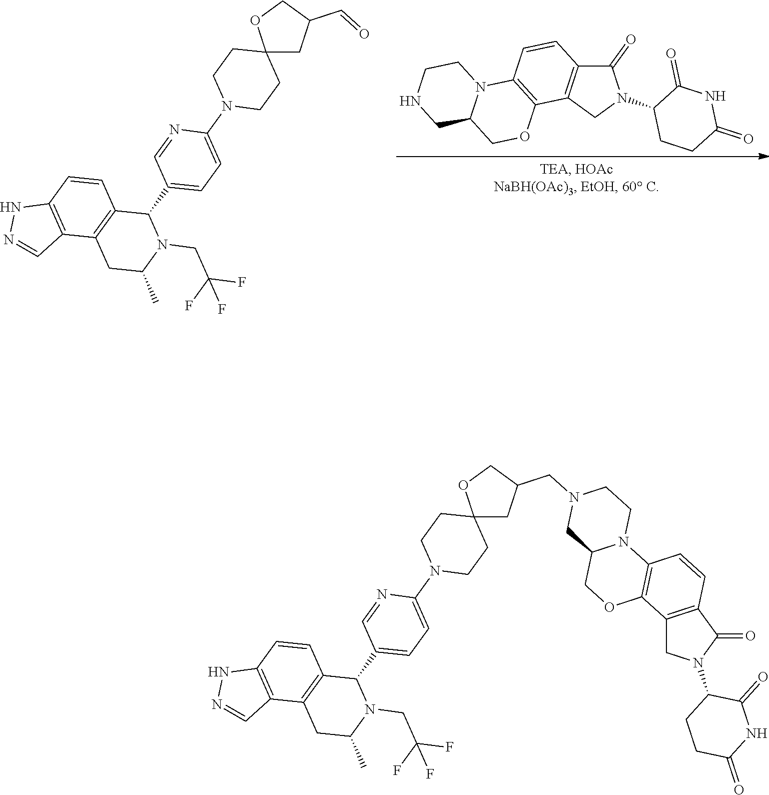 [SMILES: C[C@@H]1Cc2c(ccc3[nH]ncc23)[C@H](c2ccc(N3CCC4(CC3)CC(C=O)CO4)nc2)N1CC(F)(F)F ; O=C1CC[C@H](N2Cc3c(ccc4c3OC[C@H]3CNCCN43)C2=O)C(=O)N1 ; C[C@@H]1Cc2c(ccc3[nH]ncc23)[C@H](c2ccc(N3CCC4(CC3)CC(CN3CCN5c6ccc7c(c6OC[C@H]5C3)CN([C@H]3CCC(=O)NC3=O)C7=O)CO4)nc2)N1CC(F)(F)F]

To a mixture of 8-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-1-oxa-8-azaspiro[4.5]decane-3-carbaldehyde (30 mg, 0.06 mmol, 1.0 eq) and(S)-3-((R)-1-oxo-1,3,5,5a,6,7,8,9-octahydro-2H-pyrazino[11,2′: 4,5][1,4]oxazino[2,3-c]isoindol-2-yl)piperidine-2,6-dione (23 mg, 0.06 mmol, 1.0 eq) in EtOH (5 mL) was added TEA (0.1 mL) and HOAc (0.2 mL), the mixture was stirred at 50° C. for 1 hour under N₂. The mixture was added NaBH (OAc) 3 (50 mg, 0.23 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford(S)-3-((R)-7-(((R)-8-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-1-oxa-8-azaspiro[4.5]decan-3-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (3.11 mg) as a white solid.

LCMS purity: 100% (UV at 254 nm), 854.2 [M+1]⁺.

¹H NMR (400 MHZ, DMSO) δ 13.05 (s, 1H), 10.93 (s, 1H), 8.32 (s, 1H), 8.08 (s, 1H), 7.77 (s, 1H), 7.30 (d, J=8.7 Hz, 1H), 7.22-7.17 (m, 1H), 7.01 (d, J=8.5 Hz, 1H), 6.88 (d, J=8.7 Hz, 1H), 6.76 (d, J=8.9 Hz, 1H), 5.05-5.00 (m, 1H), 4.95 (s, 1H), 4.36 (d, J=9.9 Hz, 1H), 4.26 (d, J=17.0 Hz, 1H), 4.10 (d, J=16.7 Hz, 1H), 3.98-3.90 (m, 2H), 3.83 (d, J=10.7 Hz, 1H), 3.61-3.57 (m, 2H), 3.46 (s, 2H), 3.18-3.10 (m, 2H), 3.00 (d, J=12.0 Hz, 2H), 2.91 (d, J=12.2 Hz, 2H), 2.84 (d, J=17.7 Hz, 2H), 2.74 (s, 1H), 2.67 (s, 1H), 2.35 (d, J=13.8 Hz, 3H), 2.15-2.06 (m, 1H), 1.97 (dd, J=14.7, 7.0 Hz, 2H), 1.77-1.70 (m, 1H), 1.62-1.53 (m, 4H), 1.33 (dd, J=26.5, 13.1 Hz, 2H), 1.08 (d, J=6.6 Hz, 3H).

Compound A24. (S)-3-((S)-3-((1-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)piperidin-4-yl)methyl)-8-oxo-1,2,3,4,4a,5,8,10-octahydro-9H-pyrazino[1′,2′: 4,5][1,4]oxazino[2,3-f]isoindol-9-yl)piperidine-2,6-dione

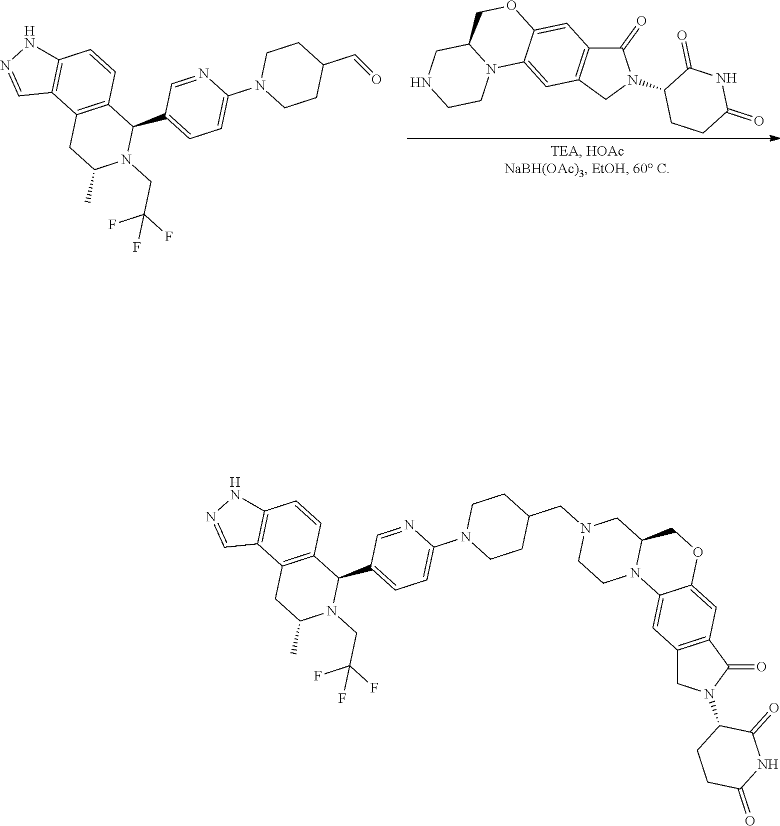 [SMILES: C[C@@H]1Cc2c(ccc3[nH]ncc23)[C@@H](c2ccc(N3CCC(C=O)CC3)nc2)N1CC(F)(F)F ; O=C1CC[C@H](N2Cc3cc4c(cc3C2=O)OC[C@@H]2CNCCN42)C(=O)N1 ; C[C@@H]1Cc2c(ccc3[nH]ncc23)[C@@H](c2ccc(N3CCC(CN4CCN5c6cc7c(cc6OC[C@@H]5C4)C(=O)N([C@H]4CCC(=O)NC4=O)C7)CC3)nc2)N1CC(F)(F)F]

To a mixture of 1-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)piperidine-4-carbaldehyde (17 mg, 0.04 mmol, 1.0 eq) and(S)-3-((R)-1-oxo-1,3,5,5a,6,7,8,9-octahydro-2H-pyrazino[1′,2′: 4,5][1,4]oxazino[2,3-e]isoindol-2-yl)piperidine-2,6-dione (15 mg, 0.04 mmol, 1.0 eq) in EtOH (5 mL) was added TEA (0.1 mL) and HOAc (0.2 mL), the mixture was stirred at 50° C. for 1 hour under N₂. The mixture was added NaBH (OAc) 3 (32 mg, 0.15 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford(S)-3-((S)-3-((1-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)piperidin-4-yl)methyl)-8-oxo-1,2,3,4,4a,5,8,10-octahydro-9H-pyrazino[11,2′: 4,5][1,4]oxazino[2,3-f]isoindol-9-yl)piperidine-2,6-dione (2.15 mg) as a white solid.

LCMS purity: 100% (UV at 254 nm), 798.2 [M+1]⁺.

¹H NMR (400 MHZ, DMSO) δ 13.04 (s, 1H), 10.93 (s, 1H), 8.18 (s, 1H), 8.08 (s, 1H), 7.77 (s, 1H), 7.30 (d, J=8.6 Hz, 1H), 7.21 (d, J=6.8 Hz, 1H), 7.04 (s, 1H), 6.93 (s, 1H), 6.88 (d, J=8.6 Hz, 1H), 6.74 (d, J=8.9 Hz, 1H), 5.02 (dd, J=13.2, 5.0 Hz, 1H), 4.95 (s, 1H), 4.26 (dd, J=19.1, 11.3 Hz, 4H), 4.15 (d, J=16.7 Hz, 1H), 3.96-3.87 (m, 1H), 3.81 (d, J=10.7 Hz, 1H), 3.53 (dd, J=16.1, 9.7 Hz, 2H), 3.06-2.96 (m, 3H), 2.93 (d, J=6.4 Hz, 3H), 2.80-2.67 (m, 4H), 2.35 (d, J=12.9 Hz, 1H), 2.23-2.15 (m, 2H), 2.12-2.06 (m, 1H), 2.03-1.91 (m, 2H), 1.81-1.70 (m, 4H), 1.24 (s, 2H), 1.08 (d, J=6.6 Hz, 3H).

### Compound A25. (S)-3-((R)-7-((7-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

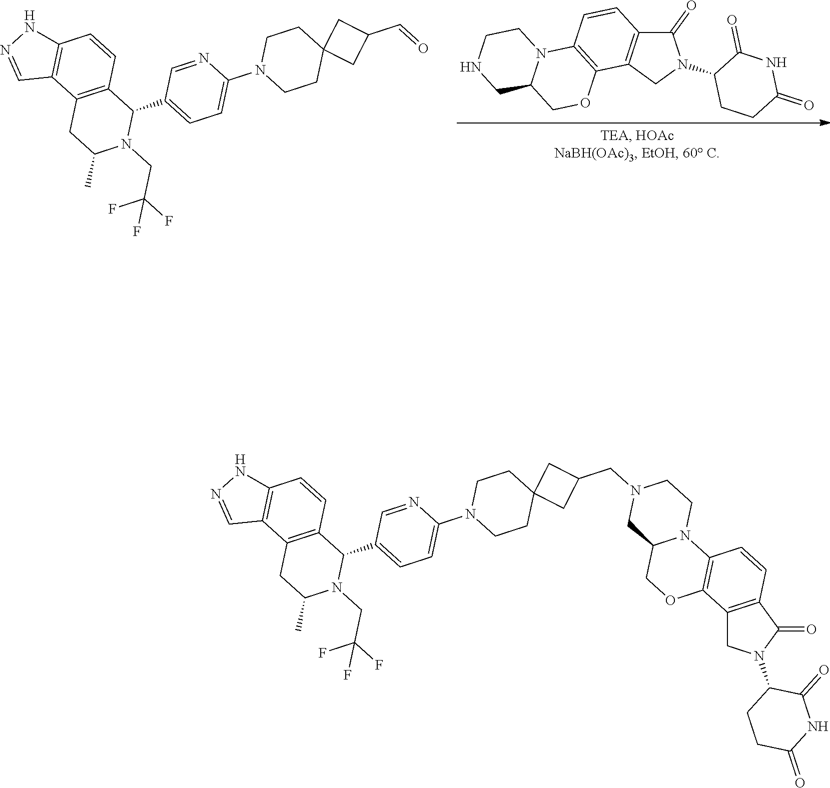 [SMILES: C[C@@H]1Cc2c(ccc3[nH]ncc23)[C@H](c2ccc(N3CCC4(CC3)CC(C=O)C4)nc2)N1CC(F)(F)F ; O=C1CC[C@H](N2Cc3c(ccc4c3OC[C@H]3CNCCN43)C2=O)C(=O)N1 ; C[C@@H]1Cc2c(ccc3[nH]ncc23)[C@H](c2ccc(N3CCC4(CC3)CC(CN3CCN5c6ccc7c(c6OC[C@H]5C3)CN([C@H]3CCC(=O)NC3=O)C7=O)C4)nc2)N1CC(F)(F)F]

To a mixture of 7-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonane-2-carbaldehyde (25 mg, 0.05 mmol, 1.0 eq) and(S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione (20 mg, 0.05 mmol, 1.0 eq) in EtOH (5 mL) was added TEA (0.1 mL) and HOAc (0.2 mL), the mixture was stirred at 50° C. for 1 hour under N₂. The mixture was added NaBH (OAc) 3 (43 mg, 0.20 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford(S)-3-((R)-7-((7-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonan-2-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (1.85 mg) as a white solid.

LCMS purity: 100% (UV at 254 nm), 838.2 [M+1]⁺.

¹H NMR (400 MHZ, MeOD) δ 8.15 (s, 1H), 8.06 (d, J=9.5 Hz, 1H), 7.43 (d, J=8.6 Hz, 2H), 7.38 (d, J=8.3 Hz, 1H), 7.29 (s, 1H), 7.16 (d, J=8.5 Hz, 1H), 7.07 (d, J=8.7 Hz, 1H), 5.36 (t, J=4.6 Hz, 2H), 5.11 (d, J=11.1 Hz, 2H), 4.50-4.43 (m, 1H), 4.38 (d, J=7.8 Hz, 1H), 4.33-4.26 (m, 1H), 4.17 (dd, J=11.1, 7.3 Hz, 1H), 3.74-3.70 (m, 2H), 3.62 (t, J=6.9 Hz, 3H), 3.08 (d, J=5.4 Hz, 1H), 3.02-2.93 (m, 2H), 2.91-2.88 (m, 1H), 2.81 (t, J=4.8 Hz, 1H), 2.28 (t, J=10.5 Hz, 3H), 2.24-2.19 (m, 3H), 2.05 (dd, J=12.5, 6.5 Hz, 4H), 1.92 (dd, J=7.1, 4.7 Hz, 2H), 1.82-1.76 (m, 3H), 1.65-1.61 (m, 2H), 1.21 (d, J=6.6 Hz, 3H), 0.93 (d, J=6.6 Hz, 3H).

### Compound A26. (S)-3-((R)-7-((7-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonan-2-yl)methyl)-1-oxo-1,3,5,5a,6,7,8,9-octahydro-2H-pyrazino[1′,2′: 4,5][1,4]oxazino[2,3-e]isoindol-2-yl)piperidine-2,6-dione

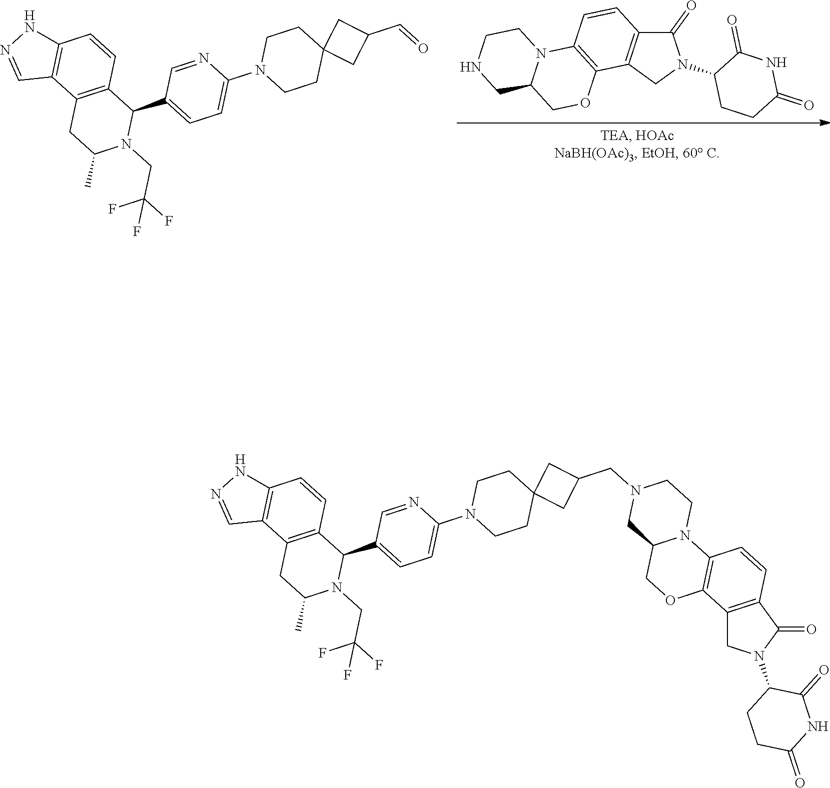 [SMILES: C[C@@H]1Cc2c(ccc3[nH]ncc23)[C@@H](c2ccc(N3CCC4(CC3)CC(C=O)C4)nc2)N1CC(F)(F)F ; O=C1CC[C@H](N2Cc3c(ccc4c3OC[C@H]3CNCCN43)C2=O)C(=O)N1 ; C[C@@H]1Cc2c(ccc3[nH]ncc23)[C@@H](c2ccc(N3CCC4(CC3)CC(CN3CCN5c6ccc7c(c6OC[C@H]5C3)CN([C@H]3CCC(=O)NC3=O)C7=O)C4)nc2)N1CC(F)(F)F]

To a mixture of 7-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonane-2-carbaldehyde (25 mg, 0.05 mmol, 1.0 eq) and(S)-3-((R)-1-oxo-1,3,5,5a,6,7,8,9-octahydro-2H-pyrazino[11,2′: 4,5][1,4]oxazino[2,3-e]isoindol-2-yl)piperidine-2,6-dione (20 mg, 0.05 mmol, 1.0 eq) in EtOH (5 mL) was added TEA (0.1 mL) and HOAc (0.2 mL), the mixture was stirred at 50° C. for 1 hour under N₂. The mixture was added NaBH (OAc) 3 (43 mg, 0.20 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford(S)-3-((R)-7-((7-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonan-2-yl)methyl)-1-oxo-1,3,5,5a,6,7,8,9-octahydro-2H-pyrazino[11,2′: 4,5][1,4]oxazino[2,3-e]isoindol-2-yl)piperidine-2,6-dione (4.03 mg) as a white solid.

LCMS purity: 100% (UV at 254 nm), 838.2 [M+1]⁺.

¹H NMR (400 MHZ, MeOD) δ 8.16 (s, 1H), 8.06 (dd, J=9.7, 1.9 Hz, 1H), 7.46-7.36 (m, 3H), 7.29 (s, 1H), 7.16 (d, J=8.3 Hz, 1H), 7.07 (d, J=8.6 Hz, 1H), 5.36 (t, J=4.8 Hz, 1H), 5.11 (d, J=11.6 Hz, 2H), 4.47 (dd, J=11.1, 2.4 Hz, 1H), 4.38 (d, J=7.9 Hz, 1H), 4.28 (dd, J=9.1, 3.7 Hz, 1H), 4.17 (dd, J=11.1, 7.0 Hz, 1H), 3.71 (d, J=4.4 Hz, 2H), 3.62 (s, 2H), 3.54 (dd, J=18.2, 11.7 Hz, 2H), 3.26-3.23 (m, 1H), 3.10-3.05 (m, 1H), 2.98 (d, J=8.8 Hz, 1H), 2.90 (d, J=13.4 Hz, 2H), 2.79 (d, J=15.6 Hz, 1H), 2.50 (dd, J=13.8, 5.5 Hz, 1H), 2.31-2.21 (m, 3H), 2.05 (d, J=5.8 Hz, 1H), 1.93 (s, 1H), 1.82-1.77 (m, 3H), 1.65-1.61 (m, 1H), 1.36-1.31 (m, 8H), 1.21 (d, J=6.7 Hz, 3H).

The rest of compounds were prepared in a manner analogous to Compound A17 by reductive amination.

TABLE 4

| Compound | | Calcd. | Found | LCMS |
|---|---|---|---|---|
| No | ¹HNMR | (M + H)⁺ | (M + H)⁺ | method |
| A1 | ¹HNMR (400 MHz, DMSO) δ 11.40- | 847.4 | 847.4 | C |
| | 11.39 (m, 1H), 10.88(s, 1H), 8.71(d, | | | |
| | J = 8.4 Hz, 1H), 8.13 (s, 1H), 7.73 (d, | | | |
| | J = 6.4 Hz, 1H), 7.44 (d, J = 10.0 Hz, 1H), | | | |
| | 7.33 (d, J = 8.0 Hz, 1H), 6.82 (d, J = 8.0 Hz, | | | |
| | 1H), 6.64 (d, J-9.2 Hz, 2H), 4.92-4.89 | | | |
| | (m, 2H), 4.78-4.75 (m, 1H), 4.74-4.65 (m, | | | |
| | 5H), 3.81-3.78 (m, 2H), 3.67-3.53 (m, | | | |
| | 3H), 3.44-3.32 (m, 8H), 3.05-3.04 (m, | | | |
| | 2H), 2.80-2.78 (m, 1H), 2.56-2.55 (m, | | | |
| | 1H), 2.10-1.99 (m, 2H), 1.56-1.46 (m, | | | |
| | 9H), 1.28-1.23 (m, 3H). | | | |
| A2 | ¹H NMR (400 MHz, DMSO) δ 12.98 (s, | 815.4 | 815.5 | F |
| | 1H), 10.93 (s, 1H), 8.06 (s, 1H), 7.30- | | | |
| | 7.10 (m, 2H), 7.02 (d, J = 8.2 Hz, 1H), | | | |
| | 6.73 (d, J = 8.6 Hz, 1H), 6.52 (d, J = 13.4 | | | |
| | Hz, 2H), 5.99-5.60 (m, 1H), 5.16 (s, | | | |
| | 1H), 5.07-4.99 (m, 1H), 4.40-4.21 (m, | | | |
| | 2H), 4.10 (d, J = 16.8 Hz, 1H), 4.03- | | | |
| | 3.93 (m, 1H), 3.88-3.69 (m, 3H), 3.50- | | | |
| | 3.45 (m, 1H), 3.22-3.15 (m, 2H), 3.07- | | | |
| | 2.84 (m, 5H), 2.80-2.63 (m, 4H), 2.62- | | | |
| | 2.53 (m, 1H), 2.45-2.31 (m, 1H), 2.26- | | | |
| | 2.07 (m, 3H), 2.00-1.89 (m, 1H), 1.84- | | | |
| | 1.68 (m, 4H), 1.21-0.99 (m, 5H). | | | |
| A3 | ¹H NMR (400 MHz, DMSO-d6) δ 12.98 | 857.4 | 857.3 | A |
| | (s, 1H), 11.14 (s, 1H), 8.06 (s, 1H), 7.99 | | | |
| | (s, 2H), 7.24-7.21 (m, 2H), 7.11 (s, 1H), | | | |
| | 6.98 (s, 1H), 6.71 (d, J = 8.4 Hz, 1H), | | | |
| | 6.55 (s, 1H), 6.52 (s, 1H), 5.95-5.68 (m, | | | |
| | 1H), 5.16 (t, J = 8.8 Hz, 2H), 3.22-3.18 | | | |
| | (m, 4H), 3.06-3.00 (m, 1H), 2.92-2.87 | | | |
| | (m, 2H), 2.67-2.59 (m, 3H).. 2.13-2.05 | | | |
| | (m, 2H), 1.57-1.46 (m, 10H), 1.25-1.15 | | | |
| | (m, 4H), 1.04 (d, J = 6.8 Hz, 3H). | | | |
| A4 | ¹H NMR (400 MHz, DMSO) δ 12.96 (s, | 828.4 | 828.3 | C |
| | 1H), 10.93 (s, 1H), 8.14 (s, 1H), 8.05 (s, | | | |
| | 1H), 7.23 (s, 1H), 7.18 (dd, J = 8.4, 4.1 | | | |
| | Hz, 2H), 7.06 (d, J = 8.9 Hz, 1H), 7.01 | | | |
| | (d, J = 7.9 Hz, 1H), 6.79 (d, J = 8.1 Hz, | | | |
| | 1H), 5.02 (d, J = 15.8 Hz, 1H), 4.86 (s, | | | |
| | 1H), 4.34 (d, J = 4.5 Hz, 1H), 4.26 (d, J = | | | |
| | 16.9 Hz, 1H), 4.09 (d, J = 17.2 Hz, 1H), | | | |
| | 3.95 (t, J = 7.8 Hz, 1H), 3.80 (d, J = 6.6 | | | |
| | Hz, 1H), 3.67 (d, J = 11.9 Hz, 2H), 3.16- | | | |
| | 3.11 (m, 4H), 3.03 (d, J = 8.7 Hz, 3H), | | | |
| | 2.89 (dd, J = 10.7, 10.2 Hz, 4H), 2.73- | | | |
| | 2.64 (m, 4H), 2.33 (s, 1H), 2.11 (dd, J = | | | |
| | 18.3, 7.6 Hz, 1H), 1.98-1.93 (m, 2H), | | | |
| | 1.67 (d, J = 6.8 Hz, 2H), 1.61-1.40 (m, | | | |
| | 4H), 1.01 (d, J = 6.4 Hz, 3H), 0.68 (d, J = | | | |
| | 19.6 Hz, 1H), 0.52-0.42 (m, 1H). | | | |
| A5 | ¹H NMR (400 MHz, DMSO) δ 12.94 (s, | 827.4 | 827.4 | D |
| | 1H), 10.96 (s, 1H), 8.38 (s, 1H), 8.13 (s, | | | |
| | 1H), 8.05 (s, 1H), 7.45 (s, 1H), 7.21 (dd, | | | |
| | J = 24.8, 8.6 Hz, 2H), 7.06 (d, J = 8.8 Hz, | | | |
| | 1H), 7.00 (s, 1H), 6.79 (d, J = 8.5 Hz, | | | |
| | 1H), 5.07 (d, J = 7.9 Hz, 1H), 4.87 (s, | | | |
| | 1H), 4.45 (s, 2H), 4.34 (d, J = 16.8 Hz, | | | |
| | 1H), 4.20 (d, J = 17.3 Hz, 1H), 3.70- | | | |
| | 3.64 (m, 1H), 3.12 (d, J = 5.8 Hz, 2H), | | | |
| | 3.02 (d, J = 14.5 Hz, 3H), 2.96-2.85 (m, | | | |
| | 3H), 2.80 (d, J = 11.1 Hz, 2H), 2.69- | | | |
| | 2.60 (m, 3H), 2.33 (s, 1H), 1.94 (dd, J = | | | |
| | 19.3, 11.1 Hz, 5H), 1.67 (s, 3H), 1.56 (s, | | | |
| | 2H), 1.48-1.39 (m, 2H), 1.24 (s, 3H), | | | |
| | 1.01 (d, J = 6.5 Hz, 3H), 0.89 (dd, J = | | | |
| | 25.5, 3.0 Hz, 2H), 0.67 (t, J = 10.0 Hz, | | | |
| | 1H), 0.46 (dd, J = 16.6, 6.4 Hz, 1H). | | | |
| A6 | ¹H NMR (400 MHz, DMSO) δ 12.96 (s, | 809.4 | 809.0 | E |
| | 1H), 10.93 (s, 1H), 8.16 (s, 2H), 8.05 (s, | | | |
| | 1H), 7.25-7.16 (m, 2H), 7.01 (d, J = 8.6 | | | |
| | Hz, 1H), 6.68 (d, J = 8.6 Hz, 1H), 6.56 (s, | | | |
| | 1H), 6.45 (t, J = 9.4 Hz, 1H), 6.31 (d, J = | | | |
| | 6.6 Hz, 1H), 5.92 (t, J = 15.8 Hz, 2H), | | | |
| | 5.26 (s, 1H), 5.08-4.94 (m, 1H), 4.35 (s, | | | |
| | 1H), 4.26 (d, J = 16.8 Hz, 1H), 4.11 (d, J = | | | |
| | 10.2 Hz, 1H), 3.98 (d, J = 10.4 Hz, | | | |
| | 1H), 3.87 (s, 3H), 3.67 (d, J = 11.6 Hz, | | | |
| | 2H), 3.04-2.88 (m, 6H), 2.65-2.57 (m, | | | |
| | 4H), 2.44-2.35 (m, 1H), 2.14-1.92 (m, | | | |
| | 3H), 1.83-1.64 (m, 4H), 1.21 (d, J = 9.8 | | | |
| | Hz, 3H), 1.04 (d, J = 6.4 Hz, 4H). | | | |
| A7 | ¹H NMR (400 MHz, DMSO) δ 12.95 (s, | 847.4 | 847.3 | B |
| | 1H), 10.99 (s, 1H), 8.41 (s, 2H), 8.14 (s, | | | |
| | 1H), 8.05 (s, 1H), 7.72 (d, J = 7.8 Hz, | | | |
| | 1H), 7.58 (s, 1H), 7.47 (d, J = 7.8 Hz, | | | |
| | 1H), 7.24 (d, J = 6.1 Hz, 1H), 7.18 (d, J = | | | |
| | 8.7 Hz, 1H), 7.06 (d, J = 8.8 Hz, 1H), | | | |
| | 6.79 (d, J = 8.2 Hz, 1H), 5.11 (d, J = 8.9 | | | |
| | Hz, 1H), 4.86 (s, 1H), 4.51-4.25 (m, | | | |
| | 3H), 3.12 (s, 3H), 3.06-2.99 (m, 4H), | | | |
| | 2.90 (dd, J = 23.5, 11.5 Hz, 3H), 2.66 (t, | | | |
| | J = 15.4 Hz, 4H), 2.40 (d, J = 3.8 Hz, | | | |
| | 3H), 2.33 (s, 1H), 2.25 (t, J = 9.3 Hz, | | | |
| | 1H), 2.14 (t, J = 14.2 Hz, 1H), 1.98 (dd, | | | |
| | J = 29.8, 12.4 Hz, 3H), 1.67 (d, J = 6.7 Hz, | | | |
| | 2H), 1.55 (s, 2H), 1.47-1.40 (m, 2H), | | | |
| | 1.01 (d, J = 6.4 Hz, 3H), 0.86 (dd, J = | | | |
| | 14.1, 6.5 Hz, 1H), 0.65 (d, J = 8.0 Hz, | | | |
| | 1H), 0.52-0.43 (m, 1H). | | | |
| A8 | ¹H NMR (400 MHz, DMSO) δ 12.97 (s, | 769.4 | 769.3 | F |
| | 1H), 10.94 (s, 1H), 8.05 (s, 1H), 7.28- | | | |
| | 7.09 (m, 2H), 7.02 (d, J = 7.8 Hz, 1H), | | | |
| | 6.75-6.40 (m, 4H), 6.33 (s, 1H), 5.93 (s, | | | |
| | 1H), 5.33 (s, 1H), 5.03 (d, J = 14.4 Hz, | | | |
| | 1H), 4.42-3.90 (m, 5H), 3.88 (s, 2H), | | | |
| | 3.70 (s, 2H), 2.95 (s, 4H), 2.32-1.91 (m, | | | |
| | 8H), 1.77 (d, J = 11.6 Hz, 5H), 1.24 (s, | | | |
| | 7H), 1.03 (s, 2H). | | | |
| A9 | ¹H NMR (400 MHz, DMSO) δ 12.99 (s, | 799.4 | 799.2 | A |
| | 1H), 10.84 (s, 1H), 8.72 (d, J = 8.4 Hz, | | | |
| | 1H), 8.41-8.25 (m, 3H), 8.15-7.99 (m, | | | |
| | 2H), 7.85 (d, J = 8.8 Hz, 1H), 7.46-7.36 | | | |
| | (m, 1H), 7.30-7.18 (m, 2H), 7.07 (d, J = | | | |
| | 8.6 Hz, 1H), 6.82 (d, J = 8.8 Hz, 1H), | | | |
| | 4.99 (s, 1H), 4.81-4.68 (m, 1H), 3.32 (s, | | | |
| | 7H), 3.11 (d, J = 18.2 Hz, 3H), 2.88- | | | |
| | 2.70 (m, 3H), 2.18 (d, J = 12.8 Hz, 1H), | | | |
| | 2.02-1.93 (m, 4H), 1.61 (d, J = 19.0 Hz, | | | |
| | 4H), 1.46 (d, J = 10.2 Hz, 2H), 1.23 (s, | | | |
| | 6H), 1.09 (d, J = 6.6 Hz, 3H). | | | |
| A10 | ¹H NMR (400 MHz, DMSO) δ 12.98 (s, | 799.4 | 799.2 | A |
| | 1H), 10.76 (s, 1H), 8.29-7.98 (m, 3H), | | | |
| | 7.33-7.19 (m, 2H), 7.07 (d, J = 8.6 Hz, | | | |
| | 1H), 6.94 (d, J = 8.4 Hz, 2H), 6.82 (d, J = | | | |
| | 8.6 Hz, 1H), 6.60 (d, J = 8.4 Hz, 2H), | | | |
| | 5.63 (d, J = 7.2 Hz, 1H), 4.99 (s, 1H), | | | |
| | 4.25 (s, 1H), 3.67-3.47 (m, 9H), 3.18- | | | |
| | 3.04 (m, 5H), 2.96-2.87 (m, 3H), 2.83- | | | |
| | 2.67 (m, 2H), 2.29 (d, J = 12.0 Hz, 1H), | | | |
| | 2.08-1.89 (m, 5H), 1.82 (d, J = 12.4 Hz, | | | |
| | 1H), 1.70-1.58 (m, 5H), 1.47-1.40 (m, | | | |
| | 2H), 1.24 (s, 1H), 1.10 (s, 1H). | | | |
| A11 | ¹H NMR (400 MHz, DMSO) δ 12.94 (s, | 828.4 | 828.3 | C |
| | 1H), 10.93 (s, 1H), 8.21 (s, 1H), 8.04 (s, | | | |
| | 1H), 7.66 (d, J = 2.6 Hz, 1H), 7.17 (d, J = | | | |
| | 8.4 Hz, 2H), 7.02 (dd, J = 8.3, 6.1 Hz, | | | |
| | 2H), 6.77 (d, J = 8.7 Hz, 1H), 6.72 (dd, | | | |
| | J = 8.5, 2.6 Hz, 1H), 5.03 (dd, J = 13.2, 5.0 | | | |
| | Hz, 1H), 4.84 (s, 1H), 4.36 (d, J = 8.3 Hz, | | | |
| | 1H), 4.26 (d, J = 16.8 Hz, 1H), 4.10 (d, | | | |
| | J = 16.8 Hz, 1H), 4.01-3.91 (m, 1H), 3.82 | | | |
| | (d, J = 11.6 Hz, 1H), 3.69 (dd, J = 11.6, | | | |
| | 5.4 Hz, 1H), 3.53 (d, J = 19.8 Hz, 4H), | | | |
| | 3.21 (dd, J = 16.4, 4.6 Hz, 2H), 2.94 | | | |
| | (ddd, J = 31.0, 19.3, 11.9 Hz, 5H), 2.79- | | | |
| | 2.69 (m, 1H), 2.68-2.53 (m, 2H), 2.39 | | | |
| | (ddd, J = 26.5, 13.4, 4.6 Hz, 1H), 2.20- | | | |
| | 2.03 (m, 3H), 1.93 (dd, J = 23.8, 8.7 Hz, | | | |
| | 3H), 1.71 (d, J = 10.5 Hz, 3H), 1.47 (t, | | | |
| | J = 11.6 Hz, 2H), 1.01 (d, J = 6.5 Hz, 3H), | | | |
| | 0.97-0.81 (m, 3H), 0.73-0.63 (m, 1H), | | | |
| | 0.52-0.42 (m, 1H). | | | |
| A12 | ¹H NMR (400 MHz, DMSO) δ 13.17 (s, | 828.4 | 828.3 | C |
| | 1H), 10.95 (s, 1H), 10.12 (s, 1H), 8.27 (s, | | | |
| | 1H), 8.16 (s, 1H), 7.38 (s, 2H), 7.19 (s, | | | |
| | 2H), 7.04 (s, 1H), 6.91 (d, J = 7.7 Hz, | | | |
| | 1H), 5.04 (dd, J = 13.3, 4.9 Hz, 1H), 4.41- | | | |
| | 4.15 (m, 5H), 4.10-3.93 (m, 2H), 3.57 | | | |
| | (s, 4H), 3.14 (s, 7H), 2.99-2.79 (m, | | | |
| | 3H), 2.71 (dd, J = 18.1, 6.2 Hz, 1H), 2.59 | | | |
| | (d, J = 18.0 Hz, 1H), 2.38 (dd, J = 17.9, | | | |
| | 9.3 Hz, 1H), 2.10-1.95 (m, 3H), 1.75- | | | |
| | 1.50 (m, 7H), 1.27 (d, J = 19.3 Hz, 3H), | | | |
| | 1.14 (dd, J = 21.0, 8.6 Hz, 2H), 0.83 (dd, | | | |
| | J = 32.7, 25.5 Hz, 2H). | | | |
| A13 | ¹H NMR (400 MHz, DMSO) δ 13.24 (s, | 828.4 | 828.3 | D |
| | 1H), 10.94 (s, 1H), 10.15 (s, 1H), 8.30 (s, | | | |
| | 1H), 8.19 (s, 1H), 7.46-7.36 (m, 2H), | | | |
| | 7.25-7.17 (m, 2H), 7.03 (s, 1H), 6.95 (t, | | | |
| | J = 11.8 Hz, 1H), 5.85 (s, 1H), 5.04 (dd, | | | |
| | J = 13.1, 5.0 Hz, 1H), 4.38-4.16 (m, 6H), | | | |
| | 4.03 (d, J = 7.1 Hz, 1H), 3.91-3.71 (m, | | | |
| | 4H), 3.55 (s, 5H), 3.21-3.09 (m, 5H), | | | |
| | 2.97-2.82 (m, 2H), 2.77-2.66 (m, 1H), | | | |
| | 2.58 (d, J = 17.4 Hz, 1H), 2.40-2.32 (m, | | | |
| | 1H), 2.06 (d, J = 8.9 Hz, 2H), 1.96 (d, J = | | | |
| | 5.9 Hz, 1H), 1.69 (s, 2H), 1.64-1.53 (m, | | | |
| | 4H), 1.37 (s, 3H), 1.24-1.14 (m, 2H), | | | |
| | 0.94-0.75 (m, 2H). | | | |
| A14 | ¹H NMR (400 MHz, DMSO) δ 13.23 (s, | 828.4 | 828.3 | D |
| | 1H), 10.94 (s, 1H), 10.11 (s, 1H), 8.30 (s, | | | |
| | 1H), 8.19 (s, 1H), 7.46-7.36 (m, 2H), | | | |
| | 7.21 (d, J = 13.3 Hz, 2H), 7.03 (s, 1H), | | | |
| | 6.95 (t, J = 9.4 Hz, 1H), 5.87 (s, 1H), | | | |
| | 5.05 (dd, J = 13.2, 5.1 Hz, 1H), 4.37- | | | |
| | 4.27 (m, 3H), 4.18 (dd, J = 25.9, 14.5 Hz, | | | |
| | 3H), 4.05 (dd, J = 11.2, 6.6 Hz, 2H), 3.94- | | | |
| | 3.70 (m, 4H), 3.55 (d, J = 19.6 Hz, 5H), | | | |
| | 3.15 (s, 4H), 2.91 (t, J = 15.2 Hz, 2H), | | | |
| | 2.76-2.66 (m, 1H), 2.58 (d, J = 18.1 Hz, | | | |
| | 1H), 2.40-2.31 (m, 1H), 2.06 (dd, J = | | | |
| | 16.6, 7.7 Hz, 2H), 2.00-1.92 (m, 1H), | | | |
| | 1.69 (s, 2H), 1.64-1.54 (m, 4H), 1.37 | | | |
| | (d, J = 5.7 Hz, 3H), 1.19 (d, J = 18.6 Hz, | | | |
| | 2H), 0.85 (dt, J = 23.1, 10.4 Hz, 2H). | | | |
| A15 | ¹H NMR (400 MHz, MeOD) δ 8.03 (d, | 759.4 | 759.3 | D |
| | J = 2.8 Hz, 1H), 8.00 (s, 1H), 7.25 (d, J = | | | |
| | 6.2 Hz, 1H), 7.14 (t, J = 8.4 Hz, 2H), | | | |
| | 6.95 (d, J = 8.0 Hz, 2H), 6.73 (d, J = 9.0 | | | |
| | Hz, 1H), 6.62 (d, J = 8.5 Hz, 2H), 5.04 (s, | | | |
| | 1H), 4.17 (dd, J = 11.8, 4.7 Hz, 1H), 3.87 | | | |
| | (t, J = 10.2 Hz, 1H), 3.43 (dd, J = 29.3, | | | |
| | 15.0 Hz, 3H), 3.14 (s, 4H), 3.05 (s, 2H), | | | |
| | 2.96 (d, J = 16.3 Hz, 2H), 2.73-2.60 (m, | | | |
| | 5H), 2.22 (d, J = 9.7 Hz, 1H), 2.09 (t, J = | | | |
| | 7.6 Hz, 2H), 1.96 (d, J = 14.8 Hz, 2H), | | | |
| | 1.83 (d, J = 13.0 Hz, 2H), 1.74 (d, J = 5.2 | | | |
| | Hz, 2H), 1.61 (d, J = 8.9 Hz, 3H), 1.19 (s, | | | |
| | 2H), 1.07 (d, J = 6.5 Hz, 3H), 0.92-0.81 | | | |
| | (m, 2H), 0.43 (dt, J = 17.1, 10.8 Hz, 2H). | | | |
| A16 | ¹H NMR (400 MHz, DMSO) δ 12.94 (s, | 828.4 | 828.3 | C |
| | 1H), 10.93 (s, 1H), 8.30 (s, 1H), 8.04 (s, | | | |
| | 1H), 7.66 (d, J = 2.7 Hz, 1H), 7.17 (d, J = | | | |
| | 8.4 Hz, 2H), 7.02 (dd, J = 8.4, 4.9 Hz, | | | |
| | 2H), 6.77 (d, J = 8.7 Hz, 1H), 6.72 (dd, | | | |
| | J = 8.6, 2.7 Hz, 1H), 5.03 (dd, J = 13.2, 5.0 | | | |
| | Hz, 1H), 4.84 (s, 1H), 4.36 (d, J = 8.4 Hz, | | | |
| | 1H), 4.26 (d, J = 16.8 Hz, 1H), 4.10 (d, | | | |
| | J = 16.9 Hz, 1H), 4.03-3.94 (m, 1H), 3.83 | | | |
| | (d, J = 11.7 Hz, 1H), 3.70 (d, J = 5.3 Hz, | | | |
| | 1H), 3.55 (s, 2H), 3.50 (s, 2H), 3.25- | | | |
| | 3.14 (m, 3H), 3.05-2.96 (m, 1H), 2.92 | | | |
| | (d, J = 13.6 Hz, 3H), 2.78-2.58 (m, 3H), | | | |
| | 2.38 (dd, J = 20.6, 11.9 Hz, 1H), 2.11 | | | |
| | (dd, J = 26.9, 8.3 Hz, 3H), 1.92 (dd, J = | | | |
| | 18.9, 9.1 Hz, 3H), 1.71 (t, J = 10.4 Hz, | | | |
| | 3H), 1.47 (t, J = 11.6 Hz, 3H), 1.01 (d, | | | |
| | J = 6.5 Hz, 3H), 0.97-0.80 (m, 4H), 0.72- | | | |
| | 0.63 (m, 1H), 0.46 (dt, J = 11.3, 8.0 Hz, 1H). | | | |
| A17 | ¹H NMR (400 MHz, DMSO) δ 13.04 (s, | 838.4 | 838.2 | D |
| | 1H), 10.93 (s, 1H), 8.22 (dt, J = 12.1, | | | |
| | 10.0 Hz, 1H), 8.08 (s, 1H), 7.73 (d, J = | | | |
| | 2.0 Hz, 1H), 7.29 (d, J = 8.5 Hz, 1H), | | | |
| | 7.17 (d, J = 8.4 Hz, 1H), 7.01 (d, J = 8.5 | | | |
| | Hz, 1H), 6.86 (d, J = 8.5 Hz, 1H), 6.27 | | | |
| | (d, J = 8.7 Hz, 1H), 5.32 (t, J = 4.9 Hz, | | | |
| | 1H), 5.06-4.93 (m, 2H), 4.38-4.33 (m, | | | |
| | 1H), 4.28-4.23 (m, 1H), 4.10 (d, J = | | | |
| | 17.1 Hz, 1H), 3.99-3.93 (m, 1H), 3.82 | | | |
| | (dd, J = 11.0, 2.3 Hz, 1H), 3.54 (s, 3H), | | | |
| | 3.01-2.84 (m, 7H), 2.67 (s, 1H), 2.33 (s, | | | |
| | 1H), 2.13 (dd, J = 9.7, 6.4 Hz, 2H), 1.99- | | | |
| | 1.95 (m, 2H), 1.86 (dd, J = 10.6, 3.5 | | | |
| | Hz, 2H), 1.70 (t, J = 8.2 Hz, 2H), 1.45 | | | |
| | (dd, J = 18.7, 7.7 Hz, 3H), 1.24 (s, 4H), | | | |
| | 1.07 (d, J = 6.6 Hz, 3H), 0.96-0.92 (m, | | | |
| | 1H), 0.85 (t, J = 5.1 Hz, 1H). | | | |
| A18 | ¹H NMR (400 MHz, MeOD) δ 8.14 (s, | 838.4 | 838.3 | D |
| | 1H), 7.87 (s, 1H), 7.79 (d, J = 9.2 Hz, | | | |
| | 1H), 7.36 (dd, J = 12.9, 8.5 Hz, 2H), 7.15 | | | |
| | (d, J = 8.4 Hz, 1H), 6.99 (d, J = 8.7 Hz, | | | |
| | 1H), 6.78 (d, J = 9.4 Hz, 1H), 5.17 (s, | | | |
| | 1H), 5.11 (dd, J = 13.2, 5.2 Hz, 2H), 4.51- | | | |
| | 4.42 (m, 1H), 4.38 (d, J = 8.0 Hz, 2H), | | | |
| | 4.29-4.12 (m, 2H), 4.00 (d, J = 24.5 Hz, | | | |
| | 4H), 3.72 (t, J = 9.2 Hz, 3H), 3.62-3.43 | | | |
| | (m, 2H), 3.27 (d, J = 13.4 Hz, 3H), 3.11 | | | |
| | (s, 2H), 2.89 (dd, J = 13.2, 3.5 Hz, 2H), | | | |
| | 2.58-2.43 (m, 1H), 2.13 (d, J = 15.3 Hz, | | | |
| | 2H), 1.90 (t, J = 10.7 Hz, 2H), 1.71 (t, J = | | | |
| | 11.7 Hz, 2H), 1.39 (d, J = 5.9 Hz, 3H), | | | |
| | 1.33 (d, J = 14.6 Hz, 3H), 1.19 (dd, J = | | | |
| | 22.5, 13.9 Hz, 3H). | | | |
| A19 | ¹H NMR (400 MHz, MeOD) δ 8.39 (s, | 798.4 | 798.1 | C |
| | 1H), 7.99 (s, 1H), 7.68 (s, 1H), 7.31 (d, | | | |
| | J = 7.3 Hz, 1H), 7.24 (d, J = 8.3 Hz, 1H), | | | |
| | 7.19 (d, J = 8.4 Hz, 1H), 6.89 (dd, J = | | | |
| | 17.7, 8.5 Hz, 2H), 6.68 (d, J = 8.8 Hz, | | | |
| | 1H), 4.98 (dd, J = 13.5, 5.3 Hz, 1H), 4.91 | | | |
| | (s, 1H), 4.24 (dd, J = 17.3, 9.3 Hz, 3H), | | | |
| | 4.12 (d, J = 12.3 Hz, 2H), 3.93 (t, J = 9.4 | | | |
| | Hz, 1H), 3.73 (d, J = 11.3 Hz, 1H), 3.33 | | | |
| | (dd, J = 19.5, 12.4 Hz, 2H), 2.98 (d, J = | | | |
| | 17.5 Hz, 2H), 2.87 (d, J = 9.1 Hz, 2H), | | | |
| | 2.83-2.73 (m, 5H), 2.67 (d, J = 15.2 Hz, | | | |
| | 1H), 2.37 (dd, J = 23.7, 15.1 Hz, 1H), | | | |
| | 2.19 (d, J = 6.0 Hz, 2H), 2.15-2.02 (m, | | | |
| | 2H), 1.83-1.73 (m, 4H), 1.17 (d, J = | | | |
| | 19.3 Hz, 3H), 1.06 (d, J = 6.7 Hz, 3H). | | | |
| A20 | ¹H NMR (400 MHz, MeOD) δ 8.00 (s, | 798.4 | 798.2 | C |
| | 1H), 7.54 (s, 1H), 7.50 (d, J = 9.2 Hz, | | | |
| | 1H), 7.26 (d, J = 8.6 Hz, 1H), 7.02 (s, | | | |
| | 1H), 6.96 (s, 1H), 6.88 (t, J = 8.8 Hz, | | | |
| | 2H), 4.99 (d, J = 5.1 Hz, 1H), 4.94 (s, | | | |
| | 1H), 4.23 (d, J = 9.9 Hz, 3H), 4.13 (d, J = | | | |
| | 12.5 Hz, 2H), 3.89 (dd, J = 18.1, 10.3 | | | |
| | Hz, 2H), 3.35 (dd, J = 15.3, 9.8 Hz, 2H), | | | |
| | 3.10-2.95 (m, 2H), 2.92 (d, J = 13.6 Hz, | | | |
| | 3H), 2.80 (dd, J = 18.7, 11.3 Hz, 2H), | | | |
| | 2.67 (d, J = 15.6 Hz, 1H), 2.51 (d, J = 6.5 | | | |
| | Hz, 3H), 2.41-2.15 (m, 2H), 2.14-1.89 | | | |
| | (m, 3H), 1.82 (s, 2H), 1.19 (s, 3H), 1.07 | | | |
| | (d, J = 6.6 Hz, 3H). | | | |
| A21 | ¹H NMR (400 MHz, DMSO) δ 13.05 (s, | 854.4 | 854.2 | C |
| | 1H), 10.93 (s, 1H), 8.32 (s, 1H), 8.08 (s, | | | |
| | 1H), 7.77 (s, 1H), 7.30 (d, J = 8.7 Hz, | | | |
| | 1H), 7.22-7.17 (m, 1H), 7.01 (d, J = 8.5 | | | |
| | Hz, 1H), 6.88 (d, J = 8.7 Hz, 1H), 6.76 | | | |
| | (d, J = 8.9 Hz, 1H), 5.05-5.00 (m, 1H), | | | |
| | 4.95 (s, 1H), 4.36 (d, J = 9.9 Hz, 1H), | | | |
| | 4.26 (d, J = 17.0 Hz, 1H), 4.10 (d, J = | | | |
| | 16.7 Hz, 1H), 3.98-3.90 (m, 2H), 3.83 | | | |
| | (d, J = 10.7 Hz, 1H), 3.61-3.57 (m, 2H), | | | |
| | 3.46 (s, 2H), 3.18-3.10 (m, 2H), 3.00 | | | |
| | (d, J = 12.0 Hz, 2H), 2.91 (d, J = 12.2 Hz, | | | |
| | 2H), 2.84 (d, J = 17.7 Hz, 2H), 2.74 (s, | | | |
| | 1H), 2.67 (s, 1H), 2.35 (d, J = 13.8 Hz, | | | |
| | 3H), 2.15-2.06 (m, 1H), 1.97 (dd, J = | | | |
| | 14.7, 7.0 Hz, 2H), 1.77-1.70 (m, 1H), | | | |
| | 1.62-1.53 (m, 4H), 1.33 (dd, J = 26.5, | | | |
| | 13.1 Hz, 2H), 1.08 (d, J = 6.6 Hz, 3H). | | | |
| A22 | ¹H NMR (400 MHz, MeOD) δ 7.98 (s, | 854.4 | 854.3 | C |
| | 1H), 7.67 (s, 1H), 7.32 (dd, J = 9.0, 2.0 | | | |
| | Hz, 1H), 7.21 (dd, J = 19.4, 8.4 Hz, 2H), | | | |
| | 6.88 (dd, J = 17.2, 8.6 Hz, 2H), 6.69 (d, | | | |
| | J = 9.0 Hz, 1H), 4.97 (dd, J = 13.4, 5.0 Hz, | | | |
| | 1H), 4.91 (s, 1H), 4.24 (dd, J = 17.9, 7.7 | | | |
| | Hz, 3H), 3.92 (t, J = 9.6 Hz, 2H), 3.74 (d, | | | |
| | J = 10.8 Hz, 1H), 3.53 (dd, J = 16.2, 8.1 | | | |
| | Hz, 3H), 3.46-3.33 (m, 3H), 3.05-2.86 | | | |
| | (m, 4H), 2.85-2.71 (m, 4H), 2.69-2.56 | | | |
| | (m, 2H), 2.36 (d, J = 7.1 Hz, 3H), 2.17 | | | |
| | (dd, J = 14.8, 11.8 Hz, 1H), 2.05-1.90 | | | |
| | (m, 2H), 1.77 (dt, J = 18.3, 10.7 Hz, 1H), | | | |
| | 1.62 (s, 3H), 1.38 (dd, J = 12.6, 8.3 Hz, | | | |
| | 1H), 1.19 (s, 2H), 1.05 (d, J = 6.6 Hz, 3H). | | | |
| A23 | ¹H NMR (400 MHz, MeOD) δ 8.47 (s, | 798.4 | 798.2 | C |
| | 1H), 8.08 (s, 1H), 7.77 (s, 1H), 7.41 (d, | | | |
| | J = 7.1 Hz, 1H), 7.31 (dd, J = 19.2, 8.4 Hz, | | | |
| | 2H), 6.98 (dd, J = 17.9, 8.5 Hz, 2H), 6.77 | | | |
| | (d, J = 8.9 Hz, 1H), 5.07 (dd, J = 12.6, | | | |
| | 4.1 Hz, 1H), 5.01 (s, 1H), 4.34 (dd, J = | | | |
| | 16.4, 9.3 Hz, 3H), 4.21 (d, J = 11.7 Hz, | | | |
| | 2H), 4.10-3.97 (m, 1H), 3.83 (d, J = | | | |
| | 12.2 Hz, 1H), 3.41 (dd, J = 16.1, 10.4 | | | |
| | Hz, 2H), 3.08 (dt, J = 12.6, 8.6 Hz, 2H), | | | |
| | 3.02-2.93 (m, 2H), 2.92-2.84 (m, 4H), | | | |
| | 2.83-2.69 (m, 2H), 2.47 (dt, J = 13.8, | | | |
| | 9.0 Hz, 1H), 2.29 (d, J = 6.3 Hz, 2H), | | | |
| | 2.24-2.06 (m, 2H), 1.85 (t, J = 11.3 Hz, | | | |
| | 4H), 1.25 (dd, J = 22.4, 9.4 Hz, 3H), 1.15 | | | |
| | (d, J = 6.6 Hz, 3H). | | | |
| A24 | ¹H NMR (400 MHz, DMSO) δ 13.04 (s, | 798.4 | 798.2 | C |
| | 1H), 10.93 (s, 1H), 8.18 (s, 1H), 8.08 (s, | | | |
| | 1H), 7.77 (s, 1H), 7.30 (d, J = 8.6 Hz, | | | |
| | 1H), 7.21 (d, J = 6.8 Hz, 1H), 7.04 (s, | | | |
| | 1H), 6.93 (s, 1H), 6.88 (d, J = 8.6 Hz, | | | |
| | 1H), 6.74 (d, J = 8.9 Hz, 1H), 5.02 (dd, | | | |
| | J= 13.2, 5.0 Hz, 1H), 4.95 (s, 1H), 4.26 | | | |
| | (dd, J = 19.1, 11.3 Hz, 4H), 4.15 (d, J = | | | |
| | 16.7 Hz, 1H), 3.96-3.87 (m, 1H), 3.81 | | | |
| | (d, J = 10.7 Hz, 1H), 3.53 (dd, J = 16.1, | | | |
| | 9.7 Hz, 2H), 3.06-2.96 (m, 3H), 2.93 | | | |
| | (d, J = 6.4 Hz, 3H), 2.80-2.67 (m, 4H), | | | |
| | 2.35 (d, J = 12.9 Hz, 1H), 2.23-2.15 (m, | | | |
| | 2H), 2.12-2.06 (m, 1H), 2.03-1.91 (m, | | | |
| | 2H), 1.81-1.70 (m, 4H), 1.24 (s, 2H), | | | |
| | 1.08 (d, J = 6.6 Hz, 3H). | | | |
| A25 | ¹H NMR (400 MHz, MeOD) δ 8.15 (s, | 838.4 | 838.2 | C |
| | 1H), 8.06 (d, J = 9.5 Hz, 1H), 7.43 (d, J = | | | |
| | 8.6 Hz, 2H), 7.38 (d, J = 8.3 Hz, 1H), | | | |
| | 7.29 (s, 1H), 7.16 (d, J = 8.5 Hz, 1H), | | | |
| | 7.07 (d, J = 8.7 Hz, 1H), 5.36 (1, J = 4.6 | | | |
| | Hz, 2H), 5.11 (d, J = 11.1 Hz, 2H), 4.50- | | | |
| | 4.43 (m, 1H), 4.38 (d, J = 7.8 Hz, 1H), | | | |
| | 4.33-4.26 (m, 1H), 4.17 (dd, J = 11.1, | | | |
| | 7.3 Hz, 1H), 3.74-3.70 (m, 2H), 3.62 (t, | | | |
| | J = 6.9 Hz, 3H), 3.08 (d, J = 5.4 Hz, 1H), | | | |
| | 3.02-2.93 (m, 2H), 2.91-2.88 (m, 1H), | | | |
| | 2.81 (t, J = 4.8 Hz, 1H), 2.28 (t, J = 10.5 | | | |
| | Hz, 3H), 2.24-2.19 (m, 3H), 2.05 (dd, | | | |
| | J = 12.5, 6.5 Hz, 4H), 1.92 (dd, J = 7.1, 4.7 | | | |
| | Hz, 2H), 1.82-1.76 (m, 3H), 1.65-1.61 | | | |
| | (m, 2H), 1.21 (d, J = 6.6 Hz, 3H), 0.93 | | | |
| | (d, J = 6.6 Hz, 3H). | | | |
| A26 | ¹H NMR (400 MHz, MeOD) δ 8.16 (s, | 838.4 | 838.2 | C |
| | 1H), 8.06 (dd, J = 9.7, 1.9 Hz, 1H), 7.46- | | | |
| | 7.36 (m, 3H), 7.29 (s, 1H), 7.16 (d, J = | | | |
| | 8.3 Hz, 1H), 7.07 (d, J = 8.6 Hz, 1H), | | | |
| | 5.36 (t, J = 4.8 Hz, 1H), 5.11 (d, J = 11.6 | | | |
| | Hz, 2H), 4.47 (dd, J = 11.1, 2.4 Hz, 1H), | | | |
| | 4.38 (d, J = 7.9 Hz, 1H), 4.28 (dd, J = | | | |
| | 9.1, 3.7 Hz, 1H), 4.17 (dd, J = 11.1, 7.0 | | | |
| | Hz, 1H), 3.71 (d, J = 4.4 Hz, 2H), 3.62 (s, | | | |
| | 2H), 3.54 (dd, J = 18.2, 11.7 Hz, 2H), | | | |
| | 3.26-3.23 (m, 1H), 3.10-3.05 (m, 1H), | | | |
| | 2.98 (d, J = 8.8 Hz, 1H), 2.90 (d, J = 13.4 | | | |
| | Hz, 2H), 2.79 (d, J = 15.6 Hz, 1H), 2.50 | | | |
| | (dd, J = 13.8, 5.5 Hz, 1H), 2.31-2.21 | | | |
| | (m, 3H), 2.05 (d, J = 5.8 Hz, 1H), 1.93 (s, | | | |
| | 1H), 1.82-1.77 (m, 3H), 1.65-1.61 (m, | | | |
| | 1H), 1.36-1.31 (m, 8H), 1.21 (d, J = 6.7 | | | |
| | Hz, 3H). | | | |

### For Compound B1-B126

Some additional compounds were prepared in a manner analogous to Compound A17 by reductive amination.

TABLE 5
Characterization Data for Compounds
Prepared According to Compound A17

| Compound No | LC-MS (M + H)⁺ |
|---|---|
| B16 | 815.23 |
| B18 | 765.87 |
| B29 | 828.39 |
| B30 | 828.42 |
| B46 | 870.46 |
| B47 | 828.30 |
| B52 | 839.33 |
| B53 | 850.45 |
| B54 | 849.43 |
| B55 | 849.50 |
| B56 | 848.40 |
| B57 | 837.34 |
| B58 | 851.41 |
| B59 | 851.40 |
| B60 | 837.31 |
| B61 | 851.41 |
| B62 | 797.16 |
| B63 | 770.18 |
| B65 | 801.18 |
| B66 | 819.16 |
| B67 | 786.85 |
| B77 | 838.31 |
| B79 | 865.31 |
| B80 | 865.01 |
| B81 | 865.03 |
| B82 | 849.42 |
| B83 | 820.40 |
| B84 | 849.46 |
| B85 | 849.42 |
| B86 | 820.46 |
| B87 | 820.43 |
| B88 | 820.45 |
| B89 | 827.36 |
| B90 | 787.32 |
| B91 | 854.36 |
| B92 | 841.46 |
| B93 | 843.38 |
| B94 | 854.36 |
| B95 | 853.43 |
| B96 | 826.36 |
| B97 | 826.35 |
| B98 | 856.30 |
| B99 | 815.30 |
| B104 | 815.40 |
| B105 | 815.40 |
| B106 | 838.40 |
| B107 | 838.40 |
| B108 | 838.40 |
| B109 | 838.40 |
| B110 | 838.40 |
| B112 | 839.40 |
| B113 | 839.40 |
| B114 | 814.40 |
| B115 | 881.40 |
| B116 | 881.46 |
| B117 | 880.39 |
| B118 | 839.40 |
| B119 | 812.82 |
| B120 | 788.30 |
| B121 | 788.40 |
| B122 | 788.40 |
| B123 | 839.40 |
| B124 | 838.29 |
| B125 | 869.32 |
| B126 | 868.35 |
| B127 | 869.39 |
| B128 | 867.40 |

Compound B20:3-((S)-3-(2-(7-(4-((6S,8R)-7-(2,2-difluoroethyl)-8-methyl-6,7,8,9-tetrahydro-3H-pyrazolo[4,3-f]isoquinolin-6-yl)-3,5-difluorophenyl)-2,7-diazaspiro[3.5]nonan-2-yl)-2-oxoethyl)-8-oxo-1,2,3,4,4a,5,8,10-octahydro-9H-pyrazino[1′,2′: 4,5][1,4]oxazino[2,3-f]isoindol-9-yl)piperidine-2,6-dione

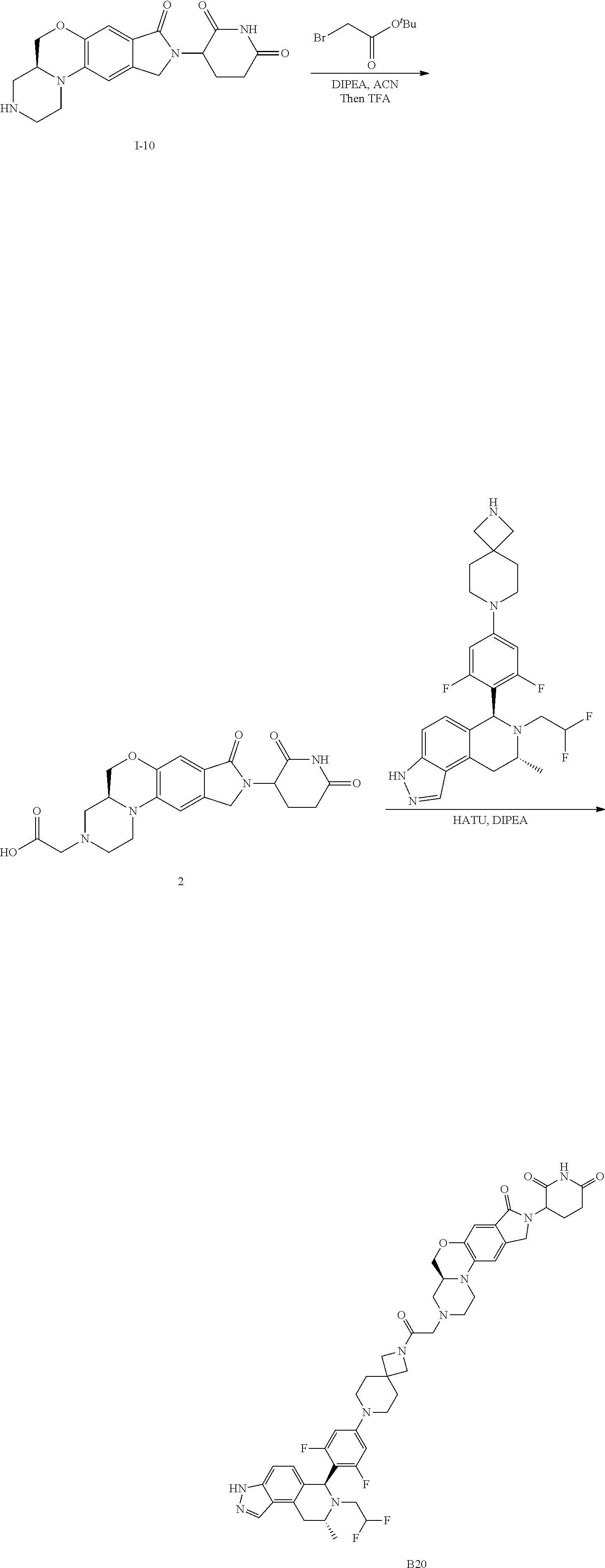

Step 1:2-((4aS)-9-(2,6-dioxopiperidin-3-yl)-8-oxo-1,2,4a,5,9,10-hexahydro-8H-pyrazino[1,2′: 4,5][1,4]oxazino[2,3-f]isoindol-3 (4H)-yl) acetic acid

To a solution of 1 (50 mg, 1.0 equiv) in acetonitrile (6 mL) was added DIPEA (148 μL, 8.0 equiv) and tert-butyl 2-bromoacetate (17.2 μL, 1.1 equiv). The reaction mixture was stirred overnight. Then the solvent was removed, and the residue was purified by pre-HPLC. The fraction containing product was concentrated, followed by adding TFA. 2 h Later, TFA was removed under reduced pressure and compound 2 was obtained as a white solid (45 mg, yiled=80.4%) after lyophilization. LC-MS (ESI) m/z: 415.16 [M+1].

Step 2:3-((S)-3-(2-(7-(4-((6S,8R)-7-(2,2-difluoroethyl)-8-methyl-6,7,8,9-tetrahydro-3H-pyrazolo[4,3-f]isoquinolin-6-yl)-3,5-difluorophenyl)-2,7-diazaspiro[3.5]nonan-2-yl)-2-oxoethyl)-8-oxo-1,2,3,4,4a,5,8,10-octahydro-9H-pyrazino[1′,2′: 4,5][1,4]oxazino[2,3-f]isoindol-9-yl)piperidine-2,6-dione (B20)

To a solution of 2 (6 mg, 1.0 equiv) in DMF (2.0 mL) was added DIPEA (12 μL, 6.0 equiv) and HATU (4.3, 1.0 equiv). 10 Min later, compound 2 (8.2 mg, 1.2 equiv) was added and the reaction mixture was stirred for 10-15 min. Then the reaction mixture was inmediately purified by pre-HPLC to obtain the title compound B20 as a white solid 8.2 mg. LCMS (ESI) m/z: 884.37 [M+1].

Compound B21:3-((S)-3-(2-(7-(4-((6S,8R)-7-(2,2-difluoroethyl)-8-methyl-6,7,8,9-tetrahydro-3H-pyrazolo[4,3-f]isoquinolin-6-yl)-3,5-difluorophenyl)-2,7-diazaspiro[3.5]nonan-2-yl) acetyl)-8-oxo-1,2,3,4,4a,5,8,10-octahydro-9H-pyrazino[1′,2′: 4,5][1,4]oxazino[2,3-f]isoindol-9-yl)piperidine-2,6-dione

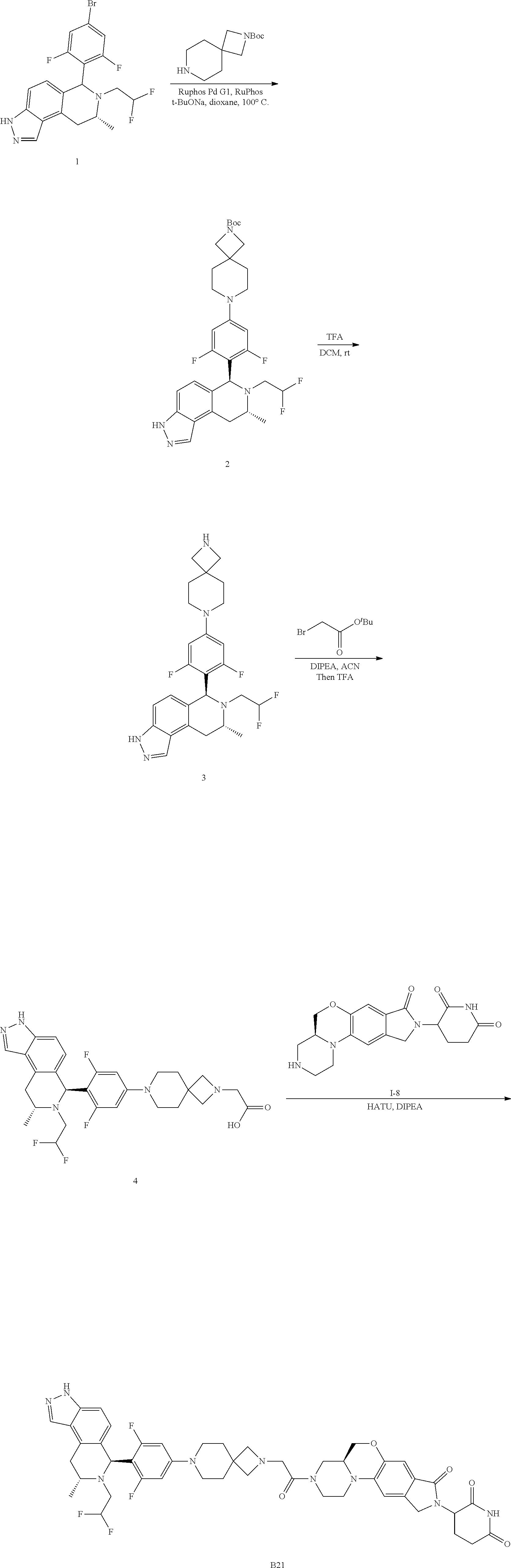

### Step 1: tert-butyl 7-(4-((6S,8R)-7-(2,2-difluoroethyl)-8-methyl-6,7,8,9-tetrahydro-3H-pyrazolo[4,3-f]isoquinolin-6-yl)-3,5-difluorophenyl)-2,7-diazaspiro[3.5]nonane-2-carboxylate

To a mixture of intermediate 1 (300 mg, 1.0 eq), tert-butyl 3,9-diazaspiro[5.5]undecane-3-carboxylate (345 mg, 2.0 eq), RuPhos Pd G1 (83 mg, 0.15 eq), RuPhos (47.5 mg, 0.15 eq) and t-BuONa (260 mg, 4.0 eq) in dioxane (12 mL) was degassed and purged with N2 3 times, and then the mixture was stirred at 100° C. for 6 h. LC-MS showed the starting material 1 was consumed completely, and a main peak with desired MS was formed. The mixture was cooled, diluted with DCM/MeOH, filtered through Celite, the filter cake was washed with DCM, and the filtrated was concentrated under reduced pressure. The residue was purified by silica gel flash chromatography (PE: EA=100:0 to 60:40), and the crude product was obtained as a yellow oil. LC-MS (ESI) m/z: 588.11 [M+1]

### Step 2: (6S,8R)-6-(2,6-difluoro-4-(2,7-diazaspiro[3.5]nonan-7-yl)phenyl)-7-(2,2-difluoroethyl)-8-methyl-6,7,8,9-tetrahydro-3H-pyrazolo[4,3-f]isoquinoline

To a solution of 2 (90 mg) in DCM (4 mL) was added TFA (2 mL). The reaction mixture was stirred for 1 h, then concentrated under reduced pressure. The residue was lyophilized, and compound 3 (90 mg) was obtained as a yellow solid. LC-MS (ESI) m/z: 488.14 [M+1]

### Step 3:2-(7-(4-((6S,8R)-7-(2,2-difluoroethyl)-8-methyl-6,7,8,9-tetrahydro-3H-pyrazolo[4,3-f]isoquinolin-6-yl)-3,5-difluorophenyl)-2,7-diazaspiro[3.5]nonan-2-yl) acetic acid

To a solution of 3 (30 mg, 1.0 equiv) in acetonitrile (4 mL) was added DIPEA (58 μL, 8.0 equiv) and tert-butyl 2-bromoacetate (6.8 μL, 1.1 equiv). The reaction mixture was stirred overnight. Then the solvent was removed, and the residue was purified by pre-HPLC. The fraction containing product was concentrated, followed by adding TFA. 2 h Later, TFA was removed under reduced pressure and compound 4 was obtained as a light-yellow solid after lyophilization. LCMS (ESI) m/z: 546.17 [M+1].

### Step 4:3-((S)-3-(2-(7-(4-((6S,8R)-7-(2,2-difluoroethyl)-8-methyl-6,7,8,9-tetrahydro-3H-pyrazolo[4,3-f]isoquinolin-6-yl)-3,5-difluorophenyl)-2,7-diazaspiro[3.5]nonan-2-yl) acetyl)-8-oxo-1,2,3,4,4a,5,8,10-octahydro-9H-pyrazino[1′,2′: 4,5][1,4]oxazino[2,3-f]isoindol-9-yl)piperidine-2,6-dione

To a solution of 4 (10.1 mg, 1.2 equiv) in DMF (2.0 mL) was added DIPEA (13.3 μL, 6.0 equiv) and HATU (4.9 mg, 1.0 equiv). 10 Min later, intermediate 1-8 (6.0 mg, 1.0 equiv) was added and the reaction mixture was stirred for 10-15 min. Then the reaction mixture was inmediately purified by pre-HPLC to obtain the title compound B21 as a white solid 8.5 mg. LC-MS (ESI) m/z: 884.36 [M+1].

The following examples were prepared in a manner analogous to compounds B20 and B21.

TABLE 6
Characterization Data for Compounds
Prepared According to B20 and B21

| Compound No | LC-MS (M + H)⁺ |
|---|---|
| B22 | 884.54 |
| B23 | 898.34 |
| B26 | 830.40 |
| B27 | 830.36 |
| B28 | 843.37 |
| B31 | 844.32 |
| B32 | 844.40 |
| B33 | 884.15 |
| B34 | 856.29 |
| B35 | 813.41 |
| B36 | 870.46 |
| B37 | 870.43 |
| B38 | 856.39 |
| B39 | 870.42 |
| B40 | 870.31 |
| B41 | 870.33 |
| B42 | 870.51 |
| B43 | 813.30 |
| B64 | 866.36 |
| B78 | 866.38 |
| B100 | 830.30 |
| B101 | 830.30 |

### Compound B126: (3S)-3-(1′-((9-(5-((6S,8R)-8-methyl-7-(2,2,2-trifluoroethyl)-6,7,8,9-tetrahydro-3H-pyrazolo[4,3-f]isoquinolin-6-yl)pyrimidin-2-yl)-1-oxa-9-azaspiro[5.5]undecan-3-yl)methyl)-6-oxo-6,8-dihydro-2H,7H-spiro[furo [2,3-e]isoindole-3,4′-piperidin]-7-yl)piperidine-2,6-dione

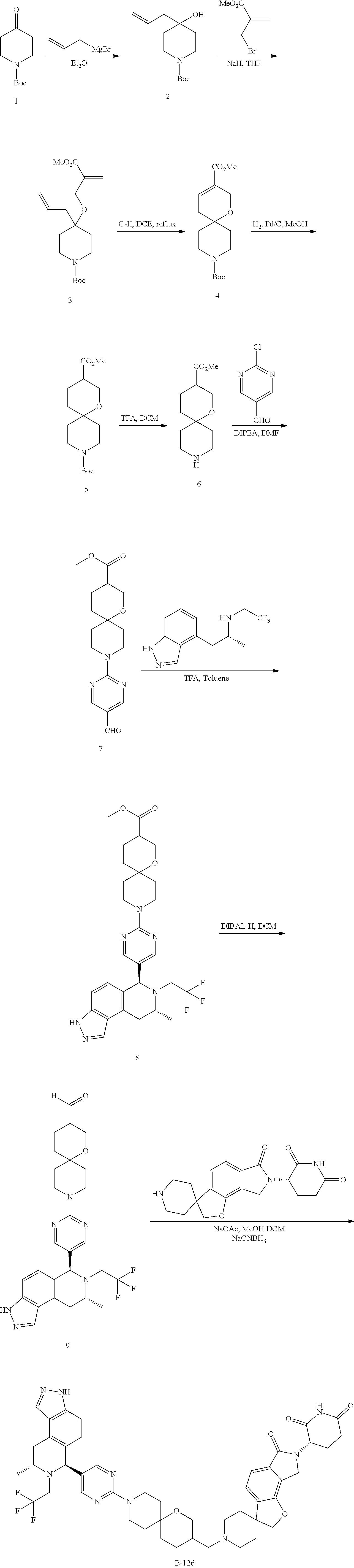

### Step 1: tert-butyl 4-allyl-4-hydroxypiperidine-1-carboxylate

Allyl magnesium bromide (IM sol. in Et20, 26 mL) was added at 0° C. to a solution of N-Boc-4-piperidone (1, 4.03 g, 20 mmol) in Et₂O (80 mL). It was stirred for 10 min. The reaction mixture was warmed to room temperature and stir for 4 h. Followed by quenching by addition of sat. aq. NH₄CL. It was then extract with EtOAc. The organic phase was separated and washed twice with water then brine, then dried over sodium sulfate, filtered and concentrated in vacuo. The crude mixture was purified using column chromatography on silica gel (0% to 100% ethyl acetate in hexanes. The desired compound 2 (4.84 g, ~ 90% yield) was obtained as a colorless oil. ¹H NMR: (400 MHZ, CDCl₃) δ 5.77-5.94 (m, 1H), 5.19 (dd, J=10.4, 1.8 Hz, 1H), 5.14 (dd, J=17.1, 1.9 Hz, 1H), 3.81 (dt, J=13.4, 3.3 Hz, 2H), 3.08-3.24 (m, 2H), 2.23 (d, J=7.6 Hz, 2H), 1.53 (dd, J=10.4, 4.8 Hz, 4H), 1.46 (s, 9H),

### Step 2: tert-butyl 4-allyl-4-((2-(methoxycarbonyl) allyl)oxy) piperidine-1-carboxylate

A 60% oil dispersion of sodium hydride (0.438 g, 1.2 eq) was added to a solution of tert-butyl 4-allyl-4-hydroxypiperidine-1-carboxylate (2, 2.2 g, 1 eq) in anhydrous DMF (10 mL/mmol) and the mixture cooled to 0° C. The mixture was warmed to room temperature over 1 hour and methyl 2-(bromomethyl) acrylate (1.63 g, I eq) in DMF was added dropwise to the solution over 5 minutes. The mixture was stirred for 12 h. The reaction mixture cooled down to 0° C., a saturated solution of ammonium chloride was added to the reaction mixture and the mixture was diluted with ethyl acetate. The organic phase was separated and washed twice with water then brine, then dried over sodium sulfate, filtered and concentrated in vacuo. The crude mixture was purified using column chromatography on silica gel (0% to 100% ethyl acetate in hexanes, Rf: 0.3; 30% EA/Hx). The desired compound 3 was obtained as a colorless oil. Yield: 60-70%

### Step 3:9-(tert-butyl) 3-methyl 1-oxa-9-azaspiro[5.5]undec-3-ene-3,9-dicarboxylate

tert-butyl 4-{[2-(methoxycarbonyl)prop-2-cn-1-yl]oxy}-4-(prop-2-cn-1-yl)pipcridine-1-carboxylate (3, 340 mg, 1 eq) in anhydrous 1,2-dichloroethane (20 mL/mmol) was combined with G-II (0.05 eq) and the mixture was heated at 50° C. for 4 h. The mixture was cooled to room temperature and quenched by passing air. It was then filtered and evaporated and purified by flash. tert-butyl 3-oxo-1-oxa-9-azaspiro[5.5]undecane-9-carboxylate (4) was obtained as an oil. Yield: ~80%

### Step 4:9-(tert-butyl) 3-methyl 1-oxa-9-azaspiro[5.5]undecane-3,9-dicarboxylate

Pd/C (100 mg, 10% wt.) was added to a solution of compound 4 (1 μm, 3.31 mmol) in MeOH (33 mL, 10 mL/mmol). The reaction mixture was degassed with H₂ and stirred under a H₂ atmosphere for 12 h at room temperature. The mixture was then filtered through celite and washed with MeOH. Concentration under reduced pressure followed by purification by flash chromatography (0% to 100% ethyl acetate in hexanes) gave the desired compound 5 in 60% yield.

### Step 5: methyl 1-oxa-9-azaspiro[5.5]undecane-3-carboxylate

To a solution of 5 (300 mg) in DCM (5 mL) was added TFA (2.5 mL). The reaction mixture was stirred overnight, then concentrated under reduced pressure and used for the next steps without further purification.

### Step 6: methyl 9-(5-formylpyrimidin-2-yl)-1-oxa-9-azaspiro[5.5]undecane-3-carboxylate

methyl 1-oxa-9-azaspiro[5.5]undecane-3-carboxylate (1 μm, 4.4 mmol) was added to a solution of 2-chloropyrimidine-5-carbaldehyde (600 mg, 4.4 mmol) in acetonitrile (20 mL). To it 2 mL of DIPEA was added and the reaction mixture was stirred at 90° C. for 4 h. The mixture was cooled to room temperature and evaporated and purified by flash. methyl 9-(5-formylpyrimidin-2-yl)-1-oxa-9-azaspiro[5.5]undecane-3-carboxylate (7) was obtained as an yellow solid. Yield: ~80%

### Steps 7: methyl 9-(5-((6S,8R)-8-methyl-7-(2,2,2-trifluoroethyl)-6,7,8,9-tetrahydro-3H-pyrazolo[4,3-f]isoquinolin-6-yl)pyrimidin-2-yl)-1-oxa-9-azaspiro[5.5]undecane-3-carboxylate

To a mixture of (R)-1-(1H-indazol-4-yl)-N-(2,2,2-trifluoroethyl)propan-2-amine (286 mg, 1.0 mmol, 1 eq) in TFA (1 mL) and toluene (10 mL) was added methyl 9-(5-formylpyrimidin-2-yl)-1-oxa-9-azaspiro[5.5]undecane-3-carboxylate (370 mg, 1.0 mmol, 1 eq). The mixture was stirred at 100° C. for overnight. LCMS showed the reaction was completed. The reaction was concentrated and purified by flash. The product methyl 9-(5-((6S,8R)-8-methyl-7-(2,2,2-trifluoroethyl)-6,7,8,9-tetrahydro-3H-pyrazolo[4,3-f]isoquinolin-6-yl)pyrimidin-2-yl)-1-oxa-9-azaspiro[5.5]undccanc-3-carboxylate (8) was obtained as an yellow solid. LC/MS (ESI) m/z: 573.37.

### Steps 8:9-(5-((6S,8R)-8-methyl-7-(2,2,2-trifluoroethyl)-6,7,8,9-tetrahydro-3H-pyrazolo[4,3-f]isoquinolin-6-yl)pyrimidin-2-yl)-1-oxa-9-azaspiro[5.5]undecane-3-carbaldehyde

To a solution of 8 (558 mg, 1.0 mmol, 10 mL/mmol) in DCM at −78° C., 1.5 mL of DIBAL-H (1.0 M in DCM) was added dropwise. Then, the temperature was slowly increased to −20° C. and stirred for 6 h. After that, the reaction was slowly quenched with satd. Na₂SO₄ at 0° C. and was then filtered and washed several times with EtOAc. Purification by flash chromatography to obtain the desired product (9) LCMS (ESI) m/z: 546.20 [M+18].

### Steps 9: (3S)-3-(1′-((9-(5-((6S,8R)-8-methyl-7-(2,2,2-trifluoroethyl)-6,7,8,9-tetrahydro-3H-pyrazolo[4,3-f]isoquinolin-6-yl)pyrimidin-2-yl)-1-oxa-9-azaspiro[5.5]undecan-3-yl)methyl)-6-oxo-6,8-dihydro-2H,7H-spiro[furo [2,3-e]isoindole-3,4′-piperidin]-7-yl)piperidine-2,6-dione

To a mixture of compound 9 (53 mg, 0.1 eq.) in methanol (5 mL) and dichloromethane (5 mL) was added(S)-3-(6-oxo-6,8-dihydro-2H,7H-spiro[furo [2,3-e]isoindole-3,4′-piperidin]-7-yl) piperidine-2,6-dione (35 mg, 0.1 eq.), and AcONa (24 mg, 0.3 eq.). The mixture was stirred at 25° C. for 20 mins, then sodium cyanoborohydride (0.2 mL, 0.2 eq., 1M in THF) was added and the mixture was further stirred for 10 mins. LCMS showed the reaction was complete. It was purified by pre-HPLC, and the desired product (B126) was obtained as white solid. LCMS (ESI) m/z: 868.40 [M+1].

### FOR COMPOUNDS B1 to B126

TABLE 7
Characterization Data for B1-B126

| Compound No. | LC-MS ([M + H]+) |
|---|---|
| B1 | 854.06 |
| B2 | 854.10 |
| B3 | 840.11 |
| B4 | 827.23 |
| B5 | 855.12 |
| B6 | 855.12 |
| B7 | 854.25 |
| B8 | 854.08 |
| B9 | 838.35 |
| B10 | 837.35 |
| B11 | 835.31 |
| B12 | 841.10 |
| B13 | 827.32 |
| B14 | 798.32 |
| B15 | 855.11 |
| B16 | 815.23 |
| B17 | 885.03 |
| B18 | 765.87 |
| B19 | 841.01 |
| B20 | 884.37 |
| B21 | 884.36 |
| B22 | 884.54 |
| B23 | 898.34 |
| B24 | 841.39 |
| B25 | 841.36 |
| B26 | 830.40 |
| B27 | 830.36 |
| B28 | 843.37 |
| B29 | 828.39 |
| B30 | 828.42 |
| B31 | 844.32 |
| B32 | 844.40 |
| B33 | 884.15 |
| B34 | 856.29 |
| B35 | 813.41 |
| B36 | 870.46 |
| B37 | 870.43 |
| B38 | 856.39 |
| B39 | 870.42 |
| B40 | 870.31 |
| B41 | 870.33 |
| B42 | 870.51 |
| B43 | 813.30 |
| B44 | 813.32 |
| B45 | 813.32 |
| B46 | 870.46 |
| B47 | 828.30 |
| B48 | 838.33 |
| B49 | 820.32 |
| B50 | 820.19 |
| B51 | 849.33 |
| B52 | 839.33 |
| B53 | 850.45 |
| B54 | 849.43 |
| B55 | 849.50 |
| B56 | 848.40 |
| B57 | 837.34 |
| B58 | 851.41 |
| B59 | 851.40 |
| B60 | 837.31 |
| B61 | 851.41 |
| B62 | 797.16 |
| B63 | 770.18 |
| B64 | 866.36 |
| B65 | 801.18 |
| B66 | 819.16 |
| B67 | 786.85 |
| B68 | 849.41 |
| B69 | 835.37 |
| B70 | 841.37 |
| B71 | 827.39 |
| B72 | 871.48 |
| B73 | 843.18 |
| B74 | 824.34 |
| B75 | 854.40 |
| B76 | 824.16 |
| B77 | 838.31 |
| B78 | 866.38 |
| B79 | 865.31 |
| B80 | 865.01 |
| B81 | 865.03 |
| B82 | 849.42 |
| B83 | 820.40 |
| B84 | 849.46 |
| B85 | 849.42 |
| B86 | 820.46 |
| B87 | 820.43 |
| B88 | 820.45 |
| B89 | 827.36 |
| B90 | 787.32 |
| B91 | 854.36 |
| B92 | 841.46 |
| B93 | 843.38 |
| B94 | 854.36 |
| B95 | 853.43 |
| B96 | 826.36 |
| B97 | 826.35 |
| B98 | 856.30 |
| B99 | 815.30 |
| B100 | 830.30 |
| B101 | 830.30 |
| B102 | 852.40 |
| B103 | 881.40 |
| B104 | 815.40 |
| B105 | 815.40 |
| B106 | 838.40 |
| B107 | 838.40 |
| B108 | 838.40 |
| B109 | 838.40 |
| B110 | 838.40 |
| B111 | 840.30 |
| B112 | 839.40 |
| B113 | 839.40 |
| B114 | 814.40 |
| B115 | 881.40 |
| B116 | 881.46 |
| B117 | 880.39 |
| B118 | 839.40 |
| B119 | 812.82 |
| B120 | 788.30 |
| B121 | 788.40 |
| B122 | 788.40 |
| B123 | 839.40 |
| B124 | 838.30 |
| B125 | 869.32 |
| B126 | 868.35 |
| B127 | 869.39 |
| B128 | 867.40 |

### II. BIOLOGICAL ASSAYS

### For Compounds A1-A26

In vitro Assay: IC₅₀ Measurements for binding to CRBN/DDB1

The binding potency was determined using HTRF assay technology (Perkin Elmer). Compounds were serially diluted in DMSO and 0.2 μL volume was transferred to white 384-well plate. The reaction was conducted in total volume of 20 μL with addition of 2 nM His tagged CRBN+DDB−DLS7+CXU4 (Wuxi, catalogue #RP210521GA) to compounds followed by addition of 60 nM Fluorescent probe Cy⁵-labeled Thalidomide (Tenova Pharma, catalogue #T52461), and 0.4 nM of MAb Anti-6HIS Tb cryptate Gold (Cisbio, catalogue #61HI2TLA in the assay buffer (50 mM HEPES pH 7.5, 1 mM TCEP, 0.01% Brij-35, 50 mM NaCl, and 0.1% BSA). After one hour incubation at room temperature, the HTRF signals were read on Envision reader (Perkin Elemer). Data were analyzed using XLfit using four parameters dose response curve to determine IC₅₀ₛ and shown in Table 8.

TABLE 8
CRBN binding IC₅₀

| Compound No. | CRBN HTRF IC₅₀ (nM) |
|---|---|
| A1 | D |
| A2 | B |
| A3 | C |
| A4 | A |
| A5 | B |
| A6 | B |
| A7 | B |
| A8 | C |
| A9 | B |
| A10 | B |
| A11 | D |
| A12 | C |
| A13 | A |
| A14 | B |
| A15 | C |
| A16 | A |
| A17 | B |
| A18 | B |
| A19 | C |
| A20 | A |
| A21 | A |
| A22 | B |
| A23 | C |
| A24 | B |
| A25 | B |

Note:
IC₅₀: “A”: <50 nM; “B”: 50-500 nM; “C”; >500 and <5000 nM; “D”: >=5000 nM.

In vitro Assay: IC₅₀ Measurements for binding to ERα_LBD (GST)·

Final assay conditions:
1. ERα_LBD (GST) protein: 4 nM
2. Tb Anti-GST: 2 nM
3. Fluormone ES2 Green tracer: 3 nM
4. Incubation time: 60 min
5. DMSO: 1%
6. Assay buffer: Adding 1M DTT to Nuclear receptor Buffer K for final 5 mM DTT.
7. ZPE: 1% DMSO
8. HPE: 1 μM ARV_471
9. LanthaScreen® TR-FRET ERα Competitive Binding Assay (ThermoFisher, #A¹⁵⁸⁸⁷)

100× Compound preparation:
1) Cherry pick 2 μL 10 mM compound stock to column 1 of a 384 intermediate plate
2) Add 18 μL DMSO to column 1 to dilute compound to 1 mM.
3) Transfer 10 μL 1 mM compound to column 1 of a LDV plate.
2) Add 6 μl DMSO to column 2-10 of the LDV plate.
3) Compounds undergo 3-fold serial dilution (3 μL+6 μL) in DMSO.
4) Transfer 120 nL compound solution to assay plate.

ZPE: 120 nL 100% DMSO

Procedure:

Prepare complete nuclear receptor buffer K by adding 1 M DTT to nuclear receptor buffer K for a final concentration of 5 mM DTT. Complete nuclear receptor buffer K must be prepared fresh daily. Prepare 2× protein solution using complete nuclear receptor buffer K containing 8 nM ERα_LBD (GST) and 4 nM Tb Anti-GST. Then, prepare 2× Fluormone ES2 Green tracer (6 nM) using complete nuclear receptor buffer K. Add 6 μL 2× Fluormone ES2 green tracer into a compound plate (PerkinElmer 6008289) by dragonfly with one-tips-addition. Subsequently, add 6 μL 2× protein solution into the plate. Briefly and gently mix the 384-well plate on a plate shaker and incubate at room temperature protected from light for 60 min. The plate is sealed with a cover to minimize evaporation. Read the plate at wavelengths of 520 nm and 495 nm. Calculate the TR-FRET ratio by dividing the emission signal at 520 nm by the emission signal at 495 nm. Generate a binding curve by plotting the emission ratio vs. the log [ligand]. To determine the IC₅₀ value, fit the data using XL-fit for a sigmoidal dose-response. The data of selected compounds were shown in Table 9.

TABLE 9
ERα binding IC₅₀

| Compound No | ERα HTRF IC₅₀ (nM) |
|---|---|
| A4 | A |
| A5 | A |
| A8 | B |
| A11 | B |
| A15 | A |
| A16 | A |
| A17 | A |
| A19 | A |
| A21 | A |
| A22 | A |
| A24 | A |
| A25 | A |
| A26 | A |

Note:
IC₅₀: “A”: <10 nM; “B”: >=10 nM.

In-cell Western (ICW) assays in MCF-7 and T47D cell lines.

Reagents and Consumables for ICW
1) MCF-7 from HDB
2) T47D from HDB
3)CS—FBS, BI, Cat #04-201-1
4) phenol red-free RPMI1640, Thermo, Cat #11835
5) P/S, Biosera Liquid, Cat #XC-A⁴¹²²
6) 384-well cell plate (black), Corning, Cat #3764
7) PFA, Electron Microscopy Sciences, Cat #15710
8) Intercept (PBS) Blocking Buffer, Licor, Cat #927-70001
9) Triton X-100, Sigma, Cat #X-100
10) ER antibody, CST, Cat #13258
11) IRDye 800CW Goat anti-Rabbit IgG, LiCor, Cat #926-32211
12) CellTag 700 Stain, Licor, Cat #926-41090
13)Odyssey® DLx Imaging System, LiCor
14) En Vision, PerkinElmer

Procedures for ICW assays

In vitro Assay: MCF-7 and T47D ICW assay

Day 1: MCF-7 and T47D cell (From HDB) were seeded in 384-well black plate with phenol red-free RPMI1640+10% CS-FBS+1% P/S medium (1*104 for MCF-7 and 1.5*104 for T47D cells/well, 30 ul medium) for overnight at 37° C., 5% CO₂ incubator.

Day 2: Cells were treated at desired compound concentrations (0.02 to 300 nM) and DMSO as vehicle control for 16 hrs at 37° C., 5% CO₂ incubator.

Day 3: After 16 hrs of compounds treatment, cells were fixed by 4% PFA and permeabilized with elution buffer (0.1% Triton X-100 in 1% PBS Solution). Subsequently, cells were blocked with Intercept (PBS) Blocking Buffer (Li—COR, Odyssey Blocking Buffer), and were stained with ER (1:500, Cell signaling) primary antibody for overnight at cold room.

Day 4: Remove the buffer, add IRDye 800CW Goat anti-Rabbit IgG Secondary Antibody (1:2000) and CellTag 700 Stain (1:500) in Intercept (PBS) Blocking Buffer. Finally, cell plate is placed in incubator to dry. Image and signal were captured on Odyssey® DLx Imaging System. Data was further analyzed using XLfit using four parameters dose response curve to determine DC₅₀ and Dₘₐₓ.

Data analysis

### Data are Analyzed by Image Studio V5.2 and XLfit.

Half maximal degradation concentration values (DC₅₀) and maximal degradation percentage (Dₘₐₓ, %) of ER are summarized in Table 10.

TABLE 10
ER degradation by in-cell western (ICW) assays

| Compound | ER MCF7 ICW | ER MCF7 ICW | ER T47D ICW | ER T47D ICW |
|---|---|---|---|---|
| No | DC₅₀ (nM) | Dₘₐₓ (%) | DC₅₀ (nM) | Dₘₐₓ (%) |
| A2 | A | B | A | B |
| A3 | A | B | A | B |
| A4 | A | B | A | A |
| A5 | A | B | A | B |
| A6 | A | B | A | B |
| A7 | A | B | A | B |
| A8 | A | A | A | A |
| A9 | A | B | A | B |
| A10 | A | B | A | B |
| A11 | A | A | A | A |
| A12 | A | A | A | A |
| A13 | A | C | A | B |
| A14 | A | B | A | A |
| A15 | A | B | A | B |
| A16 | A | A | A | A |
| A17 | A | A | A | A |
| A18 | B | B | B | B |
| A19 | A | B | A | B |
| A20 | C | C | C | B |
| A21 | A | A | A | A |
| A22 | A | B | A | B |
| A23 | B | B | B | B |
| A24 | A | B | A | A |
| A25 | A | A | A | A |
| A26 | A | A | A | A |

Note:
DC₅₀: “A”: <1 nM; “B”: 1-10 nM; “C”: >10 and <100 nM; “D”: >=100 nM.
Dₘₐₓ: “A”: >=75%; “B”: >50% and <75%; “C”: 25%-50%; “D”: <25%.

CellTiter-Glo® (CTG) assays in MCF-7 and T47D cell lines.

Reagents and Consumables for CTG
1) MCF-7 from HDB
2) T47D from HDB
3)CS—FBS, BI, Cat #04-201-1
4) phenol red-free RPMI1640, Thermo, Cat #11835
5) P/S, Biosera Liquid, Cat #XC-A⁴¹²²
6) 384-well cell plate (white), Corning, Cat #3765
7) Cell TiterGlo reagent, Promega, Cat #G7573
8) En Vision, PerkinElmer.

Medium
1) Cell culture medium: phenol red-free RPMI1640+10% CS—FBS,1% P/S

Procedures for CTG assay

In vitro Assay: MCF-7 and T47D CTG assay

Day-1: MCF-7 and T47D cell (From HDB) were cultured in 384-well white plate with phenol red-free RPMI1640+10% CS-FBS+1% P/S medium (1,000 cells/well) for overnight at 37° C., 5% CO₂ incubator.

Day 0: Cells were treated at desired compound concentrations (0.5 to 10000 nM) (DMSO and Staurosporine as control) for Day 6 at 37° C.,5% CO₂ incubator.

Day 0 and Day 6: add Cell TiterGlo reagent and read on En Vision after 30 min incubation for data generation.
Data analysis

### Data are Analyzed by Image Studio V5.2 and XLfit.

Half maximal inhibitory concentration values (IC₅₀) of MCF-7 and T47D cell proliferation are summarized in Table 11.

TABLE 11
CellTiter-Glo ® (CTG) IC₅₀

| Compound No | MCF7 IC₅₀ (nM) | T47D IC₅₀ (nM) |
|---|---|---|
| A26 | B | B |
| A6 | A | A |
| A21 | A | B |
| A19 | A | A |
| A15 | A | A |
| A17 | C | B |
| A24 | C | C |

Note:
IC₅₀: “A”: <10 nM; “B”: 10-50 nM; “C”: >50 nM.

### For Compound B1-B126

In-cell western blot analysis. a. seed cells in black-sided/clear bottom 96- or 384-well plates at 40,000 or 10,000 cells/well, overnight; b. add diluted compounds (final 0.5% DMSO), 16 hours. 16 h later, remove medium, add 100 μL or 25 μL of 3.7-4.0% formaldehyde (PBS: FA=9:1), RT 20 min, no shaking; c. wash with PBS, and permeabilized with 100 μL or 25 μL/well of 1×PBS+0.1% Triton X-100 10 minutes; d. block with 100 μL or 25 μL Licor blocking buffer (Li-Cor), RT 1h, moderate shaking; d. Add 100 μL or 25 μL of anti-ER (cs-8644, 1:500-1,000)+GAPDH (Millipore MAB374, 1:1000) in Block+0.05% Tween 20. RT 2h, gentle shaking. Negative control: cells plus secondary antibodies (no primary antibodies); e. wash×4 with PBS +0.05-0.1% Tween 20, gentel shaking; f. anti-rabbit-680 and anti-mouse-800 (both 1:1000 in LiCor block+0.05% Tween20, RT 1h, gentle shaking, no light. LI-COR: 0.2% to reduce background; g. wash×4 with PBS+0.05% Tween 20, gental shaking; h. add 100 μL or 25 μL of PBS to each well and read on CLX plate reader. The relative ER percentage in treated cells were obtained by comparing the values of treated wells to those in untreated and DMSO-treated wells as 100%.

Western Blot Analysis. Western blot analysis was performed essentially as described previously. The cells treated with indicated compounds were lysed in Radioimmunoprecipitation Assay Protein Lysis and Extraction Buffer (25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail (Roche Diagnostics, Mannheim, Germany). Equal amounts of total protein were electrophoresed through 10% SDS-polyacrylamide gels after determination of protein concentration by BCA assay (Fisher Scientific, Pittsburgh, PA). The separated protein bands were transferred onto PVDF membranes (GE Healthcare Life Sciences, Marlborough, MA) and blotted against different antibodies, as indicated. The blots were scanned, and the band intensities were quantified using GelQuant.NET software provided by biochemlabsolutions.com. The relative mean intensity of target proteins was expressed after normalization to the intensity of glyceraldehyde-3-phosphate dehydrogenase bands.

Cell Growth Assay. The cells were seeded at 1500/well in 96 well plates overnight. One day after the seeding, they were treated with indicated doses of compounds respectively. 4 days after the compound treatment, 10% WST-8 reagent was added to the culture medium and incubate in a CO2 incubator at 37° C. for 2.5 hours. Before reading, the plate was mixed gently on an orbital shaker for one minute to ensure homogeneous distribution of color. The absorbance was measured of each sample using a microplate reader at a wavelength of 450 nm. The relative absorbance was calculated against the vehicle control from three individually repeats.

In vivo pharmacodynamic and efficacy studies. To develop breast cancer cell line xenografts, mice was given 4 μg/ml 17β-Estradiol in 0.05% EtOH dringking water for 1 week, followed with 8 μg/ml 17β-Estradiol in 0.1% EtOH drinking water thereafter. Five million cells in 50% Matrigel were injected subcutaneously into SCID mice. when tumors reached 100-400 mm³, mice were treated with vehicle control (5% DMSO, 10% solutol, 85% Water) or indicated dose of the drugs, sacrificed at indicated time-points, and tumor tissue was harvested for analysis. For in vivo efficacy experiments, when tumors reached 80-200 mm³, mice were randomized into groups. vehicle control (5% DMSO, 10% solutol, 85% Water) was given at the dose and with the duration indicated. Tumor sizes and animal weights were measured 2-3 times per week. Tumor volume (mm³)=(length× width2)/2. Tumor growth inhibition was calculated as TGI (%)=(Vc−Vt)/(Vc−Vo)×100, where Vc, Vt are the median of control and treated groups at the end of the study and Vo at the start. All the in vivo studies were performed under an animal protocol (PRO00005315) approved by the University Committee on Use and Care of Animals of the University of Michigan, in accordance with the recommendations in the Guide for the Care and Use of Laboratory Animals of the National Institutes of Health.

TABLE 10
Biological Data for Compounds B1-B126

| | Traditional Western | | Cell growth |
|---|---|---|---|
| | Degradation potency | ICW | inhibition in T47D |
| Compound No. | (DC₅₀) | DC₅₀ (nM) | cell line IC₅₀ (nM) |
| B1 | A | | |
| B2 | A | | |
| B3 | C | | |
| B4 | A | | |
| B5 | B | | |
| B6 | A | | |
| B7 | C | | |
| B8 | C | | |
| B9 | A | A | |
| B10 | B | | |
| B11 | A | | |
| B12 | B | | |
| B13 | A | | |
| B14 | C | | |
| B15 | A | A | A |
| B16 | B | | A |
| B17 | A | | |
| B18 | B | | |
| B19 | A | | |
| B20 | | A | A |
| B21 | | A | A |
| B22 | | A | A |
| B23 | | A | A |
| B24 | B | | B |
| B25 | A | | B |
| B26 | | A | A |
| B27 | | B | A |
| B28 | | B | A |
| B29 | | A | A |
| B30 | | A | B |
| B31 | | B | A |
| B32 | | A | A |
| B33 | | A | A |
| B34 | | A | A |
| B35 | | B | C |
| B36 | | A | A |
| B37 | | A | A |
| B38 | | A | A |
| B39 | | A | A |
| B40 | | A | A |
| B41 | | A | A |
| B42 | | A | A |
| B43 | | B | B |
| B44 | | A | A |
| B45 | | B | B |
| B46 | | A | A |
| B47 | | | A |
| B48 | | C | C |
| B49 | | B | C |
| B50 | | C | NA |
| B51 | | B | C |
| B52 | | A | A |
| B53 | | B | A |
| B54 | | A | B |
| B55 | | C | B |
| B56 | | C | |
| B57 | | A | A |
| B58 | | C | A |
| B59 | | A | B |
| B60 | | A | A |
| B61 | | A | A |
| B62 | | C | A |
| B63 | | A | A |
| B64 | | A | A |
| B65 | B | | |
| B66 | B | | |
| B67 | A | A | A |
| B68 | | A | B |
| B69 | | A | A |
| B70 | B | | |
| B71 | B | | |
| B72 | | A | B |
| B73 | | B | A |
| B74 | | | B |
| B75 | | | A |
| B76 | | A | B |
| B77 | | B | B |
| B78 | | A | A |
| B79 | | A | A |
| B80 | | A | A |
| B81 | | A | A |
| B82 | | B | A |
| B83 | | C | C |
| B84 | | C | C |
| B85 | | B | C |
| B86 | | A | B |
| B87 | | A | B |
| B88 | | A | B |
| B89 | | C | C |
| B90 | | B | B |
| B91 | | A | B |
| B92 | | C | C |
| B93 | | B | B |
| B94 | | A | A |
| B95 | | C | A |
| B96 | | A | A |
| B97 | | A | A |
| B98 | | A | A |
| B99 | | B | C |
| B100 | | B | B |
| B101 | | A | B |
| B102 | | C | |
| B103 | C | | |
| B104 | B | B | |
| B105 | C | | |
| B106 | | A | A |
| B107 | | B | A |
| B108 | | A | A |
| B109 | | C | B |
| B110 | | C | B |
| B111 | B | | B |
| B112 | | A | A |
| B113 | | A | |
| B114 | | C | A |
| B115 | | A | A |
| B116 | | A | A |
| B117 | | A | A |
| B118 | | B | |
| B119 | | B | A |
| B120 | | C | |
| B121 | | C | |
| B122 | | C | |
| B123 | | A | |
| B124 | | A | A |
| B125 | | A | A |
| B126 | | A | A |
| B127 | | A | A |
| B128 | | A | A |

Note:
DC₅₀: “A”: <10 nM; “B”: 10-100 nM; “C”: >100 nM
IC₅₀: “A”: <1 nM; “B”: 1-10 nM; “C”: >10 nM

### In Vitro Efficacy Studies

### ER Degradation in Breast Cancer Cell Lines

ER degradation is measured using several different breast cancer cell lines in multiple cellular assays. Cell lines to be used for this purpose include, but are not limited to, MCF-7 cells (ATCC, catalog #HB-22), T47D cells (ATCC, catalog #HTB-133), or CAMA1 cells (ATCC, catalog #HTB-21) expressing wild type ER, or breast cancer cell lines expressing clinically relevant ER gene mutations, such as MCF-7 cells engineered to express Q380E, Y537S, or D538G ER. Endogenous ER in breast cancer cell lines is measured using Western blot, in-cell Western assay or HiBiT assay in cells engineered to express a HiBiT-tagged version of ER. ER degradation is measured at times, e.g., between 2 and 24 hours. Cells are treated with vehicle control (DMSO) or the compound at various concentrations (e.g., ranging from 0.005 nM to 100 nM). Some assays are conducted in the presence of estradiol, while other assays are conducted in the absence of estradiol. The compounds of this disclosure are expected to degrade ER protein in breast cancer cell lines.

### Cell Growth Inhibition in Breast Cancer Cell Lines

Cell growth inhibition is measured using several different cell lines (e.g., the ones mentioned above) to test whether ER degradation with the compounds of this disclosure impacts cell growth inhibition in breast cancer cell lines. Cells are treated with vehicle control (DMSO) or the compound at various concentrations (e.g., ranging from 0.003 nM to 100 nM) for about 144 hours. Briefly cells per well are plated in each well of a 384-well plate. 24 hours later, the compound is dispensed into the wells. 144 hours after compound is added to wells, CellTiter-Glo (Promega) is added to wells and plates are read on an EnVision® Plate Reader (Perkin Elmer). The compounds of this disclosure are expected to inhibit or retard cell growth in breast cancer cell lines.

### In Vivo Pharmacokinetic and Pharmacodynamic (PKPD) and Efficacy Studies

### ER Degradation in MCF-7 Tumor Model

To evaluate the ability of compounds of this disclosure to reduce ER protein levels in vivo, an orthotopic human breast cancer MCF7 xenograft model in female NOD/SCID mice is used. Each mouse is implanted subcutaneously with estrogen pellets at the right flank before the tumor inoculation. Each mouse is inoculated at the right third mammary fat pad with MCF7 tumor cells (2×10⁷) in 0.2 mL of PBS with Matrigel (1:1) for tumor development. Mice are treated with vehicle control (e.g., 5% DMSO, 10% solutol, 85% water) or the compound for 6 or 24 hours past the 3rd dose once tumors reach 400-500 mm³. Tumors are harvested at given times, bisected and flash frozen. Half of the tumor is analyzed for compound concentration in the tumor or plasma and the other half is analyzed using Western blot to quantify the extent of ER degradation. The compounds of this disclosure are expected to demonstrate dose-dependent ER degradation in MCF-7 tumor model.

Tumor growth inhibition and regression in mice

To evaluate the ability of compounds of this disclosure to inhibit tumor growth and/or cause tumor growth regression in vivo, the MCF-7 human breast carcinoma female athymic nude mouse model is used. Mice are supplemented with 10 μg/mL 17 beta-estradiol in their drinking water 3 days prior to cell implantation and then for the duration of the study. Mice are injected with 1×107 MCF-7 tumor cells in PBS subcutaneously in the flank. Mice are treated with vehicle control (e.g., 5% DMSO, 10% solutol, 85% Water) or the compound once tumors reach 150-200 mm³, and sacrificed when tumor volume reaches 2000 mm³ or at the end of the study (whichever occurs first). Tumor sizes and animal weights and caliper measurements of tumors are collected 2-3 times per week. Tumor volume (mm³)=(length×width²)/2. Tumor growth inhibition is calculated using DT/PC TGI (%)=(1-((Tₑ−T₀)/(Cₑ−C₀)))*100, where ^{D}T/^{D}C is the difference (delta) or change in test vs control TGI; Tₑ=Test tumor volume endpoint, T₀=Test tumor volume at start of dosing, Cₑ=Vehicle control tumor volume endpoint, C₀=Vehicle control tumor volume at start of dosing. Tumor growth regression is calculated using % Tumor Regression=−(1−(Te/T0))*100) where Te=Test tumor volume (TV) endpoint, Test T0=TV at start of dosing. The compounds of this disclosure are expected to inhibit tumor growth and induce tumor shrinkage over a range of doses.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula I: 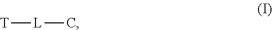
or a pharmaceutically acceptable salt, solvate, or stereoisomer thereof,
wherein:
C is of Formula I-1 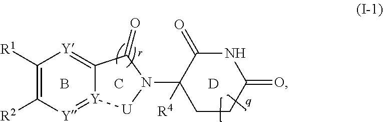
wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O) ₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R^{z} is *-Cy²-, wherein * denotes attachment to L;
-Cy²- is C₃₋₁₂ carbocyclylene or 3- to 12-membered heterocyclylene, wherein the carbocyclylene or heterocyclylene is optionally substituted with one or more R^{u}; or
R¹ and R², together with the intervening carbon atoms, form Ring A attached to L, wherein Ring A is optionally substituted C₃₋₁₂ carbocycle or 5- to 16-membered heterocycle;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form Ring A attached to L, wherein Ring A is optionally substituted 5- to 16-membered heterocycle;
provided that R¹ and R², and R² and R³, do not both form Ring A attached to L;
Y′ is N or CR^{Y}′;
R^{Y}′ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R″; [symbol]
denotes an optional covalent bond between Y and U;
when the bond between Y and U is absent:
r is 0 or 1;
Y is N or CR^{Y};
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R″;
U is hydrogen or C₁₋₆ alkyl optionally substituted with one or more R^{u};
when the bond between Y and U is present:
r is 1;
Y is C;
U is —CH₂—, —C(═O)—, —(C═O)—N(R^{U})—*, or —N═C(R^{U})—*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl; and
q is an integer from 0 to 2,
provided that when the bond between Y and U is present, and U is —CH₂— or —C(═O)—, then either R¹ and R², or R² and R³, form Ring A attached to L,
T is of Formula I-2: 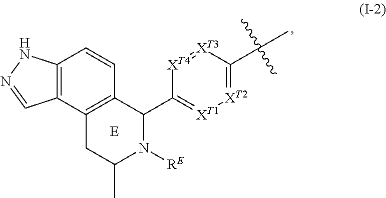
wherein:
each of X^{T1}, X^{T2}, X^{T3}, and X^{T4} is independently N or CR^{T};
each occurrence of R^{T} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
R^{E} is C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene-C₃₋₁₂ carbocyclyl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
L is of Formula I-3: 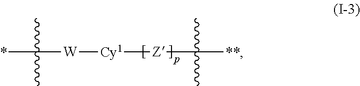
wherein:
* denotes attachment to T and ** denotes attachment to C;
W is absent; or
W is C₁₋₃ alkylene, —O—, —NR^{W}—, or —(C═O)—, wherein the alkylene is optionally substituted by one or more R^{u};
Cy¹ is absent; or
Cy¹ is 6-membered heteroarylene, C₆ arylene, C₃₋₁₂ membered carbocyclylene, or 3- to 12-membered heterocyclylene, wherein the arylenc, heteroarylenc, carbocyclylene, or heterocyclylene is optionally substituted by one or more R″;
Z′ is absent; or
each Z′ is independently C₁₋₃ alkylene, —O—, —NR^{W}—, —(C═O)—, C₃₋₁₂ membered carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted with one or more R^{u}; and
p is an integer selected from 0 to 8,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{e} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

2. The compound of claim 1, wherein
1) when the bond between Y and U is present, U is —CH₂— or —C(═O)—, and r is 1, then Ring A is not 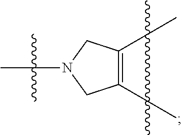
and
2) the compound is not

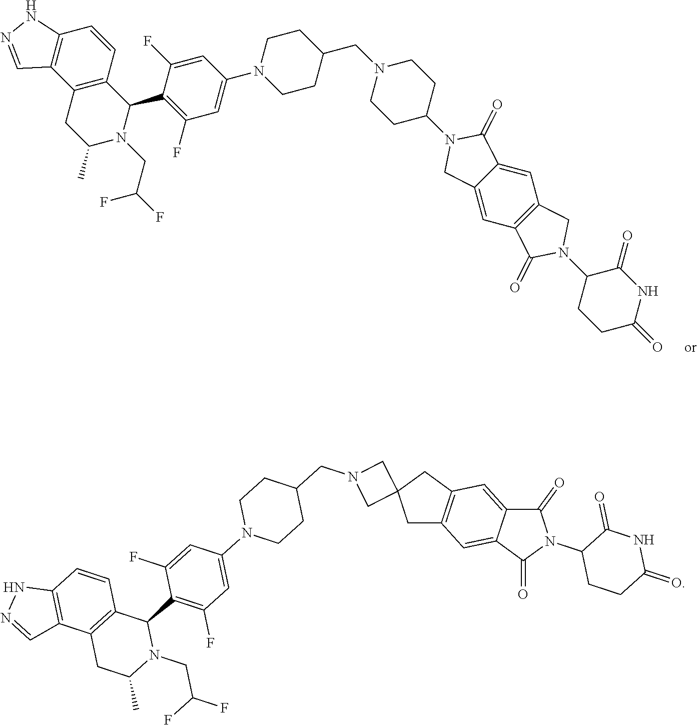 [SMILES: C[C@@H]1Cc2c(ccc3[nH]ncc23)[C@@H](c2c(F)cc(N3CCC(CN4CCC(N5Cc6cc7c(cc6C5=O)CN(C5CCC(=O)NC5=O)C7=O)CC4)CC3)cc2F)N1CC(F)F ; C[C@@H]1Cc2c(ccc3[nH]ncc23)[C@@H](c2c(F)cc(N3CCC(CN4CC5(Cc6cc7c(cc6C5)C(=O)N(C5CCC(=O)NC5=O)C7=O)C4)CC3)cc2F)N1CC(F)F]

3. The compound of claim 1 or 2, wherein when the bond between Y and U is present, U is —CH₂— or —C(═O)—, and r is 1, then Ring A is not

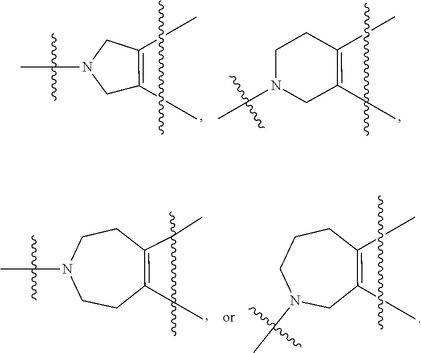

4. The compound of any one of claims 1-3, wherein C is of Formula I-1-i

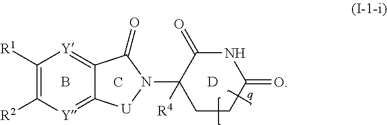

5. The compound of claim 4, wherein U is —CH₂— or —C(═O)—.

6. The compound of any one of claims 1-3, wherein C is of Formula I-1-ii

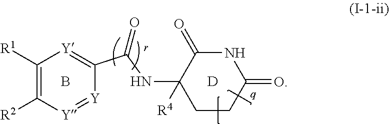

7. The compound of claim 6, wherein Y is N.

8. The compound of claim 6, wherein Y is CR^{Y}, and R^{Y} is hydrogen, halogen, or C₁₋₆ alkoxy, wherein the alkoxy is optionally substituted with one or more R^{u}.

9. The compound of any one of claims 1-8, wherein R¹ and R², together with the intervening carbon atoms, form Ring A attached to L, wherein the Ring A is optionally substituted 5- to 16-membered heterocycle.

10. The compound of claim 9, wherein Ring A is optionally substituted 7- to 16-membered fused heterocycle.

11. The compound of claim 10, wherein Ring A is 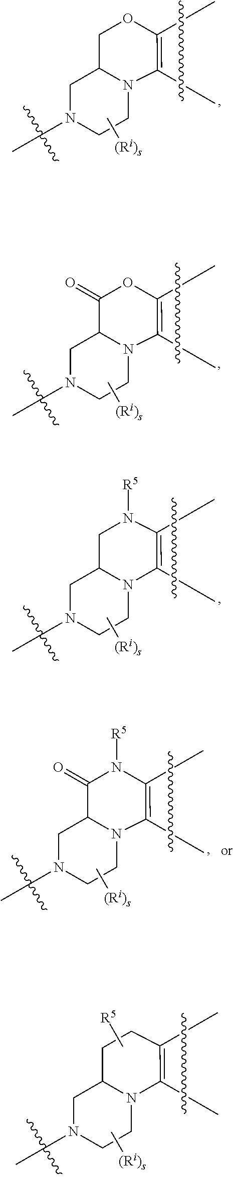
wherein:
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

12. The compound of claim 9, wherein Ring A is optionally substituted 7- to 16-membered spiro heterocycle.

13. The compound of claim 12, wherein Ring A is: 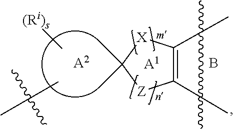
wherein:
Ring A² is C₃₋₈ carbocycle or 3- to 8-membered heterocycle;
each X is independently —C(R^{X1})₂—, —NR^{X2}—, —O—, —S—, —S(═O)—, or —S(═O)₂—;
each Z is independently —C(R^{Z1})₂—, —NR^{Z2}—, —O—, —S—, —S(═O)—, or —S(═O)₂—;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R^{Z2} is independently hydrogen or C₁₋₆ alkyl optionally substituted with one or more R^{u};
m′ and n′ are independently an integer selected from 0-3, wherein m′ and n′ are not both 0;
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkonyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits,
provided that when none of m′ and n′ is 0, then Ring A¹ is 4- to 9-membered heterocycle.

14. The compound of claim 13, wherein Ring A is:
1) 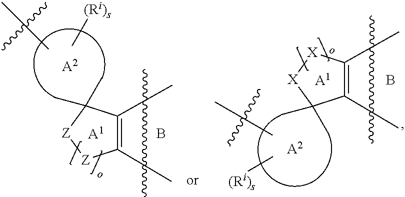
wherein o is 0 or 1; or
2)

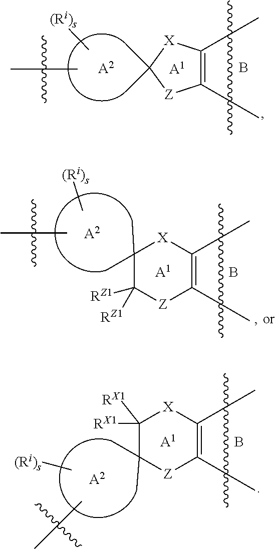

15. The compound of claim 9, wherein Ring A is optionally substituted 5- to 6-membered heterocycle.

16. The compound of claim 15, wherein Ring A is 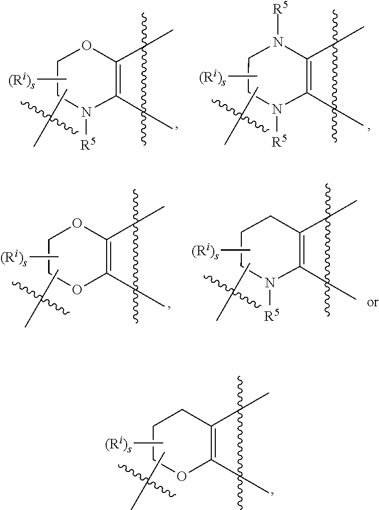
wherein:
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S (═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

17. The compound of any one of claims 9-16, wherein Y″ is N.

18. The compound of any one of claims 9-16, wherein Y″ is CR³, and R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

19. The compound of claim 18, wherein R³ is hydrogen, halogen, or C₁₋₆ alkoxy, wherein the alkoxy is optionally substituted with one or more R^{u}.

20. The compound of any one of claims 1-8, wherein R² and R³, together with the intervening carbon atoms, form Ring A attached to L, wherein the Ring A is optionally substituted 5- to 16-membered heterocycle.

21. The compound of claim 20, wherein Ring A is optionally substituted 7- to 16-membered fused heterocycle.

22. The compound of claim 21, wherein Ring A is 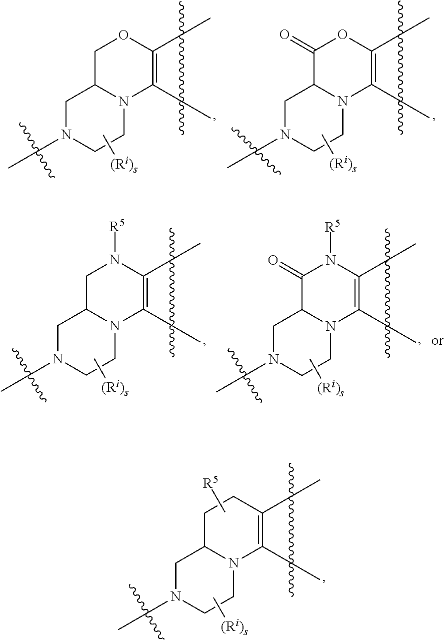
wherein:
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

23. The compound of claim 20, wherein Ring A is optionally substituted 7- to 16-membered spiro heterocycle.

24. The compound of claim 23, wherein Ring A is: 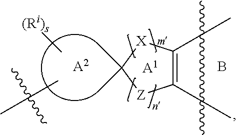
wherein:
Ring A² is C₃₋₈ carbocycle or 3- to 8-membered heterocycle;
each X is independently —C(R^{X1})₂—, —NR^{X2}, —O—, —S—, —S(═O)—, or —S(═O)₂—;
each Z is independently —C(R^{Z1})₂—, —NR^{Z2}—, —O—, —S—, —S(═O)—, or —S(═O)₂—;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₆ carbocyclyl, 3- to 6-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal RZI together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R^{Z2} is independently hydrogen or C₁₋₆ alkyl optionally substituted with one or more R^{u};
m′ and n′ are independently an integer selected from 0-3, wherein m′ and n′ are not both 0;
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits,
provided that when none of m′ and n′ is 0, then Ring A¹ is 4- to 9-membered heterocycle.

25. The compound of claim 24, wherein Ring A is:
1) 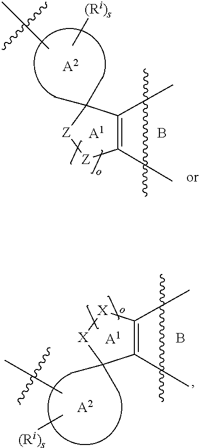
wherein o is 0 or 1; or
2)

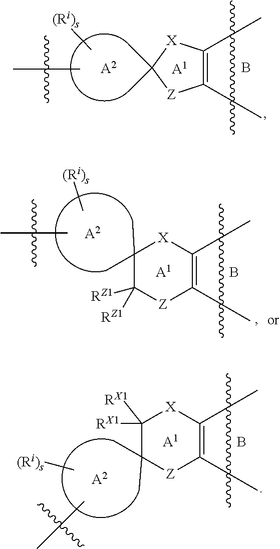

26. The compound of claim 20, wherein Ring A is optionally substituted 5- to 6-membered heterocycle.

27. The compound of claim 26, wherein Ring A is 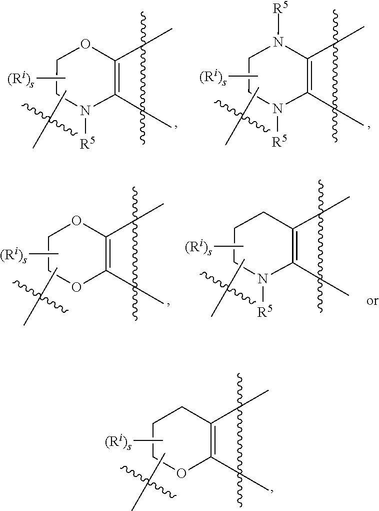
wherein:
R⁵ is hydrogen or C₁₋₆ alkyl optionally substituted with one or more R^{u};
each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S (═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

28. The compound of any one of claims 20-27, wherein R¹ is hydrogen, halogen, —CN, —NO₂, —OH, —NH₂, C₁₋₆ alkyl, C₁₋₆ alkoxy, C₁₋₆ alkylamino, C₂₋₆ alkonyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

29. The compound of claim 28, wherein R¹ is hydrogen, halogen, or C₁₋₆ alkoxy, wherein the alkoxy is optionally substituted with one or more R^{u}.

30. The compound of any one of claims 9-29, wherein each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

31. The compound of claim 30, wherein s is 0.

32. The compound of claim 1, wherein C is of Formula I-1-ii

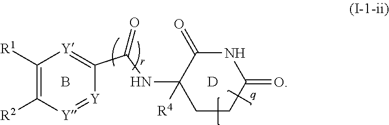

33. The compound of claim 32, wherein R² is *-Cy²-, wherein * denotes attachment to L.

34. The compound of claim 32 or 33, wherein -Cy²- is C₅₋₁₂ fused carbocyclylene or 5- to 12-membered fused heterocyclylene, wherein the carbocyclylene or heterocyclylene is optionally substituted with one or more R^{u}.

35. The compound of any one of claims 32-34, wherein R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

36. The compound of claim 35, wherein R¹ is hydrogen, halogen, or C₁₋₆ alkoxy, wherein the alkoxy is optionally substituted with one or more R^{u}.

37. The compound of any one of claims 32-36, wherein Y is N.

38. The compound of any one of claims 32-36, wherein Y is CR^{Y}, and R^{Y} is hydrogen, halogen, or C₁₋₆ alkoxy, wherein the alkoxy is optionally substituted with one or more R^{u}.

39. The compound of any one of claims 32-36, wherein Y″ is CR³, and R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

40. The compound of claim 39, wherein R³ is hydrogen, halogen, or C₁₋₆ alkoxy, wherein the alkoxy is optionally substituted with one or more R^{u}.

41. The compound of any one of claims 32-40, wherein r is 0.

42. The compound of any one of claims 32-40, wherein r is 1.

43. The compound of any one of claims 1-42, wherein R⁴ is hydrogen.

44. The compound of any one of claims 1-43, wherein q is 1.

45. The compound of any one of claims 1-44, wherein each of X^{T1}, X^{T2}, X^{T3}, and X^{T4} is CR^{T}.

46. The compound of claim 45, wherein X^{T1} and X^{T4} are CF, and X^{T2} and X^{T3} are CH; or one of X^{T1} and X^{T4} is CF or C(OCH₃), the other one of X^{T1} and X^{T4} is CH, and each of X^{T2} and X^{T3} is CH.

47. The compound of any one of claims 1-44, wherein one of X^{T1}, X^{T2}, X^{T3}, and X^{T4} is N.

48. The compound of claim 46, wherein one of X^{T1} and X^{T4} is N, the other one of X^{T1} and X^{T4} is CH, and each of X^{T2} and X^{T3} is CH; or one of X^{T2} and X^{T3} is N, the other one of X^{T2} and X^{T3} is CH, and each of X^{T1} and X^{T4} is CH.

49. The compound of any one of claims 1-44, wherein two of X^{T1}, X^{T2}, X^{T3}, and X^{T4} are N.

50. The compound of claim 49, wherein each of X^{T1} and X^{T4} is CH, and each of X^{T2} and X^{T3} is N.

51. The compound of any one of claims 45-50, wherein each R^{T} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

52. The compound of claim 51, wherein each R^{T} is independently hydrogen, C₁₋₆ alkoxy, or halogen.

53. The compound of any one of claims 45-52, wherein R^{E} is C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, or —(C₁₋₆ alkylene-C₃₋₁₂ carbocyclyl), wherein the alkyl, alkenyl, alkynyl, aryl, heteroaryl, carbocyclyl, or heterocyclyl is optionally substituted with one or more R^{u}.

54. The compound of claim 53, wherein R^{E} is C₁₋₆ alkyl or —(C₁₋₆ alkylene-C₃₋₁₂ carbocyclyl), wherein the alkyl or carbocyclyl is optionally substituted with one or more R^{u}.

55. The compound of claim 54, wherein R^{E} is

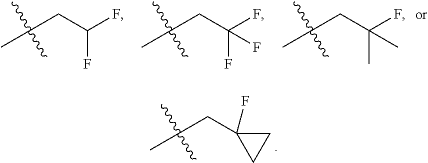

56. The compound of any one of claims 1-55, wherein Cy¹ is C₃₋₁₂ carbocyclylene or 3- to 12-membered heterocyclylene, wherein the carbocyclylene or heterocyclylene is optionally substituted by one or more R^{u}.

57. The compound of any one of claims 1-55, wherein Cyl is 3- to 12-membered heterocyclylene, wherein the heterocyclylene is optionally substituted by one or more R^{u}.

58. The compound of claim 57, wherein Cy¹ is 3- to 12-membered heterocyclylene is selected from morpholinylene, piperidinylene, piperazinylene, 7-azaspiro[3.5]nonanylene, 2,7-diazaspiro[3.5]nonanylene, 2-azaspiro[3.5]nonanylene, 2,7-diazaspiro[3.5]nonanylene, 1-oxa-8-azaspiro[4.5]decenylene, 2-oxa-8-azaspiro[4.5]decenylene, 5-oxa-2-azaspiro[3.4]octanylene, 6-oxa-2-azaspiro[3.4]octanylene, 3,9-diazaspiro[5.5]undecanylene, 5-oxa-2-azaspiro[3.5]nonanylene, 1-oxa-9-azaspiro[5.5]undecanylene, 1-oxa-4,9-diazaspiro[5.5]undecanylene, 2,6-diazaspiro[3.3]heptanylene, 2-azaspiro[3.3]heptanylene, 1,5-dioxa-9-azaspiro[5.5]undecanylene, 1,4-dioxa-9-azaspiro[5.5]undecanylene, 5,9-dioxa-2-azaspiro[3.5]nonanylene, 5,8-dioxa-2-azaspiro[3.5]nonanylene, 6-oxa-2-azaspiro[3.5]nonanylene, 1-oxa-7-azaspiro[3.5]nonanylene, 5-oxa-2-azaspiro[3.6]decenylene, 5-oxa-2-azaspiro[3.6]decenylene, 5,9-dioxa-2-azaspiro[3.6]decenylene, 5,8-dioxa-2-azaspiro[3.6]decenylene, and 6,9-dioxa-2-azaspiro[3.6]decenylene, wherein the heterocyclylene is optionally substituted by one or more R^{u}.

59. The compound of claim 57, wherein Cy¹ is 3- to 12-membered heterocyclylene is selected from: 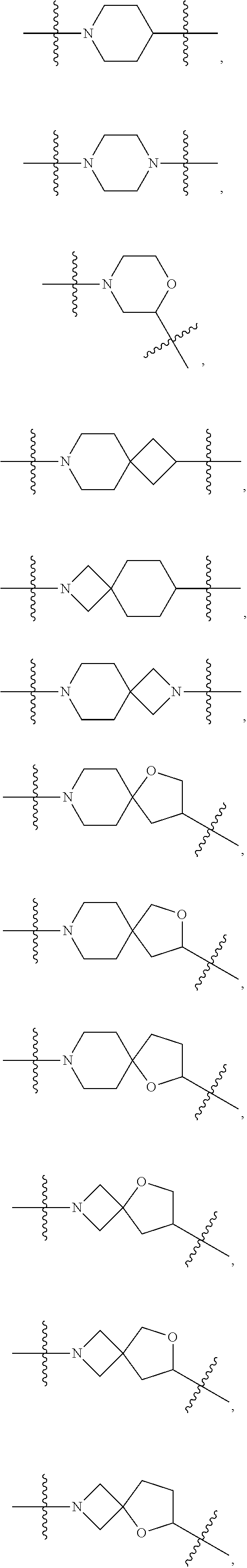 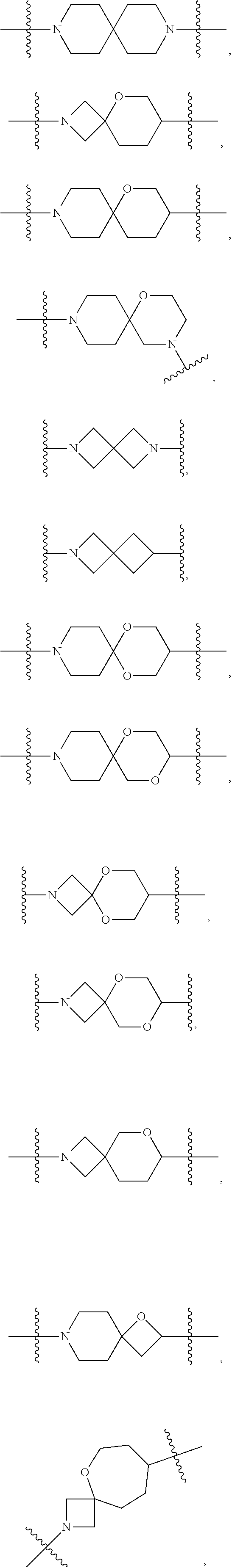 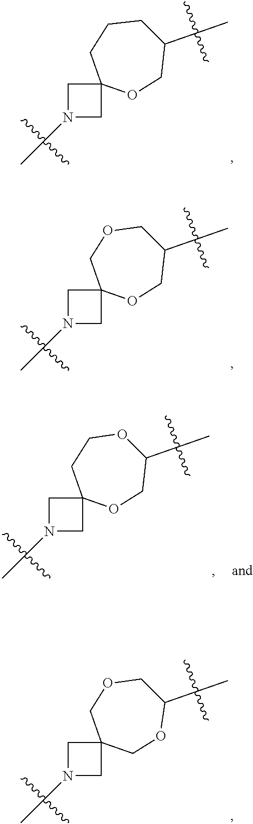 [SMILES: *C1CCCC2(CN(*)C2)OC1 ; *C1COCC2(CN(*)C2)OC1 ; *C1COC2(CCO1)CN(*)C2 ; *C1COCC2(CO1)CN(*)C2]
wherein the heterocyclylene is optionally substituted by one or more R^{u}.

60. The compound of any one of claims 56-59, wherein W is absent.

61. The compound of any one of claims 56-60, wherein Z′ is absent.

62. The compound of any one of claims 56-60, wherein Z′ is —C(═O)—, C₁₋₆ alkylene, *—O—(C₁₋₆ alkylene)-, *—(C₁₋₆ alkylene)-O—, *—C(═O)—(C₁₋₆ alkylene)-, *—(C₁₋₆ alkylene)-C(═O)—, 3- to 12-membered heterocyclylene, *—C(═O)-(3- to 12-membered heterocyclylene)-, *-(3- to 12-membered heterocyclylene)-C(═O)—, *-(3- to 12-membered heterocyclylene)-(C₁₋₆ alkylene)-, *—(C₁₋₆ alkylene)-(3- to 12-membered heterocyclylene)-, *—(C₁₋₆ alkylene)-(3- to 12-membered heterocyclylene)-(C₁₋₆ alkylene)-, *—(C₁₋₆ alkylene)-(3- to 12-membered heterocyclylene)-(C(═O))—, *—(C(═O))-(3- to 12-membered heterocyclylene)-(C₁₋₆ alkylene)-, *-(3- to 12-membered heterocyclylene)-(C₁₋₆ alkylene)-(C(═O))—, *—(C(═O))—(C₁₋₆ alkylene)-(3- to 12-membered heterocyclylene)-, *—(C₁₋₆ alkylene)-(C(═O))-(3- to 12-membered heterocyclylene)-, or *-(3- to 12-membered heterocyclylene)-(C(═O))—(C₁₋₆ alkylene)-, wherein the alkylene or heterocyclylene is optionally substituted by one or more R^{u}, and *denotes attachment to C.

63. The compound of claim 62, wherein Z′ is C₁₋₆ alkylene, *—C(═O)—(C₁₋₆ alkylene)-, *—(C₁₋₆ alkylene)-C(═O)—, 3- to 12-membered heterocyclylene, or *-(3- to 12-membered heterocyclylene)-(C₁₋₆ alkylene)-, wherein the alkylene or heterocyclylene is optionally substituted by one or more R^{u}, and *denotes attachment to C.

64. A compound selected from the compounds in Tables 1 and 2 or a pharmaceutically acceptable salt thereof.

65. A compound selected from the compounds in the following table

| | |
|---|---|
| 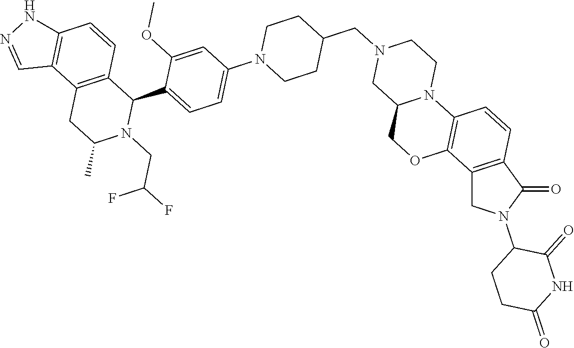 | 3-((R)-7-((1-(4-((6S,8R)-7-(2,2- difluoroethyl)-8-methyl-6,7,8,9- tetrahydro-3H-pyrazolo[[1.1]4,3- f]isoquinolin-6-yl)-3- methoxyphenyl)piperidin-4- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| 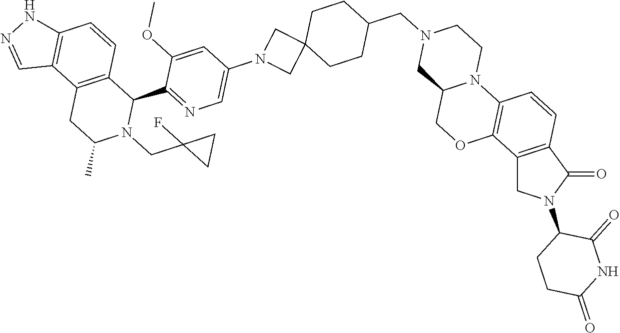 | (R)-3-((R)-7-((2-(6-((6S,8R)-7-((1- fluorocyclopropyl)methyl)-8-methyl- 6,7,8,9-tetrahydro-3H-pyrazolo[4,3- f]isoquinolin-6-yl)pyridin-3-yl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 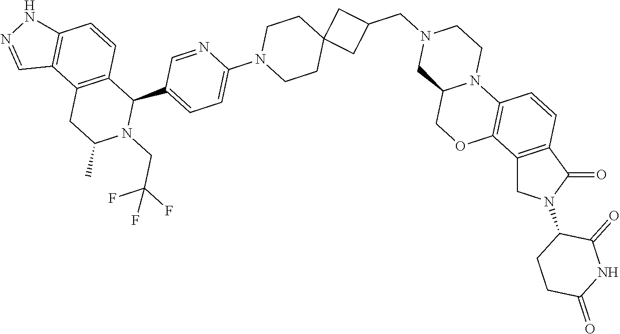 | (S)-3-((R)-7-((7-(5-((6S,8R)-8- methyl-7-(2,2,2-trifluoroethyl)- 6,7,8,9-tetrahydro-3H-pyrazolo[4,3- f]isoquinolin-6-yl)pyridin-2-yl)-7- azaspiro[3.5]nonan-2-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 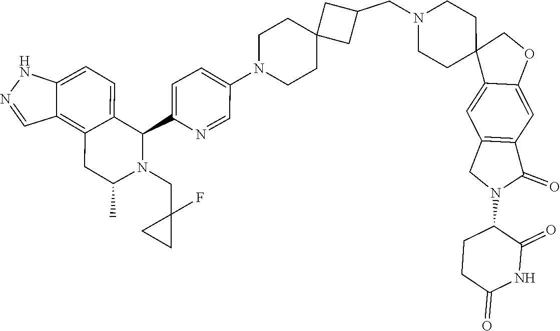 | (S)-3-(1′-((7-(6-((6S,8R)-7-((1- fluorocyclopropyl)methyl)-8-methyl- 6,7,8,9-tetrahydro-3H-pyrazolo[4,3- f]isoquinolin-6-yl)pyridin-2-yl)-7- azaspiro[3.5]nonan-2-yl)methyl)-7- oxo-5,7-dihydro-2H,6H- spiro[furo[2,3-f]isoindole-3,4′- piperidin]-6-yl)piperidine-2,6-dione |
| 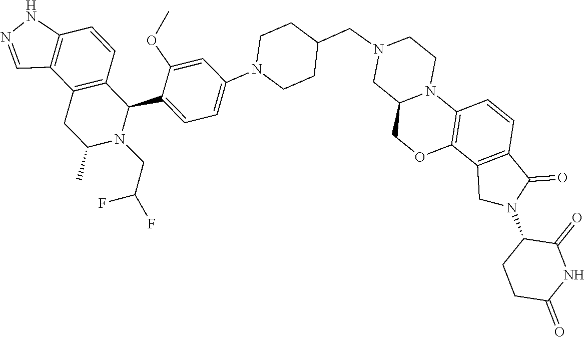 | (S)-3-((R)-7-((1-(4-((6S,8R)-7-(2,2- diflouoroethyl)-8-methyl-6,7,8,9- tetrahydro-3H-pyrazolo[4,3- f]isoquinolin-6-yl)-3- methoxyphenyl)piperidin-4- yl)methyl)-1-oxo-1,3,5,5a,6,7,8,9- octahydro-2H- pyrazino[1′,2′:4,5][1,4]oxazino[2,3- e]isindol-2-yl)piperidine-2,6-dione |
| 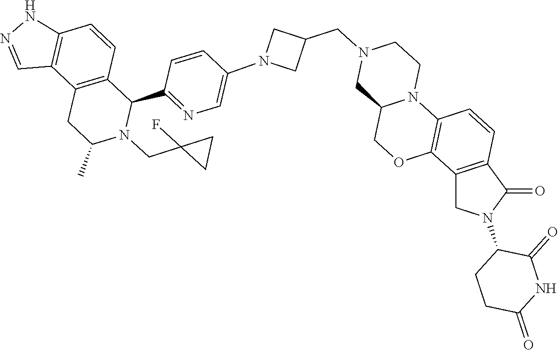 | (S)-3-((R)-7-((7-(6-((6S,8R)-7-((1- fluorocyclopropyl)methyl)-8-methyl- 6,7,8,9-tetrahydro-3H-pyrazolo[4,3- f]isoquinolin-6-yl)pyridin-3-yl)-7- azaspiro[3.5]nonan-2-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 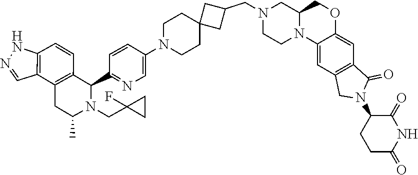 | (R)-3-((S)-3-((7-(6-((6S,8R)-7-((1- fluorocyclopropyl)methyl)-8-methyl- 6,7,8,9-tetrahydro-3H-pyrazolo[4,3- f]isoquinolin-6-yl)pyridin-3-yl)-7- azaspiro[3.5]nonan-2-yl)methyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| 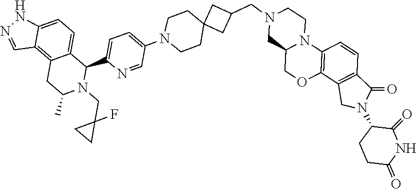 | (S)-3-((R)-7-((7-(6-((6S,8R)-7-((1- fluorocyclopropyl)methyl)-8-methyl- 6,7,8,9-tetrahydro-3H-pyrazolo[4,3- f]isoquinolin-6-yl)pyridin-3-yl)-7- azaspiro[3.5]nonan-2-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 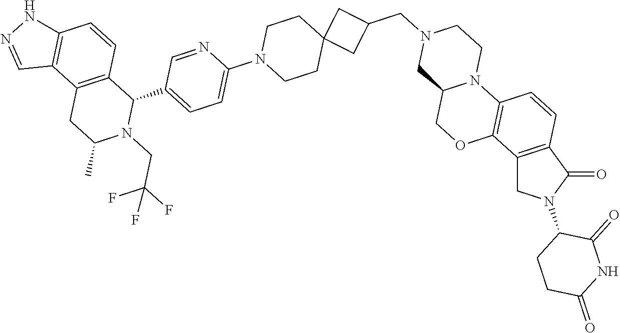 | (S)-3-((R)-7-((7-(5-((6R,8R)-8- methyl-7-(2,2,2-trifluoroethyl)- 6,7,8,9-tetrahydro-3H-pyrazolo[4,3- f]isoquinolin-6-yl)pyridin-2-yl)-7- azaspiro[3.5]nonan-2-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 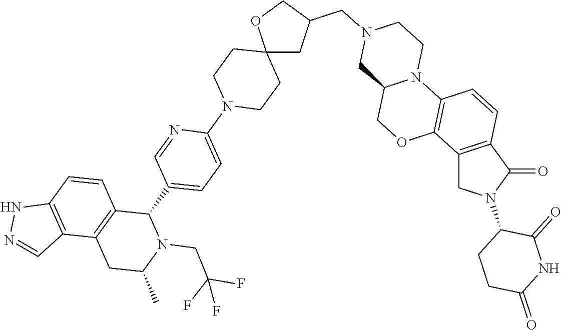 | (3S)-3-((5aR)-7-((8-(5-((6R,8R)-8- methyl-7-(2,2,2-trifluoroethyl)- 6,7,8,9-tetrahydro-3H-pyrazolo[4,3- f]isoquinolin-6-yl)pyridin-2-yl)-1- oxa-8-azaspiro[4.5]decan-3- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| 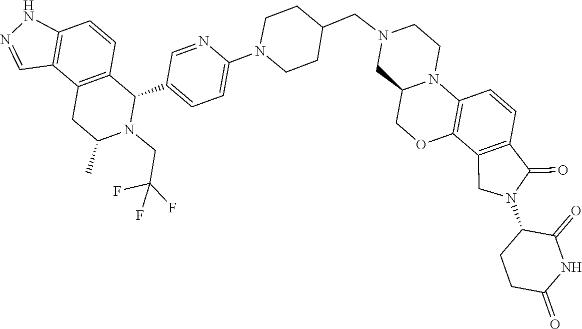 | (S)-3-((R)-7-((1-(5-((6R,8R)-8- methyl-7-(2,2,2-trifluoroethyl)- 6,7,8,9-tetrahydro-3H-pyrazolo[4,3- f]isoquinolin-6-yl)pyridin-2- yl)piperidin-4-yl)methyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| 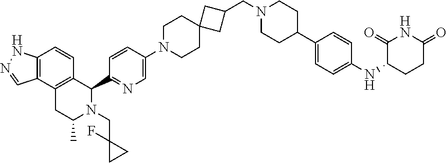 | (S)-3-((4-(1-((7-(6-((6S,8R)-7-((1- fluorocyclopropyl)methyl)-8-methyl- 6,7,8,9-tetrahydro-3H-pyrazolo[4,3- f]isoquinolin-6-yl)pyridin-3-yl)-7- azaspiro[3.5]nonan-2- yl)methyl)piperidin-4- yl)phenyl)amino)piperidine-2,6-dione |
| 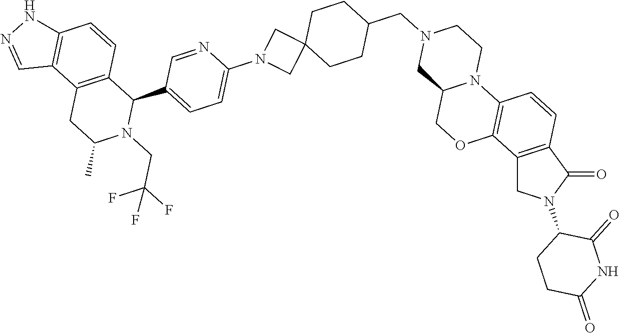 | (S)-3-((R)-7-((2-(5-((6S,8R)-8- methyl-7-(2,2,2-trifluoroethyl)- 6,7,8,9-tetrahydro-3H-pyrazolo[4,3- f]isoquinolin-6-yl)pyridin-2-yl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 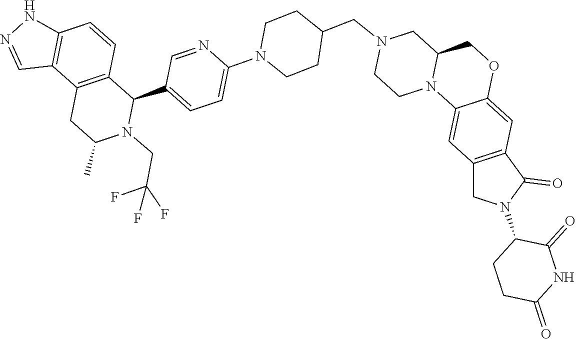 | (S)-3-((S)-3-((1-(5-((6S,8R)-8- methyl-7-(2,2,2-trifluoroethyl)- 6,7,8,9-tetrahydro-3H-pyrazolo[4,3- f]isoquinolin-6-yl)pyridin-2- yl)piperidin-4-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| 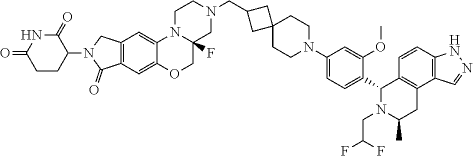 | 3-((R)-3-((7-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-7- azaspiro[3.5]nonan-2-yl)methyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| 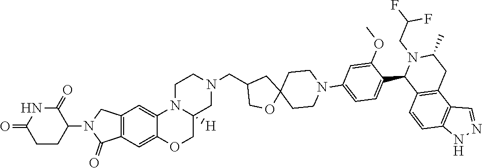 | 3-((4aS)-3-((8-(4-((6S,8R)-7-(2,2- difluoroethyl)-8-methyl-6,7,8,9- tetrahydro-3H-pyrazolo[4,3- f]isoquinolin-6-yl)-3- methoxyphenyl)-1-oxa-8- azaspiro[4.5]decan-3-yl)methyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| 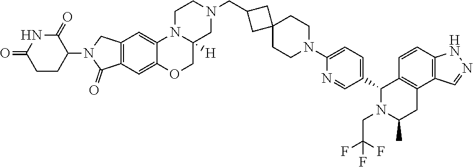 | 3-((S)-3-((7-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)-7- azaspiro[3.5]nonan-2-yl)methyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| 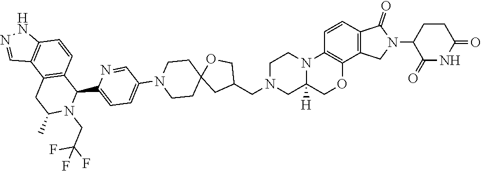 | 3-((5aR)-7-((8-(6-((6S,8R)-8-methyl- 7-(2,2,2-trifluoroethyl)-6,7,8,9- tetrahydro-3H-pyrazolo[4,3- f]isoquinolin-6-yl)pyridin-3-yl)-1- oxa-8-azaspiro[4.5]decan-3- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| 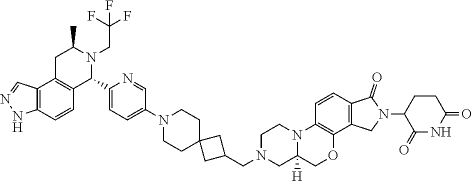 | 3-((R)-7-((7-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)-7- azaspiro[3.5]nonan-2-yl)mcthyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 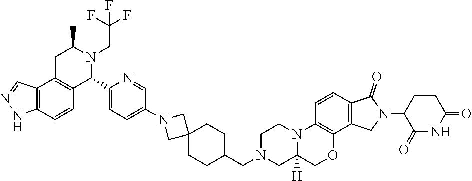 | 3-((R)-7-((2-(6-((6S,8R)-8-methyl-7- (2,2,2-trifluoroethyl)-6,7,8,9- tetrahydro-3H-pyrazolo[4,3- f]isoquinolin-6-yl)pyridin-3-yl)-2- azaspiro[3.5]nonan-7-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 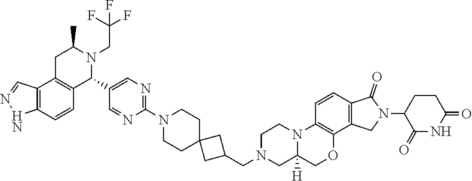 | 3-((R)-7-((7-(5-((6S,8R)-8-methyl-7- (2,2,2-trifluoroethyl)-6,7,8,9- tetrahydro-3H-pyrazolo[4,3- f]isoquinolin-6-yl)pyrimidin-2-yl)-7- azaspiro[3.5]nonan-2-yl)methyl)-1- oxo-1,3,5,5a,6,7,8,9-octahydro-2H- pyrazino[1′,2′:4,5][1,4]oxazino[2,3- e]isoindol-2-yl)piperidine-2,6-dione |
| 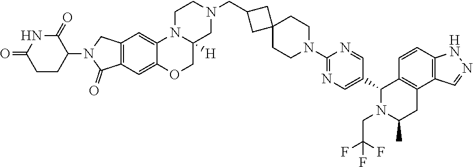 | 3-((S)-3-((7-(5-((6S,8R)-8-methyl-7- (2,2,2-trifluoroethyl)-6,7,8,9- tetrahydro-3H-pyrazolo[4,3- f]isoquinolin-6-yl)pyrimidin-2-yl)-7- azaspiro[3.5]nonan-2-yl)methyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| 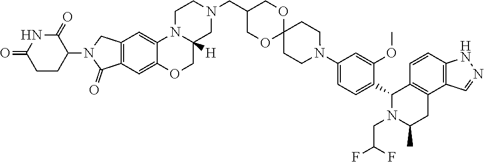 | 3-((R)-3-((9-(4-((6S,8R)-7-(2,2- difluorocthyl)-8-mcthyl-6,7,8,9- tetrahydro-3H-pyrazolo[4,3- f]isoquinolin-6-yl)-3- methoxyphenyl)-1,5-dioxa-9- azaspiro[5.5]undecan-3-yl)methyl)-8- oxo-1,2,3,4,4a,5,8,10-octahydro-9H- pyrazino[1′,2′:4,5][1,4]oxazino[2,3- f]isoindol-9-yl)piperidine-2,6-dione |
| 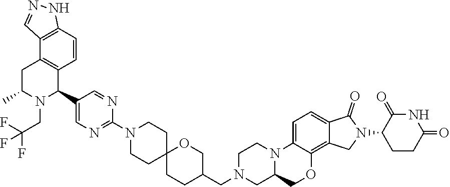 | (3S)-3-((5aR)-7-((9-(5-((6S,8R)-8- methyl-7-(2,2,2-trifluoroethyl)- 6,7,8,9-tetrahydro-3H-pyrazolo[4,3- f]isoquinolin-6-yl)pyrimidin-2-yl)-1- oxa-9-azaspiro[5.5]undccan-3- yl)methyl)-1-oxo-1,3,5,5a,6,7,8,9- octahydro-2H- pyrazino[1′,2′:4,5][1,4]oxazino[2,3- e]isoindol-2-yl)piperidine-2,6-dione |
| 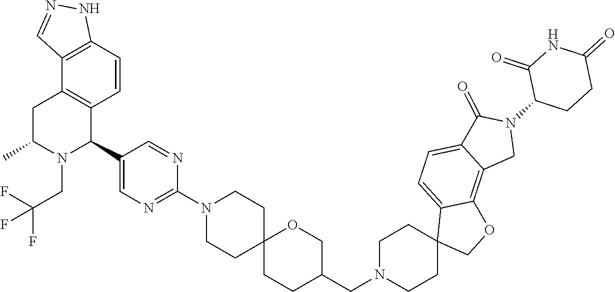 | (3S)-3-(1′-((9-(5-((6S,8R)-8-methyl- 7-(2,2,2-trifluoroethyl)-6,7,8,9- tetrahydro-3H-pyrazolo[4,3- f]isoquinolin-6-yl)pyrimidin-2-yl)-1- oxa-9-azaspiro[5.5]undecan-3- yl)methyl)-6-oxo-6,8-dihydro- 2H,7H-spiro[furo[2,3-e]isoindole- 3,4′-pipcridin]-7-yl)pipcridinc-2,6- dione |
| 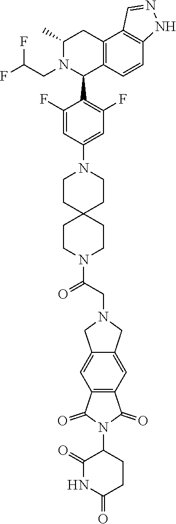 | 6-(2-(9-(4-((6S,8R)-7-(2,2- difluoroethyl)-8-methyl-6,7,8,9- tetrahydro-3H-pyrazolo[4,3- f]isoquinolin-6-yl)-3,5- difluorophenyl)-3,9- diazaspiro[5.5]undecan-3-yl)-2- oxoethyl)-2-(2,6-dioxopiperidin-3- yl)-6,7-dihydropyrrolo[3,4- f]isoindole-1,3(2H,5H)-dione |
| 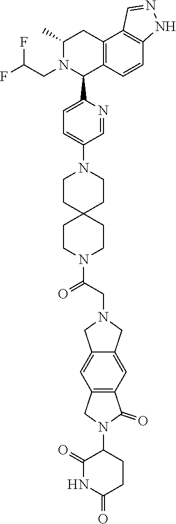 | 6-(2-(9-(6-((6S,8R)-7-(2,2- difluoroethyl)-8-methyl-6,7,8,9- tetrahydro-3H-pyrazolo[4,3- f]isoquinolin-6-yl)pyridin-3-yl)-3,9- diazaspiro[5.5]undecan-3-yl)-2- oxoethyl)-2-(2,6-dioxopiperidin-3- yl)-6,7-dihydropyrrolo[3,4- f]isoindole-1,3(2H,5H)-dione |

66. A pharmaceutical composition comprising the compound of any one of claims 1-65, and a pharmaceutically acceptable excipient.

67. A method of degrading an estrogen receptor protein in a patient or biological sample comprising contacting said patient or biological sample with a compound of any one of claims 1-65.

68. Use of a compound of any one of claims 1-65 in the manufacture of a medicament for degrading an estrogen receptor protein in a patient or biological sample.

69. A compound of any one of claims 1-65 for use in degrading an estrogen receptor protein in a patient or biological sample.

70. A method of treating a disease or disorder comprising administering to a patient in need thereof a compound of any one of claims 1-65.

71. Use of a compound of any one of claims 1-65 in the manufacture of a medicament for treating a disease or disorder.

72. A compound of any one of claims 1-65 for use in treating a disease or disorder.

73. The method, use, or compound for use of any one of claims 70-72, wherein the disease or disorder is an estrogen receptor-mediated disease or disorder.

74. The method, use, or compound for use of any one of claims 70-72, wherein the disease or disorder is breast cancer, lung cancer, ovarian cancer, endometrial cancer, prostate cancer, or esophageal cancer.
